SEQUENCE LISTING (151 sequences)

SEQ ID NO: 1 - protein, 460 aa, Stevia rebaudiana
MAEQQKIKKSPHVLLIPFPLQGHINPFIQFGKRLISKGVKTTLVTTIHTLNSTLNHSNTTTTSIEIQAISDGCDEGGFMSAGESYLETFKQVGSKSLADLIKKLQSEGTTIDAIIYDSMTEWVLDVAIEFGIDGGSFFTQACVVNSLYYHVHKGLISLPLGETVSVPGFPVLQRWETPLILQNHEQIQSPWSQMLFGQFANIDQARWVFTNSFYKLEEEVIEWTRKIWNLKVIGPTLPSMYLDKRLDDDKDNGFNLYKANHHECMNWLDDKPKESVVYVAFGSLVKHGPEQVEEITRALIDSDVNFLWVIKHKEEGKLPENLSEVIKTGKGLIVAWCKQLDVLAHESVGCFVTHCGFNSTLEAISLGVPVVAMPQFSDQTTNAKLLDEILGVGVRVKADENGIVRRGNLASCIKMIMEEERGVIIRKNAVKWKDLAKVAVHEGGSSDNDIVEFVSELIKA

SEQ ID NO: 2 - DNA, 1383 bp, Stevia rebaudiana
  source 1..1383: mol_type = genomic DNA
atggcagagcaacaaaagatcaaaaagtcacctcacgtcttacttattccatttcctctgcaaggacatatcaacccattcatacaatttgggaaaagattgattagtaagggtgtaaagacaacactggtaaccactatccacactttgaattctactctgaaccactcaaatactactactacaagtatagaaattcaagctatatcagacggatgcgatgagggtggctttatgtctgccggtgaatcttacttggaaacattcaagcaagtgggatccaagtctctggccgatctaatcaaaaagttacagagtgaaggcaccacaattgacgccataatctacgattctatgacagagtgggttttagacgttgctatcgaatttggtattgatggaggttcctttttcacacaagcatgtgttgtgaattctctatactaccatgtgcataaagggttaatctctttaccattgggtgaaactgtttcagttccaggttttccagtgttacaacgttgggaaaccccattgatcttacaaaatcatgaacaaatacaatcaccttggtcccagatgttgtttggtcaattcgctaacatcgatcaagcaagatgggtctttactaattcattctataagttagaggaagaggtaattgaatggactaggaagatctggaatttgaaagtcattggtccaacattgccatcaatgtatttggacaaaagacttgatgatgataaagataatggtttcaatttgtacaaggctaatcatcacgaatgtatgaattggctggatgacaaaccaaaggaatcagttgtatatgttgctttcggctctcttgttaaacatggtccagaacaagttgaggagattacaagagcacttatagactctgacgtaaactttttgtgggtcattaagcacaaagaggaggggaaactgccagaaaacctttctgaagtgataaagaccggaaaaggtctaatcgttgcttggtgtaaacaattggatgttttagctcatgaatctgtaggctgttttgtaacacattgcggattcaactctacactagaagccatttccttaggcgtacctgtcgttgcaatgcctcagttctccgatcagacaaccaacgctaaacttttggacgaaatactaggggtgggtgtcagagttaaagcagacgagaatggtatcgtcagaagagggaacctagcttcatgtatcaaaatgatcatggaagaggaaagaggagttatcataaggaaaaacgcagttaagtggaaggatcttgcaaaggttgccgtccatgaaggcggctcttcagataatgatattgttgaatttgtgtccgaactaatcaaagcctaa

SEQ ID NO: 3 - protein, 481 aa, Stevia rebaudiana
MDAMATTEKKPHVIFIPFPAQSHIKAMLKLAQLLHHKGLQITFVNTDFIHNQFLESSGPHCLDGAPGFRFETIPDGVSHSPEASIPIRESLLRSIETNFLDRFIDLVTKLPDPPTCIISDGFLSVFTIDAAKKLGIPVMMYWTLAACGFMGFYHIHSLIEKGFAPLKDASYLTNGYLDTVIDWVPGMEGIRLKDFPLDWSTDLNDKVLMFTTEAPQRSHKVSHHIFHTFDELEPSIIKTLSLRYNHIYTIGPLQLLLDQIPEEKKQTGITSLHGYSLVKEEPECFQWLQSKEPNSVVYVNFGSTTVMSLEDMTEFGWGLANSNHYFLWIIRSNLVIGENAVLPPELEEHIKKRGFIASWCSQEKVLKHPSVGGFLTHCGWGSTIESLSAGVPMICWPYSWDQLTNCRYICKEWEVGLEMGTKVKRDEVKRLVQELMGEGGHKMRNKAKDWKEKARIAIAPNGSSSLNIDKMVKEITVLARN

SEQ ID NO: 4 - DNA, 1446 bp, Stevia rebaudiana
  source 1..1446: mol_type = genomic DNA
atggatgcaatggcaactactgagaaaaagcctcatgtgatcttcattccatttcctgcacaatctcacataaaggcaatgctaaagttagcacaactattacaccataagggattacagataactttcgtgaataccgacttcatccataatcaatttctggaatctagtggccctcattgtttggacggagccccagggtttagattcgaaacaattcctgacggtgtttcacattccccagaggcctccatcccaataagagagagtttactgaggtcaatagaaaccaactttttggatcgtttcattgacttggtcacaaaacttccagacccaccaacttgcataatctctgatggctttctgtcagtgtttactatcgacgctgccaaaaagttgggtatcccagttatgatgtactggactcttgctgcatgcggtttcatgggtttctatcacatccattctcttatcgaaaagggttttgctccactgaaagatgcatcatacttaaccaacggctacctggatactgttattgactgggtaccaggtatggaaggtataagacttaaagattttcctttggattggtctacagaccttaatgataaagtattgatgtttactacagaagctccacaaagatctcataaggtttcacatcatatctttcacacctttgatgaattggaaccatcaatcatcaaaaccttgtctctaagatacaatcatatctacactattggtccattacaattacttctagatcaaattcctgaagagaaaaagcaaactggtattacatccttacacggctactctttagtgaaagaggaaccagaatgttttcaatggctacaaagtaaagagcctaattctgtggtctacgtcaacttcggaagtacaacagtcatgtccttggaagatatgactgaatttggttggggccttgctaattcaaatcattactttctatggattatcaggtccaatttggtaataggggaaaacgccgtattacctccagaattggaggaacacatcaaaaagagaggtttcattgcttcctggtgttctcaggaaaaggtattgaaacatccttctgttggtggtttccttactcattgcggttggggctctacaatcgaatcactaagtgcaggagttccaatgatttgttggccatattcatgggaccaacttacaaattgtaggtatatctgtaaagagtgggaagttggattagaaatgggaacaaaggttaaacgtgatgaagtgaaaagattggttcaggagttgatgggggaaggtggccacaagatgagaaacaaggccaaagattggaaggaaaaagccagaattgctattgctcctaacgggtcatcctctctaaacattgataagatggtcaaagagattacagtcttagccagaaactaa

SEQ ID NO: 5 - protein, 473 aa, Stevia rebaudiana
MATSDSIVDDRKQLHVATFPWLAFGHILPYLQLSKLIAEKGHKVSFLSTTRNIQRLSSHISPLINVVQLTLPRVQELPEDAEATTDVHPEDIPYLKKASDGLQPEVTRFLEQHSPDWIIYDYTHYWLPSIAASLGISRAHFSVTTPWAIAYMGPSADAMINGSDGRTTVEDLTTPPKWFPFPTKVCWRKHDLARLVPYKAPGISDGYRMGLVLKGSDCLLSKCYHEFGTQWLPLLETLHQVPVVPVGLLPPEIPGDEKDETWVSIKKWLDGKQKGSVVYVALGSEVLVSQTEVVELALGLELSGLPFVWAYRKPKGPAKSDSVELPDGFVERTRDRGLVWTSWAPQLRILSHESVCGFLTHCGSGSIVEGLMFGHPLIMLPIFGDQPLNARLLEDKQVGIEIPRNEEDGCLTKESVARSLRSVVVEKEGEIYKANARELSKIYNDTKVEKEYVSQFVDYLEKNARAVAIDHES

SEQ ID NO: 6 - DNA, 1422 bp, Stevia rebaudiana
  source 1..1422: mol_type = genomic DNA
atggctacatctgattctattgttgatgacaggaagcagttgcatgtggctactttcccttggcttgctttcggtcatatactgccttacctacaactatcaaaactgatagctgaaaaaggacataaagtgtcattcctttcaacaactagaaacattcaaagattatcttcccacatatcaccattgattaacgtcgttcaattgacacttccaagagtacaggaattaccagaagatgctgaagctacaacagatgtgcatcctgaagatatcccttacttgaaaaaggcatccgatggattacagcctgaggtcactagattccttgagcaacacagtccagattggatcatatacgactacactcactattggttgccttcaattgcagcatcactaggcatttctagggcacatttcagtgtaaccacaccttgggccattgcttacatgggtccatccgctgatgctatgattaacggcagtgatggtagaactaccgttgaagatttgacaaccccaccaaagtggtttccatttccaactaaagtctgttggagaaaacacgacttagcaagactggttccatacaaggcaccaggaatctcagacggctatagaatgggtttagtccttaaagggtctgactgcctattgtctaagtgttaccatgagtttgggacacaatggctaccacttttggaaacattacaccaagttcctgtcgtaccagttggtctattacctccagaaatccctggtgatgagaaggacgagacttgggtttcaatcaaaaagtggttagacgggaagcaaaaaggctcagtggtatatgtggcactgggttccgaagttttagtatctcaaacagaagttgtggaacttgccttaggtttggaactatctggattgccatttgtctgggcctacagaaaaccaaaaggccctgcaaagtccgattcagttgaattgccagacggctttgtcgagagaactagagatagagggttggtatggacttcatgggctccacaattgagaatcctgagtcacgaatctgtgtgcggtttcctaacacattgtggttctggttctatagttgaaggactgatgtttggtcatccacttatcatgttgccaatctttggtgaccagcctttgaatgcacgtctgttagaagataaacaagttggaattgaaatcccacgtaatgaggaagatggatgtttaaccaaggagtctgtggccagatcattacgttccgttgtcgttgaaaaggaaggcgaaatctacaaggccaatgcccgtgaactttcaaagatctacaatgacacaaaagtagagaaggaatatgtttctcaatttgtagattacctagagaaaaacgctagagccgtagctattgatcatgaatcctaa

SEQ ID NO: 7 - protein, 458 aa, Stevia rebaudiana
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 8 - DNA, 1377 bp, Stevia rebaudiana
  source 1..1377: mol_type = genomic DNA
atggaaaacaagaccgaaacaacagttagacgtaggcgtagaatcattctgtttccagtaccttttcaagggcacatcaatccaatactacaactagccaacgttttgtactctaaaggtttttctattacaatctttcacaccaatttcaacaaaccaaaaacatccaattacccacatttcacattcagattcatacttgataatgatccacaagatgaacgtatttcaaacttacctacccacggtcctttagctggaatgagaattccaatcatcaatgaacatggtgccgatgagcttagaagagaattagagttacttatgttggcatccgaagaggacgaggaagtctcttgtctgattactgacgctctatggtactttgcccaatctgtggctgatagtttgaatttgaggagattggtactaatgacatccagtctgtttaactttcacgctcatgttagtttaccacaatttgacgaattgggatacttggaccctgatgacaagactaggttagaggaacaggcctctggttttcctatgttgaaagtcaaagatatcaagtctgcctattctaattggcaaatcttgaaagagatcttaggaaagatgatcaaacagacaaaggcttcatctggagtgatttggaacagtttcaaagagttagaagagtctgaattggagactgtaatcagagaaattccagcaccttcattcctgataccattaccaaaacatttgactgcttcctcttcctctttgttggatcatgacagaacagtttttcaatggttggaccaacaaccacctagttctgttttgtacgtgtcatttggtagtacttctgaagtcgatgaaaaggacttccttgaaatcgcaagaggcttagtcgatagtaagcagtcattcctttgggtcgtgcgtccaggtttcgtgaaaggctcaacatgggtcgaaccacttccagatggttttctaggcgaaagaggtagaatagtcaaatgggttcctcaacaggaagttttagctcatggcgctattggggcattctggactcattccggatggaattcaactttagaatcagtatgcgaaggggtacctatgatcttttcagattttggtcttgatcaaccactgaacgcaagatacatgtctgatgttttgaaagtgggtgtatatctagaaaatggctgggaaaggggtgaaatagctaatgcaataagacgtgttatggttgatgaagagggggagtatatcagacaaaacgcaagagtgctgaagcaaaaggccgacgtttctctaatgaagggaggctcttcatacgaatccttagaatctcttgtttcctacatttcatcactgtaa

SEQ ID NO: 9 - DNA, 1422 bp, Stevia rebaudiana
  source 1..1422: mol_type = genomic DNA
atggctaccagtgactccatagttgacgaccgtaagcagcttcatgttgcgacgttcccatggcttgctttcggtcacatcctcccttaccttcagctttcgaaattgatagctgaaaagggtcacaaagtctcgtttctttctaccaccagaaacattcaacgtctctcttctcatatctcgccactcataaatgttgttcaactcacacttccacgtgtccaagagctgccggaggatgcagaggcgaccactgacgtccaccctgaagatattccatatctcaagaaggcttctgatggtcttcaaccggaggtcacccggtttctagaacaacactctccggactggattatttatgattatactcactactggttgccatccatcgcggctagcctcggtatctcacgagcccacttctccgtcaccactccatgggccattgcttatatgggaccctcagctgacgccatgataaatggttcagatggtcgaaccacggttgaggatctcacgacaccgcccaagtggtttccctttccgaccaaagtatgctggcggaagcatgatcttgcccgactggtgccttacaaagctccggggatatctgatggataccgtatggggctggttcttaagggatctgattgtttgctttccaaatgttaccatgagtttggaactcaatggctacctcttttggagacactacaccaagtaccggtggttccggtgggattactgccaccggaaatacccggagacgagaaagatgaaacatgggtgtcaatcaagaaatggctcgatggtaaacaaaaaggcagtgtggtgtacgttgcattaggaagcgaggttttggtgagccaaaccgaggttgttgagttagcattgggtctcgagctttctgggttgccatttgtttgggcttatagaaaaccaaaaggtcccgcgaagtcagactcggtggagttgccagacgggttcgtggaacgaactcgtgaccgtgggttggtctggacgagttgggcacctcagttacgaatactgagccatgagtcggtttgtggtttcttgactcattgtggttctggatcaattgtggaagggctaatgtttggtcaccctctaatcatgctaccgatttttggggaccaacctctgaatgctcgattactggaggacaaacaggtgggaatcgagataccaagaaatgaggaagatggttgcttgaccaaggagtcggttgctagatcactgaggtccgttgttgtggaaaaagaaggggagatctacaaggcgaacgcgagggagctgagtaaaatctataacgacactaaggttgaaaaagaatatgtaagccaattcgtagactatttggaaaagaatgcgcgtgcggttgccatcgatcatgagagttaa

SEQ ID NO: 10 - protein, 473 aa, Stevia rebaudiana
MATSDSIVDDRKQLHVATFPWLAFGHILPYLQLSKLIAEKGHKVSFLSTTRNIQRLSSHISPLINVVQLTLPRVQELPEDAEATTDVHPEDIPYLKKASDGLQPEVTRFLEQHSPDWIIYDYTHYWLPSIAASLGISRAHFSVTTPWAIAYMGPSADAMINGSDGRTTVEDLTTPPKWFPFPTKVCWRKHDLARLVPYKAPGISDGYRMGLVLKGSDCLLSKCYHEFGTQWLPLLETLHQVPVVPVGLLPPEVPGDEKDETWVSIKKWLDGKQKGSVVYVALGSEVLVSQTEVVELALGLELSGLPFVWAYRKPKGPAKSDSVELPDGFVERTRDRGLVWTSWAPQLRILSHESVCGFLTHCGSGSIVEGLMFGHPLIMLPIFGDQPLNARLLEDKQVGIEIPRNEEDGCLTKESVARSLRSVVVEKEGEIYKANARELSKIYNDTKVEKEYVSQFVDYLEKNTRAVAIDHES

SEQ ID NO: 11 - DNA, 1422 bp, Stevia rebaudiana
  source 1..1422: mol_type = genomic DNA
atggctaccagtgactccatagttgacgaccgtaagcagcttcatgttgcgacgttcccatggcttgctttcggtcacatcctcccttaccttcagctttcgaaattgatagctgaaaagggtcacaaagtctcgtttctttctaccaccagaaacattcaacgtctctcttctcatatctcgccactcataaatgttgttcaactcacacttccacgtgtccaagagctgccggaggatgcagaggcgaccactgacgtccaccctgaagatattccatatctcaagaaggcttctgatggtcttcaaccggaggtcacccggtttctagaacaacactctccggactggattatttatgattatactcactactggttgccatccatcgcggctagcctcggtatctcacgagcccacttctccgtcaccactccatgggccattgcttatatgggaccctcagctgacgccatgataaatggttcagatggtcgaaccacggttgaggatctcacgacaccgcccaagtggtttccctttccgaccaaagtatgctggcggaagcatgatcttgcccgactggtgccttacaaagctccggggatatctgatggataccgtatggggctggttcttaagggatctgattgtttgctttccaaatgttaccatgagtttggaactcaatggctacctcttttggagacactacaccaagtaccggtggttccggtgggattactgccaccggaagtacccggagacgagaaagatgaaacatgggtgtcaatcaagaaatggctcgatggtaaacaaaaaggcagtgtggtgtacgttgcattaggaagcgaggttttggtgagccaaaccgaggttgttgagttagcattgggtctcgagctttctgggttgccatttgtttgggcttatagaaaaccaaaaggtcccgcgaagtcagactcggtggagttgccagacgggttcgtggaacgaactcgtgaccgtgggttggtctggacgagttgggcacctcagttacgaatactgagccatgagtcggtttgtggtttcttgactcattgtggttctggatcaattgtggaagggctaatgtttggtcaccctctaatcatgctaccgatttttggggaccaacctctgaatgctcgattactggaggacaaacaggtgggaatcgagataccaagaaatgaggaagatggttgcttgaccaaggagtcggttgctagatcactgaggtccgttgttgtggaaaaagaaggggagatctacaaggcgaacgcgagggagctgagtaaaatctataacgacactaaggttgaaaaagaatatgtaagccaattcgtagactatttggaaaagaatacgcgtgcggttgccatcgatcatgagagttaa

SEQ ID NO: 12 - protein, 473 aa, Stevia rebaudiana
MATSDSIVDDRKQLHVATFPWLAFGHILPFLQLSKLIAEKGHKVSFLSTTRNIQRLSSHISPLINVVQLTLPRVQELPEDAEATTDVHPEDIQYLKKAVDGLQPEVTRFLEQHSPDWIIYDFTHYWLPSIAASLGISRAYFCVITPWTIAYLAPSSDAMINDSDGRTTVEDLTTPPKWFPFPTKVCWRKHDLARMEPYEAPGISDGYRMGMVFKGSDCLLFKCYHEFGTQWLPLLETLHQVPVVPVGLLPPEIPGDEKDETWVSIKKWLDGKQKGSVVYVALGSEALVSQTEVVELALGLELSGLPFVWAYRKPKGPAKSDSVELPDGFVERTRDRGLVWTSWAPQLRILSHESVCGFLTHCGSGSIVEGLMFGHPLIMLPLFGDQPLNARLLEDKQVGIEIPRNEEDGCLTKESVARSLRSVVVENEGEIYKANARELSKIYNDTKVEKEYVSQFVDYLEKNARAVAIDHES

SEQ ID NO: 13 - DNA, 1422 bp, Stevia rebaudiana
  source 1..1422: mol_type = genomic DNA
atggctaccagtgactccatagttgacgaccgtaagcagcttcatgttgcgacgttcccatggcttgctttcggtcacatcctccctttccttcagctttcgaaattgatagctgaaaagggtcacaaagtctcgtttctttctaccaccagaaacattcaacgtctctcttctcatatctcgccactcataaatgttgttcaactcacacttccacgtgtccaagagctgccggaggatgcagaggcgaccactgacgtccaccctgaagatattcaatatctcaagaaggctgttgatggtcttcaaccggaggtcacccggtttctagaacaacactctccggactggattatttatgattttactcactactggttgccatccatcgcggctagcctcggtatctcacgagcctacttctgcgtcatcactccatggaccattgcttatttggcaccctcatctgacgccatgataaatgattcagatggtcgaaccacggttgaggatctcacgacaccgcccaagtggtttccctttccgaccaaagtatgctggcggaagcatgatcttgcccgaatggagccttacgaagctccagggatatctgatggataccgtatggggatggtttttaagggatctgattgtttgcttttcaaatgttaccatgagtttggaactcaatggctacctcttttggagacactacaccaagtaccggtggttccggtgggattactgccgccggaaatacccggagacgagaaagatgaaacatgggtgtcaatcaagaaatggctcgatggtaaacaaaaaggcagtgtggtgtacgttgcattaggaagcgaggctttggtgagccaaaccgaggttgttgagttagcattgggtctcgagctttctgggttgccatttgtttgggcttatagaaaaccaaaaggtcccgcgaagtcagactcggtggagttgccagacgggttcgtggaacgaactcgtgaccgtgggttggtctggacgagttgggcacctcagttacgaatactgagccatgagtcggtttgtggtttcttgactcattgtggttctggatcaattgtggaagggctaatgtttggtcaccctctaatcatgctaccgctttttggggaccaacctctgaatgctcgattactggaggacaaacaggtgggaatcgagataccaagaaatgaggaagatggttgcttgaccaaggagtcggttgctagatcactgaggtccgttgttgtggaaaacgaaggggagatctacaaggcgaacgcgagggagctgagtaaaatctataacgacactaaggtggaaaaagaatatgtaagccaattcgtagactatttggaaaagaatgcgcgtgcggttgccatcgatcatgagagttaa

SEQ ID NO: 14 - protein, 485 aa, Stevia rebaudiana
MYNVTYHQNSKAMATSDSIVDDRKQLHVATFPWLAFGHILPFLQLSKLIAEKGHKVSFLSTTRNIQRLSSHISPLINVVQLTLPRVQELPEDAEATTDVHPEDIQYLKKAVDGLQPEVTRFLEQHSPDWIIYDFTHYWLPSIAASLGISRAYFCVITPWTIAYLAPSSDAMINDSDGRTTVEDLTTPPKWFPFPTKVCWRKHDLARMEPYEAPGISDGYRMGMVFKGSDCLLFKCYHEFGTQWLPLLETLHQVPVVPVGLLPPEIPGDEKDETWVSIKKWLDGKQKGSVVYVALGSEALVSQTEVVELALGLELSGLPFVWAYRKPKGPAKSDSVELPDGFVERTRDRGLVWTSWAPQLRILSHESVCGFLTHCGSGSIVEGLMFGHPLIMLPIFCDQPLNARLLEDKQVGIEIPRNEEDGCLTKESVARSLRSVVVENEGEIYKANARALSKIYNDTKVEKEYVSQFVDYLEKNARAVAIDHES

SEQ ID NO: 15 - DNA, 1458 bp, Stevia rebaudiana
  source 1..1458: mol_type = genomic DNA
atgtacaacgttacttatcatcaaaattcaaaagcaatggctaccagtgactccatagttgacgaccgtaagcagcttcatgttgcgacgttcccatggcttgctttcggtcacatcctccctttccttcagctttcgaaattgatagctgaaaagggtcacaaagtctcgtttctttctaccaccagaaacattcaacgtctctcttctcatatctcgccactcataaatgttgttcaactcacacttccacgtgtccaagagctgccggaggatgcagaggcgaccactgacgtccaccctgaagatattcaatatctcaagaaggctgttgatggtcttcaaccggaggtcacccggtttctagaacaacactctccggactggattatttatgattttactcactactggttgccatccatcgcggctagcctcggtatctcacgagcctacttctgcgtcatcactccatggaccattgcttatttggcaccctcatctgacgccatgataaatgattcagatggtcgaaccacggttgaggatctcacgacaccgcccaagtggtttccctttccgaccaaagtatgctggcggaagcatgatcttgcccgaatggagccttacgaagctccggggatatctgatggataccgtatggggatggtttttaagggatctgattgtttgcttttcaaatgttaccatgagtttggaactcaatggctacctcttttggagacactacaccaagtaccggtggttccggtgggattactgccgccggaaatacccggagacgagaaagatgaaacatgggtgtcaatcaagaaatggctcgatggtaaacaaaaaggcagtgtggtgtacgttgcattaggaagcgaggctttggtgagccaaaccgaggttgttgagttagcattgggtctcgagctttctgggttgccatttgtttgggcttatagaaaaccaaaaggtcccgcgaagtcagactcggtggagttgccagacgggttcgtggaacgaactcgtgaccgtgggttggtctggacgagttgggcacctcagttacgaatactgagccacgagtcagtttgtggtttcttgactcattgtggttctggatcaattgtggaagggctaatgtttggtcaccctctaatcatgctaccgattttttgtgaccaacctctgaatgctcgattactggaggacaaacaggtgggaatcgagataccaagaaatgaggaagatggttgcttgaccaaggagtcggttgctagatcactgaggtccgttgttgtggaaaacgaaggggagatctacaaggcgaacgcgagggcgctgagtaaaatctataacgacactaaggtggaaaaagaatatgtaagccaattcgtagactatttggaaaagaatgcgcgtgcggttgccatcgatcatgagagttaa

SEQ ID NO: 16 - protein, 473 aa, Stevia rebaudiana
MATSDSIVDDRKQLHVATFPWLAFGHILPFLQLSKLIAEKGHKVSFLSTTRNIQRLSSHISPLINVVQLTLPRVQELPEDAEATTDVHPEDIQYLKKAVDGLQPEVTRFLEQHSPDWIIYDFTHYWLPSIAASLGISRAYFCVITPWTIAYLAPSSDAMINDSDGRTTVEDLTTPPKWFPFPTKVCWRKHDLARMEPYEAPGISDGYRMGMVFKGSDCLLFKCYHEFGTQWLPLLETLHQVPVVPVGLLPPEIPGDEKDETWVSIKKWLDGKQKGSVVYVALGSEALVSQTEVVELALGLELSGLPFVWAYRKPKGPAKSDSVELPDGFVERTRDRGLVWTSWAPQLRILSHESVCGFLTHCGSGSIVEGLMFGHPLIMLPIFCDQPLNARLLEDKQVGIEIPRNEEDGCLTKESVARSLRSVVVENEGEIYKANARALSKIYNDTKVEKEYVSQFVDYLEKNARAVAIDHES

SEQ ID NO: 17 - DNA, 1422 bp, Stevia rebaudiana
  source 1..1422: mol_type = genomic DNA
atggctaccagtgactccatagttgacgaccgtaagcagcttcatgttgcgacgttcccatggcttgctttcggtcacatcctccctttccttcagctttcgaaattgatagctgaaaagggtcacaaagtctcgtttctttctaccaccagaaacattcaacgtctctcttctcatatctcgccactcataaatgttgttcaactcacacttccacgtgtccaagagctgccggaggatgcagaggcgaccactgacgtccaccctgaagatattcaatatctcaagaaggctgttgatggtcttcaaccggaggtcacccggtttctagaacaacactctccggactggattatttatgattttactcactactggttgccatccatcgcggctagcctcggtatctcacgagcctacttctgcgtcatcactccatggaccattgcttatttggcaccctcatctgacgccatgataaatgattcagatggtcgaaccacggttgaggatctcacgacaccgcccaagtggtttccctttccgaccaaagtatgctggcggaagcatgatcttgcccgaatggagccttacgaagctccggggatatctgatggataccgtatggggatggtttttaagggatctgattgtttgcttttcaaatgttaccatgagtttggaactcaatggctacctcttttggagacactacaccaagtaccggtggttccggtgggattactgccgccggaaatacccggagacgagaaagatgaaacatgggtgtcaatcaagaaatggctcgatggtaaacaaaaaggcagtgtggtgtacgttgcattaggaagcgaggctttggtgagccaaaccgaggttgttgagttagcattgggtctcgagctttctgggttgccatttgtttgggcttatagaaaaccaaaaggtcccgcgaagtcagactcggtggagttgccagacgggttcgtggaacgaactcgtgaccgtgggttggtctggacgagttgggcacctcagttacgaatactgagccacgagtcagtttgtggtttcttgactcattgtggttctggatcaattgtggaagggctaatgtttggtcaccctctaatcatgctaccgattttttgtgaccaacctctgaatgctcgattactggaggacaaacaggtgggaatcgagataccaagaaatgaggaagatggttgcttgaccaaggagtcggttgctagatcactgaggtccgttgttgtggaaaacgaaggggagatctacaaggcgaacgcgagggcgctgagtaaaatctataacgacactaaggtggaaaaagaatatgtaagccaattcgtagactatttggaaaagaatgcgcgtgcggttgccatcgatcatgagagttaa

SEQ ID NO: 18 - DNA, 1086 bp, synthetic construct
  misc_feature 1..1086: note = Description of Artificial Sequence: Synthetic polynucleotide
  source 1..1086: mol_type = other DNA
atggctttggtaaacccaaccgctcttttctatggtacctctatcagaacaagacctacaaacttactaaatccaactcaaaagctaagaccagtttcatcatcttccttaccttctttctcatcagttagtgcgattcttactgaaaaacatcaatctaatccttctgagaacaacaatttgcaaactcatctagaaactcctttcaactttgatagttatatgttggaaaaagtcaacatggttaacgaggcgcttgatgcatctgtcccactaaaagacccaatcaaaatccatgaatccatgagatactctttattggcaggcggtaagagaatcagaccaatgatgtgtattgcagcctgcgaaatagtcggaggtaatatccttaacgccatgccagccgcatgtgccgtggaaatgattcatactatgtctttggtgcatgacgatcttccatgtatggataatgatgacttcagaagaggtaaacctatttcacacaaggtctacggggaggaaatggcagtattgaccggcgatgctttactaagtttatctttcgaacatatagctactgctacaaagggtgtatcaaaggatagaatcgtcagagctataggggagttggcccgttcagttggctccgaaggtttagtggctggacaagttgtagatatcttgtcagagggtgctgatgttggattagatcacctagaatacattcacatccacaaaacagcaatgttgcttgagtcctcagtagttattggcgctatcatgggaggaggatctgatcagcagatcgaaaagttgagaaaattcgctagatctattggtctactattccaagttgtggatgacattttggatgttacaaaatctaccgaagagttggggaaaacagctggtaaggatttgttgacagataagacaacttacccaaagttgttaggtatagaaaagtccagagaatttgccgaaaaacttaacaaggaagcacaagagcaattaagtggctttgatagacgtaaggcagctcctttgatcgcgttagccaactacaatgcgtaccgtcaaaattga

SEQ ID NO: 19 - DNA, 1029 bp, synthetic construct
  misc_feature 1..1029: note = Description of Artificial Sequence: Synthetic polynucleotide
  source 1..1029: mol_type = other DNA
atggctgagcaacaaatatctaacttgctgtctatgtttgatgcttcacatgctagtcagaaattagaaattactgtccaaatgatggacacataccattacagagaaacgcctccagattcctcatcttctgaaggcggttcattgtctagatacgacgagagaagagtctctttgcctctcagtcataatgctgcctctccagatattgtatcacaactatgtttttccactgcaatgtcttcagagttgaatcacagatggaaatctcaaagattaaaggtggccgattctccttacaactatatcctaacattaccatcaaaaggaattagaggtgcctttatcgattccctgaacgtatggttggaggttccagaggatgaaacatcagtcatcaaggaagttattggtatgctccacaactcttcattaatcattgatgacttccaagataattctccacttagaagaggaaagccatctacccatacagtcttcggccctgcccaggctatcaatactgctacttacgttatagttaaagcaatcgaaaagatacaagacatagtgggacacgatgcattggcagatgttacgggtactattacaactattttccaaggtcaggccatggacttgtggtggacagcaaatgcaatcgttccatcaatacaggaatacttacttatggtaaacgataaaaccggtgctctctttagactgagtttggagttgttagctctgaattccgaagccagtatttctgactctgctttagaaagtttatctagtgctgtttccttgctaggtcaatacttccaaatcagagacgactatatgaacttgatcgataacaagtatacagatcagaaaggcttctgcgaagatcttgatgaaggcaagtactcactaacacttattcatgccctccaaactgattcatccgatctactgaccaacatcctttcaatgagaagagtgcaaggaaagttaacggcacaaaagagatgttggttctggaaatga

SEQ ID NO: 20 - DNA, 903 bp, synthetic construct
  misc_feature 1..903: note = Description of Artificial Sequence: Synthetic polynucleotide
  source 1..903: mol_type = other DNA
atggaaaagactaaggagaaagcagaacgtatcttgctggagccatacagatacttattacaactaccaggaaagcaagtccgttctaaactatcacaagcgttcaatcactggttaaaagttcctgaagataagttacaaatcattattgaagtcacagaaatgctacacaatgcttctttactgatcgatgatatagaggattcttccaaactgagaagaggttttcctgtcgctcattccatatacggggtaccaagtgtaatcaactcagctaattacgtctacttcttgggattggaaaaagtattgacattagatcatccagacgctgtaaagctattcaccagacaacttcttgaattgcatcaaggtcaaggtttggatatctattggagagacacttatacttgcccaacagaagaggagtacaaagcaatggttctacaaaagactggcggtttgttcggacttgccgttggtctgatgcaacttttctctgattacaaggaggacttaaagcctctgttggataccttgggcttgtttttccagattagagatgactacgctaacttacattcaaaggaatattcagaaaacaaatcattctgtgaagatttgactgaagggaagtttagttttccaacaatccacgccatttggtcaagaccagaatctactcaagtgcaaaacattctgcgtcagagaacagagaatattgacatcaaaaagtattgtgttcagtacttggaagatgttggttcttttgcttacacaagacatacacttagagaattagaggcaaaagcatacaagcaaatagaagcctgtggaggcaatccttctctagtggcattggttaaacatttgtccaaaatgttcaccgaggaaaacaagtaa

SEQ ID NO: 21 - DNA, 1020 bp, synthetic construct
  misc_feature 1..1020: note = Description of Artificial Sequence: Synthetic polynucleotide
  source 1..1020: mol_type = other DNA
atggcaagattctattttcttaacgcactattgatggttatctcattacaatcaactacagccttcactccagctaaacttgcttatccaacaacaacaacagctctaaatgtcgcctccgccgaaacttctttcagtctagatgaatacttggcctctaagataggacctatagagtctgccttggaagcatcagtcaaatccagaattccacagaccgataagatctgcgaatctatggcctactctttgatggcaggaggcaagagaattagaccagtgttgtgtatcgctgcatgtgagatgttcggtggatcccaagatgtcgctatgcctactgctgtggcattagaaatgatacacacaatgtctttgattcatgatgatttgccatccatggataacgatgacttgagaagaggtaaaccaacaaaccatgtcgttttcggcgaagatgtagctattcttgcaggtgactctttattgtcaacttccttcgagcacgtcgctagagaaacaaaaggagtgtcagcagaaaagatcgtggatgttatcgctagattaggcaaatctgttggtgccgagggccttgctggcggtcaagttatggacttagaatgtgaagctaaaccaggtaccacattagacgacttgaaatggattcatatccataaaaccgctacattgttacaagttgctgtagcttctggtgcagttctaggtggtgcaactcctgaagaggttgctgcatgcgagttgtttgctatgaatataggtcttgcctttcaagttgccgacgatatccttgatgtaaccgcttcatcagaagatttgggtaaaactgcaggcaaagatgaagctactgataagacaacttacccaaagttattaggattagaagagagtaaggcatacgcaagacaactaatcgatgaagccaaggaaagtttggctccttttggagatagagctgcccctttattggccattgcagatttcattattgatagaaagaattga

SEQ ID NO: 22 - DNA, 1068 bp, synthetic construct
  misc_feature 1..1068: note = Description of Artificial Sequence: Synthetic polynucleotide
  source 1..1068: mol_type = other DNA
atgcacttagcaccacgtagagtccctagaggtagaagatcaccacctgacagagttcctgaaagacaaggtgccttgggtagaagacgtggagctggctctactggctgtgcccgtgctgctgctggtgttcaccgtagaagaggaggaggcgaggctgatccatcagctgctgtgcatagaggctggcaagccggtggtggcaccggtttgcctgatgaggtggtgtctaccgcagccgccttagaaatgtttcatgcttttgctttaatccatgatgatatcatggatgatagtgcaactagaagaggctccccaactgttcacagagccctagctgatcgtttaggcgctgctctggacccagatcaggccggtcaactaggagtttctactgctatcttggttggagatctggctttgacatggtccgatgaattgttatacgctccattgactccacatagactggcagcagtactaccattggtaacagctatgagagctgaaaccgttcatggccaatatcttgatataactagtgctagaagacctgggaccgatacttctcttgcattgagaatagccagatataagacagcagcttacacaatggaacgtccactgcacattggtgcagccctggctggggcaagaccagaactattagcagggctttcagcatacgccttgccagctggagaagccttccaattggcagatgacctgctaggcgtcttcggtgatccaagacgtacagggaaacctgacctagatgatcttagaggtggaaagcatactgtcttagtcgccttggcaagagaacatgccactccagaacagagacacacattggatacattattgggtacaccaggtcttgatagacaaggcgcttcaagactaagatgcgtattggtagcaactggtgcaagagccgaagccgaaagacttattacagagagaagagatcaagcattaactgcattgaacgcattaacactgccacctcctttagctgaggcattagcaagattgacattagggtctacagctcatcctgcctaa

SEQ ID NO: 23 - DNA, 993 bp, synthetic construct
  misc_feature 1..993: note = Description of Artificial Sequence: Synthetic polynucleotide
  source 1..993: mol_type = other DNA
atgtcatatttcgataactacttcaatgagatagttaattccgtgaacgacatcattaagtcttacatctctggcgacgtaccaaaactatacgaagcctcctaccatttgtttacatcaggaggaaagagactaagaccattgatccttacaatttcttctgatcttttcggtggacagagagaaagagcatactatgctggcgcagcaatcgaagttttgcacacattcactttggttcacgatgatatcatggatcaagataacattcgtagaggtcttcctactgtacatgtcaagtatggcctacctttggccattttagctggtgacttattgcatgcaaaagcctttcaattgttgactcaggcattgagaggtctaccatctgaaactatcatcaaggcgtttgatatctttacaagatctatcattatcatatcagaaggtcaagctgtcgatatggaattcgaagatagaattgatatcaaggaacaagagtatttggatatgatatctcgtaaaaccgctgccttattctcagcttcttcttccattggggcgttgatagctggagctaatgataacgatgtgagattaatgtccgatttcggtacaaatcttgggatcgcatttcaaattgtagatgatatacttggtttaacagctgatgaaaaagagctaggaaaacctgttttcagtgatatcagagaaggtaaaaagaccatattagtcattaagactttagaattgtgtaaggaagacgagaaaaagattgtgttaaaagcgctaggcaacaagtcagcatcaaaggaagagttgatgagttctgctgacataatcaaaaagtactcattggattacgcctacaacttagctgagaaatactacaaaaacgccatcgattctctaaatcaagtttcaagtaaaagtgatattccagggaaggcattgaaatatcttgctgaattcaccatcagaagacgtaagtaa

SEQ ID NO: 24 - DNA, 894 bp, synthetic construct
  misc_feature 1..894: note = Description of Artificial Sequence: Synthetic polynucleotide
  source 1..894: mol_type = other DNA
atggtcgcacaaactttcaacctggatacctacttatcccaaagacaacaacaagttgaagaggccctaagtgctgctcttgtgccagcttatcctgagagaatatacgaagctatgagatactccctcctggcaggtggcaaaagattaagacctatcttatgtttagctgcttgcgaattggcaggtggttctgttgaacaagccatgccaactgcgtgtgcacttgaaatgatccatacaatgtcactaattcatgatgacctgccagccatggataacgatgatttcagaagaggaaagccaactaatcacaaggtgttcggggaagatatagccatcttagcgggtgatgcgcttttagcttacgcttttgaacatattgcttctcaaacaagaggagtaccacctcaattggtgctacaagttattgctagaatcggacacgccgttgctgcaacaggcctcgttggaggccaagtcgtagaccttgaatctgaaggtaaagctatttccttagaaacattggagtatattcactcacataagactggagccttgctggaagcatcagttgtctcaggcggtattctcgcaggggcagatgaagagcttttggccagattgtctcattacgctagagatataggcttggcttttcaaatcgtcgatgatatcctggatgttactgctacatctgaacagttggggaaaaccgctggtaaagaccaggcagccgcaaaggcaacttatccaagtctattgggtttagaagcctctagacagaaagcggaagagttgattcaatctgctaaggaagccttaagaccttacggttcacaagcagagccactcctagcgctggcagacttcatcacacgtcgtcagcattaa

SEQ ID NO: 25 - DNA, 1116 bp, synthetic construct
  misc_feature 1..1116: note = Description of Artificial Sequence: Synthetic polynucleotide
  source 1..1116: mol_type = other DNA
atggcctctgttactttgggttcctggatcgtcgtccaccaccataaccatcaccatccatcatctatcctaactaaatctcgttcaagatcctgtcctattacactaaccaaaccaatctcttttcgttcaaagagaacagtttcctctagtagttctatcgtgtcctctagtgtcgtcactaaggaagacaatctgagacagtctgaaccttcttcctttgatttcatgtcatatatcattactaaggcagaactagtgaataaggctcttgattcagcagttccattaagagagccattgaaaatccatgaagcaatgagatactctcttctagctggcgggaagagagtcagacctgtactctgcatagcagcgtgcgaattagttggtggcgaggaatcaaccgctatgcctgccgcttgtgctgtagaaatgattcatacaatgtcactgatacacgatgatttgccatgtatggataacgatgatctgagaaggggtaagccaactaaccataaggttttcggcgaagatgttgccgtcttagctggtgatgctttgttatctttcgcgttcgaacatttggcatccgcaacatcaagtgatgttgtgtcaccagtaagagtagttagagcagttggagaactggctaaagctattggaactgagggtttagttgcaggtcaagtcgtcgatatctcttccgaaggtcttgatttgaatgatgtaggtcttgaacatctcgaattcatccatcttcacaagacagctgcacttttagaagccagtgcggttctcggcgcaattgttggcggagggagtgatgacgaaattgagagattgaggaagtttgctagatgtataggattactgttccaagtagtagacgatatactagatgtgacaaagtcttccaaagagttgggaaaaacagctggtaaagatttgattgccgacaaattgacctaccctaagattatggggctagaaaaatcaagagaatttgccgagaaactcaatagagaggcgcgtgatcaactgttgggtttcgattctgataaagttgcaccactcttagccttagccaactacatcgcttacagacaaaactaa

SEQ ID NO: 26 - DNA, 1086 bp, Stevia rebaudiana
  source 1..1086: mol_type = genomic DNA
atggctcttgtaaatcccacagctttgttctatggaacctccataagaaccagacccacaaacttgctcaacccgacccaaaaacttcgacccgtttcctcgtcttctttgccttccttctcttcagtttctgcaatcttgacggaaaaacaccaatcaaacccatcagaaaacaataacttgcaaacccatctcgaaacaccattcaatttcgactcttacatgctggagaaagtaaacatggtgaatgaagctctggacgcctcggttccactcaaagacccgataaagatccatgaatccatgcggtactcccttctagctggcgggaaacgcatccgaccgatgatgtgcatcgccgcttgcgaaatagtcggaggcaacatattaaacgccatgccagctgcatgcgcggtcgagatgattcacaccatgtcactagttcatgacgaccttccatgcatggataacgacgacttccgacgtggaaaaccaataagccacaaggtgtacggtgaagaaatggcggttctaaccggggacgcgttactctcattatccttcgaacatatcgcgaccgcgacaaaaggcgtatccaaagacaggatcgtccgagccattggtgaactcgcaaggtccgttggctcggagggtttggtcgccggtcaggtggttgatattttatccgaaggggctgatgttgggttagaccacttggagtatattcatatacacaagactgcaatgttgcttgagagctcggtcgtgatcggcgcgatcatgggcggtgggtctgaccaacagatcgaaaagttgcgaaagtttgcgagatcgattggtttgttgtttcaggtggtagatgatattcttgatgtcacaaagtcgactgaggaattggggaaaacggcgggaaaagatttgctgacggacaagacaacgtatccgaagttgttggggatcgaaaaatcgagagaatttgcggagaaattaaacaaggaagcgcaagaacaattgtcggggtttgatcgccgcaaggcggctccgttaattgcccttgctaattacaatgcttataggcaaaactga

SEQ ID NO: 27 - DNA, 1029 bp, Gibberella fujikuroi
  source 1..1029: mol_type = genomic DNA
atggctgaacaacagatctccaaccttctttcaatgtttgatgcttctcacgcaagccagaagttggagattacggttcagatgatggatacctaccattacagagaaactcctccagactcttcctcttcagaaggcggttccttatctcgctatgatgagcgacgggtctcccttccgctctctcacaatgcagcctccccagacatagtctcccagttatgcttctcaacagctatgagctcggagctcaatcacaggtggaagtcacagcgcctcaaggttgctgactctccctacaactacatcctgactcttccatctaaaggtattcgtggggctttcattgactcactgaatgtctggctcgaggtccccgaagacgagacctcggtgatcaaagaggtgattggcatgctccacaactcgtctctcataatcgatgacttccaagacaactccccacttcggcggggcaagccatctacacatactgtcttcggtccagcacaagcaatcaacacagcaacatatgtcatcgtcaaggccatcgagaaaatacaggatatcgtcggtcacgatgcattggcagatgtaactggcactataaccacaatcttccagggtcaggcaatggatctgtggtggactgctaatgccattgttccgtctatccaagaatatctcctgatggtcaatgacaagactggtgccctgttcaggttatcgcttgaactactggcgctgaactctgaagcatccatcagtgacagcgcgcttgaatctctcagcagcgctgtctcactgctcgggcagtatttccagataagagatgattacatgaatctcattgacaacaagtatactgatcagaaaggattttgcgaggatctggacgaggggaaatactcgttgactctaatccatgctctgcagaccgactccagcgaccttctcaccaacatcttatcgatgagaagagtccaaggaaaacttacggcgcagaaaagatgctggttttggaagtga

SEQ ID NO: 28 - DNA, 903 bp, Mus musculus
  source 1..903: mol_type = genomic DNA
atggagaaaactaaagagaaagctgagaggattcttctagagccctataggtacttacttcagttaccaggtaaacaggtgagaagcaaactttcacaggcatttaatcactggctgaaagttccagaagacaagctacagattatcattgaagtgactgaaatgttgcataatgccagtttactcattgatgatattgaagacagttcaaagctccgacgtggtttcccagtggctcacagcatctatggtgtcccatctgtcattaattctgccaattacgtctacttccttggactggaaaaagtcttaacccttgatcacccggatgcggtgaagcttttcacacgccagcttctggaacttcatcagggacaaggcctcgatatttactggagggacacctacacttgtccaactgaagaagaatataaagccatggtgttgcagaagacaggtggtttgtttggattagcagtaggtcttatgcagctgttctctgattacaaagaagatctaaagccactgcttgacacacttgggctctttttccagattagagatgattatgccaatctacactccaaagaatacagtgaaaacaaaagtttctgtgaagacttgacagaagggaagttctcattccccactatccatgccatttggtcaaggccagaaagcacccaggtacagaacatcctgcgccagagaacagagaatatagatattaaaaagtattgtgtgcagtacctggaggatgtaggttcttttgcatacactcgacacactcttagagagcttgaagctaaagcctacaaacaaattgaggcctgtggtgggaacccttcactagtggctttagtcaagcacttaagtaagatgttcacagaagaaaataaataa

SEQ ID NO: 29 - DNA, 1020 bp, Thalassiosira pseudonana
  source 1..1020: mol_type = genomic DNA
atggctcgtttctacttcctgaacgctctcctcatggtgatttctttacaaagcaccacggcattcaccccggcaaaactcgcctacccaacaaccaccactgcattaaacgttgcctctgccgaaacatcatttagcctcgatgaatacctagcctccaaaatcggacccattgaatcagctctcgaggcatctgtcaaatctcgcattcctcaaactgacaagatatgcgagtctatggcatactcactcatggctggaggaaagcgtatccgtcccgttttgtgcattgctgcttgtgaaatgtttgggggaagtcaagatgtggctatgccgacggctgtggctttggagatgattcatactatgagtcttattcatgacgatttgccttcaatggacaacgatgatctccgacgaggaaagccaactaatcatgttgtctttggagaggatgttgctattcttgctggggattctcttctcagtacgtcttttgaacatgttgcccgtgaaaccaaaggagtgtcagctgaaaagattgtagatgttatcgctcgcctcgggaagtctgtgggtgcagagggtcttgctggtggacaggttatggatcttgagtgtgaggcgaagccaggaactaccctcgacgatctcaagtggattcacattcacaaaactgccactcttcttcaagtggcagtggcatcaggtgctgttcttggaggggccacaccagaggaggttgctgcttgtgaactgttcgcaatgaatattggacttgccttccaggtcgctgatgatattttggacgtgacggcatcgagtgaggatcttggcaaaactgctggaaaggatgaagccacagataagacaacttatcctaagcttttgggattggaggagagtaaggcatacgctcgacaactcatagacgaagcaaaggaatctttggctcctttcggtgatcgtgctgctccattgttggcaattgccgactttatcattgatcgaaagaactag

SEQ ID NO: 30 - DNA, 1068 bp, Streptomyces clavuligerus
  source 1..1068: mol_type = genomic DNA
atgcacctggctccccgccgagtaccgcgcggccgtcgaagcccacctgaccgcgttcctgaacgccaaggagcgctcggccgccgccggggggccggttccacaggatgtgcccgcgctgctgcgggagttcatcggcgccggggggggggggaagcggatccgtccgctgctgtgcatcgcggctggcaggccggcggcggaacaggactgccggacgaggtggtgtccacagcggcggcgctggagatgttccacgcgttcgcgctgatccacgacgacatcatggatgactccgcgaccaggcgcggcagcccgacggtgcaccgggcactcgccgaccggctcggcgccgctctcgaccccgaccaagccggacaactgggggtgagcacggcgatcctcgtcggggacctcgccctgacctggtcggacgaactgctgtacgctcccctgaccccccaccggctggccgcggtactgcccctggtcacggccatgcgcgcggaaacggtccacggccagtacctggacatcacctccgcccgccggcccggcacggacacctcactggcgctgcgaatcgcgcgctacaaaaccgctgcttacaccatggaacgccccctgcacatcggagcagcgctcgccggcgcacgaccggaactcctggcagggctcagcgcctacgcgctgccggcgggcgaggcattccagctcgccgacgacctcctgggagtgttcggcgatccacggagaaccggcaaacccgacctcgacgacctccgcggcggcaagcacaccgtcctcgtggccctcgcccgggaacacgccacacctgaacagcggcacaccctggacaccctgctcggcacaccaggcctcgaccggcagggcgcgtcccggctgcgctgcgtcctcgtcgccaccggggcccgggcggaagccgaacgcctgatcaccgaacggcgcgaccaggccctcaccgcgctcaacgccctgacactgcccccaccgctcgccgaggcactcgcccgcctcaccctcgggagtaccgcacacccggcctga

SEQ ID NO: 31 - DNA, 993 bp, Sulfulobus acidicaldarius
  source 1..993: mol_type = genomic DNA
atgagttactttgacaactattttaatgagattgttaattctgtaaacgacattattaagagctatatatctggagatgttcctaaactatatgaagcctcatatcatttgtttacatctggaggtaagaggttaagaccattaatcttaactatatcatcagatttattcggaggacagagagaaagagcttattatgcaggtgcagctattgaagttcttcatacttttacgcttgtgcatgatgatattatggatcaagataatatcagaagagggttacccacagtccacgtgaaatacggcttacccttagcaatattagctggggatttactacatgcaaaggcttttcagctcttaacccaggctcttagaggtttgccaagtgaaaccataattaaggctttcgatattttcactcgttcaataataattatatccgaaggacaggcagtagatatggaatttgaggacagaattgatataaaggagcaggaataccttgacatgatctcacgtaagacagctgcattattctcggcatcctcaagtataggcgcacttattgctggtgctaatgataatgatgtaagactgatgtctgatttcggtacgaatctaggtattgcatttcagattgttgacgatatcttaggtctaacagcagacgaaaaggaacttggaaagcctgtttttagtgatattagggagggtaaaaagactatacttgtaataaaaacactggagctttgtaaagaggacgagaagaagattgtcctaaaggcgttaggtaataagtcagcctcaaaagaagaattaatgagctcagcagatataattaagaaatactctttagattatgcatacaatttagcagagaaatattataaaaatgctatagactctttaaatcaagtctcctctaagagtgatatacctggaaaggctttaaaatatctagctgaatttacgataagaaggagaaaataa

SEQ ID NO: 32 - DNA, 894 bp, Synechococcus sp.
  source 1..894: mol_type = genomic DNA
ttggttgcccaaaccttcaacctggacacctacttgagccaacgccagcaacaggtggaagaggcgctttctgcggcattggttcccgcctatccggagcgcatttacgaggcgatgcgctacagcctgctggcgggggggaaacgcctgaggccgatcctctgtctggcggcctgtgagttggccggcggctctgtggagcaggccatgcccaccgcctgcgccctggagatgatccacaccatgtcgctgatccacgacgatctgccggcgatggacaacgacgattttcgccgcggcaagcccaccaatcacaaggtattcggcgaggatatcgccattttggcaggagatgccctgttggcctatgcctttgagcatatcgccagccaaacgcggggggtgccgccgcagttggtgctgcaagtcattgcccgcattggccatgctgtggcggcaaccggcttggtagggggccaggtggtggatctggagtccgaaggcaaagccatttccctagaaactttggagtacatccacagtcacaagacgggtgctctgctggaggcctcggtggtttcgggagggatcctggcaggggccgatgaggagctgctggcgcggctgagccactacgctcgggacatcggcctggcttttcagatcgtggacgacattttggatgttactgccaccagcgagcaactgggcaaaacggcaggcaaggatcaagctgccgccaaagccacctaccccagcttgttgggcctagaggcttcccggcagaaagctgaggaactgatccaatcggccaaggaggcgttgcgcccctacggatcccaggccgagcccctgttggctctggccgatttcatcacccgccgccagcattga

SEQ ID NO: 33 - DNA, 1116 bp, Arabidopsis thaliana
  source 1..1116: mol_type = genomic DNA
atggcttcagtgactctaggttcatggattgttgttcaccaccacaatcatcatcatccatcttcaatccttaccaaatccagatccagatcttgtcctataactcttactaaacccatctcctttcgatcaaaacgcaccgtttcatcatcttcttcaatcgtttcttcttccgttgttacaaaagaagacaatctacgccaatctgaaccatcctctttcgatttcatgtcgtacatcatcaccaaagccgaattagtcaacaaagctttagattcagctgttcctctccgtgagccactcaagatccacgaagcgatgcgttactctcttctcgccggtggcaaaagagttagaccagttctctgcatcgctgcttgtgaactcgtcggaggtgaagaatcaaccgctatgccagcagcttgcgccgtcgagatgattcacaccatgtcgttgatccacgacgatctcccttgtatggataacgacgatctccgccgtggaaaaccgaccaaccacaaagtgtttggtgaagacgtcgctgttttagccggagacgcgcttctctctttcgctttcgagcatttagcttcggcgacgagttctgatgttgtttctccggtgagagtggttcgagccgttggagaattggctaaagcgataggaacagaagggttagtggcgggtcaagtcgtggatattagtagtgaagggttagatttaaacgacgtcggtttagagcatttggagtttatccatttgcataaaacggcggcgttgcttgaagcttctgctgttttgggagctattgttggtggaggaagtgatgatgagattgagaggttaagaaagtttgcgagatgtattggtttgttgtttcaggtggttgatgatatcttggatgtgacgaaatcgtcgaaagagttagggaaaactgctgggaaagatttgattgctgataagttgacgtatcctaagattatgggtttggagaaatcgagagagtttgctgagaaattgaatagagaggctcgtgatcagcttttagggtttgattctgataaggttgctcctttgttggctttggctaattacattgcctatagacagaactga

SEQ ID NO: 34 - DNA, 2364 bp, synthetic construct
  misc_feature 1..2364: note = Description of Artificial Sequence: Synthetic polynucleotide
  source 1..2364: mol_type = other DNA
atgaaaaccgggtttatctcaccagcaacagtatttcatcacagaatctcaccagcgaccactttcagacatcacttatcacctgctactacaaactctacaggcattgtcgccttaagagacatcaacttcagatgtaaagcagtttctaaagagtactctgatctgttgcagaaagatgaggcttctttcacaaaatgggacgatgacaaggtgaaagatcatcttgataccaacaaaaacttatacccaaatgatgagattaaggaatttgttgaatcagtaaaggctatgttcggtagtatgaatgacggggagataaacgtctctgcatacgatactgcatgggttgctttggttcaagatgtcgatggatcaggtagtcctcagttcccttcttctttagaatggattgccaacaatcaattgtcagatggatcatggggagatcatttgctgttctcagctcacgatagaatcatcaacacattagcatgcgttattgcacttacaagttggaatgttcatccttctaagtgtgaaaaaggtttgaattttctgagagaaaacatttgcaaattagaagatgaaaacgcagaacatatgccaattggttttgaagtaacattcccatcactaattgatatcgcgaaaaagttgaacattgaagtacctgaggatactccagcacttaaagagatctacgcacgtagagatatcaagttaactaagatcccaatggaagttcttcacaaggtacctactactttgttacattctttggaaggaatgcctgatttggagtgggaaaaactgttaaagctacaatgtaaagatggtagtttcttgttttccccatctagtaccgcattcgccctaatgcaaacaaaagatgagaaatgcttacagtatctaacaaatatcgtcactaagttcaacggtggcgtgcctaatgtgtacccagtcgatttgtttgaacatatttgggttgttgatagactgcagagattggggattgccagatacttcaaatcagagataaaagattgtgtagagtatatcaataagtactggaccaaaaatggaatttgttgggctagaaatactcacgttcaagatatcgatgatacagccatgggattcagagtgttgagagcgcacggttatgacgtcactccagatgtttttagacaatttgaaaaagatggtaaattcgtttgctttgcagggcaatcaacacaagccgtgacaggaatgtttaacgtttacagagcctctcaaatgttgttcccaggggagagaattttggaagatgccaaaaagttctcttacaattacttaaaggaaaagcaaagtaccaacgaattgctggataaatggataatcgctaaagatctacctggtgaagttggttatgctctggatatcccatggtatgcttccttaccaagattggaaactcgttattaccttgaacaatacggcggtgaagatgatgtctggataggcaagacattatacagaatgggttacgtgtccaataacacatatctagaaatggcaaagctggattacaataactatgttgcagtccttcaattagaatggtacacaatacaacaatggtacgtcgatattggtatagagaagttcgaatctgacaacatcaagtcagtcctggtttcttactacttggctgcggcttcaatattcgaacctgagagatctaaggagagaatcgcttgggcaaagacaacaatcttagtcgataagatcacatcaattttcgattcctctcagtcaagtaaggaagatattactgcctttattgacaagtttcgtaacaagtcctcctctaaaaagcactctatcaacggtgaaccatggcatgaagttatggtagctttgaaaaagaccttacacggctttgctctggatgctcttatgactcattctcaagatatacatccacagttacatcaagcctgggaaatgtggttgactaaactacaagacggcgtagatgttactgctgagctaatggtccaaatgatcaacatgactgctggcagatgggtatcaaaggaattacttactcatccacaatatcaaagattgtctactgtgacaaattctgtgtgtcacgatattaccaaacttcacaatttcaaggagaattccaccacagtggattcaaaggttcaggaactagtccagttggtttttagtgacacaccagatgatttggatcaagatatgaaacaaacattcctgacagtgatgaagacattctactacaaggcgtggtgtgatccaaacactataaacgatcatatatctaaagttttcgaaatcgtaatttga

SEQ ID NO: 35 - DNA, 1584 bp, synthetic construct
  misc_feature 1..1584: note = Description of Artificial Sequence: Synthetic polynucleotide
  source 1..1584: mol_type = other DNA
atgcctgatgcacacgatgctccacctccacaaataagacagagaacactagtagatgaggctacccaactgctaactgagtccgcagaagatgcatggggtgaagtcagtgtgtcagaatacgaaacagcaaggctagttgcccatgctacatggttaggtggacacgccacaagagtggccttccttctggagagacaacacgaagacgggtcatggggtccaccaggtggatataggttagtccctacattatctgctgttcacgcattattgacatgtcttgcctctcctgctcaggatcatggcgttccacatgatagacttttaagagctgttgacgcaggcttgactgccttgagaagattggggacatctgactccccacctgatactatagcagttgagctggttatcccatctttgctagagggcattcaacacttactggaccctgctcatcctcatagtagaccagccttctctcaacatagaggctctcttgtttgtcctggtggactagatgggagaactctaggagctttgagatcacacgccgcagcaggtacaccagtaccaggaaaagtctggcacgcttccgagactttgggcttgagtaccgaagctgcttctcacttgcaaccagcccaaggtataatcggtggctctgctgctgccacagcaacatggctaaccagggttgcaccatctcaacagtcagattctgccagaagataccttgaggaattacaacacagatactctggcccagttccttccattacccctatcacatacttcgaaagagcatggttattgaacaattttgcagcagccggtgttccttgtgaggctccagctgctttgttggattccttagaagcagcacttacaccacaaggtgctcctgctggagcaggattgcctccagatgctgatgatacagccgctgtgttgcttgcattggcaacacatgggagaggtagaagaccagaagtactgatggattacaggactgacgggtatttccaatgctttattggggaaaggactccatcaatttcaacaaacgctcacgtattggaaacattagggcatcatgtggcccaacatccacaagatagagccagatacggatcagccatggataccgcatcagcttggctgctggcagctcaaaagcaagatggctcttggttagataaatggcatgcctcaccatactacgctactgtttgttgcacacaagccctagccgctcatgcaagtcctgcaactgcaccagctagacagagagctgtcagatgggttttagccacacaaagatccgatggcggttggggtctatggcattcaactgttgaagagactgcttatgccttacagatcttggccccaccttctggtggtggcaatatcccagtccaacaagcacttactagaggcagagcaagattgtgtggagccttgccactgactcctttatggcatgataaggatttgtatactccagtaagagtagtcagagctgccagagctgctgctctgtacactaccagagatctattgttaccaccattgtaa

SEQ ID NO: 36 - DNA, 1551 bp, synthetic construct
  misc_feature 1..1551: note = Description of Artificial Sequence: Synthetic polynucleotide
  source 1..1551: mol_type = other DNA
atgaacgccctatccgaacacattttgtctgaattgagaagattattgtctgaaatgagtgatggcggatctgttggtccatctgtgtatgatacggcccaggccctaagattccacggtaacgtaacaggtagacaagatgcatatgcttggttgatcgcccagcaacaagcagatggaggttggggctctgccgactttccactctttagacatgctccaacatgggctgcacttctcgcattacaaagagctgatccacttcctggcgcagcagacgcagttcagaccgcaacaagattcttgcaaagacaaccagatccatacgctcatgccgttcctgaggatgcccctattggtgctgaactgatcttgcctcagttttgtggagaggctgcttggttgttgggaggtgtggccttccctagacacccagccctattaccattaagacaggcttgtttagtcaaactgggtgcagtcgccatgttgccttcaggacacccattgctccactcctgggaggcatggggtacttctccaacaacagcctgtccagacgatgatggttctataggtatctcaccagcagctacagccgcctggagagcccaggctgtgaccagaggctcaactcctcaagtgggcagagctgacgcatacttacaaatggcttcaagagcaacgagatcaggcatagaaggagtcttccctaatgtttggcctataaacgtattcgaaccatgctggtcactgtacactctccatcttgccggtctgttcgcccatccagcactggctgaggctgtaagagttatcgttgctcaacttgaagcaagattgggagtgcatggcctcggaccagctttacattttgctgccgacgctgatgatactgcagttgccttatgcgttctgcatttggctggcagagatcctgcagttgacgcattgagacattttgaaattggtgagctctttgttacattcccaggagagagaaatgctagtgtctctacgaacattcacgctcttcatgctttgagattgttaggtaaaccagctgccggagcaagtgcatacgtcgaagcaaatagaaatccacatggtttgtgggacaacgaaaaatggcacgtttcatggctttatccaactgcacacgccgttgcagctctagctcaaggcaagcctcaatggagagatgaaagagcactagccgctctactacaagctcaaagagatgatggtggttggggagctggtagaggatccactttcgaggaaaccgcctacgctcttttcgctttacacgttatggacggatctgaggaagccacaggcagaagaagaatcgctcaagtcgtcgcaagagccttagaatggatgctagctagacatgccgcacatggattaccacaaacaccactctggattggtaaggaattgtactgtcctactagagtcgtaagagtagctgagctagctggcctgtggttagcattaagatggggtagaagagtattagctgaaggtgctggtgctgcaccttaa

SEQ ID NO: 37 - DNA, 2364 bp, Stevia rebaudiana
  source 1..2364: mol_type = genomic DNA
atgaagaccggcttcatctctcccgccaccgtcttccaccaccgtatttctccggcaaccaccttccgccaccacctttctccggcgaccaccaactccactggaattgtagctcttagagacatcaacttccggtgtaaagcggtatccaaagagtactctgatttactacaaaaagatgaggcttcatttaccaagtgggacgatgacaaagtgaaggaccatttggacacaaataagaatttgtatccaaacgatgagatcaaggagtttgttgagagcgtgaaagcaatgtttggttctatgaatgacggagaaataaatgtgtcagcgtatgatacggcttgggttgcactcgtgcaagatgttgatggaagtggttcccctcaatttccatcaagtttggagtggatcgcgaacaatcaactctcagatgggtcttggggcgatcatttgttattttcggctcatgataggatcattaacacgttggcatgtgttatagcgcttacttcttggaacgtccatccaagtaaatgtgaaaaaggactgaattttcttagagaaaacatatgtaaactcgaagacgagaacgcggaacatatgccaattggttttgaagtcacgttcccgtcgctaatagatatcgcaaagaagctaaatattgaagttcctgaggatactcctgccttaaaagaaatttatgcaagaagagacataaaactcacaaagataccaatggaagtattgcacaaagtgcccacaactttacttcatagtttggaaggaatgccagatttggaatgggaaaaacttctgaaattgcaatgcaaagatggatcatttctgttttctccatcatctactgcttttgcactcatgcaaacaaaagatgaaaagtgtcttcagtatttgacaaatattgttaccaaattcaatggtggagttccgaatgtgtacccggtggatctattcgaacatatttgggtagttgatcgacttcaacgacttgggattgctcgttatttcaaatcagagatcaaagattgcgttgaatatattaacaagtattggacaaagaatgggatttgttgggcaagaaacacgcacgtacaagatattgatgataccgcaatgggatttagggttttaagagcacatggttatgatgttactccagatgtatttcgacaatttgagaaggatggtaaattcgtatgtttcgctggacagtcaacacaagccgtcaccggaatgttcaatgtgtatagagcgtcacaaatgctctttcccggagaaagaattcttgaagatgcaaagaaattttcatataattatttgaaagaaaaacaatcgacaaatgagcttcttgataaatggatcatcgccaaagacttacctggagaggttggatatgcgctagacataccatggtatgcaagcttaccgcgactcgagacaagatattacttagagcaatacgggggcgaggatgatgtttggattggaaaaactctatacaggatgggatatgtgagcaataatacgtaccttgaaatggccaaattggactacaataactatgtggccgtgcttcaactcgaatggtacactatccagcaatggtatgttgatatcggtatcgaaaagtttgaaagtgacaatatcaaaagcgtattagtgtcgtattacttggctgcagccagcatattcgagccggaaaggtccaaggaacgaatcgcgtgggctaaaaccaccatattagttgacaagatcacctcaatttttgattcatcacaatcctcaaaagaggacataacagcctttatagacaaatttaggaacaaatcgtcttctaagaagcattcaataaatggagaaccatggcacgaggtgatggttgcactgaaaaagaccctacacggcttcgctttggatgcactcatgactcatagtcaagacatccacccgcaactccatcaagcttgggagatgtggttgacgaaattgcaagatggagtagatgtgacagcggaattaatggtacaaatgataaatatgacagctggtcgttgggtatccaaagaacttttaactcatcctcaataccaacgcctctcaaccgtcacaaatagtgtgtgtcacgatataactaagctccataacttcaaggagaattccacgacggtagactcgaaagttcaagaactagtgcaacttgtgtttagcgacacgcccgatgatcttgatcaggatatgaaacagacgtttctaaccgtcatgaaaaccttctactacaaggcgtggtgtgatccgaacacgataaatgaccatatctccaaggtgttcgagattgtaatatga

SEQ ID NO: 38 - DNA, 1584 bp, Streptomyces clavuligerus
  source 1..1584: mol_type = genomic DNA
ttgcccgacgcgcatgatgcccctccgcctcagatacgacagcggacccttgtcgatgaggcgacgcaactcctcacggagtcggccgaggacgcctggggtgaggtgtccgtgtccgagtacgaaacggcgcggctggtggcccacgccacctggctcggcggtcacgccacacgggtggccttcctgctggagcggcagcatgaggacggctcgtggggcccgcccggcgggtaccgtctcgtacccacgctgagtgccgtacacgccctgctcacctgtctggcgtctcccgcgcaggaccacggagtgcctcatgaccggctcctgcgcgcagttgacgcgggcctgacggcactgcgtcgtcttgggacgagcgacagcccgccggacaccattgcggtcgaactggtcataccctcgctccttgagggcatccagcacctcctggacccggcgcacccgcattcccgacccgctttttcgcaacaccgcggcagcctcgtctgccccgggggcctcgacggccgcacgctgggggccttgcgctcccacgccgcagccggcacacctgtcccgggcaaggtgtggcacgcctcggaaaccttggggctatcgaccgaggcagcctcccaccttcaacccgcccagggcatcatcggtggctccgccgccgcgacagcaacatggctcaccagggtcgccccgtcgcaacagagcgacagcgcacggcgctacctggaagaactccagcaccgatacagcggcccggtgccctccatcaccccgatcacctatttcgaacgggcctggctgctcaacaacttcgctgccgcgggggttccatgcgaggctccggcagcccttctcgacagcctggaggcagcgctcacaccacagggcgctccagcgggtgcgggactgccgccggacgcggatgacaccgccgccgttctgctggcgcttgccacgcacggccgcgggcgccgtcccgaggtcctcatggactaccgcacggacggctacttccagtgcttcatcggcgaacgcaccccttccatcagcaccaatgcccatgtcctggagacgctcggtcaccacgtcgcccaacaccctcaggacagggcccgatacggctcagccatggacaccgcatcagcgtggctcctcgcggctcagaagcaggatggcagctggctcgacaagtggcacgcctccccctactacgccaccgtctgctgcacccaggcactggcagcccacgcttcccctgccaccgcccccgcacggcagcgtgctgtgcggtgggtgctggcaacacaacgctcggacggcggctggggcctgtggcactccacggtcgaggagaccgcctacgccctgcagatcctcgccccaccttccggcggcgggaacatccccgtgcaacaggcgctcaccagggggcgcgcccgcctctgcggcgctttgccgctgactcccctatggcatgacaaggacctgtacacgccggtacgtgtcgtccgcgccgcccgtgccgccgccctgtacaccacccgtgacctgcttctgccgcccctgtga

SEQ ID NO: 39 - DNA, 1551 bp, Bradyrhizobium japonicum
  source 1..1551: mol_type = genomic DNA
gtgaacgcgctgtccgaacatatcctttccgaattgcgccgcctgctgagcgaaatgagcgatggcggcagcgtcggtccgtccgtctacgacacggcgcaggcgctgcgcttccacggcaacgtcaccggtcggcaggacgcatacgcgtggctcatcgcgcagcaacaggccgacggcggatggggaagcgcggacttcccgctgttccgccatgcgcccacgtgggcggcgttactggcattgcagcgtgccgatcctcttcccggagctgcggacgcagtccagactgcaacgaggttcctccagcgccagcccgatccctacgcacatgcggtgccagaagacgcgccgatcggcgcggagctgatcctgccgcagttttgcggtgaggccgcatggttgctgggtggcgtagcgtttccgcgccatcctgcgctgttgccattgcggcaagcgtgcctggtcaagctgggggcggtggcgatgttgccgagcggccatccgttgctacactcctgggaagcctgggggacgtcgccgaccaccgcatgcccggatgacgacggcagcatcggcatcagtccggcggccaccgccgcgtggcgtgcccaggccgtgacacgggggagcacgccgcaggtcgggcgcgccgatgcgtatctgcagatggcatcgcgggcgacgcgcagcggcatcgaaggtgtctttcccaacgtctggccgatcaatgtgttcgagccatgctggtcgctgtacaccctgcatctggccgggcttttcgcgcatcccgcgctcgcggaggcggttcgcgtgatcgtcgcgcagctcgaggcccgtctgggcgtgcacggtctgggcccggccttgcacttcgcggctgatgcggacgacaccgccgttgcgttgtgcgtcctgcaccttgcaggccgtgacccggcggtcgatgcgttgcgccatttcgaaatcggcgagctgttcgtcaccttccccggcgaacgcaatgcctcggtgtcgaccaacattcatgccctgcatgcgttgcgactgttgggaaagcccgccgcgggcgccagcgcgtacgtcgaggccaatcgcaacccgcacggtctatgggacaacgaaaaatggcacgtttcgtggctgtatcccaccgcgcatgcggtcgctgcgctggcgcaaggcaagccccagtggcgagatgagcgcgcgctggcggcgctgctgcaggcgcagcgcgacgacggtggctggggcgcgggtcgcgggtccacgttcgaggaaaccgcctatgcgctgtttgcgttgcacgtgatggatgggagcgaagaggcgacagggcgccggcgcatcgcgcaggtggtggcgcgtgcgctggagtggatgctcgcccgccatgcggcgcatggattgccgcagacgccgctgtggatcggcaaggaactgtattgccccactcgggtcgtgcgcgtggccgaactcgccgggttgtggctggcgcttcgttgggggcggcgcgtcctggccgagggggcaggagcggcgccatga

SEQ ID NO: 40 - DNA, 2355 bp, synthetic construct
  misc_feature 1..2355: note = Description of Artificial Sequence: Synthetic polynucleotide
  source 1..2355: mol_type = other DNA
atgaatttgagtttgtgtatagcatctccactattgaccaaatctaatagaccagctgctttatcagcaattcatacagctagtacatcccatggtggccaaaccaaccctacgaatctgataatcgatacgaccaaggagagaatacaaaaacaattcaaaaatgttgaaatttcagtttcttcttatgatactgcgtgggttgccatggttccatcacctaattctccaaagtctccatgtttcccagaatgtttgaattggctgattaacaaccagttgaatgatggatcttggggtttagtcaatcacacgcacaatcacaaccatccacttttgaaagattctttatcctcaactttggcttgcatcgtggccctaaagagatggaacgtaggtgaggatcagattaacaaggggcttagtttcattgaatctaacttggcttccgcgactgaaaaatctcaaccatctccaataggattcgatatcatctttccaggtctgttagagtacgccaaaaatctagatatcaacttactgtctaagcaaactgatttctcactaatgttacacaagagagaattagaacaaaagagatgtcattcaaacgaaatggatggttacctagcttatatctctgaaggtcttggtaatctttacgattggaatatggtgaaaaagtaccagatgaaaaatggctcagttttcaattccccttctgcaactgcggcagcattcattaaccatcaaaatccaggatgcctgaactatttgaattcactactagacaaattcggcaacgcagttccaactgtataccctcacgatttgtttatcagattgagtatggtggatacaattgaaagacttggtatatcccaccactttagagtcgagatcaaaaatgttttggatgagacataccgttgttgggtggagagagatgaacaaatctttatggatgttgtgacgtgcgcgttggcctttagattgttgcgtattaacggttacgaagttagtccagatccacttgccgaaattacaaacgaattagctttaaaggatgaatacgccgctcttgaaacatatcatgcgtcacatatcctttaccaagaggacttatcatctggaaaacaaattcttaaatctgctgatttcctgaaggaaatcatatccactgatagtaatagactgtccaaactgatccataaagaggttgaaaatgcacttaagttccctattaacaccggcttagaacgtattaacacaagacgtaacatccagctttacaacgtagacaatactagaatcttgaaaaccacttaccattcttccaacatatcaaacactgattacctaagattagctgttgaagatttctacacatgtcagtctatctatagagaagagctgaaaggattagagagatgggtcgttgagaataagctagatcaattgaaatttgccagacaaaagacagcttattgttacttctcagttgccgccactttatcaagtccagaattgtcagatgcacgtatttcttgggctaaaaacggaattttgacaactgttgttgatgatttctttgatattggcgggacaatcgacgaattgacaaacctgattcaatgcgttgaaaagtggaatgtcgatgtcgataaagactgttgctcagaacatgttagaatactgttcttggctctgaaagatgctatctgttggatcggggatgaggctttcaaatggcaagctagagatgtgacgtctcacgtcattcaaacctggctagaactgatgaactctatgttgagagaagcaatttggactagagatgcatacgttcctacattaaacgagtatatggaaaacgcttatgtctcctttgctttgggtcctatcgttaagcctgccatatactttgtaggaccaaagctatccgaggaaatcgtcgaatcatcagaataccataacttgttcaagttaatgtccacacaaggcagattacttaatgatattcattctttcaaaagagagtttaaggaaggaaagttaaatgctgttgctctgcatctttctaatggcgaaagtggtaaagtcgaagaggaagtagttgaggaaatgatgatgatgatcaaaaacaagagaaaggagttgatgaaactaatcttcgaagagaacggttcaattgttcctagagcatgtaaggatgcattttggaacatgtgtcatgtgctaaactttttctacgcaaacgacgatggttttactgggaacacaatactagatacagtaaaagacatcatatacaaccctttggtcttagtaaacgaaaacgaggagcaaagataa

SEQ ID NO: 41 - DNA, 2355 bp, synthetic construct
  misc_feature 1..2355: note = Description of Artificial Sequence: Synthetic polynucleotide
  source 1..2355: mol_type = other DNA
atgaatctgtccctttgtatagctagtccactgttgacaaaatcttctagaccaactgctctttctgcaattcatactgccagtactagtcatggaggtcaaacaaacccaacaaatttgataatcgatactactaaggagagaatccaaaagctattcaaaaatgttgaaatctcagtatcatcttatgacaccgcatgggttgcaatggtgccatcacctaattccccaaaaagtccatgttttccagagtgcttgaattggttaatcaataatcagttaaacgatggttcttggggtttagtcaaccacactcataaccacaatcatccattattgaaggactctttatcatcaacattagcctgtattgttgcattgaaaagatggaatgtaggtgaagatcaaatcaacaagggtttatcattcatagaatccaatctagcttctgctaccgacaaatcacaaccatctccaatcgggttcgacataatcttccctggtttgctggagtatgccaaaaaccttgatatcaacttactgtctaaacaaacagatttctctttgatgctacacaaaagagagttagagcagaaaagatgccattctaacgaaattgacgggtacttagcatatatctcagaaggtttgggtaatttgtatgactggaacatggtcaaaaagtatcagatgaaaaatggatccgtattcaattctccttctgcaactgccgcagcattcattaatcatcaaaaccctgggtgtcttaactacttgaactcactattagataagtttggaaatgcagttccaacagtctatcctttggacttgtacatcagattatctatggttgacactatagagagattaggtatttctcatcatttcagagttgagatcaaaaatgttttggacgagacatacagatgttgggtcgaaagagatgagcaaatctttatggatgtcgtgacctgcgctctggcttttagattgctaaggatacacggatacaaagtatctcctgatcaactggctgagattacaaacgaactggctttcaaagacgaatacgccgcattagaaacataccatgcatcccaaatactttaccaggaagacctaagttcaggaaaacaaatcttgaagtctgcagatttcctgaaaggcattctgtctacagatagtaataggttgtctaaattgatacacaaggaagtagaaaacgcactaaagtttcctattaacactggtttagagagaatcaatactaggagaaacattcagctgtacaacgtagataatacaaggattcttaagaccacctaccatagttcaaacatttccaacacctattacttaagattagctgtcgaagacttttacacttgtcaatcaatctacagagaggagttaaagggcctagaaagatgggtagttcaaaacaagttggatcaactgaagtttgctagacagaagacagcatactgttatttctctgttgctgctaccctttcatccccagaattgtctgatgccagaataagttgggccaaaaatggtattcttacaactgtagtcgatgatttctttgatattggaggtactattgatgaactgacaaatcttattcaatgtgttgaaaagtggaacgtggatgtagataaggattgctgcagtgaacatgtgagaatacttttcctggctctaaaagatgcaatatgttggattggcgacgaggccttcaagtggcaagctagagatgttacatctcatgtcatccaaacttggcttgaactgatgaactcaatgctaagagaagcaatctggacaagagatgcatacgttccaacattgaacgaatacatggaaaacgcttacgtctcatttgccttgggtcctattgttaagccagccatatactttgttgggccaaagttatccgaagagattgttgagtcttccgaatatcataacctattcaagttaatgtcaacacaaggcagacttctgaacgatatccactccttcaaaagagaattcaaggaaggtaagctaaacgctgttgctttgcacttgtctaatggtgaatctggcaaagtggaagaggaagtcgttgaggaaatgatgatgatgatcaaaaacaagagaaaggaattgatgaaattgattttcgaggaaaatggttcaatcgtacctagagcttgtaaagatgctttttggaatatgtgccatgttcttaacttcttttacgctaatgatgatggcttcactggaaatacaatattggatacagttaaagatatcatctacaacccacttgttttggtcaatgagaacgaggaacaaagataa

SEQ ID NO: 42 - DNA, 1773 bp, synthetic construct
  misc_feature 1..1773: note = Description of Artificial Sequence: Synthetic polynucleotide
  source 1..1773: mol_type = other DNA
atggctatgccagtgaagctaacacctgcgtcattatccttaaaagctgtgtgctgcagattctcatccggtggccatgctttgagattcgggagtagtctgccatgttggagaaggacccctacccaaagatctacttcttcctctactactagaccagctgccgaagtgtcatcaggtaagagtaaacaacatgatcaggaagctagtgaagcgactatcagacaacaattacaacttgtggatgtcctggagaatatgggaatatccagacattttgctgcagagataaagtgcatactagacagaacttacagatcttggttacaaagacacgaggaaatcatgctggacactatgacatgtgctatggcttttagaatcctaagattgaacggatacaacgtttcatcagatgaactataccacgttgtagaggcatctggtctgcataattctttgggtgggtatcttaacgataccagaacactacttgaattacacaaggcttcaacagttagtatctctgaggatgaatctatcttagattcaattggctctagatccagaacattgcttagagaacaattggagtctggtggcgcactgagaaagccttctttattcaaagaggttgaacatgcactggatggacctttttacaccacacttgatagacttcatcataggtggaatattgaaaacttcaacattattgagcaacacatgttggagactccatacttatctaaccagcatacatcaagggatatcctagcattgtcaattagagatttttcctcctcacaattcacttatcaacaagagctacagcatctggagagttgggttaaggaatgtagattagatcaactacagttcgcaagacagaaattagcgtacttttacctatcagccgcaggcaccatgttttctcctgagctttctgatgcgagaacattatgggccaaaaacggggtgttgacaactattgttgatgatttctttgatgttgccggttctaaagaggaattggaaaacttagtcatgctggtcgaaatgtgggatgaacatcacaaagttgaattctattctgagcaggtcgaaatcatcttctcttccatctacgattctgtcaaccaattgggtgagaaggcctctttggttcaagacagatcaattacaaaacaccttgttgaaatatggttagacttgttaaagtccatgatgacggaagttgaatggagactgtcaaaatacgtgcctacagaaaaggaatacatgattaatgcctctcttatcttcggcctaggtccaatcgttttaccagctttgtatttcgttggtccaaagatttcagaaagtatagtaaaggacccagaatatgatgaattgttcaaactaatgtcaacatgtggtagattgttgaatgacgtgcaaacgttcgaaagagaatacaatgagggtaaactgaattctgtcagtctattggttcttcacggaggcccaatgtctatttcagacgcaaagaggaaattacaaaagcctattgatacgtgtagaagagatcttctttctttggtccttagagaagagtctgtagtaccaagaccatgtaaggaactattctggaaaatgtgtaaagtgtgctatttcttttactcaacaactgatgggttttctagtcaagtcgaaagagcaaaagaggtagacgctgtcataaatgagccactgaagttgcaaggttctcatacactggtatctgatgtttaa

SEQ ID NO: 43 - DNA, 2232 bp, synthetic construct
  misc_feature 1..2232: note = Description of Artificial Sequence: Synthetic polynucleotide
  source 1..2232: mol_type = other DNA
atgcagaacttccatggtacaaaggaaaggatcaaaaagatgtttgacaagattgaattgtccgtttcttcttatgatacagcctgggttgcaatggtcccatcccctgattgcccagaaacaccttgttttccagaatgtactaaatggatcctagaaaatcagttgggtgatggtagttggtcacttcctcatggcaatccacttctagttaaagatgcattatcttccactcttgcttgtattctggctcttaaaagatggggaatcggtgaggaacagattaacaaaggactgagattcatagaactcaactctgctagtgtaaccgataacgaacaacacaaaccaattggatttgacattatctttccaggtatgattgaatacgctatagacttagacctgaatctaccactaaaaccaactgacattaactccatgttgcatcgtagagcccttgaattgacatcaggtggaggcaaaaatctagaaggtagaagagcttacttggcctacgtctctgaaggaatcggtaagctgcaagattgggaaatggctatgaaataccaacgtaaaaacggatctctgttcaatagtccatcaacaactgcagctgcattcatccatatacaagatgctgaatgcctccactatattcgttctcttctccagaaatttggaaacgcagtccctacaatataccctctcgatatctatgccagactttcaatggtagatgccctggaacgtcttggtattgatagacatttcagaaaggagagaaagttcgttctggatgaaacatacagattttggttgcaaggagaagaggagattttctccgataacgcaacctgtgctttggccttcagaatattgagacttaatggttacgatgtctctcttgaagatcacttctctaactctctgggcggttacttaaaggactcaggagcagctttagaactgtacagagccctccaattgtcttacccagacgagtccctcctggaaaagcaaaattctagaacttcttacttcttaaaacaaggtttatccaatgtctccctctgtggtgacagattgcgtaaaaacataattggagaggtgcatgatgctttaaacttttccgaccacgctaacttacaaagattagctattcgtagaaggattaagcattacgctactgacgatacaaggattctaaaaacttcctacagatgctcaacaatcggtaaccaagattttctaaaacttgcagtggaagatttcaatatctgtcaatcaatacaaagagaggaattcaagcatattgaaagatgggtcgttgaaagacgtctagacaagttaaagttcgctagacaaaaagaggcctattgctatttctcagccgcagcaacattgtttgcccctgaattgtctgatgctagaatgtcttgggccaaaaatggtgtattgacaactgtggttgatgatttcttcgatgtcggaggctctgaagaggaattagttaacttgatagaattgatcgagcgttgggatgtgaatggcagtgcagatttttgtagtgaggaagttgagattatctattctgctatccactcaactatctctgaaataggtgataagtcatttggctggcaaggtagagatgtaaagtctcaagttatcaagatctggctggacttattgaaatcaatgttaactgaagctcaatggtcttcaaacaagtctgttcctaccctagatgagtatatgacaaccgcccatgtttcattcgcacttggtccaattgtacttccagccttatacttcgttggcccaaagttgtcagaagaggttgcaggtcatcctgaactactaaacctctacaaagtcacatctacttgtggcagactactgaatgattggagaagttttaagagagaatccgaggaaggtaagctcaacgctattagtttatacatgatccactccggtggtgcttctacagaagaggaaacaatcgaacatttcaaaggtttgattgattctcagagaaggcaactgttacaattggtgttgcaagagaaggatagtatcatacctagaccatgtaaagatctattttggaatatgattaagttattacacactttctacatgaaagatgatggcttcacctcaaatgagatgaggaatgtagttaaggcaatcattaacgaaccaatctcactggatgaattatga

SEQ ID NO: 44 - DNA, 2355 bp, Stevia rebaudiana
  source 1..2355: mol_type = genomic DNA
atgaatctttcactatgcatcgcgtcccctttgttaaccaaatcaaatcgacccgcggctctgtcagctattcatacagcatcaacttcacatggtggacaaactaatcccactaatctgatcattgatacaaccaaagaacggatccaaaaacagtttaaaaatgtagaaatttctgtttcttcatatgacacagcatgggtagccatggtcccttctccaaactcacccaaatcgccttgtttccctgagtgtctcaattggttaattaataatcagcttaatgatggttcatggggtcttgttaatcacactcataatcataatcacccgttgcttaaagattctctatcttcaacattagcatgtattgttgcattaaaaagatggaatgttggggaagatcaaataaataaaggtctaagttttattgagtcaaatcttgcttcagctactgaaaaaagtcaaccatctcccattggttttgacatcatatttcctggtttgcttgagtatgcgaaaaacttggacataaacctcctttcaaaacaaacagattttagtttgatgctacataagagggaattggagcaaaaaagatgccattcaaatgagatggatggatacttggcgtatatctctgaaggactcggtaatttatatgattggaatatggtgaagaaatatcagatgaaaaatggttctgttttcaactcaccatcagcaacagctgctgctttcattaatcatcaaaatcctggttgtcttaattatttaaattcacttttggacaagtttggtaatgcagtcccaacagtttatcctcatgatttatttatccgactttctatggttgacacaattgaaagattaggaatttcacaccatttcagagtggaaattaaaaatgttttagatgaaacatacagatgttgggtggaacgagatgagcaaatattcatggatgttgtaacatgtgctttagcctttcggttattaaggatcaatgggtatgaagtttccccagatccattggctgaaattactaatgaattagctttgaaagacgaatatgcagctcttgaaacatatcatgcgtcacatatattataccaagaggatttatcttctggaaaacaaatcttgaagtcagctgatttcctcaaagagataatatccactgattcaaacaggctttctaaattaattcacaaagaggtggaaaatgctcttaagttccctatcaataccggtttagaacgcataaacactagacgaaatatacagctttacaatgtagacaatacaagaattctgaaaactacatatcactcatcaaatattagtaacactgattacctaaggttggctgttgaagatttctacacctgccaatctatttatcgtgaagaattaaaaggtcttgaaaggtgggtggtagagaataagttggaccagctcaagtttgctaggcaaaagaccgcctactgttatttctctgttgctgcaacactttcgtctcccgaattatcagatgcgcgtatttcatgggccaaaaatggcatattaactacagtagttgatgacttttttgatatcggtggtacaatcgatgaattgaccaacctgattcaatgtgttgaaaaatggaatgtagatgtcgacaaggattgttgttcagagcatgttcggattttatttttagcattaaaagatgcaatctgttggattggagatgaagcttttaaatggcaagcgcgcgatgtaactagccatgttattcaaacttggttggaactaatgaatagtatgttgagagaagctatatggacaagagatgcttatgtgccaacattaaatgaatatatggaaaacgcttacgtgtcatttgcattaggcccgattgtcaagccggctatttactttgtggggcccaaattatcagaggagattgttgaaagctctgaatatcataatctatttaagctaatgagcacgcagggtcgacttctaaacgatatccatagcttcaagagggaatttaaggaaggcaaattaaacgcggtagcattgcatttgagtaacggagaaagtgggaaagtggaagaagaggttgtggaggagatgatgatgatgattaaaaacaagaggaaagaattaatgaaattaatttttgaagaaaatggtagcattgttcctagagcttgtaaagatgcattttggaacatgtgtcacgtgttgaattttttttacgcaaacgatgacgggtttactggaaacacgattcttgatactgtgaaggacatcatttacaacccgttggtgcttgtgaatgaaaatgaagaacaaaggtaa

SEQ ID NO: 45 - DNA, 2355 bp, Stevia rebaudiana
  source 1..2355: mol_type = genomic DNA
atgaatctttcactatgcattgcgtcccctttgttaaccaaatcaagtcgacccacggctctgtcagctattcatacagcatcaacttcacatggtggacaaactaatcccactaatctgatcattgatacaaccaaagaacggatccaaaaactgtttaaaaatgtagaaatttctgtttcttcatatgacacagcatgggtagccatggtcccttctccaaactcacccaaatcgccttgtttccctgagtgtctcaattggttaattaataatcagcttaatgatggttcatggggtcttgttaatcacactcataatcataatcacccgttgcttaaagattctctatcttcaacattagcatgtattgttgcattaaaaagatggaatgttggggaagatcaaataaataaaggtctaagttttattgagtcaaatcttgcttcagcaactgacaaaagtcaaccatctcccattggttttgatatcatatttcctggtttgcttgagtatgcgaaaaacttggacataaacctcctttcaaaacaaacagattttagtttgatgctacataagagggaattggagcaaaaaagatgccattcaaatgagattgatggatacttggcgtatatctctgaaggactcggtaatttatatgattggaatatggtgaagaaatatcagatgaaaaatggttctgttttcaactcaccatcagcaacagcagctgctttcattaatcatcaaaatcccggttgtcttaattatttaaattcacttttggacaagtttggtaatgcagtcccaacagtttatcctcttgatttatatatccggctttctatggttgacacaattgaaagattaggaatttcacaccatttcagagtggaaattaaaaatgttttagatgaaacatacagatgttgggtggaacgagatgagcaaatattcatggatgttgtaacatgtgctttagcctttcggttattaaggatccacgggtataaagtctccccagatcaattggctgaaattactaatgaattagctttcaaagacgaatacgcagctcttgaaacatatcatgcatcacagatattataccaagaggatttatcttctggaaaacaaatcttgaagtcagctgatttcctcaaagggatattatccactgattcaaacaggctttctaaattaattcacaaagaggtggaaaatgctcttaagttccctatcaataccggtttagaacgcataaacactagacgaaatatacagctttacaatgtagacaatacaagaattctgaaaactacatatcactcatcaaatattagtaacacttattacctaaggttggctgttgaagatttctacacctgccaatctatttatcgtgaagaattaaaaggtcttgaaaggtgggtggtacagaataagttggaccagctcaagtttgctaggcaaaagaccgcctactgttatttctctgttgctgcaacactttcgtctcccgaattatcagatgcgcgtatttcatgggccaaaaatggcatattaactacagtagttgatgacttttttgatatcggtggtacaatcgatgaattgaccaacctgattcaatgtgttgaaaaatggaatgtagatgtcgacaaggattgttgttcagagcatgttcggattttatttttagcattaaaagatgcaatctgttggattggagatgaagcttttaaatggcaagcgcgcgatgtaactagccatgttattcaaacttggttggaactaatgaatagtatgttgagagaagctatatggacaagagatgcttatgtgccaacattaaatgaatatatggaaaacgcttacgtgtcatttgcattaggcccgattgtcaagccggctatttactttgtggggcccaaattatcagaggagattgttgaaagctctgaatatcataatctatttaagctaatgagcacgcagggtcgacttctaaacgatatccatagcttcaagagggaatttaaggaaggcaaattaaacgcggtagcattgcatttgagtaacggagaaagtgggaaagtggaagaagaggttgtggaggagatgatgatgatgattaaaaacaagaggaaagaattaatgaaattaatttttgaagaaaatggtagcattgttcctagagcttgtaaagatgcattttggaacatgtgtcacgtgttgaattttttttacgcaaacgatgacgggtttactggaaacacgattcttgatactgtgaaggacatcatttacaacccgttggtgcttgtgaatgaaaatgaagaacaaaggtaa

SEQ ID NO: 46 - DNA, 1773 bp, Zea mays
  source 1..1773: mol_type = genomic DNA
atggccatgccagtgaagctgactcctgcctccctctcgctgaaggcggtctgctgccgcttcagctccggagggcatgcgctgcgcttcggctcgtcgctaccgtgctggaggaggacgccgacgcaacggagcacgtcgtcgtctacgacgcgccctgcggctgaggttagctctggcaaaagcaagcagcacgatcaagaagcatcggaggctacgataagacagcagctccagctagtcgatgtgcttgagaacatggggatttctcggcattttgctgctgaaatcaaatgcatccttgacaggacatacagaagttggttacagagacatgaggaaattatgctggacacaatgacctgtgcgatggcatttcgtattctaaggttgaatggatacaatgtctcttctgatgagttgtatcatgttgttgaagcttccggactccataattcacttggaggatatctcaatgatacaagaaccttgttagaattacacaaggcctcgacagttagtatctctgaagatgagtctatcctggatagcataggctcaaggtcacgtaccttactgagggaacaactagagtctggtggtgctctacgaaaaccttcactctttaaagaggtggaacatgctctggacggtcccttctacaccacattggaccgtctacaccataggtggaacatcgaaaatttcaatattatagagcagcacatgctagagacaccatacttgtcaaatcaacataccagtagagatattctagcgttgagtattagagacttcagttcctctcagtttacttaccagcaagaacttcaacatcttgaaagctgggtgaaagagtgcaggttagaccagctacaatttgcgcgacagaagttggcatacttctacttgtctgctgctggcaccatgttctctcctgagctgtctgatgctcgaactttgtgggccaaaaatggtgtgctcacaactattgttgacgacttctttgatgttgcgggatcaaaagaagaacttgaaaaccttgtcatgttggttgagatgtgggacgagcatcacaaagttgagttctactcagaacaagtagagattatattttcttcaatttatgactcagttaaccaacttggtgaaaaggcttctttggtacaagaccgcagtattaccaaacacctagtagaaatatggttggatttgctaaagtctatgatgacagaggtagagtggcgtttgagcaaatatgtgccaacagagaaggaatacatgataaatgcatctttaatatttggactaggccccattgtattgccagcattatattttgttgggccaaagatctcagagtctattgttaaagatccagaatatgatgaattgttcaaactgatgagcacatgtggtcgcctcttgaatgatgttcagacttttgagagggagtacaacgagggcaagttgaatagtgtttctctcctcgttcttcatggtggccccatgtccatatcagacgccaaaaggaaattacagaagcccatagacacatgcagaagagacctcctaagtttagttcttcgtgaagaaagtgttgttcctaggccctgcaaggaattattttggaaaatgtgcaaggtgtgctacttcttctactcgacgacggatgggtttagctcacaagtggagagggctaaagaagtggatgcggtgatcaatgagccactaaagctacaaggaagtcatacgctggtgtctgatgtgtga

SEQ ID NO: 47 - DNA, 2232 bp, Populus trichocarpa
  source 1..2232: mol_type = genomic DNA
atgcagaactttcatggaactaaggaaaggatcaagaagatgtttgataagattgaattgtcagtgtcttcatatgacactgcttgggtggcaatggtcccatctccagattgtccggaaactccttgttttccagagtgcacaaaatggattttggaaaatcaacttggtgatggctcctggagtcttcctcatggcaatccattattagttaaggatgctctttcatctacattagcgtgcatccttgcattgaagcgatggggtatcggtgaagaacaaataaataaaggccttcgatttattgagttgaattccgcttcagttacggataacgagcaacataaaccaattggatttgatataatatttcctggcatgattgaatatgccatagatttggatttgaacctccctttgaagccgacagatataaattccatgctccacaggagggctttggagcttacaagtggcggtggcaagaacttggagggaagaagagcctacttagcatatgtttcggaaggaattggaaaattacaggattgggaaatggccatgaaatatcaaagaaagaatggatcactgttcaattcaccatccaccacagcagctgcctttattcatattcaagatgctgagtgtctccattatattcgttcactcttacagaagtttgggaatgcagttccaaccatttatcctttggatatatatgctcgtctttctatggttgatgctcttgaaaggttgggaatcgatcggcattttaggaaggaaagaaaatttgttttggacgaaacataccgattttggttgcagggggaggaagagatattttctgataatgccacttgtgctttggcatttaggatattacgtttgaacggatatgatgtctctctagaagatcatttctctaattcactgggaggatatttgaaggattcgggagctgccttagagttgtacagagctctgcagctaagttatccagatgaatcacttctggaaaaacaaaattctcggacaagctatttcctgaaacagggattatccaacgtttcactttgtggagataggcttcgtaaaaatattatcggagaggtgcatgatgctctcaatttttctgaccatgcaaatttgcaacgcttagctatcagaagaagaattaaacattatgctacagatgatacgaggattttgaaaacttcgtatcgttgttcgactattggtaaccaggattttctcaaattggctgtagaagacttcaatatctgtcaatcaatacagcgtgaagaatttaaacatatcgagaggtgggttgtagagaggagactggacaagctaaagtttgctaggcagaaggaggcctactgttacttctctgctgcagcaactctcttcgctccagaactatctgatgcacgcatgtcatgggcaaaaaatggtgtgcttactactgttgttgatgacttctttgatgttggtggttctgaagaagaactggtaaaccttattgaattgattgagaggtgggatgtcaatggcagtgctgatttttgttctgaggaagttgagatcatatattcggcaattcacagcactataagtgagataggagacaaatctttcggatggcaaggacgcgatgtgaaaagtcaggttatcaagatttggttggatttgctcaaatccatgttgacagaagcacaatggtcaagtaacaaatcagtgccgacccttgatgaatatatgacaactgcacatgtatcgttcgctctagggcctattgttcttccagctctgtattttgtggggcctaagctttcagaggaggttgctggacatcctgaattgcttaatctatacaaggttacgagcacttgcgggcgtctgctcaatgactggagaagctttaagagagaatctgaagaagggaaattgaatgccatctcattgtacatgattcacagcggtggtgcttcaactgaagaagagaccatcgaacattttaaaggattgatcgacagccagagaagacaattgcttcaattagttttgcaggaaaaggatagtataattcctagaccctgcaaggatttgttttggaacatgataaaattattgcacacgttctacatgaaggatgatggattcacttcaaacgagatgagaaatgttgtcaaggcaataataaatgaacccatctctctagatgaattataa

SEQ ID NO: 48 - DNA, 2952 bp, synthetic construct
  misc_feature 1..2952: note = Description of Artificial Sequence: Synthetic polynucleotide
  source 1..2952: mol_type = other DNA
atggaatttgatgaaccattggttgacgaagcaagatctttagtgcagcgtactttacaagattatgatgacagatacggcttcggtactatgtcatgtgctgcttatgatacagcctgggtgtctttagttacaaaaacagtcgatgggagaaaacaatggcttttcccagagtgttttgaatttctactagaaacacaatctgatgccggaggatgggaaatcgggaattcagcaccaatcgacggtatattgaatacagctgcatccttacttgctctaaaacgtcacgttcaaactgagcaaatcatccaacctcaacatgaccataaggatctagcaggtagagctgaacgtgccgctgcatctttgagagcacaattggctgcattggatgtgtctacaactgaacacgtcggttttgagataattgttcctgcaatgctagacccattagaagccgaagatccatctctagttttcgattttccagctaggaaacctttgatgaagattcatgatgctaagatgagtagattcaggccagaatacttgtatggcaaacaaccaatgaccgccttacattcattagaggctttcataggcaaaatcgacttcgataaggtaagacaccaccgtacccatgggtctatgatgggttctccttcatctaccgcagcctacttaatgcacgcttcacaatgggatggtgactcagaggcttaccttagacacgtgattaaacacgcagcagggcagggaactggtgctgtaccatctgctttcccatcaacacattttgagtcatcttggattcttaccacattgtttagagctggattttcagcttctcatcttgcctgtgatgagttgaacaagttggtcgagatacttgagggctcattcgagaaggaaggtggggcaatcggttacgctccagggtttcaagcagatgttgatgatactgctaaaacaataagtacattagcagtccttggaagagatgctacaccaagacaaatgatcaaggtatttgaagctaatacacattttagaacataccctggtgaaagagatccttctttgacagctaattgtaatgctctatcagccttactacaccaaccagatgcagcaatgtatggatctcaaattcaaaagattaccaaatttgtctgtgactattggtggaagtctgatggtaagattaaagataagtggaacacttgctacttgtacccatctgtcttattagttgaggttttggttgatcttgttagtttattggagcagggtaaattgcctgatgttttggatcaagagcttcaatacagagtcgccatcacattgttccaagcatgtttaaggccattactagaccaagatgccgaaggatcatggaacaagtctatcgaagccacagcctacggcatccttatcctaactgaagctaggagagtttgtttcttcgacagattgtctgagccattgaatgaggcaatccgtagaggtatcgctttcgccgactctatgtctggaactgaagctcagttgaactacatttggatcgaaaaggttagttacgcacctgcattattgactaaatcctatttgttagcagcaagatgggctgctaagtctcctttaggcgcttccgtaggctcttctttgtggactccaccaagagaaggattggataagcatgtcagattattccatcaagctgagttattcagatcccttccagaatgggaattaagagcctccatgattgaagcagctttgttcacaccacttctaagagcacatagactagacgttttccctagacaagatgtaggtgaagacaaatatcttgatgtagttccattcttttggactgccgctaacaacagagatagaacttacgcttccactctattcctttacgatatgtgttttatcgcaatgttaaacttccagttagacgaattcatggaggccacagccggtatcttattcagagatcatatggatgatttgaggcaattgattcatgatcttttggcagagaaaacttccccaaagagttctggtagaagtagtcagggcacaaaagatgctgactcaggtatagaggaagacgtgtcaatgtccgattcagcttcagattcccaggatagaagtccagaatacgacttggttttcagtgcattgagtacctttacaaaacatgtcttgcaacacccatctatacaaagtgcctctgtatgggatagaaaactacttgctagagagatgaaggcttacttacttgctcatatccaacaagcagaagattcaactccattgtctgaattgaaagatgtgcctcaaaagactgatgtaacaagagtttctacatctactactaccttctttaactgggttagaacaacttccgcagaccatatatcctgcccatactccttccactttgtagcatgccatctaggcgcagcattgtcacctaaagggtctaacggtgattgctatccttcagctggtgagaagttcttggcagctgcagtctgcagacatttggccaccatgtgtagaatgtacaacgatcttggatcagctgaacgtgattctgatgaaggtaatttgaactccttggacttccctgaattcgccgattccgcaggaaacggagggatagaaattcagaaggccgctctattaaggttagctgagtttgagagagattcatacttagaggccttccgtcgtttacaagatgaatccaatagagttcacggtccagccggtggtgatgaagccagattgtccagaaggagaatggcaatccttgaattcttcgcccagcaggtagatttgtacggtcaagtatacgtcattagggatatttccgctcgtattcctaaaaacgaggttgagaaaaagagaaaattggatgatgctttcaattga

SEQ ID NO: 49 - DNA, 2646 bp, synthetic construct
  misc_feature 1..2646: note = Description of Artificial Sequence: Synthetic polynucleotide
  source 1..2646: mol_type = other DNA
atggcttctagtacacttatccaaaacagatcatgtggcgtcacatcatctatgtcaagttttcaaatcttcagaggtcaaccactaagatttcctggcactagaaccccagctgcagttcaatgcttgaaaaagaggagatgccttaggccaaccgaatccgtactagaatcatctcctggctctggttcatatagaatagtaactggcccttctggaattaaccctagttctaacgggcacttgcaagagggttccttgactcacaggttaccaataccaatggaaaaatctatcgataacttccaatctactctatatgtgtcagatatttggtctgaaacactacagagaactgaatgtttgctacaagtaactgaaaacgtccagatgaatgagtggattgaggaaattagaatgtactttagaaatatgactttaggtgaaatttccatgtccccttacgacactgcttgggtggctagagttccagcgttggacggttctcatgggcctcaattccacagatctttgcaatggattatcgacaaccaattaccagatggggactggggcgaaccttctcttttcttgggttacgatagagtttgtaatactttagcctgtgtgattgcgttgaaaacatggggtgttggggcacaaaacgttgaaagaggaattcagttcctacaatctaacatatacaagatggaggaagatgacgctaatcatatgccaataggattcgaaatcgtattccctgctatgatggaagatgccaaagcattaggtttggatttgccatacgatgctactattttgcaacagatttcagccgaaagagagaaaaagatgaaaaagatcccaatggcaatggtgtacaaatacccaaccactttacttcactccttagaaggcttgcatagagaagttgattggaataagttgttacaattacaatctgaaaatggtagttttctttattcacctgcttcaaccgcatgcgccttaatgtacactaaggacgttaaatgttttgattacttaaaccagttgttgatcaagttcgaccacgcatgcccaaatgtatatccagtcgatctattcgaaagattatggatggttgacagattgcagagattagggatctccagatactttgaaagagagattagagattgtttacaatacgtctacagatattggaaagattgtggaatcggatgggcttctaactcttccgtacaagatgttgatgatacagccatggcgtttagacttttaaggactcatggtttcgacgtaaaggaagattgctttagacagtttttcaaggacggagaattcttctgcttcgcaggccaatcatctcaagcagttacaggcatgtttaatctttcaagagccagtcaaacattgtttccaggagaatctttattgaaaaaggctagaaccttctctagaaacttcttgagaacaaagcatgagaacaacgaatgtttcgataaatggatcattactaaagatttggctggtgaagtcgagtataacttgaccttcccatggtatgcctctttgcctagattagaacataggacatacttagatcaatatggaatcgatgatatctggataggcaaatctttatacaaaatgcctgctgttaccaacgaagttttcctaaagttggcaaaggcagactttaacatgtgtcaagctctacacaaaaaggaattggaacaagtgataaagtggaacgcgtcctgtcaattcagagatcttgaattcgccagacaaaaatcagtagaatgctattttgctggtgcagccacaatgttcgaaccagaaatggttcaagctagattagtctgggcaagatgttgtgtattgacaactgtcttagacgattactttgaccacgggacacctgttgaggaacttagagtgtttgttcaagctgtcagaacatggaatccagagttgatcaacggtttgccagagcaagctaaaatcttgtttatgggcttatacaaaacagttaacacaattgcagaggaagcattcatggcacagaaaagagacgtccatcatcatttgaaacactattgggacaagttgataacaagtgccctaaaggaggccgaatgggcagagtcaggttacgtcccaacatttgatgaatacatggaagtagctgaaatttctgttgctctagaaccaattgtctgtagtaccttgttctttgcgggtcatagactagatgaggatgttctagatagttacgattaccatctagttatgcatttggtaaacagagtcggtagaatcttgaatgatatacaaggcatgaagagggaggcttcacaaggtaagatctcatcagttcaaatctacatggaggaacatccatctgttccatctgaggccatggcgatcgctcatcttcaagagttagttgataattcaatgcagcaattgacatacgaagttcttaggttcactgcggttccaaaaagttgtaagagaatccacttgaatatggctaaaatcatgcatgccttctacaaggatactgatggattctcatcccttactgcaatgacaggattcgtcaaaaaggttcttttcgaacctgtgcctgagtaa

SEQ ID NO: 50 - DNA, 2952 bp, Phomopsis amygdali
  source 1..2952: mol_type = genomic DNA
atggagttcgatgaaccacttgtggacgaggcgaggtccttggtccaaagaaccctgcaagattatgacgaccgctatggctttggcactatgagctgtgcggcctatgacacagcatgggtatcgctggtgactaaaacagtcgatgggcgtaaacaatggttgttccctgagtgcttcgaatttctcctagaaacgcagtccgatgctggcggctgggaaatcggcaacagcgcacccatcgatgggatccttaacactgctgcttcactgctggcattgaagcgccacgtccaaacagagcagattattcagccgcaacacgaccataaagacctggccgggcgtgcggaaagagcggcggcgtctttgcgagcacagttggcggctctggatgtgtcgacaacggagcatgtgggcttcgaaatcatcgtcccggccatgctcgaccctctcgaggccgaagacccgtctttggtgttcgactttccagcacgcaaaccactgatgaagatccacgacgctaagatgtcgcgattccgaccagagtacctctacggtaaacagccgatgacggcattgcattcgctcgaggcctttatcgggaaaatagacttcgacaaagtacggcatcacaggacacacggttcgatgatggggtcgccctcgtcgacggctgcatacctgatgcatgcttctcagtgggacggcgactctgaggcctatctacgccatgtcatcaagcacgcagctggccagggcaccggagctgttccgagtgcatttccttcgacgcatttcgagtcttcttggattttgacaacattgtttcgagctgggttctcagcctctcatctagcatgcgacgaattaaacaagctggtagagatcctcgaaggctcatttgagaaagaagggggagccatcggttatgctcctgggtttcaagcagatgtggatgataccgcaaagaccatctccactttggctgtgcttgggagagatgccactccccggcaaatgatcaaggtttttgaagccaatacacactttcggacttaccctggtgaaagagatccaagcttgactgccaattgcaacgcgctctcggctcttcttcaccagccagacgcagcaatgtacggcagccagatccagaagatcacaaagtttgtttgtgactactggtggaaaagtgacggcaaaatcaaggacaagtggaatacctgctacttgtatccatcggtcctcctcgtcgaggtgttagtagaccttgtgtccctgttggagcaaggaaagctacccgacgtgctggatcaggagctgcaatacagggtcgccattacgttattccaggcctgcttgcgaccgctacttgatcaagatgctgaaggttcatggaacaaatccattgaagccacagcctacggcattctaatccttacggaggcgcggcgagtatgcttttttgaccgtctgagtgagcctctgaatgaggctattcgacgcgggattgcgtttgcagattcgatgagcggtactgaagctcagctgaattatatatggatcgagaaagtgagctacgcacctgctcttctgaccaaatcatacctcctcgcagctcggtgggcggcaaagtccccgcttggcgcttccgttggatccagcctttggacgcctccaagagaaggcttggataagcacgtccgtctattccaccaggcagagctcttcaggtcgttgccggagtgggagctgcgcgcgtccatgatcgaggcagccctgttcactcctttgctgcgtgcgcataggctggatgtatttccacgccaagacgtcggcgaggacaagtacctggacgttgtgccgttcttctggacggccgccaataaccgcgatcgcacgtacgcatccactctgtttctgtatgacatgtgctttatcgccatgcttaacttccagctggatgagttcatggaggctacagcgggaatcctcttccgggaccatatggatgatttgcgccaactcatccacgacctgcttgccgaaaagacgagccccaagtcatcgggcagaagtagccaaggaaccaaagacgcggactcgggcatcgaagaagacgtttctatgagcgactcagcgtcagactcccaggaccgcagccctgaatacgacctggtcttctctgcgctctctaccttcaccaaacatgtcctgcagcacccttcaatccagtcagccagtgtctgggataggaaactactcgctcgcgagatgaaagcatacctcctagctcatattcaacaggctgaggacagcacgcccttgagtgagctcaaggacgtccctcaaaaaactgacgtgacacgcgtctcaacgtccacaacgactttcttcaactgggtacgcacaacatccgcagaccacatatcctgcccatattcattccatttcgtggcgtgtcacctcggcgccgcgctgagccccaagggcagcaacggcgactgttacccgtcagccggtgaaaagttcctcgcggccgccgtatgccgccatttggccacgatgtgccgcatgtacaatgacttgggatcggcggagcgcgacagtgacgagggaaatttgaattcactcgactttcccgagttcgccgactcagcggggaatggtgggattgagatccagaaagctgccttgctcaggctggccgagttcgaacgcgactcgtatctcgaggctttccggcgacttcaggatgaaagcaaccgcgttcacggaccggctggtggggatgaagccagactcagcaggcggcgcatggccatccttgagttctttgcccagcaggtggacttgtatggccaggtctacgttattcgcgatatctcggccaggattccaaagaacgaggttgagaagaaaaggaaactagatgatgctttcaattag

SEQ ID NO: 51 - DNA, 2646 bp, Physcomitrella patens
  source 1..2646: mol_type = genomic DNA
atggcttccagcaccttgatacagaatcgctcttgtggcgttacgtcaagcatgtcttcctttcagatttttcgagggcaacctctacgttttccaggcactagaactcctgctgcagttcaatgcctaaagaagcgtcgatgtttgcgacctactgaatcagtcctcgagagctctcctggtagcggatcttacaggattgtaactggaccctccggcatcaatccttcttcaaacggccacttgcaagaggggtcccttactcacagacttccgatacccatggaaaaatccattgataacttccagtctactttgtacgtatcagacatatggtcagaaaccttgcaaagaacggaatgtttgttgcaggtgactgagaatgtacagatgaacgagtggattgaggaaatcagaatgtacttccgaaatatgacactgggggaaatatccatgtctccatacgacacagcttgggtagcgcgagtgccagcgctggatggctcacatggccctcagttccatcggtctttgcagtggattattgataatcagctcccggatggcgattggggtgaaccgtctcttttccttggatacgatcgcgtttgcaacactctcgcctgtgtaattgccctgaaaacttggggtgttggggctcagaacgtagagcgtggaatccagtttctgcaatctaacatctacaaaatggaggaagatgacgccaatcatatgccgattggatttgagattgtcttcccagcgatgatggaagatgccaaggcactgggactggatttaccatacgatgccactatcttgcaacaaatctcggctgaaagagagaagaaaatgaaaaagattcctatggcgatggtgtacaagtaccccactactttgctgcattctctggaaggcctgcaccgggaagtggactggaacaagctcctccagctacagtccgagaatggctcctttctgtattcacccgcatccactgcatgcgcacttatgtacacaaaagatgtgaagtgcttcgactacttgaaccagctcctcatcaagttcgaccacgcttgtccaaacgtgtaccccgttgatctcttcgagcgtttgtggatggtagaccgcctacaaaggctgggaatatcccgctacttcgagcgagaaatcagagactgtctacaatatgtataccgatactggaaggattgtggtattggctgggcaagcaattcgtccgtgcaggacgtggacgacacggccatggccttccgccttctccgcacacacggattcgacgtcaaggaggactgcttcagacagtttttcaaagatggtgagttcttctgcttcgccggccagtccagccaagccgtcacgggaatgttcaacctcagcagagcatcgcaaacgctcttcccaggggaatcactcctaaaaaaggccagaaccttttccagaaactttttgagaaccaagcatgaaaacaatgaatgcttcgacaagtggataatcacgaaggatctagcgggcgaggtggaatacaatctcacattcccctggtatgctagccttcctcgtcttgagcatcgcacctacttggaccaatatgggattgatgatatctggattggcaagtcgctctacaaaatgccggccgtcaccaacgaagtgtttctcaaattggccaaagccgacttcaacatgtgccaagctcttcacaagaaggaactcgagcaggtcatcaaatggaatgccagctgccaatttagagacctcgagtttgctagacagaaatccgtggagtgctacttcgcaggcgctgcaaccatgtttgagcccgaaatggtgcaggcgaggctcgtttgggcacgctgttgcgtgctcaccaccgttctagacgattacttcgatcacggtacacctgtggaagagcttcgggtttttgtgcaggccgtaaggacttggaatcccgagctcatcaacggactacctgagcaagccaagattctctttatgggactgtacaagactgtgaacactatcgccgaggaggcattcatggcacagaaacgagacgtacatcatcatctcaagcattactgggacaaattgatcacttcagctttgaaagaagccgaatgggcagagtccggctacgtccccaccttcgacgagtatatggaagtcgctgaaatctccgtcgcactagagcccattgtatgtagcactctcttcttcgccggccataggctcgatgaggatgtgcttgacagttatgactaccatcttgtcatgcatctcgtcaaccgcgtaggtcgcatcctcaacgacatccaaggaatgaagagggaagccagccaagggaagatatcgagcgtgcagatctacatggaggagcatccaagtgtgccttcagaggccatggccatcgctcatctgcaggaattggtcgacaactccatgcaacagctgacatacgaagtgctgcgcttcactgcagtcccgaagtcctgtaagagaatccatttaaacatggcgaagatcatgcacgctttctacaaggacactgatgggttttcgtcactgacagccatgacagggtttgtgaagaaggtgctcttcgagccagtacctgaatag

SEQ ID NO: 52 - DNA, 1542 bp, synthetic construct
  misc_feature 1..1542: note = Description of Artificial Sequence: Synthetic polynucleotide
  source 1..1542: mol_type = other DNA
atggatgctgtgacgggtttgttaactgtcccagcaaccgctataactattggtggaactgctgtagcattggcggtagcgctaatcttttggtacctgaaatcctacacatcagctagaagatcccaatcaaatcatcttccaagagtgcctgaagtcccaggtgttccattgttaggaaatctgttacaattgaaggagaaaaagccatacatgacttttacgagatgggcagcgacatatggacctatctatagtatcaaaactggggctacaagtatggttgtggtatcatctaatgagatagccaaggaggcattggtgaccagattccaatccatatctacaaggaacttatctaaagccctgaaagtacttacagcagataagacaatggtcgcaatgtcagattatgatgattatcataaaacagttaagagacacatactgaccgccgtcttgggtcctaatgcacagaaaaagcatagaattcacagagatatcatgatggataacatatctactcaacttcatgaattcgtgaaaaacaacccagaacaggaagaggtagaccttagaaaaatctttcaatctgagttattcggcttagctatgagacaagccttaggaaaggatgttgaaagtttgtacgttgaagacctgaaaatcactatgaatagagacgaaatctttcaagtccttgttgttgatccaatgatgggagcaatcgatgttgattggagagacttctttccatacctaaagtgggtcccaaacaaaaagttcgaaaatactattcaacaaatgtacatcagaagagaagctgttatgaaatctttaatcaaagagcacaaaaagagaatagcgtcaggcgaaaagctaaatagttatatcgattaccttttatctgaagctcaaactttaaccgatcagcaactattgatgtccttgtgggaaccaatcattgaatcttcagatacaacaatggtcacaacagaatgggcaatgtacgaattagctaaaaaccctaaattgcaagataggttgtacagagacattaagtccgtctgtggatctgaaaagataaccgaagagcatctatcacagctgccttacattacagctattttccacgaaacactgagaagacactcaccagttcctatcattcctctaagacatgtacatgaagataccgttctaggcggctaccatgttcctgctggcacagaacttgccgttaacatctacggttgcaacatggacaaaaacgtttgggaaaatccagaggaatggaacccagaaagattcatgaaagagaatgagacaattgattttcaaaagacgatggccttcggtggtggtaagagagtttgtgctggttccttgcaagcccttttaactgcatctattgggattgggagaatggttcaagagttcgaatggaaactgaaggatatgactcaagaggaagtgaacacgataggcctaactacacaaatgttaagaccattgagagctattatcaaacctaggatctaa

SEQ ID NO: 53 - DNA, 1530 bp, synthetic construct
  misc_feature 1..1530: note = Description of Artificial Sequence: Synthetic polynucleotide
  source 1..1530: mol_type = other DNA
atggcatttttctctatgatttcaattttgttgggatttgttatttcttctttcatcttcatctttttcttcaaaaagttacttagttttagtaggaaaaacatgtcagaagtttctactttgccaagtgttccagtagtgcctggttttccagttattgggaatttgttgcaactaaaggagaaaaagcctcataaaactttcactagatggtcagagatatatggacctatctactctataaagatgggttcttcatctcttattgtattgaacagtacagaaactgctaaggaagcaatggtcactagattttcatcaatatctaccagaaaattgtcaaacgccctaacagttctaacctgcgataagtctatggtcgccacttctgattatgatgacttccacaaattagttaagagatgtttgctaaatggacttcttggtgctaatgctcaaaagagaaaaagacactacagagatgctttgattgaaaatgtgagttccaagctacatgcacacgctagagatcatccacaagagccagttaactttagagcaattttcgaacacgaattgtttggtgtagcattaaagcaagccttcggtaaagacgtagaatccatatacgtcaaggagttaggcgtaacattatcaaaagatgaaatctttaaggtgcttgtacatgatatgatggagggtgcaattgatgtagattggagagatttcttcccatatttgaaatggatccctaataagtcttttgaagctaggatacaacaaaagcacaagagaagactagctgttatgaacgcacttatacaggacagattgaagcaaaatgggtctgaatcagatgatgattgttaccttaacttcttaatgtctgaggctaaaacattgactaaggaacagatcgcaatccttgtctgggaaacaatcattgaaacagcagatactaccttagtcacaactgaatgggccatatacgagctagccaaacatccatctgtgcaagataggttgtgtaaggagatccagaacgtgtgtggtggagagaaattcaaggaagagcagttgtcacaagttccttaccttaacggcgttttccatgaaaccttgagaaaatactcacctgcaccattagttcctattagatacgcccacgaagatacacaaatcggtggctaccatgttccagctgggtccgaaattgctataaacatctacgggtgcaacatggacaaaaagagatgggaaagaccagaagattggtggccagaaagattcttagatgatggcaaatatgaaacatctgatttgcataaaacaatggctttcggagctggcaaaagagtgtgtgccggtgctctacaagcctccctaatggctggtatcgctattggtagattggtccaagagttcgaatggaaacttagagatggtgaagaggaaaatgtcgatacttatgggttaacatctcaaaagttatacccactaatggcaatcatcaatcctagaagatcctaa

SEQ ID NO: 54 - DNA, 1578 bp, synthetic construct
  misc_feature 1..1578: note = Description of Artificial Sequence: Synthetic polynucleotide
  source 1..1578: mol_type = other DNA
atgagtaagtctaatagtatgaattctacatcacacgaaaccctttttcaacaattggtcttgggtttggaccgtatgccattgatggatgttcactggttgatctacgttgctttcggcgcatggttatgttcttatgtgatacatgttttatcatcttcctctacagtaaaagtgccagttgttggatacaggtctgtattcgaacctacatggttgcttagacttagattcgtctgggaaggtggctctatcataggtcaagggtacaataagtttaaagactctattttccaagttaggaaattgggaactgatattgtcattataccacctaactatattgatgaagtgagaaaattgtcacaggacaagactagatcagttgaacctttcattaatgattttgcaggtcaatacacaagaggcatggttttcttgcaatctgacttacaaaaccgtgttatacaacaaagactaactccaaaattggtttccttgaccaaggtcatgaaggaagagttggattatgctttaacaaaagagatgcctgatatgaaaaatgacgaatgggtagaagtagatatcagtagtataatggtgagattgatttccaggatctccgccagagtctttctagggcctgaacactgtcgtaaccaggaatggttgactactacagcagaatattcagaatcacttttcattacagggtttatcttaagagttgtacctcatatcttaagaccattcatcgcccctctattaccttcatacaggactctacttagaaacgtttcaagtggtagaagagtcatcggtgacatcataagatctcagcaaggggatggtaacgaagatatactttcctggatgagagatgctgccacaggagaggaaaagcaaatcgataacattgctcagagaatgttaattctttctttagcatcaatccacactactgcgatgaccatgacacatgccatgtacgatctatgtgcttgccctgagtacattgaaccattaagagatgaagttaaatctgttgttggggcttctggctgggacaagacagcgttaaacagatttcataagttggactccttcctaaaagagtcacaaagattcaacccagtattcttattgacattcaatagaatctaccatcaatctatgaccttatcagatggcactaacattccatctggaacacgtattgctgttccatcacacgcaatgttgcaagattctgcacatgtcccaggtccaaccccacctactgaatttgatggattcagatatagtaagatacgttctgatagtaactacgcacaaaagtacctattctccatgaccgattcttcaaacatggctttcggatacggcaagtatgcttgtccaggtagattttacgcgtctaatgagatgaaactaacattagccattttgttgctacaatttgagttcaaactaccagatggtaaaggtcgtcctagaaatatcactatcgattctgatatgattccagacccaagagctagactttgcgtcagaaaaagatcacttagagatgaatga

SEQ ID NO: 55 - DNA, 1500 bp, synthetic construct
  misc_feature 1..1500: note = Description of Artificial Sequence: Synthetic polynucleotide
  source 1..1500: mol_type = other DNA
atggaagatcctactgtcttatatgcttgtcttgccattgcagttgcaactttcgttgttagatggtacagagatccattgagatccatcccaacagttggtggttccgatttgcctattctatcttacatcggcgcactaagatggacaagacgtggcagagagatacttcaagagggatatgatggctacagaggatctacattcaaaatcgcgatgttagaccgttggatcgtgatcgcaaatggtcctaaactagctgatgaagtcagacgtagaccagatgaagagttaaactttatggacggattaggagcattcgtccaaactaagtacaccttaggtgaagctattcataacgatccataccatgtcgatatcataagagaaaaactaacaagaggccttccagccgtgcttcctgatgtcattgaagagttgacacttgcggttagacagtacattccaacagaaggtgatgaatgggtgtccgtaaactgttcaaaggccgcaagagatattgttgctagagcttctaatagagtctttgtaggtttgcctgcttgcagaaaccaaggttacttagatttggcaatagactttacattgtctgttgtcaaggatagagccatcatcaatatgtttccagaattgttgaagccaatagttggcagagttgtaggtaacgccaccagaaatgttcgtagagctgttccttttgttgctccattggtggaggaaagacgtagacttatggaagagtacggtgaagactggtctgaaaaacctaatgatatgttacagtggataatggatgaagctgcatccagagatagttcagtgaaggcaatcgcagagagattgttaatggtgaacttcgcggctattcatacctcatcaaacactatcactcatgctttgtaccaccttgccgaaatgcctgaaactttgcaaccacttagagaagagatcgaaccattagtcaaagaggagggctggaccaaggctgctatgggaaaaatgtggtggttagattcatttctaagagaatctcaaagatacaatggcattaacatcgtatctttaactagaatggctgacaaagatattacattgagtgatggcacatttttgccaaaaggtactctagtggccgttccagcgtattctactcatagagatgatgctgtctacgctgatgccttagtattcgatcctttcagattctcacgtatgagagcgagagaaggtgaaggtacaaagcaccagttcgttaatacttcagtcgagtacgttccatttggtcacggaaagcatgcttgtccaggaagattcttcgccgcaaacgaattgaaagcaatgttggcttacattgttctaaactatgatgtaaagttgcctggtgacggtaaacgtccattgaacatgtattggggtccaacagttttgcctgcaccagcaggccaagtattgttcagaaagagacaagttagtctataa

SEQ ID NO: 56 - DNA, 1542 bp, Stevia rebaudiana
  source 1..1542: mol_type = genomic DNA
atggatgccgtcaccggtttgctgacagttccggcaaccgcaataaccatcggcggtacggccgtcgcactcgccgtcgctctgatattctggtacctcaaaagctacacatctgcacgcaggagccaatcaaaccatctccctcgggttcccgaggtacctggtgtgccattattggggaatttattgcagttgaaggagaagaaaccttacatgacttttacaagatgggcggcaacttatggtccgatttattcgattaaaaccggagcaacttctatggtggtcgtcagttcaaatgaaattgcaaaggaggcattggttaccagatttcaatctatctcaaccagaaacctatcaaaggcattaaaggttctcacagcagataaaaccatggtggcgatgagtgattatgatgattatcataagactgtcaaacgccatatactgaccgctgttttgggaccaaatgctcagaagaaacaccgcatccatagggacatcatgatggataatatatcaacccaacttcatgaatttgttaaaaataatcctgaacaagaggaagtggatctaaggaaaatattccaatccgaactttttggattagctatgagacaagcattgggaaaggatgtggagagcttatatgttgaggatcttaaaatcaccatgaaccgagacgagatatttcaggtattggttgttgacccgatgatgggtgcaattgacgtcgactggagagatttcttcccgtatctaaagtgggtcccgaataaaaagtttgaaaacacgatccaacaaatgtatatccggagagaagctgtgatgaagtctcttattaaagaacataaaaaacgtattgcatccggagagaaattaaacagctacattgattacttgctatcggaagcacaaacgttaaccgatcaacaactacttatgtctctatgggaacctattattgaatcatcagacaccactatggttacaactgaatgggctatgtatgaacttgcaaaaaaccccaaacttcaggatcgtttgtatcgggatatcaaaagtgtttgcgggtcagagaagattacagaagaacacttgtctcaactgccatacataactgccatttttcatgaaaccttgagaaggcatagtccagttcctataattccattaagacacgtgcatgaagacacagtgttaggagggtaccatgtgccagctggaaccgagctagcggtaaacatttatggatgtaacatggataagaatgtgtgggagaatcctgaagaatggaatccagagagattcatgaaggaaaatgaaacgatagatttccagaaaacaatggcgtttggaggtggaaagcgcgtatgtgctggttcgcttcaagcattgttgactgcttccattggaattggaagaatggtgcaagagtttgagtggaaactgaaagayatgacccaagaagaagttaatacgattgggcttacgacccagatgcttcgtccactgcgggccataataaagcccaggatatga

SEQ ID NO: 57 - DNA, 1530 bp, Arabidopsis thaliana
  source 1..1530: mol_type = genomic DNA
atggccttcttctccatgatctccattctccttggctttgttatctcctccttcatcttcatcttcttcttcaagaaacttctctccttctccagaaagaacatgtctgaagtctccactctcccctctgttccagtggtaccagggtttcctgttattgggaacttgctgcaactaaaagagaagaaacctcacaagactttcactagatggtcagagatttatggtcctatttactctataaagatgggttcttcttctcttattgtcctcaattctactgagactgccaaagaggccatggtgacgcggttttcgtctatctcaacgaggaagttgtcaaatgcgttgacagtccttacttgtgacaaatctatggttgctactagtgattatgatgatttccacaagttggtgaaacggtgtctcttgaacggtcttttgggtgctaatgcacagaaacgaaaaagacattacagagatgcactcattgaaaatgtgtcttccaagttgcatgcccatgctagggaccatccacaagaacctgtaaacttcagagctatatttgagcatgagcttttcggtgtagcattgaagcaagcttttgggaaagatgtggaatccatttatgttaaagaactcggtgtgactttgtcgaaagacgagatcttcaaggttttagtacatgacatgatggaaggtgcaattgatgttgattggagagacttcttcccatacttgaaatggattccaaataaaagttttgaagcaagaatccagcaaaagcataaacgtagactcgcggtgatgaatgctctgattcaagatcgactgaagcagaatggttcagaatcggatgatgattgctatctcaacttcttgatgtcggaagcgaaaacactaaccaaggagcaaattgctatcttggtttgggagacgattatcgagacagctgacactactttggttacaactgaatgggccatctatgagctcgctaagcatccaagtgtccaagatcgtctgtgtaaagaaatccaaaatgtctgcggaggagaaaagttcaaagaagagcaattgtctcaagttccttatctcaatggagtattccatgaaacgcttaggaaatacagtcctgctcctctagtccccattcgctatgcccacgaagatacgcaaatcggaggctatcatgtccctgcaggaagtgagattgcaataaacatctatggatgcaacatggataagaagcgttgggagagaccagaggactggtggccggagcggtttcttgatgatggcaaatacgaaacgtcggatcttcacaagacaatggcgtttggagcgggaaagagggtttgtgctggtgctcttcaagcatctctcatggcaggcattgccattgggaggttagtgcaagaattcgagtggaagcttagagacggtgaagaagagaatgtggatacatatggcttgacctctcagaagctttatcctcttatggctattatcaatccaaggcgttcttaa

SEQ ID NO: 58 - DNA, 1578 bp, Gibberella fujikoroi
  source 1..1578: mol_type = genomic DNA
atgagtaagtccaacagcatgaacagtaccagccatgaaacgttattccagcagctcgtcttaggtcttgacagaatgccgctaatggacgttcactggctgatctacgtggcctttggcgcttggttatgctcttatgtcatccatgtcctatcgtcctcttctacagtcaaagtgcccgtcgtaggctaccgcagcgtctttgagcctacatggcttctccgtttgcgctttgtttgggaagggggatctatcatcggccaaggctacaacaaatttaaagactctatcttccaggtgcgaaagcttggtaccgatatcgtcatcatcccgccaaactacatcgatgaggtcagaaagctgtcccaagacaagactcgctcggtcgagcccttcatcaatgactttgcgggacagtatacacggggcatggtctttctgcaaagtgatttgcagaaccgtgtgattcagcagcggttgacgccaaaactcgtatcgttgacaaaggtaatgaaggaggagcttgactatgccttgaccaaagagatgcctgacatgaagaatgatgaatgggttgaagtcgacatttcttccatcatggtcaggctcatatcacgcatctcagccagagtgtttctcggtccagagcactgccgcaaccaagaatggttgacgaccactgcagagtacagcgagagcctgttcataactggctttattctccgcgttgtcccccatattctaagaccattcatagccccgctgctaccctcctacagaacactacttcgcaacgtctcgtcaggtcgaagagttattggagacatcattcgctcccagcaaggtgatggcaacgaggacatcctgtcatggatgagggatgctgcgacaggggaagaaaagcaaattgacaacattgcccagcggatgcttatcctgagtctcgcgtctattcacactacggcaatgacgatgacgcatgctatgtatgacttatgtgcttgccctgagtacatagagcctcttagagatgaggtcaaaagtgtcgttggcgctagtggttgggacaagacggcgttgaatcgattccacaaactcgacagctttctcaaagagtcacaacgcttcaaccccgtgttcctcttaacgttcaatcgcatttatcaccaatccatgacactctcagatggcaccaacatcccatcaggcactcgcatcgcggttccctctcacgcgatgcttcaggactcagcgcatgtcccaggcccgacgccaccaaccgagtttgatggatttagatactcaaagattcgctcagactcaaactatgcacagaaatatctcttctccatgactgattctagtaacatggcgtttgggtatgggaaatacgcctgcccagggcggttctatgcatctaatgagatgaagctgactttggcgatactccttttacaatttgagttcaagttgccagatgggaaaggaagaccacgaaatatcactattgatagtgacatgatacctgatccgagagctaggctgtgcgttaggaagcgatcactgagagatgaatga

SEQ ID NO: 59 - DNA, 1500 bp, Trametes versicolor
  source 1..1500: mol_type = genomic DNA
atggaggatcccaccgtactctacgcttgcctcgccatcgctgtcgctactttcgttgtcagatggtacagagacccgcttcggtccattcctacggttgggggctctgaccttcccatcctctcatacatcggggcgctcaggtggacccgccgcggaagagagatactgcaagaaggttatgatgggtatcgcggatccacgttcaagatcgcgatgctcgaccggtggatcgtcatcgccaacggcccaaagctcgccgacgaggtgaggaggcgtcctgacgaagagctaaacttcatggacggactgggagcgttcgtgcagacgaagtatacccttggggaagcaatccacaatgacccgtaccacgtggacattattcgtgagaagctgacgcgaggcctcccggcagtcctgccggacgtcatcgaggaactcacgctagccgttcgccagtacatcccgacggaaggagatgaatgggtcagcgtgaactgctccaaagcagcgcgggacatcgtcgcccgggcaagcaaccgcgtctttgtcgggttgcccgcttgccgcaaccagggttatctcgacctcgccattgacttcaccctgagcgttgtcaaagacagggcgatcatcaatatgttcccggagttgctgaaacctatcgtcggacgcgtggttggaaatgccactaggaacgtgcgccgcgcggtcccattcgtagcgccgttggtggaggaacgtcgccgcctcatggaggagtacggtgaggattggtcggagaaaccgaacgacatgctccagtggatcatggacgaggcagcctcgcgggactcctccgtcaaagcgatcgctgagcgtcttctcatggtcaactttgccgcaattcacacgtcgtcgaacaccatcacccacgctctttaccacctcgccgagatgccggagaccctacagccgctgcgggaagagatcgagccgctcgtcaaggaagaaggctggacgaaggccgccatgggcaagatgtggtggctcgacagcttcctgcgggagtcacagcgctacaatggcatcaacatcgtctccctgacgcgcatggccgacaaggacataacgctcagcgacggcacgttcctcccgaagggcacgctcgtcgcggtccccgcgtactcgacgcaccgcgacgacgcggtgtacgcggacgcgctggtcttcgacccgttccgcttctcccgcatgcgcgcccgcgagggcgagggcacgaagcaccagttcgtcaacacctccgtggagtacgtgcccttcggccacgggaagcacgcctgccccgggcggttcttcgcggccaacgagctgaaggcgatgctcgcgtacatcgtgctcaactacgacgtgaagctgcccggcgatggcaagcgccccctgaacatgtactggggcccgacggtcttgcctgctccggctgggcaggtgctcttccgcaagaggcaggtgtcgctgtag

SEQ ID NO: 60 - DNA, 1578 bp, synthetic construct
  misc_feature 1..1578: note = Description of Artificial Sequence: Synthetic polynucleotide
  source 1..1578: mol_type = other DNA
atgggtttgttcccattagaggattcctacgcgctggtctttgaaggactagcaataacactggctttgtactatctactgtctttcatctacaaaacatctaaaaagacatgtacacctcctaaagcatctggtgaaatcattccaattacaggaatcatattgaatctgctatctggctcaagtggtctacctattatcttagcacttgcctctttagcagacagatgtggtcctattttcaccattaggctgggtattaggagagtgctagtagtatcaaattgggaaatcgctaaggagattttcactacccacgatttgatagtttctaatagaccaaaatacttagccgctaagattcttggtttcaattatgtttcattctctttcgctccatacggcccatattgggtcggaatcagaaagattattgctacaaaactaatgtcttcttccagacttcagaagttgcaatttgtaagagtttttgaactagaaaactctatgaaatctatcagagaatcatggaaggagaaaaaggatgaagagggaaaggtattagttgagatgaaaaagtggttctgggaactgaatatgaacatagtgttaaggacagttgctggtaaacaatacactggtacagttgatgatgccgatgcaaagcgtatctccgagttattcagagaatggtttcactacactggcagatttgtcgttggagacgcttttccttttctaggttggttggacctgggcggatacaaaaagacaatggaattagttgctagtagattggactcaatggtcagtaaatggttagatgagcatcgtaaaaagcaagctaacgatgacaaaaaggaggatatggatttcatggatatcatgatctccatgacagaagcaaattcaccacttgaaggatacggcactgatactattatcaagaccacatgtatgactttgattgtttcaggagttgatacaacctcaatcgtacttacttgggccttatcacttttgttaaacaacagagatactttgaaaaaggcacaagaggaattagatatgtgcgtaggtaaaggaagacaagtcaacgagtctgatcttgttaacttgatatacttggaagcagtgcttaaagaggctttaagactttacccagcagcgttcttaggcggaccaagagcattcttggaagattgtactgttgctggttatagaattccaaagggcacctgcttgttgattaacatgtggaaactgcatagagatccaaacatttggagtgatccttgcgaattcaagccagaaagatttttgacacctaatcaaaaggatgttgatgtgatcggtatggatttcgaattgataccatttggtgccggcagaagatattgtccaggtactagattggctttacagatgttgcatatcgtattagcgacattgctgcaaaacttcgaaatgtcaacaccaaacgatgcgccagtcgatatgactgcttctgttggcatgacaaatgccaaagcatcacctttagaagtcttgctatcacctcgtgttaaatggtcctaa

SEQ ID NO: 61 - DNA, 1431 bp, synthetic construct
  misc_feature 1..1431: note = Description of Artificial Sequence: Synthetic polynucleotide
  source 1..1431: mol_type = other DNA
atgatacaagttttaactccaattctactcttcctcatcttcttcgttttctggaaagtctacaaacatcaaaagactaaaatcaatctaccaccaggttccttcggctggccatttttgggtgaaaccttagccttacttagagcaggctgggattctgagccagaaagattcgtaagagagcgtatcaaaaagcatggatctccacttgttttcaagacatcactatttggagacagattcgctgttctttgcggtccagctggtaataagtttttgttctgcaacgaaaacaaattagtggcatcttggtggccagtccctgtaaggaagttgttcggtaaaagtttactcacaataagaggagatgaagcaaaatggatgagaaaaatgctattgtcttacttgggtccagatgcatttgccacacattatgccgttactatggatgttgtaacacgtagacatattgatgtccattggaggggcaaggaggaagttaatgtatttcaaacagttaagttgtacgcattcgaattagcttgtagattattcatgaacctagatgacccaaaccacatcgcgaaactcggtagtcttttcaacattttcctcaaagggatcatcgagcttcctatagacgttcctggaactagattttactccagtaaaaaggccgcagctgccattagaattgaattgaaaaagctcattaaagctagaaaactcgaattgaaggagggtaaggcgtcttcttcacaggacttgctttctcatctattaacatcacctgatgagaatgggatgttcttgacagaagaggaaatagtcgataacattctacttttgttattcgctggtcacgatacctctgcactatcaataacacttttgatgaaaaccttaggtgaacacagtgatgtgtacgacaaggttttgaaggaacaattagaaatttccaaaacaaaggaggcttgggaatcactaaagtgggaagatatccagaagatgaagtactcatggtcagtaatctgtgaagtcatgagattgaatcctcctgtcatagggacatacagagaggcgttggttgatatcgactatgctggttacactatcccaaaaggatggaagttgcattggtcagctgtttctactcaaagagacgaagccaatttcgaagatgtaactagattcgatccatccagatttgaaggggcaggccctactccattcacatttgtgcctttcggtggaggtcctagaatgtgtttaggcaaagagtttgccaggttagaagtgttagcatttctccacaacattgttaccaactttaagtgggatcttctaatccctgatgagaagatcgaatatgatccaatggctactccagctaagggcttgccaattagacttcatccacaccaagtctaa

SEQ ID NO: 62 - DNA, 1578 bp, synthetic construct
  misc_feature 1..1578: note = Description of Artificial Sequence: Synthetic polynucleotide
  source 1..1578: mol_type = other DNA
atggagtctttagtggttcatacagtaaatgctatctggtgtattgtaatcgtcgggattttctcagttggttatcacgtttacggtagagctgtggtcgaacaatggagaatgagaagatcactgaagctacaaggtgttaaaggcccaccaccatccatcttcaatggtaacgtctcagaaatgcaacgtatccaatccgaagctaaacactgctctggcgataacattatctcacatgattattcttcttcattattcccacacttcgatcactggagaaaacagtacggcagaatctacacatactctactggattaaagcaacacttgtacatcaatcatccagaaatggtgaaggagctatctcagactaacacattgaacttgggtagaatcacccatataaccaaaagattgaatcctatcttaggtaacggaatcataacctctaatggtcctcattgggcccatcagcgtagaattatcgcctacgagtttactcatgataagatcaagggtatggttggtttgatggttgagtctgctatgcctatgttgaataagtgggaggagatggtaaagagaggcggagaaatgggatgcgacataagagttgatgaggacttgaaagatgtttcagcagatgtgattgcaaaagcctgtttcggatcctcattttctaaaggtaaggctattttctctatgataagagatttgcttacagctatcacaaagagaagtgttctattcagattcaacggattcactgatatggtctttgggagtaaaaagcatggtgacgttgatatagacgctttagaaatggaattggaatcatccatttgggaaactgtcaaggaacgtgaaatagaatgtaaagatactcacaaaaaggatctgatgcaattgattttggaaggggcaatgcgttcatgtgacggtaacctttgggataaatcagcatatagaagatttgttgtagataattgtaaatctatctacttcgcagggcatgatagtacagctgtctcagtgtcatggtgtttgatgttactggccctaaacccatcatggcaagttaagatccgtgatgaaattctgtcttcttgcaaaaatggtattccagatgccgaaagtatcccaaaccttaaaacagtgactatggttattcaagagacaatgagattataccctccagcaccaatcgtcgggagagaagcctctaaagatatcagattgggcgatctagttgttcctaaaggcgtctgtatatggacactaataccagctttacacagagatcctgagatttggggaccagatgcaaacgatttcaaaccagaaagattttctgaaggaatttcaaaggcttgtaagtatcctcaaagttacattccatttggtctgggtcctagaacatgcgttggtaaaaactttggcatgatggaagtaaaggttcttgtttccctgattgtctccaagttctctttcactctatctcctacctaccaacatagtcctagtcacaaacttttagtagaaccacaacatggggtggtaattagagtggtttaa

SEQ ID NO: 63 - DNA, 1590 bp, synthetic construct
  misc_feature 1..1590: note = Description of Artificial Sequence: Synthetic polynucleotide
  source 1..1590: mol_type = other DNA
atgtacttcctactacaatacctcaacatcacaaccgttggtgtctttgccacattgtttctctcttattgtttacttctctggagaagtagagcgggtaacaaaaagattgccccagaagctgccgctgcatggcctattatcggccacctccacttacttgcaggtggatcccatcaactaccacatattacattgggtaacatggcagataagtacggtcctgtattcacaatcagaataggcttgcatagagctgtagttgtctcatcttgggaaatggcaaaggaatgttcaacagctaatgatcaagtgtcttcttcaagacctgaactattagcttctaagttgttgggttataactacgccatgtttggtttttcaccatacggttcatactggagagaaatgagaaagatcatctctctcgaattactatctaattccagattggaactattgaaagatgttagagcctcagaagttgtcacatctattaaggaactatacaaattgtgggcggaaaagaagaatgagtcaggattggtttctgtcgagatgaaacaatggttcggagatttgactttaaacgtgatcttgagaatggtggctggtaaaagatacttctccgcgagtgacgcttcagaaaacaaacaggcccagcgttgtagaagagtcttcagagaattcttccatctctccggcttgtttgtggttgctgatgctataccttttcttggatggctcgattggggaagacacgagaagaccttgaaaaagaccgccatagaaatggattccatcgcccaggagtggcttgaggaacatagacgtagaaaagattctggagatgataattctacccaagatttcatggacgttatgcaatctgtgctagatggcaaaaatctaggcggatacgatgctgatacgattaacaaggctacatgcttaactcttatatcaggtggcagtgatactactgtagtttctttgacatgggctcttagtcttgtgttaaacaatagagatactttgaaaaaggcacaggaagagttagacatccaagtcggtaaggaaagattggttaacgagcaagacatcagtaagttagtttacttgcaagcaatagtaaaagagacactcagactttatccaccaggtcctttgggtggtttgagacaattcactgaagattgtacactaggtggctatcacgtttcaaaaggaactagattaatcatgaacttatccaagattcaaaaagatccacgtatttggtctgatcctactgaattccaaccagagagattccttacgactcataaagatgtcgatccacgtggtaaacactttgaattcattccattcggtgcaggaagacgtgcatgtcctggtatcacattcggattacaagtactacatctaacattggcatctttcttgcatgcgtttgaattttcaacaccatcaaatgagcaggttaacatgagagaatcattaggtcttacgaatatgaaatctaccccattagaagttttgatttctccaagactatcccttaattgcttcaaccttatgaaaatttga

SEQ ID NO: 64 - DNA, 1440 bp, synthetic construct
  misc_feature 1..1440: note = Description of Artificial Sequence: Synthetic polynucleotide
  source 1..1440: mol_type = other DNA
atggaacctaacttttacttgtcattactattgttgttcgtgaccttcatttctttaagtctgtttttcatcttttacaaacaaaagtccccattgaatttgccaccagggaaaatgggttaccctatcataggtgaaagtttagaattcctatccacaggctggaagggacatcctgaaaagttcatatttgatagaatgcgtaagtacagtagtgagttattcaagacttctattgtaggcgaatccacagttgtttgctgtggggcagctagtaacaaattcctattctctaacgaaaacaaactggtaactgcctggtggccagattctgttaacaaaatcttcccaacaacttcactggattctaatttgaaggaggaatctataaagatgagaaagttgctgccacagttcttcaaaccagaagcacttcaaagatacgtcggcgttatggatgtaatcgcacaaagacattttgtcactcactgggacaacaaaaatgagatcacagtttatccacttgctaaaagatacactttcttgcttgcgtgtagactgttcatgtctgttgaggatgaaaatcatgtggcgaaattctcagacccattccaactaatcgctgcaggcatcatttcacttcctatcgatcttcctggtactccattcaacaaggccataaaggcttcaaatttcattagaaaagagctgataaagattatcaaacaaagacgtgttgatctggcagagggtacagcatctccaacccaggatatcttgtcacatatgctattaacatctgatgaaaacggtaaatctatgaacgagttgaacattgccgacaagattcttggactattgataggaggccacgatacagcttcagtagcttgcacatttctagtgaagtacttaggagaattaccacatatctacgataaagtctaccaagagcaaatggaaattgccaagtccaaacctgctggggaattgttgaattgggatgacttgaaaaagatgaagtattcatggaatgtggcatgtgaggtaatgagattgtcaccacctttacaaggtggttttagagaggctataactgactttatgtttaacggtttctctattccaaaagggtggaagttatactggtccgccaactctacacacaaaaatgcagaatgtttcccaatgcctgagaaattcgatcctaccagatttgaaggtaatggtccagcgccttatacatttgtaccattcggtggaggccctagaatgtgtcctggaaaggaatacgctagattagaaatcttggttttcatgcataatctggtcaaacgttttaagtgggaaaaggttattccagacgaaaagattattgtcgatccattcccaatcccagctaaagatcttccaatccgtttgtatcctcacaaagcttaa

SEQ ID NO: 65 - DNA, 1572 bp, Stevia rebaudiana
  source 1..1572: mol_type = genomic DNA
atgggtctcttccctttggaagatagttacgcactcgtctttgaaggtttagcaataactactctagctctctactacttattatccttcatctataaaacctctaaaaagacttgtactccacctaaagcaagcggtgagcaccctataacaggccacttaaaccttcttagtggttcatccggtcttccccatctagccttagcatctttggctgaccgatgtgggcccatattcaccatccgacttggcatacgtagagttttggtggttagtaattgggaaattgctaaggagatcttcactacccatgatttgattgtttcaaaccgtcccaaatacctcgctgcaaagattttgggattcaactatgtgtccttttcgtttgctccatatggcccctattgggttggaatccgtaagatcatcgccacaaaactgatgtcaagtagcaggctccagaagcttcagtttgtccgagttttcgaactagaaaactccatgaaaagcatacgcgagtcttggaaagagaaaaaagacgaagaaggtaaagtgttggtggagatgaaaaaatggttttgggaattgaatatgaatatagttcttagaactgttgctggtaaacagtacactggaactgttgatgatgcggatgcgaagaggattagtgaattgtttagagaatggtttcattacacaggaaggtttgttgtgggagatgcttttccttttcttgggtggttggatttgggtggatataagaagaccatggaactagtggcttccagactagattccatggtctcaaaatggttagacgagcatcgcaaaaagcaggctaacgacgacaaaaaagaggacatggatttcatggacatcatgatatcgatgactgaagccaattcccctttggagggttatggtacggatacaataattaaaaccacttgcatgactcttattgtcagtggtgtagatacaacctccatcgtgctaacttgggcactctcgttactactgaacaaccgtgacactcttaagaaagctcaagaagagctagacatgtgtgtgggaaaaggtcgacaagtaaacgaatcagatctagtaaacctaatctaccttgaagccgtattaaaagaagcattgcgactatacccagcagcattccttggaggtcctagagcctttttagaagactgcaccgtggcagggtaccgtatcccaaaaggcacatgtctacttattaacatgtggaaacttcatcgtgatccaaacatatggtcagacccatgtgagtttaaaccagagaggttcttaaccccaaaccaaaaggacgtagatgttattggaatggattttgagttaatcccatttggtgcgggaagaaggtattgtccagggacacgtttggcattacaaatgttacacatagttctggccactctactacaaaactttgagatgtcaactccaaatgatgcacccgttgatatgaccgcgagtgttggaatgacaaatgcgaaggcaagtccacttgaagttctactttcgccacgtgttaagtggtcatag

SEQ ID NO: 66 - DNA, 1431 bp, Stevia rebaudiana
  source 1..1431: mol_type = genomic DNA
atgattcaagttctaacaccgatccttctcttcctcattttcttcgttttctggaaggtttacaagcaccagaaaaccaaaatcaatcttccaccgggaagcttcggatggccatttctgggcgaaactctggcactcctacgtgcaggttgggactcagagccggagagatttgttcgtgaacggatcaagaaacacggaagtcctctagtgtttaagacgtcgttgtttggcgaccgttttgcggtgttgtgtggacctgccggaaacaagttcctgttctgcaacgagaacaagctggtggcgtcgtggtggccggttccggtgaggaagcttttcggcaagtctctgctcacgattcgtggtgatgaagctaagtggatgaggaagatgttgttatcgtatctcggtcctgatgctttcgcaactcattatgccgtcaccatggacgtcgtcacccgtcggcatatcgacgttcattggcgagggaaggaagaggtgaacgtattccaaaccgttaagttatatgcctttgagcttgcatgtcgtttattcatgaacctagacgacccaaaccacattgcaaaactcggttccttgttcaacattttcttgaaaggcatcattgagcttccaatcgacgtcccagggacacgattttatagctccaaaaaagcagcagcagctatcaggattgaactaaaaaaattgattaaagcaagaaaactggaactgaaagaagggaaggcatcatcttcacaagacctcttatcacatttgcttacatctccagatgaaaatggtatgtttctaaccgaagaagagattgtagacaacatcttgttactactctttgcgggtcatgatacctcggctctttcaatcactttgctcatgaagactcttggcgaacattctgatgtttatgacaaggtgttaaaagagcaactagagatatcgaagacgaaagaagcatgggagtccctgaaatgggaggacatacaaaagatgaaatactcctggagtgttatatgtgaagtcatgagactaaatccacctgttataggaacctatagagaggcccttgtggatattgattatgcgggttataccatccccaaaggatggaagctgcactggagtgctgtatcgacacaaagggacgaggctaactttgaagacgtaacacgttttgacccatcacggtttgaaggcgcaggaccgactccattcacctttgttccgtttggaggggggcctagaatgtgtttagggaaagaatttgctcgattggaagtacttgcgtttcttcacaatattgtcaccaatttcaaatgggacctgttgatacctgatgagaaaatagaatatgatcccatggctaccccagcaaaggggcttccaattcgtcttcatccccatcaagtttga

SEQ ID NO: 67 - DNA, 1578 bp, Arabidopsis thaliana
  source 1..1578: mol_type = genomic DNA
atggagagtttggttgttcatacggtaaatgcaatttggtgcatagttattgtcggaatcttcagcgtaggttatcatgtgtatggaagagcggtggtggagcagtggaggatgcggaggagtttaaagttgcaaggcgtgaagggtcctccaccgtcgatctttaacggcaatgtgtcggagatgcaacggattcagtcggaggctaaacactgttccggcgataacatcatttctcatgactattcttcttctctatttcctcatttcgatcactggcgaaaacaatacggaaggatttacacatactcaacggggttaaagcagcacctttacataaaccacccggaaatggtgaaggagcttagccaaaccaacacacttaaccttggtagaatcactcacatcaccaaacgccttaaccccattctcggcaatggcatcatcacctctaatgggcctcattgggcccatcaacgtcgtatcattgcctatgagtttacccacgacaaaatcaagggaatggttggtttaatggtggaatctgccatgccaatgttgaacaaatgggaagagatggtgaaaagaggaggagaaatgggttgtgacataagagtggacgaagaccttaaggatgtctcagctgatgtcatcgctaaggcttgctttgggagctctttttcaaaaggcaaagcaatattctctatgattagggatcttttaaccgccattactaaacgaagcgtcctcttcagattcaatggcttcactgatatggtgtttggaagtaagaagcatggtgatgtggatattgatgcgcttgagatggaattagaatcttctatatgggaaacggttaaggagagggaaattgaatgtaaggatactcacaagaaggatctaatgcagttgatactcgagggagcgatgcgaagctgcgatggtaacttgtgggacaagtcagcctatagacggtttgtggtggacaattgcaagagcatctatttcgccggacatgattcaaccgcagtctcagtgtcttggtgccttatgctcctcgctctcaatcctagttggcaggttaaaattcgcgatgaaatcttgagttcttgcaagaatggcattcccgacgcagaatcaattcctaatctcaaaacggtgacaatggtaatacaagaaacaatgagactatacccaccagcaccaatcgtgggaagagaagcatccaaagacataagacttggagaccttgtggtgccaaaaggagtgtgcatttggacactcattcctgccttacaccgagaccccgagatctggggaccagacgcaaacgacttcaagccagagaggtttagtgagggaatctctaaggcttgcaaataccctcagtcatacatcccatttggccttggaccaagaacatgcgtaggcaaaaactttggtatgatggaagtgaaagtgcttgtttcacttattgtctcaaagttcagttttactctttccccgacttatcagcactctccaagccataaactccttgtagagcctcaacatggtgttgtcattagggttgtttga

SEQ ID NO: 68 - DNA, 1590 bp, Vitis vinifera
  source 1..1590: mol_type = genomic DNA
atgtatttccttctccaatacctaaacatcaccacggtcggagtctttgccacacttttcctttcctactgtctattattatggaggtctagagctggtaacaaaaaaatagcacctgaagctgctgctgcatggcccataatcggtcacctacacctgttagctggtggttctcatcagcttccccacataaccttgggaaacatggccgacaaatatggaccggtcttcacaattcggattgggttgcatcgagctgtggtggtaagttcttgggagatggctaaagaatgctcgaccgccaatgaccaggtttcatcctcgcgtcccgaacttttagcctcaaaacttttgggctacaactacgccatgtttggtttctctccatacggttcttactggcgtgaaatgcgcaagataatcagcctagagctactctctaacagccgcttagagctgctgaaggacgtccgagcttcagaagtggtgacatccataaaagagctatacaagctctgggcagagaaaaaaaatgaatcgggccttgtctcggtggagatgaagcagtggtttggagacttgactctgaacgtaattcttaggatggtggcagggaagcgttatttcagtgcttcagatgcaagtgaaaataaacaggcgcagaggtgccggagagtgttcagggaattctttcatttgtcagggctctttgtggtggcggacgctattccatttcttggatggctcgactgggggagacatgagaaaaccctaaagaagacagcaatagaaatggacagtattgctcaagaatggttagaggagcaccgtcggaggaaagactccggtgatgataatagtacgcaagacttcatggatgtgatgcagtcagttcttgatggcaaaaaccttggtggttacgacgctgataccatcaataaagccacatgcctgactctaatctccggaggtagcgacacaactgttgtctctctaacatgggcactctctcttgtactaaacaaccgtgacaccttaaaaaaagctcaagaagaattagacatccaagttggtaaggaaagattagtgaatgaacaagatataagtaagttggtctatctccaagccattgttaaagagacattacggttatatccaccaggaccacttggaggactacgccaatttaccgaggattgcaccttgggtggataccatgtctctaaaggcacccgtttaataatgaacctttcgaagatccaaaaggatccaagaatttggtcagatccgacagaattccaaccagagaggtttctcaccacccataaagatgttgatcctcggggaaaacattttgagtttataccatttggagctggtcgaagagcatgtccaggaataacttttggtcttcaagtattacatttaacattggctagtttcttacatgcgtttgaattttcaactccatcaaatgaacaggtcaatatgcgcgagagccttggacttacaaatatgaaatctaccccacttgaagttctcatttctccacgcttatcattgaattgttttaacctaatgaagatataa

SEQ ID NO: 69 - DNA, 1440 bp, Medicago trunculata
  source 1..1440: mol_type = genomic DNA
atggagcctaatttctatctctcccttctccttctctttgtcactttcatatctctctctctttttttcatattctacaaacagaaatctccattaaatttgccacctggtaaaatgggttacccaatcataggtgaaagccttgagttcttatcaacaggatggaaaggacatcctgaaaaattcattttcgaccgtatgcgtaaatattcctcagaactctttaaaacatcaatcgtaggagaatctacggtggtttgttgcggagcagcaagtaacaagtttttgttttcaaacgagaataaacttgtgactgcatggtggccagatagtgtaaacaaaatcttccctactacttctcttgactctaacttgaaggaagaatccatcaagatgagaaaattgcttccacaattctttaaacccgaagctctacaacgttatgttggtgtcatggatgttattgctcaaagacattttgttactcattgggataataaaaatgaaatcaccgtctaccccttggccaagaggtacacctttttgttagcttgtcggttgttcatgagcgttgaagacgagaatcatgtagcaaaatttagtgatccatttcagttaattgcggccggaatcatatctctaccaattgatttgccaggaacaccattcaacaaagctataaaggcctcaaactttataagaaaggagttgattaagatcataaagcaaaggagggtagatttggcagaagggacagcatcaccaacacaagatatattgtctcacatgttgttgacaagtgatgaaaatggaaagagtatgaatgaacttaatattgctgataagattcttggccttttgatcggaggacatgacactgctagcgtcgcatgcactttccttgtcaaatatctcggcgagttacctcacatttatgataaagtctatcaagagcaaatggaaattgcaaaatcgaaaccagcaggagaattgttgaattgggatgacctgaagaaaatgaaatactcttggaacgtagcttgtgaagtaatgagactttcccctccactccaaggaggtttcagggaagccatcactgactttatgttcaatggattctcaattcctaagggatggaagctttattggagtgcaaattcaacacataagaacgcagaatgttttcccatgccagagaaatttgacccaacaagatttgaaggaaatggaccagctccttatacttttgttccctttggtggaggaccaaggatgtgtcctggaaaagagtatgcaagattagaaatacttgttttcatgcacaatttggtgaaaaggtttaagtgggaaaaggtgattccagatgagaagattattgttgatccattccccatccctgcaaaggatcttccaattcgcctttatccacacaaagcttaa

SEQ ID NO: 70 - DNA, 2133 bp, synthetic construct
  misc_feature 1..2133: note = Description of Artificial Sequence: Synthetic polynucleotide
  source 1..2133: mol_type = other DNA
atgcaatcagattcagtcaaagtctctccatttgatttggtttccgctgctatgaatggcaaggcaatggaaaagttgaacgctagtgaatctgaagatccaacaacattgcctgcactaaagatgctagttgaaaatagagaattgttgacactgttcacaacttccttcgcagttcttattgggtgtcttgtatttctaatgtggagacgttcatcctctaaaaagctggtacaagatccagttccacaagttatcgttgtaaagaagaaagagaaggagtcagaggttgatgacgggaaaaagaaagtttctattttctacggcacacaaacaggaactgccgaaggttttgctaaagcattagtcgaggaagcaaaagtgagatatgaaaagacctctttcaaggttatcgatctagatgactacgctgcagatgatgatgaatatgaggaaaaactgaaaaaggaatccttagccttcttcttcttggccacatacggtgatggtgaacctactgataatgctgctaacttctacaagtggttcacagaaggcgacgataaaggtgaatggctgaaaaagttacaatacggagtatttggtttaggtaacagacaatatgaacatttcaacaagatcgctattgtagttgatgataaacttactgaaatgggagccaaaagattagtaccagtaggattaggggatgatgatcagtgtatagaagatgacttcaccgcctggaaggaattggtatggccagaattggatcaacttttaagggacgaagatgatacttctgtgactaccccatacactgcagccgtattggagtacagagtggtttaccatgataaaccagcagactcatatgctgaagatcaaacccatacaaacggtcatgttgttcatgatgcacagcatccttcaagatctaatgtggctttcaaaaaggaactacacacctctcaatcagataggtcttgtactcacttagaattcgatatttctcacacaggactgtcttacgaaactggcgatcacgttggcgtttattccgagaacttgtccgaagttgtcgatgaagcactaaaactgttagggttatcaccagacacatacttctcagtccatgctgataaggaggatgggacacctatcggtggtgcttcactaccaccaccttttcctccttgcacattgagagacgctctaaccagatacgcagatgtcttatcctcacctaaaaaggtagctttgctggcattggctgctcatgctagtgatcctagtgaagccgataggttaaagttcctggcttcaccagccggaaaagatgaatatgcacaatggatcgtcgccaaccaacgttctttgctagaagtgatgcaaagttttccatctgccaagcctccattaggtgtgttcttcgcagcagtagctccacgtttacaaccaagatactactctatcagttcatctcctaagatgtctcctaacagaatacatgttacatgtgctttggtgtacgagactactccagcaggcagaattcacagaggattgtgttcaacctggatgaaaaatgctgtccctttaacagagtcacctgattgctctcaagcatccattttcgttagaacatcaaatttcagacttccagtggatccaaaagttccagtcattatgataggaccaggcactggtcttgccccattcaggggctttcttcaagagagattggccttgaaggaatctggtacagaattgggttcttctatctttttctttggttgccgtaatagaaaagttgactttatctacgaggacgagcttaacaattttgttgagacaggagcattgtcagaattgatcgtcgcattttcaagagaagggactgccaaagagtacgttcagcacaagatgagtcaaaaagcctccgatatatggaaacttctaagtgaaggtgcctatctttatgtctgtggcgatgcaaagggcatggccaaggatgtccatagaactctgcatacaattgttcaggaacaagggagtctggattcttccaaggctgaattgtacgtcaaaaacttacagatgtctggaagatacttaagagatgtttggtaa

SEQ ID NO: 71 - DNA, 2079 bp, synthetic construct
  misc_feature 1..2079: note = Description of Artificial Sequence: Synthetic polynucleotide
  source 1..2079: mol_type = other DNA
atgacttctgcactttatgcctccgatcttttcaaacaattgaaaagtatcatgggaacggattctttgtccgatgatgttgtattagttattgctacaacttctctggcactggttgctggtttcgttgtcttattgtggaaaaagaccacggcagatcgttccggcgagctaaagccactaatgatccctaagtctctgatggcgaaagatgaggatgatgacttagatctaggttctggaaaaacgagagtctctatcttcttcggcacacaaaccggaacagccgaaggattcgctaaagcactttcagaagagatcaaagcaagatacgaaaaggcggctgtaaaagtaatcgatttggatgattacgctgccgatgatgaccaatatgaggaaaagttgaaaaaggaaacattggctttcttttgtgtagccacgtatggtgatggtgaaccaaccgataacgccgcaagattctacaagtggtttactgaagagaacgaaagagatatcaagttgcagcaacttgcttacggcgtttttgccttaggtaacagacaatacgagcactttaacaagataggtattgtcttagatgaagagttatgcaaaaagggtgcgaagagattgattgaagtcggtttaggagatgatgatcaatctatcgaggatgactttaatgcatggaaggaatctttgtggtctgaattagataagttacttaaggacgaagatgataaatccgttgccactccatacacagccgtcattccagaatatagagtagttactcatgatccaagattcacaacacagaaatcaatggaaagtaatgtggctaatggtaatactaccatcgatattcatcatccatgtagagtagacgttgcagttcaaaaggaattgcacactcatgaatcagacagatcttgcatacatcttgaatttgatatatcacgtactggtatcacttacgaaacaggtgatcacgtgggtgtctacgctgaaaaccatgttgaaattgtagaggaagctggaaagttgttgggccatagtttagatcttgttttctcaattcatgccgataaagaggatggctcaccactagaaagtgcagtgcctccaccatttccaggaccatgcaccctaggtaccggtttagctcgttacgcggatctgttaaatcctccacgtaaatcagctctagtggccttggctgcgtacgccacagaaccttctgaggcagaaaaactgaaacatctaacttcaccagatggtaaggatgaatactcacaatggatagtagctagtcaacgttctttactagaagttatggctgctttcccatccgctaaacctcctttgggtgttttcttcgccgcaatagcgcctagactgcaaccaagatactattcaatttcatcctcacctagactggcaccatcaagagttcatgtcacatccgctttagtgtacggtccaactcctactggtagaatccataagggcgtttgttcaacatggatgaaaaacgcggttccagcagagaagtctcacgaatgttctggtgctccaatctttatcagagcctccaacttcaaactgccttccaatccttctactcctattgtcatggtcggtcctggtacaggtcttgctccattcagaggtttcttacaagagagaatggccttaaaggaggatggtgaagagttgggatcttctttgttgtttttcggctgtagaaacagacaaatggatttcatctacgaagatgaactgaataactttgtagatcaaggagttatttcagagttgataatggctttttctagagaaggtgctcagaaggagtacgtccaacacaaaatgatggaaaaggccgcacaagtttgggacttaatcaaagaggaaggctatctatatgtctgtggtgatgcaaagggtatggcaagagatgttcacagaacacttcatactatagtccaggaacaggaaggcgttagttcttctgaagcggaagcaattgtgaaaaagttacaaacagagggaagatacttgagagatgtgtggtaa

SEQ ID NO: 72 - DNA, 2142 bp, synthetic construct
  misc_feature 1..2142: note = Description of Artificial Sequence: Synthetic polynucleotide
  source 1..2142: mol_type = other DNA
atggcagaattagatacacttgatatagtagtattaggtgttatctttttgggtactgtggcatactttactaagggtaaattgtggggtgttaccaaggatccatacgctaacggattcgctgcaggtggtgcttccaagcctggcagaactagaaacatcgtcgaagctatggaggaatcaggtaaaaactgtgttgttttctacggcagtcaaacaggtacagcggaggattacgcatcaagacttgcaaaggaaggaaagtccagattcggtttgaacactatgatcgccgatctagaagattatgacttcgataacttagacactgttccatctgataacatcgttatgtttgtattggctacttacggtgaaggcgaaccaacagataacgccgtggatttctatgagttcattactggcgaagatgcctctttcaatgagggcaacgatcctccactaggtaacttgaattacgttgcgttcggtctgggcaacaatacctacgaacactacaactcaatggtcaggaacgttaacaaggctctagaaaagttaggagctcatagaattggagaagcaggtgagggtgacgacggagctggaactatggaagaggactttttagcttggaaagatccaatgtgggaagccttggctaaaaagatgggcttggaggaaagagaagctgtatatgaacctattttcgctatcaatgagagagatgatttgacccctgaagcgaatgaggtatacttgggagaacctaataagctacacttggaaggtacagcgaaaggtccattcaactcccacaacccatatatcgcaccaattgcagaatcatacgaacttttctcagctaaggatagaaattgtctgcatatggaaattgatatttctggtagtaatctaaagtatgaaacaggcgaccatatcgcgatctggcctaccaacccaggtgaagaggtcaacaaatttcttgacattctagatctgtctggtaagcaacattccgtcgtaacagtgaaagccttagaacctacagccaaagttccttttccaaatccaactacctacgatgctatattgagataccatctggaaatatgcgctccagtttctagacagtttgtctcaactttagcagcattcgcccctaatgatgatatcaaagctgagatgaaccgtttgggatcagacaaagattacttccacgaaaagacaggaccacattactacaatatcgctagatttttggcctcagtctctaaaggtgaaaaatggacaaagataccattttctgctttcatagaaggccttacaaaactacaaccaagatactattctatctcttcctctagtttagttcagcctaaaaagattagtattactgctgttgtcgaatctcagcaaattccaggtagagatgacccattcagaggtgtagcgactaactacttgttcgctttgaagcagaaacaaaacggtgatccaaatccagctccttttggccaatcatacgagttgacaggaccaaggaataagtatgatggtatacatgttccagtccatgtaagacattctaactttaagctaccatctgatccaggcaaacctattatcatgatcggtccaggtaccggtgttgccccttttagaggcttcgtccaagagagggcaaaacaagccagagatggtgtagaagttggtaaaacactgctgttctttggatgtagaaagagtacagaagatttcatgtatcaaaaagagtggcaagagtacaaggaagctcttggcgacaaattcgaaatgattacagctttttcaagagaaggatctaaaaaggtttatgttcaacacagactgaaggaaagatcaaaggaagtttctgatcttctatcccaaaaagcatacttctacgtttgcggagacgccgcacatatggcacgtgaagtgaacactgtgttagcacagatcatagcagaaggccgtggtgtatcagaagccaagggtgaggaaattgtcaaaaacatgagatcagcaaatcaataccaagtgtgttctgatttcgtaactttacactgtaaagagacaacatacgcgaattcagaattgcaagaggatgtctggagttaa

SEQ ID NO: 73 - DNA, 2133 bp, Stevia rebaudiana
  source 1..2133: mol_type = genomic DNA
atgcaatcagattccgtaaaagtgtcgccgttcgatctcgtatctgcagctatgaacggaaaagcaatggagaaattgaacgcatcggaatcggaagatccgacgacgctaccggcgttgaagatgctggtggagaatcgcgagctgctgacactgtttacgacgtcgtttgctgtattgatcggatgtctcgtgtttttgatgtggcggagatcgtcctcgaagaaactggttcaggatccggtgccgcaggtaatcgttgttaagaagaaagagaaggagtctgaggttgatgatggcaagaagaaagtttcgatattctacggaactcaaacaggaaccgctgaaggttttgccaaggcacttgtagaggaagctaaagttcgatatgaaaagacatcctttaaagttattgatctggatgattatgctgctgatgacgatgagtatgaggagaagcttaagaaagaatctttggcgtttttctttttggcaacgtatggagatggtgaaccaacagataatgcagccaatttttacaaatggtttacagagggagatgacaaaggcgaatggctgaagaaacttcaatatggcgtgtttggcctcggtaacagacaatatgagcatttcaataagattgcaatagtggttgatgacaaactcacagaaatgggcgcaaaacgccttgttcctgtgggtcttggagatgacgatcaatgtatagaagatgactttacagcatggaaagagttagtgtggcccgagttggatcaattgttgcgtgatgaggatgacacgagtgttacgactccttacactgctgcggttttggaataccgagttgtatatcatgataaacctgcagactcgtatgcagaagatcaaactcatacaaatggtcatgttgttcatgatgctcaacatccatctagatccaatgtggcatttaaaaaggaattgcacacctctcaatctgaccggtcttgcactcatttggaatttgatatctctcacaccgggctatcatacgagacgggggatcatgttggtgtctacagtgagaatctaagtgaagttgtagatgaagctttaaaattactcggtttgtcacccgacacttatttctcagtccatgctgacaaggaagacggaacacctattggcggcgcctccttgccgccacctttccctccatgcactttaagagatgcattaacgcgctacgcagatgttttgagttctcctaaaaaggttgctttgcttgctctggctgctcatgcttctgatcctagcgaagccgatcgattaaaatttctagcatctccggctggcaaggatgaatatgctcaatggatagttgcaaaccaaagaagtcttcttgaagttatgcagtcatttccgtcagctaaaccgccacttggtgttttcttcgcagctgtcgccccacgtttacaacctcgatattactcgatttcttcttctccaaagatgtcaccaaacagaattcatgtgacttgtgcattagtttatgagacaacacctgcaggacgtattcacagaggattgtgttcaacatggatgaagaatgctgtgcctttgaccgaaagtccagattgtagtcaggcgtcgatttttgttagaacgtctaacttccgacttccggttgacccgaaagtcccggtcatcatgatcggtcccgggactgggttagcccctttcagaggttttcttcaagaacggttagctttgaaggaatctggaaccgaactcgggtcatctattttctttttcggatgcagaaaccgcaaagtggattttatatacgaagacgaactaaacaactttgtggagaccggtgctttatcggagcttattgttgcattctcccgtgaaggaaccgcaaaggagtatgtgcaacataaaatgagccagaaggcttcagatatctggaagttgctttcagagggagcatatttatatgtatgtggtgatgctaaaggcatggctaaagatgtacacagaacccttcacacaattgtacaagaacagggatctctagattcttccaaggcagaattgtatgtaaagaacctacaaatgtcgggaagatatcttcgtgatgtttggtaa

SEQ ID NO: 74 - DNA, 2079 bp, Arabidopsis thaliana
  source 1..2079: mol_type = genomic DNA
atgacttctgctttgtatgcttccgatttgtttaagcagctcaagtcaattatggggacagattcgttatccgacgatgttgtacttgtgattgcaacgacgtctttggcactagtagctggatttgtggtgttgttatggaagaaaacgacggcggatcggagcggggagctgaagcctttgatgatccctaagtctcttatggctaaggacgaggatgatgatttggatttgggatccgggaagactagagtctctatcttcttcggtacgcagactggaacagctgagggatttgctaaggcattatccgaagaaatcaaagcgagatatgaaaaagcagcagtcaaagtcattgacttggatgactatgctgccgatgatgaccagtatgaagagaaattgaagaaggaaactttggcatttttctgtgttgctacttatggagatggagagcctactgacaatgctgccagattttacaaatggtttacggaggaaaatgaacgggatataaagcttcaacaactagcatatggtgtgtttgctcttggtaatcgccaatatgaacattttaataagatcgggatagttcttgatgaagagttatgtaagaaaggtgcaaagcgtcttattgaagtcggtctaggagatgatgatcagagcattgaggatgattttaatgcctggaaagaatcactatggtctgagctagacaagctcctcaaagacgaggatgataaaagtgtggcaactccttatacagctgttattcctgaataccgggtggtgactcatgatcctcggtttacaactcaaaaatcaatggaatcaaatgtggccaatggaaatactactattgacattcatcatccctgcagagttgatgttgctgtgcagaaggagcttcacacacatgaatctgatcggtcttgcattcatctcgagttcgacatatccaggacgggtattacatatgaaacaggtgaccatgtaggtgtatatgctgaaaatcatgttgaaatagttgaagaagctggaaaattgcttggccactctttagatttagtattttccatacatgctgacaaggaagatggctccccattggaaagcgcagtgccgcctcctttccctggtccatgcacacttgggactggtttggcaagatacgcagaccttttgaaccctcctcgaaagtctgcgttagttgccttggcggcctatgccactgaaccaagtgaagccgagaaacttaagcacctgacatcacctgatggaaaggatgagtactcacaatggattgttgcaagtcagagaagtcttttagaggtgatggctgcttttccatctgcaaaacccccactaggtgtattttttgctgcaatagctcctcgtctacaacctcgttactactccatctcatcctcgccaagattggcgccaagtagagttcatgttacatccgcactagtatatggtccaactcctactggtagaatccacaagggtgtgtgttctacgtggatgaagaatgcagttcctgcggagaaaagtcatgaatgtagtggagccccaatctttattcgagcatctaatttcaagttaccatccaacccttcaactccaatcgttatggtgggacctgggactgggctggcaccttttagaggttttctgcaggaaaggatggcactaaaagaagatggagaagaactaggttcatctttgctcttctttgggtgtagaaatcgacagatggactttatatacgaggatgagctcaataattttgttgatcaaggcgtaatatctgagctcatcatggcattctcccgtgaaggagctcagaaggagtatgttcaacataagatgatggagaaggcagcacaagtttgggatctaataaaggaagaaggatatctctatgtatgcggtgatgctaagggcatggcgagggacgtccaccgaactctacacaccattgttcaggagcaggaaggtgtgagttcgtcagaggcagaggctatagttaagaaacttcaaaccgaaggaagatacctcagagatgtctggtga

SEQ ID NO: 75 - DNA, 2142 bp, Giberella fujikuroi
  source 1..2142: mol_type = genomic DNA
atggctgaactcgacactctggacatcgtcgtcctcggcgttatcttcctcggaacggttgcatactttacaaagggcaagctatggggtgttaccaaggatccctacgcgaatggcttcgctgccggcggcgcttctaagccgggtcgcacgaggaacatcgtcgaggcaatggaagaatccggcaagaactgtgttgtcttctatggttctcagaccggtactgctgaagattatgcttctcgcctcgccaaggagggtaagagtcgattcggactaaacaccatgattgccgatcttgaggactacgatttcgacaacctggataccgttcccagtgacaacattgtcatgttcgttctcgcaacttatggtgaaggtgagcctaccgataacgcggtcgacttctatgaattcattaccggcgaggatgccagcttcaatgagggcaatgatcctccgctgggcaacctcaactacgttgctttcggtctcggaaacaacacgtacgagcactacaactctatggtccgcaatgttaacaaggctctcgagaagcttggcgctcaccgcatcggtgaagctggtgagggtgatgatggtgctggtaccatggaagaggacttcttggcctggaaggatcccatgtgggaagccctcgctaagaaaatgggactggaagagcgtgaagcagtctacgagcctatttttgccattaacgaacgcgacgacctgactcctgaagccaatgaagtgtatctcggtgagcccaacaagctgcatctcgaaggcaccgccaagggaccattcaactctcacaacccctacattgcccctatcgctgaatcttatgagttgttctccgccaaggacagaaactgcctccacatggaaattgacatcagcggttctaacctcaagtacgaaactggagaccatattgctatctggcctaccaaccctggtgaggaggtcaacaaattcctggatattctcgacctctctggaaagcagcacagcgttgtcactgtcaaggctctcgagcctaccgccaaggttcctttccccaaccctacaacctacgatgccattctgcgataccacctcgagatctgcgctcctgtttcacgtcaattcgtctctactctcgccgcatttgctcccaacgatgatatcaaggctgagatgaaccgccttggcagcgataaggattatttccacgagaagactggcccgcattactacaacattgcccgtttccttgccagcgtcagcaagggcgagaagtggaccaaaatcccgttctctgccttcatcgagggtctcaccaagctccagccccgttactactccatttcttcctcgtctctggttcagcccaagaaaatctcgatcactgccgtcgttgaatcccagcagattcctggccgggatgatcctttccgtggtgttgctacaaactatctttttgccctaaagcaaaagcagaacggtgaccccaaccctgcaccttttggtcagagctacgagcttacaggcccccgcaataagtatgatggcatccacgttcctgtccatgttcgtcactccaacttcaagctcccctcggaccccggtaagcccatcatcatgattggtcctggtactggtgtcgctcccttccgcggtttcgtgcaggagcgtgctaagcaagcccgtgatggtgttgaggttggaaagacactcttgttctttggttgccgaaagtcaaccgaggatttcatgtaccaaaaggagtggcaggaatacaaggaggctcttggcgataagtttgaaatgatcaccgccttttctcgagagggctccaagaaggtttatgttcagcaccgacttaaggagcgatctaaggaggtcagcgatctgctctcccagaaggcttatttctatgtctgcggtgatgcagcccacatggcccgcgaggtcaataccgtcttggcacaaatcattgccgagggacgtggggtgtctgaggccaagggcgaggagatcgtgaagaacatgagatcagcgaaccaataccaggtatgtagtgactttgttactcttcactgcaaagaaaccacatatgctaactcagaattacaggaggatgtttggtcatag

SEQ ID NO: 76 - protein, 459 aa, Ipomoea purpurea
MGSQATTYHMAMYPWFGVGHLTGFFRLANKLAGKGHRISFLIPKNTQSKLESFNLHPHLISFVPIVVPSIPGLPPGAETTSDVPFPSTHLLMEAMDKTQNDIEIILKDLKVDVVFYDFTHWLPSLARKIGIKSVFYSTISPLMHGYALSPERRVVGKQLTEADMMKAPASFPDPSIKLHAHEARGFTARTVMKFGGDITFFDRIFTAVSESDGLAYSTCREIEGQFCDYIETQFQKPVLLAGPALPVPSKSTMEQKWSDWLGKFKEGSVIYCAFGSECTLRKDKFQELLWGLELTGMPFFAALKPPFETESVEAAIPEELKEKIQGRGIVHGEWVQQQLFLQHPSVGCFVSHCGWASLSEALVNDCQIVLLPQVGDQIINARIMSVSLKVGVEVEKGEEDGVFSRESVCKAVKAVMDEKSEIGREVRGNHDKLRGFLMNADLDSKYMDSFNQKLQDLLG

SEQ ID NO: 77 - DNA, 1380 bp, Ipomoea purpurea
  source 1..1380: mol_type = genomic DNA
atgggttctcaagctacaacttaccatatggccatgtatccatggtttggggttggacatttgactggtttcttccgtttggcaaacaaattagctggcaaaggacatagaatctcatttctaattcctaaaaacactcaatctaagttagaatctttcaaccttcatccacacttaatctcttttgtgcctatcgttgtcccaagtataccaggcctgccacctggtgcagagactacatcagatgttcctttcccaagtacacatttgctaatggaagcaatggacaagactcaaaacgatatagagattatcctgaaggatcttaaagtagatgttgttttctatgattttactcactggttgccttctctggccagaaagattggcattaagagtgtcttttactccaccatttctcctttaatgcatggatatgctttatcaccagaaagacgtgtagttggtaagcaattgacagaggcagatatgatgaaggccccagcttctttcccagacccatccattaagctacatgcacatgaagctaggggttttacagccagaaccgttatgaaattcggtggtgacatcacctttttcgatagaatattcacagcagtttccgaaagtgatggcctggcctactctacttgtagagagatcgagggacaattctgtgattacattgaaacacaattccagaagccagtcttgttagccggtccagctttgccagtcccatccaaatccactatggaacaaaagtggtcagattggttggggaaattcaaggaaggctccgtcatctactgtgctttcgggtctgaatgtacattgagaaaggacaaatttcaggaacttttatggggtttggaattgacaggaatgcctttcttcgctgctctgaagccaccttttgagactgagtctgttgaggctgctatccctgaggaactaaaggaaaagattcagggaagaggtatagtacatggagaatgggtacaacaacaattgtttcttcaacacccatctgtcgggtgcttcgtttctcactgcggctgggcaagtttatctgaagcccttgttaatgattgtcaaatcgtgttacttccacaagttggcgatcagattatcaacgccagaataatgtcagtatcacttaaagtgggcgtggaagttgaaaagggtgaggaggacggtgtcttttcaagagaatctgtgtgcaaggctgttaaagcagtaatggatgaaaaatctgaaatcggtagagaagtcagaggtaatcatgataaactgaggggtttcttgatgaatgcagacttagattcaaagtacatggattcattcaatcaaaagctacaagatttgctaggttaa

SEQ ID NO: 78 - protein, 438 aa, Bellis perennis
MDSKIDSKTFRVVMLPWLAYSHISSFLVFAKRLTNHNFHIYICSSQTNMQYLKNNLTSQYSKSIQLIELNLPSSSELPLQYHTTHGLPPHLTKTLSDDYQKSGPDFETILIKLNPHLVIYDFNQLWAPEVASTLHIPSIQLLSGCVALYALDAHLYTKPLDENLAKFPFPEIYPKNRDIPKGGSKYIERFVDCMRRSCEIILVRSTMELEGKYIDYLSKTLGKKVLPVGPLVQEASLLQDDHIWIMKWLDKKEESSVVFVCFGSEYILSDNEIEDIAYGLELSQVSFVWAIRAKTSALNGFIDRVGDKGLVIDKWVPQANILSHSSTGGFISHCGWSSTMESIRYGVPIIAMPMQFDQPYNARLMETVGAGIEVGRDGEGRLKREEIAAVVRKVVVEDSGESIREKAKELGEIMKKNMEAEVDGIVIENLVKLCEMNN

SEQ ID NO: 79 - DNA, 1317 bp, Bellis perennis
  source 1..1317: mol_type = genomic DNA
atggattctaaaatcgattcaaagacattcagagtcgttatgttgccttggcttgcatactcacacatttcatcattcctagtgtttgccaagagactaacaaatcataacttccacatctacatttgttcctctcaaacaaatatgcaatacctgaaaaacaacttgacgtctcagtattcaaaatctatacaactgattgagttgaatcttccatctagttccgaattgcctctgcagtatcatactactcacggactaccaccacaccttacgaaaacattgtctgatgattatcaaaagtccggacctgactttgaaaccattttgatcaaattgaacccacatctggtaatctacgactttaatcaactttgggctccagaggttgctagtacacttcatattccatccatacagttactgtctggttgcgtcgccttatatgccttagacgcccatctgtacacaaagccactagacgaaaacttggctaagtttcctttcccagaaatctatcctaaaaacagagatattcctaagggaggtagtaaatacatcgaaaggttcgtagactgtatgagaagatcttgtgaaatcatattagtcagaagtaccatggaacttgaaggaaaatacattgattacttgtctaagacattagggaaaaaggtgttgccagtagggcctctggtgcaagaggcttctttgttgcaagatgatcatatatggattatgaagtggttagacaaaaaggaggagtcatccgtcgtgtttgtttgttttggttctgagtacatcttatcagacaacgaaatagaagatattgcttatggcctagagttgtcccaagtaagtttcgtttgggcaataagagctaagacttctgccttaaatggcttcattgatagagtgggtgataaaggcttagtcatcgataaatgggttccacaggctaacatcttatctcactcttctactggtggattcattagtcattgcggttggtcatcaacaatggaatctattagatatggggttcctattatcgccatgccaatgcaattcgatcaaccttacaatgctaggttgatggaaactgttggtgcaggtatcgaagttggcagagatggcgaaggtagattgaaaagagaagagattgctgccgtggttagaaaggtcgttgttgaagattctggggaatccataagggagaaggcaaaggaattgggagaaatcatgaaaaaaaacatggaggccgaagtagatggtatagtgattgaaaatctagttaagctatgtgagatgaacaattaa

SEQ ID NO: 80 - DNA, 2490 bp, Zea mays
  source 1..2490: mol_type = genomic DNA
atggttttgtcttcttcttgtactacagtaccacacttatcttcattagctgtcgtgcaacttggtccttggagcagtaggattaaaaagaaaaccgatactgttgcagtaccagccgctgcaggaaggtggagaagggccttggctagagcacagcacacatcagaatccgcagctgtcgcaaagggcagcagtttgacccctatagtgagaactgacgctgagtcaaggagaacaagatggccaaccgatgacgatgacgccgaacctttagtggatgagatcagggcaatgcttacttccatgtctgatggtgacatttccgtgagcgcatacgatacagcctgggtcggattggttccaagattagacggcggtgaaggtcctcaatttccagcagctgtgagatggataagaaataaccagttgcctgacggaagttggggcgatgccgcattattctctgcctatgacaggcttatcaatacccttgcctgcgttgtaactttgacaaggtggtccctagaaccagagatgagaggtagaggactatcttttttgggtaggaacatgtggaaattagcaactgaagatgaagagtcaatgcctattggcttcgaattagcatttccatctttgatagagcttgctaagagcctaggtgtccatgacttcccttatgatcaccaggccctacaaggaatctactcttcaagagagatcaaaatgaagaggattccaaaagaagtgatgcataccgttccaacatcaatattgcacagtttggagggtatgcctggcctagattgggctaaactacttaaactacagagcagcgacggaagttttttgttctcaccagctgccactgcatatgctttaatgaataccggagatgacaggtgttttagctacatcgatagaacagtaaagaaattcaacggcggcgtccctaatgtttatccagtggatctatttgaacatatttgggccgttgatagacttgaaagattaggaatctccaggtacttccaaaaggagatcgaacaatgcatggattatgtaaacaggcattggactgaggacggtatttgttgggcaaggaactctgatgtcaaagaggtggacgacacagctatggcctttagacttcttaggttgcacggctacagcgtcagtcctgatgtgtttaaaaacttcgaaaaggacggtgaatttttcgcatttgtcggacagtctaatcaagctgttaccggtatgtacaacttaaacagagcaagccagatatccttcccaggcgaggatgtgcttcatagagctggtgccttctcatatgagttcttgaggagaaaagaagcagagggagctttgagggacaagtggatcatttctaaagatctacctggtgaagttgtgtatactttggattttccatggtacggcaacttacctagagtcgaggccagagactacctagagcaatacggaggtggtgatgacgtttggattggcaagacattgtataggatgccacttgtaaacaatgatgtatatttggaattggcaagaatggatttcaaccactgccaggctttgcatcagttagagtggcaaggactaaaaagatggtatactgaaaataggttgatggactttggtgtcgcccaagaagatgcccttagagcttattttcttgcagccgcatctgtttacgagccttgtagagctgccgagaggcttgcatgggctagagccgcaatactagctaacgccgtgagcacccacttaagaaatagcccatcattcagagaaaggttagagcattctcttaggtgtagacctagtgaagagacagatggctcctggtttaactcctcaagtggctctgatgcagttttagtaaaggctgtcttaagacttactgattcattagccagggaagcacagccaatccatggaggtgacccagaagatattatacacaagttgttaagatctgcttgggccgagtgggttagggaaaaggcagacgctgccgatagcgtgtgcaatggtagttctgcagtagaacaagagggatcaagaatggtccatgataaacagacctgtctattattggctagaatgatcgaaatttctgccggtagggcagctggtgaagcagccagtgaggacggcgatagaagaataattcaattaacaggctccatctgcgacagtcttaagcaaaaaatgctagtttcacaggaccctgaaaaaaatgaagagatgatgtctcacgtggatgacgaattgaagttgaggattagagagttcgttcaatatttgcttagactaggtgaaaaaaagactggatctagcgaaaccaggcaaacatttttaagtatagtgaaatcatgttactatgctgctcattgcccacctcatgtcgttgatagacacattagtagagtgattttcgagccagtaagtgccgcaaagtaaccgcgg

SEQ ID NO: 81 - protein, 827 aa, Zea mays
MVLSSSCTTVPHLSSLAVVQLGPWSSRIKKKTDTVAVPAAAGRWRRALARAQHTSESAAVAKGSSLTPIVRTDAESRRTRWPTDDDDAEPLVDEIRAMLTSMSDGDISVSAYDTAWVGLVPRLDGGEGPQFPAAVRWIRNNQLPDGSWGDAALFSAYDRLINTLACVVTLTRWSLEPEMRGRGLSFLGRNMWKLATEDEESMPIGFELAFPSLIELAKSLGVHDFPYDHQALQGIYSSREIKMKRIPKEVMHTVPTSILHSLEGMPGLDWAKLLKLQSSDGSFLFSPAATAYALMNTGDDRCFSYIDRTVKKFNGGVPNVYPVDLFEHIWAVDRLERLGISRYFQKEIEQCMDYVNRHWTEDGICWARNSDVKEVDDTAMAFRLLRLHGYSVSPDVFKNFEKDGEFFAFVGQSNQAVTGMYNLNRASQISFPGEDVLHRAGAFSYEFLRRKEAEGALRDKWIISKDLPGEVVYTLDFPWYGNLPRVEARDYLEQYGGGDDVWIGKTLYRMPLVNNDVYLELARMDFNHCQALHQLEWQGLKRWYTENRLMDFGVAQEDALRAYFLAAASVYEPCRAAERLAWARAAILANAVSTHLRNSPSFRERLEHSLRCRPSEETDGSWFNSSSGSDAVLVKAVLRLTDSLAREAQPIHGGDPEDIIHKLLRSAWAEWVREKADAADSVCNGSSAVEQEGSRMVHDKQTCLLLARMIEISAGRAAGEAASEDGDRRIIQLTGSICDSLKQKMLVSQDPEKNEEMMSHVDDELKLRIREFVQYLLRLGEKKTGSSETRQTFLSIVKSCYYAAHCPPHVVDRHISRVIFEPVSAAK

SEQ ID NO: 82 - DNA, 1461 bp, synthetic construct
  misc_feature 1..1461: note = Description of Artificial Sequence: Synthetic polynucleotide
  source 1..1461: mol_type = other DNA
actagtaaaatggatgcaatggcaactactgagaaaaagcctcatgtgatcttcattccatttcctgcacaatctcacataaaggcaatgctaaagttagcacaactattacaccataagggattacagataactttcgtgaataccgacttcatccataatcaatttctggaatctagtggccctcattgtttggacggagccccagggtttagattcgaaacaattcctgacggtgtttcacattccccagaggcctccatcccaataagagagagtttactgaggtcaatagaaaccaactttttggatcgtttcattgacttggtcacaaaacttccagacccaccaacttgcataatctctgatggctttctgtcagtgtttactatcgacgctgccaaaaagttgggtatcccagttatgatgtactggactcttgctgcatgcggtttcatgggtttctatcacatccattctcttatcgaaaagggttttgctccactgaaagatgcatcatacttaaccaacggctacctggatactgttattgactgggtaccaggtatggaaggtataagacttaaagattttcctttggattggtctacagaccttaatgataaagtattgatgtttactacagaagctccacaaagatctcataaggtttcacatcatatctttcacacctttgatgaattggaaccatcaatcatcaaaaccttgtctctaagatacaatcatatctacactattggtccattacaattacttctagatcaaattcctgaagagaaaaagcaaactggtattacatccttacacggctactctttagtgaaagaggaaccagaatgttttcaatggctacaaagtaaagagcctaattctgtggtctacgtcaacttcggaagtacaacagtcatgtccttggaagatatgactgaatttggttggggccttgctaattcaaatcattactttctatggattatcaggtccaatttggtaataggggaaaacgccgtattacctccagaattggaggaacacatcaaaaagagaggtttcattgcttcctggtgttctcaggaaaaggtattgaaacatccttctgttggtggtttccttactcattgcggttggggctctacaatcgaatcactaagtgcaggagttccaatgatttgttggccatattcatgggaccaacttacaaattgtaggtatatctgtaaagagtgggaagttggattagaaatgggaacaaaggttaaacgtgatgaagtgaaaagattggttcaggagttgatgggggaaggtggccacaagatgagaaacaaggccaaagattggaaggaaaaagccagaattgctattgctcctaacgggtcatcctctctaaacattgataagatggtcaaagagattacagtcttagccagaaactaagtcgac

SEQ ID NO: 83 - DNA, 1398 bp, synthetic construct
  misc_feature 1..1398: note = Description of Artificial Sequence: Synthetic polynucleotide
  source 1..1398: mol_type = other DNA
actagtaaaatggcagagcaacaaaagatcaaaaagtcacctcacgtcttacttattccatttcctctgcaaggacatatcaacccattcatacaatttgggaaaagattgattagtaagggtgtaaagacaacactggtaaccactatccacactttgaattctactctgaaccactcaaatactactactacaagtatagaaattcaagctatatcagacggatgcgatgagggtggctttatgtctgccggtgaatcttacttggaaacattcaagcaagtgggatccaagtctctggccgatctaatcaaaaagttacagagtgaaggcaccacaattgacgccataatctacgattctatgacagagtgggttttagacgttgctatcgaatttggtattgatggaggttcctttttcacacaagcatgtgttgtgaattctctatactaccatgtgcataaagggttaatctctttaccattgggtgaaactgtttcagttccaggttttccagtgttacaacgttgggaaaccccattgatcttacaaaatcatgaacaaatacaatcaccttggtcccagatgttgtttggtcaattcgctaacatcgatcaagcaagatgggtctttactaattcattctataagttagaggaagaggtaattgaatggactaggaagatctggaatttgaaagtcattggtccaacattgccatcaatgtatttggacaaaagacttgatgatgataaagataatggtttcaatttgtacaaggctaatcatcacgaatgtatgaattggctggatgacaaaccaaaggaatcagttgtatatgttgctttcggctctcttgttaaacatggtccagaacaagttgaggagattacaagagcacttatagactctgacgtaaactttttgtgggtcattaagcacaaagaggaggggaaactgccagaaaacctttctgaagtgataaagaccggaaaaggtctaatcgttgcttggtgtaaacaattggatgttttagctcatgaatctgtaggctgttttgtaacacattgcggattcaactctacactagaagccatttccttaggcgtacctgtcgttgcaatgcctcagttctccgatcagacaaccaacgctaaacttttggacgaaatactaggggtgggtgtcagagttaaagcagacgagaatggtatcgtcagaagagggaacctagcttcatgtatcaaaatgatcatggaagaggaaagaggagttatcataaggaaaaacgcagttaagtggaaggatcttgcaaaggttgccgtccatgaaggcggctcttcagataatgatattgttgaatttgtgtccgaactaatcaaagcctaagtcgac

SEQ ID NO: 84 - DNA, 1437 bp, synthetic construct
  misc_feature 1..1437: note = Description of Artificial Sequence: Synthetic polynucleotide
  source 1..1437: mol_type = other DNA
actagtaaaatggctacatctgattctattgttgatgacaggaagcagttgcatgtggctactttcccttggcttgctttcggtcatatactgccttacctacaactatcaaaactgatagctgaaaaaggacataaagtgtcattcctttcaacaactagaaacattcaaagattatcttcccacatatcaccattgattaacgtcgttcaattgacacttccaagagtacaggaattaccagaagatgctgaagctacaacagatgtgcatcctgaagatatcccttacttgaaaaaggcatccgatggattacagcctgaggtcactagattccttgagcaacacagtccagattggatcatatacgactacactcactattggttgccttcaattgcagcatcactaggcatttctagggcacatttcagtgtaaccacaccttgggccattgcttacatgggtccatccgctgatgctatgattaacggcagtgatggtagaactaccgttgaagatttgacaaccccaccaaagtggtttccatttccaactaaagtctgttggagaaaacacgacttagcaagactggttccatacaaggcaccaggaatctcagacggctatagaatgggtttagtccttaaagggtctgactgcctattgtctaagtgttaccatgagtttgggacacaatggctaccacttttggaaacattacaccaagttcctgtcgtaccagttggtctattacctccagaaatccctggtgatgagaaggacgagacttgggtttcaatcaaaaagtggttagacgggaagcaaaaaggctcagtggtatatgtggcactgggttccgaagttttagtatctcaaacagaagttgtggaacttgccttaggtttggaactatctggattgccatttgtctgggcctacagaaaaccaaaaggccctgcaaagtccgattcagttgaattgccagacggctttgtcgagagaactagagatagagggttggtatggacttcatgggctccacaattgagaatcctgagtcacgaatctgtgtgcggtttcctaacacattgtggttctggttctatagttgaaggactgatgtttggtcatccacttatcatgttgccaatctttggtgaccagcctttgaatgcacgtctgttagaagataaacaagttggaattgaaatcccacgtaatgaggaagatggatgtttaaccaaggagtctgtggccagatcattacgttccgttgtcgttgaaaaggaaggcgaaatctacaaggccaatgcccgtgaactttcaaagatctacaatgacacaaaagtagagaaggaatatgtttctcaatttgtagattacctagagaaaaacgctagagccgtagctattgatcatgaatcctaagtcgac

SEQ ID NO: 85 - DNA, 1392 bp, synthetic construct
  misc_feature 1..1392: note = Description of Artificial Sequence: Synthetic polynucleotide
  source 1..1392: mol_type = other DNA
actagtaaaatggaaaacaagaccgaaacaacagttagacgtaggcgtagaatcattctgtttccagtaccttttcaagggcacatcaatccaatactacaactagccaacgttttgtactctaaaggtttttctattacaatctttcacaccaatttcaacaaaccaaaaacatccaattacccacatttcacattcagattcatacttgataatgatccacaagatgaacgtatttcaaacttacctacccacggtcctttagctggaatgagaattccaatcatcaatgaacatggtgccgatgagcttagaagagaattagagttacttatgttggcatccgaagaggacgaggaagtctcttgtctgattactgacgctctatggtactttgcccaatctgtggctgatagtttgaatttgaggagattggtactaatgacatccagtctgtttaactttcacgctcatgttagtttaccacaatttgacgaattgggatacttggaccctgatgacaagactaggttagaggaacaggcctctggttttcctatgttgaaagtcaaagatatcaagtctgcctattctaattggcaaatcttgaaagagatcttaggaaagatgatcaaacagacaaaggcttcatctggagtgatttggaacagtttcaaagagttagaagagtctgaattggagactgtaatcagagaaattccagcaccttcattcctgataccattaccaaaacatttgactgcttcctcttcctctttgttggatcatgacagaacagtttttcaatggttggaccaacaaccacctagttctgttttgtacgtgtcatttggtagtacttctgaagtcgatgaaaaggacttccttgaaatcgcaagaggcttagtcgatagtaagcagtcattcctttgggtcgtgcgtccaggtttcgtgaaaggctcaacatgggtcgaaccacttccagatggttttctaggcgaaagaggtagaatagtcaaatgggttcctcaacaggaagttttagctcatggcgctattggggcattctggactcattccggatggaattcaactttagaatcagtatgcgaaggggtacctatgatcttttcagattttggtcttgatcaaccactgaacgcaagatacatgtctgatgttttgaaagtgggtgtatatctagaaaatggctgggaaaggggtgaaatagctaatgcaataagacgtgttatggttgatgaagagggggagtatatcagacaaaacgcaagagtgctgaagcaaaaggccgacgtttctctaatgaagggaggctcttcatacgaatccttagaatctcttgtttcctacatttcatcactgtaagtcgac

SEQ ID NO: 86 - protein, 12 aa, synthetic construct
  REGION 1..12: note = Description of Artificial Sequence: Synthetic peptide
TSFAEYWNLLSP

SEQ ID NO: 87 - DNA, 1602 bp, synthetic construct
  misc_feature 1..1602: note = Description of Artificial Sequence: Synthetic polynucleotide
  source 1..1602: mol_type = other DNA
atgaccagctttgccgagtattggaatctgttaagtcccacttcttttgcagaatattggaaccttctatcaccgacgagtttcgcggagtactggaatttgttttctccaacatcgttcgctgaatactggaacttactcagccctgctagtaaaatggatgcaatggcaactactgagaaaaagcctcatgtgatcttcattccatttcctgcacaatctcacataaaggcaatgctaaagttagcacaactattacaccataagggattacagataactttcgtgaataccgacttcatccataatcaatttctggaatctagtggccctcattgtttggacggagccccagggtttagattcgaaacaattcctgacggtgtttcacattccccagaggcctccatcccaataagagagagtttactgaggtcaatagaaaccaactttttggatcgtttcattgacttggtcacaaaacttccagacccaccaacttgcataatctctgatggctttctgtcagtgtttactatcgacgctgccaaaaagttgggtatcccagttatgatgtactggactcttgctgcatgcggtttcatgggtttctatcacatccattctcttatcgaaaagggttttgctccactgaaagatgcatcatacttaaccaacggctacctggatactgttattgactgggtaccaggtatggaaggtataagacttaaagattttcctttggattggtctacagaccttaatgataaagtattgatgtttactacagaagctccacaaagatctcataaggtttcacatcatatctttcacacctttgatgaattggaaccatcaatcatcaaaaccttgtctctaagatacaatcatatctacactattggtccattacaattacttctagatcaaattcctgaagagaaaaagcaaactggtattacatccttacacggctactctttagtgaaagaggaaccagaatgttttcaatggctacaaagtaaagagcctaattctgtggtctacgtcaacttcggaagtacaacagtcatgtccttggaagatatgactgaatttggttggggccttgctaattcaaatcattactttctatggattatcaggtccaatttggtaataggggaaaacgccgtattacctccagaattggaggaacacatcaaaaagagaggtttcattgcttcctggtgttctcaggaaaaggtattgaaacatccttctgttggtggtttccttactcattgcggttggggctctacaatcgaatcactaagtgcaggagttccaatgatttgttggccatattcatgggaccaacttacaaattgtaggtatatctgtaaagagtgggaagttggattagaaatgggaacaaaggttaaacgtgatgaagtgaaaagattggttcaggagttgatgggggaaggtggccacaagatgagaaacaaggccaaagattggaaggaaaaagccagaattgctattgctcctaacgggtcatcctctctaaacattgataagatggtcaaagagattacagtcttagccagaaactaa

SEQ ID NO: 88 - protein, 533 aa, synthetic construct
  REGION 1..533: note = Description of Artificial Sequence: Synthetic polypeptide
MTSFAEYWNLLSPTSFAEYWNLLSPTSFAEYWNLFSPTSFAEYWNLLSPASKMDAMATTEKKPHVIFIPFPAQSHIKAMLKLAQLLHHKGLQITFVNTDFIHNQFLESSGPHCLDGAPGFRFETIPDGVSHSPEASIPIRESLLRSIETNFLDRFIDLVTKLPDPPTCIISDGFLSVFTIDAAKKLGIPVMMYWTLAACGFMGFYHIHSLIEKGFAPLKDASYLTNGYLDTVIDWVPGMEGIRLKDFPLDWSTDLNDKVLMFTTEAPQRSHKVSHHIFHTFDELEPSIIKTLSLRYNHIYTIGPLQLLLDQIPEEKKQTGITSLHGYSLVKEEPECFQWLQSKEPNSVVYVNFGSTTVMSLEDMTEFGWGLANSNHYFLWIIRSNLVIGENAVLPPELEEHIKKRGFIASWCSQEKVLKHPSVGGFLTHCGWGSTIESLSAGVPMICWPYSWDQLTNCRYICKEWEVGLEMGTKVKRDEVKRLVQELMGEGGHKMRNKAKDWKEKARIAIAPNGSSSLNIDKMVKEITVLARN

SEQ ID NO: 89 - DNA, 1893 bp, synthetic construct
  misc_feature 1..1893: note = Description of Artificial Sequence: Synthetic polynucleotide
  source 1..1893: mol_type = other DNA
atgtgcaataccaacatgtctgtacctactgatggtgctgtaaccacctcacagattccagcttcggaacaagagaccctggttagaccaaagccattgcttttgaagttattaaagtctgttggtgcacaaaaagacacttatactatgaaagaggttcttttttatcttggccagtatattatgactaaacgattatatgatgagaagcaacaacatattgtatattgttcaaatgatcttctaggagatttgtttggcgtgccaagcttctctgtgaaagagcacaggaaaatatataccatgatctacaggaacttggtagtagtcaatcagcaggaatcatcggactcaggtacatctgtgagtgagaacaggtgtcaccttgaaggtgggagtgatcaaaaggaccttgtacaagagcttcaggaagagaaaccttcatcttcacatttggtttctagaccatctaccggtggtagcggatcctctggaggcagtgctagtaaaatggcagagcaacaaaagatcaaaaagtcacctcacgtcttacttattccatttcctctgcaaggacatatcaacccattcatacaatttgggaaaagattgattagtaagggtgtaaagacaacactggtaaccactatccacactttgaattctactctgaaccactcaaatactactactacaagtatagaaattcaagctatatcagacggatgcgatgagggtggctttatgtctgccggtgaatcttacttggaaacattcaagcaagtgggatccaagtctctggccgatctaatcaaaaagttacagagtgaaggcaccacaattgacgccataatctacgattctatgacagagtgggttttagacgttgctatcgaatttggtattgatggaggttcctttttcacacaagcatgtgttgtgaattctctatactaccatgtgcataaagggttaatctctttaccattgggtgaaactgtttcagttccaggttttccagtgttacaacgttgggaaaccccattgatcttacaaaatcatgaacaaatacaatcaccttggtcccagatgttgtttggtcaattcgctaacatcgatcaagcaagatgggtctttactaattcattctataagttagaggaagaggtaattgaatggactaggaagatctggaatttgaaagtcattggtccaacattgccatcaatgtatttggacaaaagacttgatgatgataaagataatggtttcaatttgtacaaggctaatcatcacgaatgtatgaattggctggatgacaaaccaaaggaatcagttgtatatgttgctttcggctctcttgttaaacatggtccagaacaagttgaggagattacaagagcacttatagactctgacgtaaactttttgtgggtcattaagcacaaagaggaggggaaactgccagaaaacctttctgaagtgataaagaccggaaaaggtctaatcgttgcttggtgtaaacaattggatgttttagctcatgaatctgtaggctgttttgtaacacattgcggattcaactctacactagaagccatttccttaggcgtacctgtcgttgcaatgcctcagttctccgatcagacaaccaacgctaaacttttggacgaaatactaggggtgggtgtcagagttaaagcagacgagaatggtatcgtcagaagagggaacctagcttcatgtatcaaaatgatcatggaagaggaaagaggagttatcataaggaaaaacgcagttaagtggaaggatcttgcaaaggttgccgtccatgaaggcggctcttcagataatgatattgttgaatttgtgtccgaactaatcaaagcctaa

SEQ ID NO: 90 - protein, 630 aa, synthetic construct [sequence omitted]

SEQ ID NO: 91 - DNA, 1932 bp, synthetic construct
  misc_feature 1..1932: note = Description of Artificial Sequence: Synthetic polynucleotide
  source 1..1932: mol_type = other DNA
atgtgcaataccaacatgtctgtacctactgatggtgctgtaaccacctcacagattccagcttcggaacaagagaccctggttagaccaaagccattgcttttgaagttattaaagtctgttggtgcacaaaaagacacttatactatgaaagaggttcttttttatcttggccagtatattatgactaaacgattatatgatgagaagcaacaacatattgtatattgttcaaatgatcttctaggagatttgtttggcgtgccaagcttctctgtgaaagagcacaggaaaatatataccatgatctacaggaacttggtagtagtcaatcagcaggaatcatcggactcaggtacatctgtgagtgagaacaggtgtcaccttgaaggtgggagtgatcaaaaggaccttgtacaagagcttcaggaagagaaaccttcatcttcacatttggtttctagaccatctaccggtggtagcggatcctctggaggcagtgctagtaaaatggctacatctgattctattgttgatgacaggaagcagttgcatgtggctactttcccttggcttgctttcggtcatatactgccttacctacaactatcaaaactgatagctgaaaaaggacataaagtgtcattcctttcaacaactagaaacattcaaagattatcttcccacatatcaccattgattaacgtcgttcaattgacacttccaagagtacaggaattaccagaagatgctgaagctacaacagatgtgcatcctgaagatatcccttacttgaaaaaggcatccgatggattacagcctgaggtcactagattccttgagcaacacagtccagattggatcatatacgactacactcactattggttgccttcaattgcagcatcactaggcatttctagggcacatttcagtgtaaccacaccttgggccattgcttacatgggtccatccgctgatgctatgattaacggcagtgatggtagaactaccgttgaagatttgacaaccccaccaaagtggtttccatttccaactaaagtctgttggagaaaacacgacttagcaagactggttccatacaaggcaccaggaatctcagacggctatagaatgggtttagtccttaaagggtctgactgcctattgtctaagtgttaccatgagtttgggacacaatggctaccacttttggaaacattacaccaagttcctgtcgtaccagttggtctattacctccagaaatccctggtgatgagaaggacgagacttgggtttcaatcaaaaagtggttagacgggaagcaaaaaggctcagtggtatatgtggcactgggttccgaagttttagtatctcaaacagaagttgtggaacttgccttaggtttggaactatctggattgccatttgtctgggcctacagaaaaccaaaaggccctgcaaagtccgattcagttgaattgccagacggctttgtcgagagaactagagatagagggttggtatggacttcatgggctccacaattgagaatcctgagtcacgaatctgtgtgcggtttcctaacacattgtggttctggttctatagttgaaggactgatgtttggtcatccacttatcatgttgccaatctttggtgaccagcctttgaatgcacgtctgttagaagataaacaagttggaattgaaatcccacgtaatgaggaagatggatgtttaaccaaggagtctgtggccagatcattacgttccgttgtcgttgaaaaggaaggcgaaatctacaaggccaatgcccgtgaactttcaaagatctacaatgacacaaaagtagagaaggaatatgtttctcaatttgtagattacctagagaaaaacgctagagccgtagctattgatcatgaatcctaa

SEQ ID NO: 92 - protein, 643 aa, synthetic construct
  REGION 1..643: note = Description of Artificial Sequence: Synthetic polypeptide
MCNTNMSVPTDGAVTTSQIPASEQETLVRPKPLLLKLLKSVGAQKDTYTMKEVLFYLGQYIMTKRLYDEKQQHIVYCSNDLLGDLFGVPSFSVKEHRKIYTMIYRNLVVVNQQESSDSGTSVSENRCHLEGGSDQKDLVQELQEEKPSSSHLVSRPSTGGSGSSGGSASKMATSDSIVDDRKQLHVATFPWLAFGHILPYLQLSKLIAEKGHKVSFLSTTRNIQRLSSHISPLINVVQLTLPRVQELPEDAEATTDVHPEDIPYLKKASDGLQPEVTRFLEQHSPDWIIYDYTHYWLPSIAASLGISRAHFSVTTPWAIAYMGPSADAMINGSDGRTTVEDLTTPPKWFPFPTKVCWRKHDLARLVPYKAPGISDGYRMGLVLKGSDCLLSKCYHEFGTQWLPLLETLHQVPVVPVGLLPPEIPGDEKDETWVSIKKWLDGKQKGSVVYVALGSEVLVSQTEVVELALGLELSGLPFVWAYRKPKGPAKSDSVELPDGFVERTRDRGLVWTSWAPQLRILSHESVCGFLTHCGSGSIVEGLMFGHPLIMLPIFGDQPLNARLLEDKQVGIEIPRNEEDGCLTKESVARSLRSVVVEKEGEIYKANARELSKIYNDTKVEKEYVSQFVDYLEKNARAVAIDHES

SEQ ID NO: 93 - DNA, 1887 bp, synthetic construct
  misc_feature 1..1887: note = Description of Artificial Sequence: Synthetic polynucleotide
  source 1..1887: mol_type = other DNA
atgtgcaataccaacatgtctgtacctactgatggtgctgtaaccacctcacagattccagcttcggaacaagagaccctggttagaccaaagccattgcttttgaagttattaaagtctgttggtgcacaaaaagacacttatactatgaaagaggttcttttttatcttggccagtatattatgactaaacgattatatgatgagaagcaacaacatattgtatattgttcaaatgatcttctaggagatttgtttggcgtgccaagcttctctgtgaaagagcacaggaaaatatataccatgatctacaggaacttggtagtagtcaatcagcaggaatcatcggactcaggtacatctgtgagtgagaacaggtgtcaccttgaaggtgggagtgatcaaaaggaccttgtacaagagcttcaggaagagaaaccttcatcttcacatttggtttctagaccatctaccggtggtagcggatcctctggaggcagtgctagtaaaatggaaaacaagaccgaaacaacagttagacgtaggcgtagaatcattctgtttccagtaccttttcaagggcacatcaatccaatactacaactagccaacgttttgtactctaaaggtttttctattacaatctttcacaccaatttcaacaaaccaaaaacatccaattacccacatttcacattcagattcatacttgataatgatccacaagatgaacgtatttcaaacttacctacccacggtcctttagctggaatgagaattccaatcatcaatgaacatggtgccgatgagcttagaagagaattagagttacttatgttggcatccgaagaggacgaggaagtctcttgtctgattactgacgctctatggtactttgcccaatctgtggctgatagtttgaatttgaggagattggtactaatgacatccagtctgtttaactttcacgctcatgttagtttaccacaatttgacgaattgggatacttggaccctgatgacaagactaggttagaggaacaggcctctggttttcctatgttgaaagtcaaagatatcaagtctgcctattctaattggcaaatcttgaaagagatcttaggaaagatgatcaaacagacaaaggcttcatctggagtgatttggaacagtttcaaagagttagaagagtctgaattggagactgtaatcagagaaattccagcaccttcattcctgataccattaccaaaacatttgactgcttcctcttcctctttgttggatcatgacagaacagtttttcaatggttggaccaacaaccacctagttctgttttgtacgtgtcatttggtagtacttctgaagtcgatgaaaaggacttccttgaaatcgcaagaggcttagtcgatagtaagcagtcattcctttgggtcgtgcgtccaggtttcgtgaaaggctcaacatgggtcgaaccacttccagatggttttctaggcgaaagaggtagaatagtcaaatgggttcctcaacaggaagttttagctcatggcgctattggggcattctggactcattccggatggaattcaactttagaatcagtatgcgaaggggtacctatgatcttttcagattttggtcttgatcaaccactgaacgcaagatacatgtctgatgttttgaaagtgggtgtatatctagaaaatggctgggaaaggggtgaaatagctaatgcaataagacgtgttatggttgatgaagagggggagtatatcagacaaaacgcaagagtgctgaagcaaaaggccgacgtttctctaatgaagggaggctcttcatacgaatccttagaatctcttgtttcctacatttcatcactgtaa

SEQ ID NO: 94 - protein, 628 aa, synthetic construct
  REGION 1..628: note = Description of Artificial Sequence: Synthetic polypeptide
MCNTNMSVPTDGAVTTSQIPASEQETLVRPKPLLLKLLKSVGAQKDTYTMKEVLFYLGQYIMTKRLYDEKQQHIVYCSNDLLGDLFGVPSFSVKEHRKIYTMIYRNLVVVNQQESSDSGTSVSENRCHLEGGSDQKDLVQELQEEKPSSSHLVSRPSTGGSGSSGGSASKMENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 95 - protein, 473 aa, Stevia rebaudiana
MATSDSIVDDRKQLHVATFPWLAFGHILPYLQLSKLIAEKGHKVSFLSTTRNIQRLSSHISPLINVVQLTLPRVQELPEDAEATTDVHPEDIPYLKKASDGLQPEVTRFLEQHSPDWIIYDYTHYWLPSIAASLGISRAHFSVTTPWAIAYMGPSADAMINGSDGRTTVEDLTTPPKWFPFPTKVCWRKHDLARLVPYKAPGISDRCRMGLVLKGSDCLLSKCYHEFGTQWLPLLETLHQVPVVPVGLLPPEIPGDEKDETWVSIKKWLDGKQKGSVVYVALGSEVLVSQTEVVELALGLELSGLPFVWAYRKPKGPAKSDSVELPDGFVERTRDRGLVWTSRAPQLRILSHESVCGFLTHCGSGSIVEGLMFGHPLIMLPIFGDQPLNARLLEDKQVGIEIPRNEEDGCLTKESVARSLRSVVVEKEGEIYKANARELSKIYNDTKVEKEYVSQFVDYLEKNARAVAIDHES

SEQ ID NO: 96 - DNA, 1422 bp, Stevia rebaudiana
  source 1..1422: mol_type = genomic DNA
atggctaccagtgactccatagttgacgaccgtaagcagcttcatgttgcgacgttcccatggcttgctttcggtcacatcctcccttaccttcagctttcgaaattgatagctgaaaagggtcacaaagtctcgtttctttctaccaccagaaacattcaacgtctctcttctcatatctcgccactcataaatgttgttcaactcacacttccacgtgtccaagagctgccggaggatgcagaggcgaccactgacgtccaccctgaagatattccatatctcaagaaggcttctgatggtcttcaaccggaggtcacccggtttctagaacaacactctccggactggattatttatgattatactcactactggttgccatccatcgcggctagcctcggtatctcacgagcccacttctccgtcaccactccatgggccattgcttatatgggaccctcagctgacgccatgataaatggttcagatggtcgaaccacggttgaggatctcacgacaccgcccaagtggtttccctttccgaccaaagtatgctggcggaagcatgatcttgcccgactggtgccttacaaagctccggggatatctgatcgatgccgtatggggctggttcttaagggatctgattgtttgctctccaaatgttaccatgagtttggaactcaatggctacctcttttggagacactacaccaagtaccggtggttccggtgggattactgccaccggaaatacccggagacgagaaagatgaaacatgggtgtcaatcaagaaatggctcgatggtaaacaaaaaggcagtgtggtgtacgttgcattaggaagcgaggttttggtgagccaaaccgaggttgttgagttagcattgggtctcgagctttctgggttgccatttgtttgggcttatagaaaaccaaaaggtcccgcgaagtcagactcggtggagttgccagacgggttcgtggaacgaactcgtgaccgtgggttggtctggacgagtcgggcacctcagttacgaatactgagccatgagtcggtttgtgggttcttgacgcattgtggttctggatcaattgtggaagggctaatgtttggtcaccctctaatcatgctaccgatttttggggaccaacctctgaatgctcgattactggaggacaaacaggtgggaatcgagataccaagaaatgaggaagatggttgcttgaccaaggagtcggttgctagatcactgaggtccgttgttgtggaaaaagaaggggagatctacaaggcgaacgcgagggagctgagtaaaatctataacgacactaaggttgaaaaagaatatgtaagccaattcgtagactatttggaaaagaatgcgcgtgcggttgccatcgatcatgagagttaa

SEQ ID NO: 97 - DNA, 1380 bp, Stevia rebaudiana
  source 1..1380: mol_type = genomic DNA
atggaaaataaaacggagaccaccgttcgccggcgccggagaataatattattcccggtaccatttcaaggccacattaacccaattcttcagctagccaatgtgttgtactctaaaggattcagtatcaccatctttcacaccaacttcaacaaacccaaaacatctaattaccctcacttcactttcagattcatcctcgacaacgacccacaagacgaacgcatttccaatctaccgactcatggtccgctcgctggtatgcggattccgattatcaacgaacacggagctgacgaattacgacgcgaactggaactgttgatgttagcttctgaagaagatgaagaggtatcgtgtttaatcacggatgctctttggtacttcgcgcaatctgttgctgacagtcttaacctccgacggcttgttttgatgacaagcagcttgtttaattttcatgcacatgtttcacttcctcagtttgatgagcttggttacctcgatcctgatgacaaaacccgtttggaagaacaagcgagtgggtttcctatgctaaaagtgaaagacatcaagtctgcgtattcgaactggcaaatactcaaagagatattagggaagatgataaaacaaacaaaagcatcttcaggagtcatctggaactcatttaaggaactcgaagagtctgagctcgaaactgttatccgtgagatcccggctccaagtttcttgataccactccccaagcatttgacagcctcttccagcagcttactagaccacgatcgaaccgtttttccatggttagaccaacaaccgtcacgttcggtactgtatgttagttttggtagtggtactgaagtactggatgagaaagatttcttggaaatagctcgtgggttggttgatagcaagcagtcgtttttatgggtggttcgacctgggtttgtcaagggttcgacgtgggtcgaaccgttgccagatgggttcttgggtgaaagaggacgtattgtgaaatgggttccacagcaagaagtgctagctcatggagcaataggcgcattctggactcatagcggatggaactctacgttggaaagcgtttgtgaaggtgttcctatgattttctcggattttgggctcgatcaaccgttgaatgctagatacatgagtgatgttttgaaggtaggggtgtatttggaaaatgggtgggaaagaggagagatagcaaatgcaataagaagagttatggtggatgaagaaggagaatacattagacagaatgcaagagttttgaaacaaaaggcagatgtttctttgatgaagggtggttcgtcttacgaatcattagagtctctagtttcttacatttcatcgttgtaa

SEQ ID NO: 98 - protein, 459 aa, Stevia rebaudiana
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFPWLDQQPSRSVLYVSFGSGTEVLDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 99 - DNA, 1380 bp, Stevia rebaudiana
  modified_base 861..863: mod_base = OTHER; note = a, c, t, g, unknown or other
  source 1..1380: mol_type = genomic DNA
atggaaaataaaacggagaccaccgttcgccggcgccggagaataatattattcccggtaccatttcaaggccacattaacccaattcttcagctagccaatgtgttgtactctaaaggattcagtatcaccatctttcacaccaacttcaacaaacccaaaacatctaattaccctcacttcactttcagattcatcctcgacaacgacccacaagacgaacgcatttccaatctaccgactcatggtccgctcgctggtatgcggattccgattatcaacgaacacggagctgacgaattacgacgcgaactggaactgttgatgttagcttctgaagaagatgaagaggtatcgtgtttaatcacggatgctctttggtacttcgcgcaatctgttgctgacagtcttaacctccgacggcttgttttgatgacaagcagcttgtttaattttcatgcacatgtttcacttcctcagtttgatgagcttggttacctcgatcctgatgacaaaacccgtttggaagaacaagcgagtgggtttcctatgctaaaagtgaaagacatcaagtctgcgtattcgaactggcaaatactcaaagagatattagggaagatgataaaacaaacaaaagcatcttcaggagtcatctggaactcatttaaggaactcgaagagtctgagctcgaaactgttatccgtgagatcccggctccaagtttcttgataccactccccaagcatttgacagcctcttccagcagcttactagaccacgatcgaaccgtttttcaatggttagaccaacaaccgccaagttcggtactgtatgttagttttggtagtactagtgaagtnnnggatgagaaagatttcttggaaatagctcgtgggttggttgatagcaagcagtcgtttttatgggtggttcgacctgggtttgtcaagggttcgacgtgggtcgaaccgttgccagatgggttcgtggccgaaagagggcgtattgtgaaatgggttccgcaacaggaagtgatagctcatggagcaatcggtgcattctggactcatagcggatggaactctacattggaaagcgtttgtgaaggtgttcctatgattttctcggattttgggctcgatcaaccgttgaatgctagatacatgagtgatgttttgaaggtaggggtgtatttggaaaatgggtgggaaagaggagagatagcaaatgcaatacgaagagttatggtggatgaagaaggagaatacattagacagaatgcaagagttttgaaacaaaaggcagatgtttctttgatgaagggtggttcatcttacgaatcattagagtctctagtttcttacatttcatcgttgtaa

SEQ ID NO: 100 - protein, 459 aa, Stevia rebaudiana
  MOD_RES 288: note = Any amino acid
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFQWLDQQPPSSVLYVSFGSTSEVXDEKDFLEIARGLVDSKQSFLWVVRPGFVKGSTWVEPLPDGFVAERGRIVKWVPQQEVIAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 101 - DNA, 1380 bp, Stevia rebaudiana
  modified_base 861..863: mod_base = OTHER; note = a, c, t, g, unknown or other
  source 1..1380: mol_type = genomic DNA
atggaaaataaaacggagaccaccgttcgccggcgccggagaataatattattcccggtaccatttcaaggccacattaacccaattcttcagctagccaatgtgttgtactctaaaggattcagtatcaccatctttcacaccaacttcaacaaacccaaaacatctaattaccctcacttcactttcagattcatcctcgacaacgacccacaagacgaacgcatttccaatctaccgactcatggtccgctcgctggtatgcggattccgattatcaacgaacacggagctgacgaattacgacgcgaactggaactgttgatgttagcttctgaagaagatgaagaggtatcgtgtttaatcacggatgctctttggtacttcgcgcaatctgttgctgacagtcttaacctccgacggcttgttttgatgacaagcagcttgtttaattttcatgcacatgtttcacttcctcagtttgatgagcttggttacctcgatcctgatgacaaaacccgtttggaagaacaagcgagtgggtttcctatgctaaaagtgaaagacatcaagtctgcgtattcgaactggcaaatactcaaagagatattagggaagatgataaaacaaacaaaagcatcttcaggagtcatctggaactcatttaaggaactcgaagagtctgagctcgaaactgttatccgtgagatcccggctccaagtttcttgataccactccccaagcatttgacagcctcttccagcagcttactagaccacgatcgaaccgtttttcaatggttagaccaacaaccgccaagttcggtactgtatgttagttttggtagtactagtgaagtnnnggatgagaaagatttcttggaaatagctcgtgggttggttgatagcaagcagtcgtttttatgggtggttcgacctgggtttgtcaagggttcgacgtgggtcgaaccgttgccagatgggttcttgggtgaaagaggacgtattgtgaaatgggttccacagcaagaagtgctagctcatggagcaataggcgcattctggactcatagcggatggaactctacgttggaaagcgtttgtgaaggtgttcctatgattttctcggattttgggctcgatcaaccgttgaatgctagatacatgagtgatgttttgaaggtaggggtgtatttggaaaatgggtgggaaagaggagagatagcaaatgcaataagaagagttatggtggatgaagaaggagaatacattagacagaatgcaagagttttgaaacaaaaggcagatgtttctttgatgaagggtggttcgtcttacgaatcattagagtctctagtttcttacatttcatcgttgtaa

SEQ ID NO: 102 - protein, 459 aa, Stevia rebaudiana
  MOD_RES 288: note = Any amino acid
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFQWLDQQPPSSVLYVSFGSTSEVXDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 103 - DNA, 1323 bp, synthetic construct
  misc_feature 1..1323: note = Description of Artificial Sequence: Synthetic polynucleotide
  source 1..1323: mol_type = other DNA
atgcattctaccagacatatcttaagacaaagggccgtcctagttacaggcgctagaacaccattcgtgaaatcatttggggctcttatgaaagcagataccttggaattggcatcagcatcagtcgctgggttgctgaacaagacctcactggaccctagagatatcgatcatatcgtttggggtaatgttgtacttcaaggatcagctcataactgcgccagagaaatagttatcgaccttaacatgcctaaaaagatcatcggtaatttgacatctatggcctgtgcttcaggcttatcttctttgtcacaagcctgtatgctaatagagggtggtcatgccgatgtcgtcattgctggcggttctgattcagtctccaacactgaagtgcctttgccaagatccgtcacttacggtctaatgatggcccaaaggaagggtgttatgggcttctttaaggaagcaggatacaacccattcaaatggtttccaggcggtattgctttaaccgaacgtagtacaggaaaaactatgggttggcatggagacttaattgctgagttaaactctatatctagagatgaccaggaagccctggctgtggcttctcatgcaaatgctgctagagcagaaaaagctgggtactttaaggaggaaattgtacctgtgacaatcgacaaaaagggcaaaaagactgaagtaacatgtgatgatgttatgcaaagagatacagaaaagatgaaggccaagatgccatcattgaagcctgttttcagaaaagagggaggtacaataacagcagccacttccagtactctgactgatggtggctctgcaatgttggttatgtcagaggaaaaggccaaaaagttgggttatccaactgatgtctgcgtgaagtcttggtatttcagtggtatcgatccttacccacaacttttgttagcaccagttctaggttggggtccagctttgaaaaaggccggattaacccctaaagatatcgatttgtacgaaattcacgaagcatttgctgcacaagttctagccacaattaagtgtttgaagtctcaggaattcttcgataggtacgctaacggtgcaaagccagtattaactgaggatattgatctttctaaactaaatgttaatggcggttccttagcacttggccacccattcgccgctacaggaggtagaatcgtaatctctctagcaaatgagttgagaagatccggaaagagacacgggctggtcagtatttgtgcagctggagggttaggcggagtagctatacttgagcatacagcaagtaagtaa

SEQ ID NO: 104 - protein, 440 aa, Leishmania infantum
MHSTRHILRQRAVLVTGARTPFVKSFGALMKADTLELASASVAGLLNKTSLDPRDIDHIVWGNVVLQGSAHNCAREIVIDLNMPKKIIGNLTSMACASGLSSLSQACMLIEGGHADVVIAGGSDSVSNTEVPLPRSVTYGLMMAQRKGVMGFFKEAGYNPFKWFPGGIALTERSTGKTMGWHGDLIAELNSISRDDQEALAVASHANAARAEKAGYFKEEIVPVTIDKKGKKTEVTCDDVMQRDTEKMKAKMPSLKPVFRKEGGTITAATSSTLTDGGSAMLVMSEEKAKKLGYPTDVCVKSWYFSGIDPYPQLLLAPVLGWGPALKKAGLTPKDIDLYEIHEAFAAQVLATIKCLKSQEFFDRYANGAKPVLTEDIDLSKLNVNGGSLALGHPFAATGGRIVISLANELRRSGKRHGLVSICAAGGLGGVAILEHTASK

SEQ ID NO: 105 - DNA, 1584 bp, synthetic construct
  misc_feature 1..1584: note = Description of Artificial Sequence: Synthetic polynucleotide
  source 1..1584: mol_type = other DNA
atggcagctgaccaattggtgaaaactgaagtcaccaagaagtcttttactgctcctgtacaaaaggcttctacaccagttttaaccaataaaacagtcatttctggatcgaaagtcaaaagtttatcatctgcgcaatcgagctcatcaggaccttcatcatctagtgaggaagatgattcccgcgatattgaaagcttggataagaaaatacgtcctttagaagaattagaagcattattaagtagtggaaatacaaaacaattgaagaacaaagaggtcgctgccttggttattcacggtaagttacctttgtacgctttggagaaaaaattaggtgatactacgagagcggttgcggtacgtaggaaggctctttcaattttggcagaagctcctgtattagcatctgatcgtttaccatataaaaattatgactacgaccgcgtatttggcgcttgttgtgaaaatgttataggttacatgcctttgcccgttggtgttataggccccttggttatcgatggtacatcttatcatataccaatggcaactacagagggttgtttggtagcttctgccatgcgtggctgtaaggcaatcaatgctggcggtggtgcaacaactgttttaactaaggatggtatgacaagaggcccagtagtccgtttcccaactttgaaaagatctggtgcctgtaagatatggttagactcagaagagggacaaaacgcaattaaaaaagcttttaactctacatcaagatttgcacgtctgcaacatattcaaacttgtctagcaggagatttactcttcatgagatttagaacaactactggtgacgcaatgggtatgaatatgatttctaaaggtgtcgaatactcattaaagcaaatggtagaagagtatggctgggaagatatggaggttgtctccgtttctggtaactactgtaccgacaaaaaaccagctgccatcaactggatcgaaggtcgtggtaagagtgtcgtcgcagaagctactattcctggtgatgttgtcagaaaagtgttaaaaagtgatgtttccgcattggttgagttgaacattgctaagaatttggttggatctgcaatggctgggtctgttggtggatttaacgcacatgcagctaatttagtgacagctgttttcttggcattaggacaagatcctgcacaaaatgttgaaagttccaactgtataacattgatgaaagaagtggacggtgatttgagaatttccgtatccatgccatccatcgaagtaggtaccatcggtggtggtactgttctagaaccacaaggtgccatgttggacttattaggtgtaagaggcccgcatgctaccgctcctggtaccaacgcacgtcaattagcaagaatagttgcctgtgccgtcttggcaggtgaattatccttatgtgctgccctagcagccggccatttggttcaaagtcatatgacccacaacaggaaacctgctgaaccaacaaaacctaacaatttggacgccactgatataaatcgtttgaaagatgggtccgtcacctgcattaaatcctaa

SEQ ID NO: 106 - protein, 527 aa, Saccharomyces cerevisiae
MAADQLVKTEVTKKSFTAPVQKASTPVLTNKTVISGSKVKSLSSAQSSSSGPSSSSEEDDSRDIESLDKKIRPLEELEALLSSGNTKQLKNKEVAALVIHGKLPLYALEKKLGDTTRAVAVRRKALSILAEAPVLASDRLPYKNYDYDRVFGACCENVIGYMPLPVGVIGPLVIDGTSYHIPMATTEGCLVASAMRGCKAINAGGGATTVLTKDGMTRGPVVRFPTLKRSGACKIWLDSEEGQNAIKKAFNSTSRFARLQHIQTCLAGDLLFMRFRTTTGDAMGMNMISKGVEYSLKQMVEEYGWEDMEVVSVSGNYCTDKKPAAINWIEGRGKSVVAEATIPGDVVRKVLKSDVSALVELNIAKNLVGSAMAGSVGGFNAHAANLVTAVFLALGQDPAQNVESSNCITLMKEVDGDLRISVSMPSIEVGTIGGGTVLEPQGAMLDLLGVRGPHATAPGTNARQLARIVACAVLAGELSLCAALAAGHLVQSHMTHNRKPAEPTKPNNLDATDINRLKDGSVTCIKS

SEQ ID NO: 107 - DNA, 3681 bp, synthetic construct
  misc_feature 1..3681: note = Description of Artificial Sequence: Synthetic polynucleotide
  source 1..3681: mol_type = other DNA
atgagagctgtccttagattgttatcaacacatactgttttctctcctattgaaacaattgtatctgttttcgtgttagctacattagcttacttccacatcttgtccggaatcaagcactcaagtttctttgcatcttctcatcctcctgctatcagacctgcttttgcacatctgaccaacggggaatgggttgccgtctcccaacatgattggactgaagcatggaagcatcctggcggttcacttgatgcattagaacttcaacaagtagttttcactttagatgacaagactcaaccatctgctgtgctagatgcatccgcaattagtcagcacttagtttccaatgttcctgcattatctggaaaagcctactcttcattgtgccaccatccaaatgtatcaggcacctcctgttttacatcagtttctggtccaggagcttcaccaatcttgacactgagttttaagcctggaactagagacgattggttaggatcattaaggaaggagaaaactatcacactagatggggttaagtacgacgttggagccggaaaaagacaagagtcaatcggcgatatggaatcatctaagtgggttgcttatgcattatcagctttggtacttagattttgggaattaacaaaggcagattccttagatatactagtggttctaactgggtacatcctaatgcacgtaacattcatgagattgttcttggcatccagagcacttggcagtaacttttggttatcagctggcatattctcctccgcaacaatttctttcctattcactttaccaatgtgtagatctatggatattccacttgatccaattgccttgacagaagccctgccattcttggtgtgtaccgtaggttttgacaaaccacttagattggcaagagctgtgatggctcatcctaatatccttaaacctcaagatgatggtaggatgaaagctgccggagatgtcattcttgaggcactggacagagttggtaacatgatattgagagattacgctttagagatcgcagttctattcgttggcgttaactccagagttggcggtcttaaggaattttgtgctgtagctgcagcattacttgctatggacagattaatgacattcacactttatacagcagtgttaaccatcatggttgaggtaaggcgtatcaaaaaggtcagagatatgactaaggctagatctagaagttcttctattaccgccgttacagccaacggcaccgccataagaggcgttttgagtagaaaatcttcaaaacaatctgtgacagaaccagagacaactaaaaacctaagacaaagagccactgattcagccatcggtgttaagggttcattgctgaaagatggaggcagattgcaggaagccgaggagaatccaatggcaagattaaagctattgttaatcgcttccttcttaacactacacatcttgaacttttgtactactttgacttcagccacagctaacgcaagacatcaaagacatccttttagaaccgttcaagaggtagtaccaattcctagagttgacattactaccccagccatagccaatatcttgtctcatctagctgtggctcaggaacctatgttcactgttgttggcagtgaacctatcgaacttcttgttaaagtcgctgctccagtctacgtccatgctctaccattggcccctgctttaagagcttcaaacactaatactggagaagctattgaaaactttatgagttcatggtctagtctggtaggtgacccagttgttagtaagtggatcgtagcattgctagctgtctctgttgcattgaatggatacttgttaaagggtatagccgcaggttccgggttggctgccatgagagctgttagatctcaaggtgttcgtttcagatctagagctagaagtatcgtaaagatatctgatgaacctgagccagagccagaacactctatcgacccagcaccagtagtgttcttcgcttccgcagcaccagctgtagaggcccctgctccagctcctgcacctgaaccagaaccaccagtcaacagaccaccaccattgactattttctcaagaccactgaacttagaaacagtggacaaaaagttacaagatgctctgccaataagatccccaccacctgttgaaccaatcactccagaatctagagaagtggaaccaacccaagtagaagtaagatctctagctgaatgtgtggatgtgttcgagaatgggccaagaccagtctcagtggctttaaagactctgaatgatgaggaagttatcctgctttgccaaacaggtaagatagctccatatgcattggttaagatgttggctgatttcgatagggccgtacgtgtcagaagagcacttattagtagagcttcacgtacaaaaactttagaaaactcactggttcctatgaaagattatgattacgccagagtcatgggtgcctgttgtgaaaacgttatcggatacatgccattaccactagggattgcaggtccattgaagattgatggcttgatgtatcctataccaatggcaaccgcagaaggtaccttggttgcatctacttctaggggctgtaaggccttaaatgctggtggaggggtcacaactgtcttgacagcagatggcatgacaagagggccagctatagactttccttccatcgtcagagctgcagaggctaaggccttcattgaatcagaagatggatacgctacaatcagggaggctttcgagtctacttctagatttgccaagttgcaaaagatcaagtgtgcactagctggtcgtactctttttgtcagatttgctactagaacaggagatgccatgggtatgaacatgatttctaaggctaccgaaaaggcacttgatgtcctgagtcacgagttccctgaaatggtcgtccttgctttgtctggtaactactgcacagacaaaaagcctgcagctatttcatggatcgaaggtaggggaaaatctattgtagcagaagcagttattcctggtaaggtcgttaagtcagtcctgaaaacaacagtcgagtctctttgcaatgtcaacactaagaaaaacctgattggttcagccatggcaggttctgttggtggtttcaacgctcatgccgccaacatcctaacagctgtgttcctagccacaggtcaggatcctgctcaaaatgtcgaatcttctaattgcatgactttaatggaaccaacaaacggcggtgaggatttgctaatgacaatttcaatgccatgtatagaggtaggaaccgttggtggagggacaattctggaaccacaaggtgcagttttggatttgttgggcgttagaggggctcaccctactaatcctggtcaaaacgctcaacagttagccagaattatcgcatcagctgtaatggcaggcgaattgtctttgataagtgccttagccgcaggtcatttggttagagctcatcttgcccacaatcgttctcaattgaatacaccaatgccatccagaccacatactcctggccctgaggatgtctcacatgtgcagcagctacctacaccatctgcatctgatgataaaggtgttacagctcaaggttacgttgtcgaagcaaaataa

SEQ ID NO: 108 - protein, 1226 aa, Ganoderma lucidum
MRAVLRLLSTHTVFSPIETIVSVFVLATLAYFHILSGIKHSSFFASSHPPAIRPAFAHLTNGEWVAVSQHDWTEAWKHPGGSLDALELQQVVFTLDDKTQPSAVLDASAISQHLVSNVPALSGKAYSSLCHHPNVSGTSCFTSVSGPGASPILTLSFKPGTRDDWLGSLRKEKTITLDGVKYDVGAGKRQESIGDMESSKWVAYALSALVLRFWELTKADSLDILVVLTGYILMHVTFMRLFLASRALGSNFWLSAGIFSSATISFLFTLPMCRSMDIPLDPIALTEALPFLVCTVGFDKPLRLARAVMAHPNILKPQDDGRMKAAGDVILEALDRVGNMILRDYALEIAVLFVGVNSRVGGLKEFCAVAAALLAMDRLMTFTLYTAVLTIMVEVRRIKKVRDMTKARSRSSSITAVTANGTAIRGVLSRKSSKQSVTEPETTKNLRQRATDSAIGVKGSLLKDGGRLQEAEENPMARLKLLLIASFLTLHILNFCTTLTSATANARHQRHPFRTVQEVVPIPRVDITTPAIANILSHLAVAQEPMFTVVGSEPIELLVKVAAPVYVHALPLAPALRASNTNTGEAIENFMSSWSSLVGDPVVSKWIVALLAVSVALNGYLLKGIAAGSGLAAMRAVRSQGVRFRSRARSIVKISDEPEPEPEHSIDPAPVVFFASAAPAVEAPAPAPAPEPEPPVNRPPPLTIFSRPLNLETVDKKLQDALPIRSPPPVEPITPESREVEPTQVEVRSLAECVDVFENGPRPVSVALKTLNDEEVILLCQTGKIAPYALVKMLADFDRAVRVRRALISRASRTKTLENSLVPMKDYDYARVMGACCENVIGYMPLPLGIAGPLKIDGLMYPIPMATAEGTLVASTSRGCKALNAGGGVTTVLTADGMTRGPAIDFPSIVRAAEAKAFIESEDGYATIREAFESTSRFAKLQKIKCALAGRTLFVRFATRTGDAMGMNMISKATEKALDVLSHEFPEMVVLALSGNYCTDKKPAAISWIEGRGKSIVAEAVIPGKVVKSVLKTTVESLCNVNTKKNLIGSAMAGSVGGFNAHAANILTAVFLATGQDPAQNVESSNCMTLMEPTNGGEDLLMTISMPCIEVGTVGGGTILEPQGAVLDLLGVRGAHPTNPGQNAQQLARIIASAVMAGELSLISALAAGHLVRAHLAHNRSQLNTPMPSRPHTPGPEDVSHVQQLPTPSASDDKGVTAQGYVVEAK

SEQ ID NO: 109 - DNA, 2667 bp, synthetic construct
  misc_feature 1..2667: note = Description of Artificial Sequence: Synthetic polynucleotide
  source 1..2667: mol_type = other DNA
atgttatcaagattgttcagaatgcatggtctatttgttgcttctcacccttgggaagtaatagttggtactgtaacattaacgatctgtatgatgtctatgaacatgtttaccggaaacaacaagatttgtggttggaattatgagtgtcctaagctggaagaggatgtgttgagttcagacatcatcatacttactataacaagatgcattgcaatattgtatatctacttccaatttcaaaaccttagacaattgggtagtaaatacatcctaggcatcgccggattgttcactattttctctagttttgttttctcaaccgtcgttattcactttttggacaaagagttaactggtttgaacgaagctctaccattcttcttgctgctggtagatttgtccagagcttccgctttagctaaattcgctctgtcctctaattctcaagatgaagttagagagaatatagcaaggggaatggccatacttggacctactttcacacttgatgcccttgtcgaatgtttggttattggggttggcacaatgtccggcgttagacagttagaaatcatgtgttgttttggctgtatgagtgtcttggctaactactttgtctttatgacattctttccagcttgcgtttctttggtattggagctgtcaagagaatcaagagaaggcagaccaatatggcaactatcacatttcgccagagtgttagaagaggaggaaaacaaacctaatcctgtcacacagagagtgaaaatgatcatgtctttgggtttagtcctagtgcatgctcattctagatggatcgcagatccatcccctcagaattctacagctgataactctaaagttagtttaggtttagatgaaaatgtaagtaagaggattgaaccttccgtgtctttgtggcaattctacttatcaaaaatgatttccatggatattgaacaagtgataacgttgtctttggctttattgttagccgttaagtacattttctttgagcaagccgaaacggaatctacattatcactgaaaaacccaattacatccccagtcgttacccagaaaaagataactgatgattgctgtagaagagatccagtgttggtcaggaatgatcaaaagttccacgccatggaggaggaaactaggaaaaacagagaaaggaaagttgaagttatcaagcctctattagcagaaaatgacacttcacatagggccactttcgttgtcggcaattcatctcttttaggtacgtcattggagctggaaacacaggaaccagaaatggaactaccagttgaaccaagaccaaatgaggaatgtttgcaaatactagagaacgctgaaaagggagccaagttcctatctgatgccgagattatccagctggtcaatgccaagcacattcctgcctacaagttggaaacccttatggagacacatgagagaggtgtgtctattaggagacaattactatctaaaaagttacctgaaccaagttccctacaatacctgccttatagagattacaattactccttggtaatgggagcttgttgtgaaaatgtcattgggtacatgccaattccagtgggtgtcgccggtccactatgtttggacggtaaggaatttcaagtacctatggcaacgactgaaggctgcttagttgcatctacaaacagaggttgtagagccattggattaggtggcggtgcttcttcaagagtcttggctgacggtatgactagaggtcctgttgtgagatttcctagggcctgtgactctgcagaagttaaggcttggttggaaactccagaaggtttcaccgtaatcaaagaggcctttgattccacatcaagggtggccagattacaaaaactacacatgtctgtcgctgggagaaatctgtatatcagatttcaatccagatccggcgacgcaatgggtatgaatatgatttcaaaagggacagaaaaggctttgtcaaagctgcaggagtatttcccagagatgcaaatcttggccgtatctggcaactattgcacagacaaaaagcctgccgccatcaactggattgaaggaagaggcaaatctgtggtttgtgaagctgtaattccagccaaagttgttagagaagtgttaaagaccacaacagaagctatgattgaagtaaacataaacaaaaacttagtagggtctgccatggctggttcaattggaggatacaacgctcatgctgccaatattgtaaccgctatctacatcgcatgtggacaagatgctgcccaaaatgtcggttcctcaaattgcatcacattgatggaagcatctggccctacaaacgaggatttgtatatcagttgcacaatgccatctatagaaatagggactgtgggaggaggaactaacttacttccacagcaagcctgcttacaaatgctgggtgtacaaggagcctgtagagataatccaggggagaacgctagacaacttgccagaattgtttgtgggacagttatggctggtgaacttagtctaatggcagctttggctgctgggcacctggtgagatctcatatgattcataatagaagtaagattaaccttcaagatttgcaaggtacgtgtacgaaaaaggctgcctaa

SEQ ID NO: 110 - protein, 888 aa, Bos taurus
MLSRLFRMHGLFVASHPWEVIVGTVTLTICMMSMNMFTGNNKICGWNYECPKLEEDVLSSDIIILTITRCIAILYIYFQFQNLRQLGSKYILGIAGLFTIFSSFVFSTVVIHFLDKELTGLNEALPFFLLLVDLSRASALAKFALSSNSQDEVRENIARGMAILGPTFTLDALVECLVIGVGTMSGVRQLEIMCCFGCMSVLANYFVFMTFFPACVSLVLELSRESREGRPIWQLSHFARVLEEEENKPNPVTQRVKMIMSLGLVLVHAHSRWIADPSPQNSTADNSKVSLGLDENVSKRIEPSVSLWQFYLSKMISMDIEQVITLSLALLLAVKYIFFEQAETESTLSLKNPITSPVVTQKKITDDCCRRDPVLVRNDQKFHAMEEETRKNRERKVEVIKPLLAENDTSHRATFVVGNSSLLGTSLELETQEPEMELPVEPRPNEECLQILENAEKGAKFLSDAEIIQLVNAKHIPAYKLETLMETHERGVSIRRQLLSKKLPEPSSLQYLPYRDYNYSLVMGACCENVIGYMPIPVGVAGPLCLDGKEFQVPMATTEGCLVASTNRGCRAIGLGGGASSRVLADGMTRGPVVRFPRACDSAEVKAWLETPEGFTVIKEAFDSTSRVARLQKLHMSVAGRNLYIRFQSRSGDAMGMNMISKGTEKALSKLQEYFPEMQILAVSGNYCTDKKPAAINWIEGRGKSVVCEAVIPAKVVREVLKTTTEAMIEVNINKNLVGSAMAGSIGGYNAHAANIVTAIYIACGQDAAQNVGSSNCITLMEASGPTNEDLYISCTMPSIEIGTVGGGTNLLPQQACLQMLGVQGACRDNPGENARQLARIVCGTVMAGELSLMAALAAGHLVRSHMIHNRSKINLQDLQGTCTKKAA

SEQ ID NO: 111 - DNA, 1704 bp, synthetic construct
  misc_feature 1..1704: note = Description of Artificial Sequence: Synthetic polynucleotide
  source 1..1704: mol_type = other DNA
atggatttgagaaggaaattaccacctaagcctccatcttcaacaacaacaaaacagccaagtcataggtcccattctcctacgccaattccaaaggcttcagatgcattgcctcttccattgtacctgaccaatacgtttttcttcactcttttcttttccgtagcatattacctgttgcataggtggagagacaagattagatccggaacacctttacacgttgtgacactgactgaactatccgcaattgtactgctgattgcttccttcatctatcttttaggctttttcggtattgattttgtgcaatctttcacatcaagagaaaatgagcaactaaacaacgatgatcacaacgtcgtgtcaacaaacaatgttttatctgatagaaggttagtttacgactatggattcgatgtgacaggagacaacgataacgataatgatgacgatgttattgtgaaaagtgtcgtttctggggaagttaattcttatagtttggaggcttccctaggagattgttacagagccgcaaagattagaaagagagccgtcgagagaattgtcgggagagaagtattaggcttgggtttcgagggatttgattatgaatctatcctggggcaatgttgtgaaatgcctatcgggtacgtccaagtgccagtaggtgtcgctggacctttattgttaaatggtggggaattcatggttccaatggctacaactgaaggctgtcttgtagcttccactaatagaggttgtaaagccatatgcttatcaggtggtgccactgccatattgctaaaagatggtatgacaagagccccagtagtgagattcgccacagctgagagagcttcacaactaaagttttacttggaagatggtgtcaatttcgatacattgtctgttgtctttaacaaaagttcaagatttgccagattgcaaaacatccaatgctcaattgccggtaaaaacttgtacattaggtttacttgctccacaggcgacgccatgggtatgaacatggtttcaaaaggagtacaaaatgtattagactttttacaaaatgattttcctgatatggacgtaattgggatctcttggaagttctgctctgacaaaaagccaacagctgtcaactggattgagggcagaggaaagtctgtcgttttccaggccgtaattaccaaaaaggtggttagaaagtctgcactgaaccctcaaacttgcacatgtagaactttgacctgtttaagaccattattggttctgctacttctggttttgctagtggacttaatgcatatgcttcatatcgtgtctgccgtgttcatcgctaccggtcaagatccagctcagaatatcgaatctagtcactgtatcactatgatggaggctgtcaacaatggtaaggatttgcacgttaatgttacgatgccatctatagaagttggcacggtgggaggtggcactcagctagcctctcaatcagcctgtttgaacttgcttggtgtaaagggtgcctgtatagaatccccaggatcaaacgcccagttgttagctagaatcgttgctggttctgttctggcaggcgaattaagtttgatgtcagctataagtgctgggcaactagttaaatctcatatgaaatacaataggtctagtagagatatgtcagcaatagcttctaaggtctaa

SEQ ID NO: 112 - protein, 567 aa, Artemisia annua
MDLRRKLPPKPPSSTTTKQPSHRSHSPTPIPKASDALPLPLYLTNTFFFTLFFSVAYYLLHRWRDKIRSGTPLHVVTLTELSAIVLLIASFIYLLGFFGIDFVQSFTSRENEQLNNDDHNVVSTNNVLSDRRLVYDYGFDVTGDNDNDNDDDVIVKSVVSGEVNSYSLEASLGDCYRAAKIRKRAVERIVGREVLGLGFEGFDYESILGQCCEMPIGYVQVPVGVAGPLLLNGGEFMVPMATTEGCLVASTNRGCKAICLSGGATAILLKDGMTRAPVVRFATAERASQLKFYLEDGVNFDTLSVVFNKSSRFARLQNIQCSIAGKNLYIRFTCSTGDAMGMNMVSKGVQNVLDFLQNDFPDMDVIGISWKFCSDKKPTAVNWIEGRGKSVVFQAVITKKVVRKSALNPQTCTCRTLTCLRPLLVLLLLVLLVDLMHMLHIVSAVFIATGQDPAQNIESSHCITMMEAVNNGKDLHVNVTMPSIEVGTVGGGTQLASQSACLNLLGVKGACIESPGSNAQLLARIVAGSVLAGELSLMSAISAGQLVKSHMKYNRSSRDMSAIASKV

SEQ ID NO: 113 - DNA, 1308 bp, synthetic construct
  misc_feature 1..1308: note = Description of Artificial Sequence: Synthetic polynucleotide
  source 1..1308: mol_type = other DNA
atgtttagaagagctatactgttaggatgctctgctgccaagacaccatggtctgagtgttctaacgctcaattagttgatgcagttaagtctagaaagatctcattctacggtcttgaacaagccttggaaccagattatagaagggctatcgaagtaaggagagaggttgtctctgaaatcgcctcacaacagccagaagcaaaaaagaagcaatccgcattgcacacaataccatttgagaattatgattggaataaggtcgttggccaaaactgtgaaaacattattggatacgtcccaataccactgggcgttgctggccctattttgattgatggtaaagagtacccaataccaatggctacaacagaaggcgctttggtcgctagtactcatagaggtgctagagctattacaagatccggaggttgtaagacattgttattaggtgaaggtatgacaagagcaccagtggttgaattgccttcattagaggaagctgggcgtttgcacaagtactgtaatgagaacttcttatctttaaaggaagcatttgaatcaactacccaatatggaaaacttaattctttaaagtgcgtactagctggtagaaaagcataccttagattcagagccactacaggcgatgctatgggcatgaacatgataacaaagggtgtagacaaagcactgtctgttctacagcaacatttcccttcaatggaaatcctagccctaagtggtaattactgtaccgacaaaaagccatctgctgtaaattggattgatggcagaggtaaatcagtggttgcagaagccactttattggctgatgttgtcgaagatactctgaaatgtacagtcgattctttggtatccttgaatatcgacaaaaaccttgttgggtcagctatggctggttctgttggaggttttaacgcccaggctgcaaacgctgtggcagccattttcattgcaaccggtcaagatcctgctcaagtggtagaaagttcaatgtgtatcactacaatgtccaaggtaggtaacgatctattgatctctgtgaccatgccttctatcgaggtcggggtcgtgggaggagggactggtcttgctgcccaaagaggatgcttagagttaatagggtgcggaggcccatctaaggagtctcctggtactaatgcccaacttctaagtagagttgttgcagctggcgttttatcagccgaactttccttgatgtccggactggcagcaggtcatctattgtcagcacatatgagattgaacagaaagaagaaataa

SEQ ID NO: 114 - protein, 435 aa, Trypanosoma cruzi
MFRRAILLGCSAAKTPWSECSNAQLVDAVKSRKISFYGLEQALEPDYRRAIEVRREVVSEIASQQPEAKKKQSALHTIPFENYDWNKVVGQNCENIIGYVPIPLGVAGPILIDGKEYPIPMATTEGALVASTHRGARAITRSGGCKTLLLGEGMTRAPVVELPSLEEAGRLHKYCNENFLSLKEAFESTTQYGKLNSLKCVLAGRKAYLRFRATTGDAMGMNMITKGVDKALSVLQQHFPSMEILALSGNYCTDKKPSAVNWIDGRGKSVVAEATLLADVVEDTLKCTVDSLVSLNIDKNLVGSAMAGSVGGFNAQAANAVAAIFIATGQDPAQVVESSMCITTMSKVGNDLLISVTMPSIEVGVVGGGTGLAAQRGCLELIGCGGPSKESPGTNAQLLSRVVAAGVLSAELSLMSGLAAGHLLSAHMRLNRKKK

SEQ ID NO: 115 - DNA, 1281 bp, synthetic construct
  misc_feature 1..1281: note = Description of Artificial Sequence: Synthetic polynucleotide
  source 1..1281: mol_type = other DNA
atgcaatccctggacaaaaactttagacacttatcaagacaacagaagttacaacagctagttgataaacaatggctatcagaggaacaattcaatattctacttaaccacccacttattgatgaagaggtagcaaactcattgatagaaaatgtcatcgcacagggcgcactgcctgttggtttactaccaaatatcatcgttgatgacaaagcatacgtcgtgcctatgatggtggaagagccatctgttgttgccgctgcttcatacggcgctaaattggtgaaccaaacaggtggtttcaaaaccgtgtcctcagaacgtatcatgataggtcaaatagtatttgatggagtcgatgataccgagaaactgtctgcagatatcaaggctcttgaaaaacaaatccatcagattgcagatgaggcttacccttctattaaggccagaggtggaggctatcaaaggatcgccatcgatacattcccagaacaacagttgctttcattgaaggttttcgttgatactaaggatgctatgggcgctaatatgttaaacacaatcctagaagcaatcacagcctttttgaaaaacgaattcccacaatctgatatcttgatgtctatcctttccaaccacgcaacagccagtgttgtcaaggtccagggtgaaatagacgttaaggatttggcaagaggagaacgtactggagaagaggtcgctaagagaatggaaagagcatctgtgttagctcaagtggacattcatagagcagcaacacacaataagggtgttatgaatggcattcatgctgtagtcttggctacaggtaatgatactagaggtgcagaagcctctgctcacgcttacgcttccaaagacggtcaatatagagggatagctacatggagatacgatcaagagagacaaaggttaataggaactatagaagttccaatgactctggccattgttggtggcggtaccaaggtactgcctattgctaaggcctctttagaactgttaaacgtagaaagtgcccaagagttgggacatgttgtcgctgccgttggactagctcaaaacttcgctgcatgtagagctttggtttccgaaggtattcaacaagggcatatgtctttgcaatacaagtctttagccatcgtagtcggggctaagggcgatgaaattgctcaggtagccgaagcactaaagcaagagccaagagcaaacactcaagttgcagagagaattttgcaagatttgagaagtcaacaataa

SEQ ID NO: 116 - protein, 426 aa, Staphylococcus aureus
MQSLDKNFRHLSRQQKLQQLVDKQWLSEEQFNILLNHPLIDEEVANSLIENVIAQGALPVGLLPNIIVDDKAYVVPMMVEEPSVVAAASYGAKLVNQTGGFKTVSSERIMIGQIVFDGVDDTEKLSADIKALEKQIHQIADEAYPSIKARGGGYQRIAIDTFPEQQLLSLKVFVDTKDAMGANMLNTILEAITAFLKNEFPQSDILMSILSNHATASVVKVQGEIDVKDLARGERTGEEVAKRMERASVLAQVDIHRAATHNKGVMNGIHAVVLATGNDTRGAEASAHAYASKDGQYRGIATWRYDQERQRLIGTIEVPMTLAIVGGGTKVLPIAKASLELLNVESAQELGHVVAAVGLAQNFAACRALVSEGIQQGHMSLQYKSLAIVVGAKGDEIAQVAEALKQEPRANTQVAERILQDLRSQQ

SEQ ID NO: 117 - DNA, 1311 bp, synthetic construct
  misc_feature 1..1311: note = Description of Artificial Sequence: Synthetic polynucleotide
  source 1..1311: mol_type = other DNA
atgcaggtcttaagattggataggagacattacaaaagtggcaagattagaagagcaatgagttctagaattcctggtttctacaaattgtcagtcgaggaaagactgaaaaaggttgctgaatttgcagggttatctgatgaggaagtgaaagctgttttgtcacaaggtttacctttggacgtagctgatagaatgatcgaaaatgtgatcggtacatttgaattaccacttggtatagcaaccaatttccttattgatggcaaggattatctaatccctatggctatagaggaaccatcagtagttgcagctgcttctaacgcagctagaatggccagagagtctggcgggtttacaactgattacacagggtccctgatgattggtcaaattcaagtcacaaaactgttgaatccaaatgcagctaagttcgaagttctacgtcaaaaagacgaaatcatagaaagagcaaatgagtgtgatccaatgttggtgaatttgggcggtggatgtaaagatatagaagcaagggtgatcgatacaatcatgggtaagatgctaattgttcatctgatcgttgatgttaaagacgctatgggtgcaaatgctgtcaacactatgtgtgaaaaagttgctcctttcatcgaacgtattactgggggaaaggtctatcttagaatcatttccaacttggctgcatatagacttgctagagcaaaggccgtttttgacaaagacgttattggcggagaggaggttgtagaagggatcatgcttgcatacgccttcgctgccgctgacccatttcgttgcgccacccacaataagggtatcatgaatggcatatcagccttaatgatcgctacaggaaacgactttagagccattgaagcaggagctcattcctatgctgcaataggtggatacaaaccactaactacctacgaagttgatagaaaaggtaatctagtaggcacaattgaaatacctatggcagtaggcgtgattggtggtgcaaccaaagtcaacccactagccaagatctctcttaagatactaggagtgaacactgctgaagagttagccagagtcgcagccgctctaggtttggctcaaaactttgctgccttaagagccttggccacagaaggtatccaaagaggtcacatggaattacatgccaggaacttagcaatcatggctggagctactggagatgaggttgacagagttgtagagattatggtgagagatggcaaaatcagattggactacgctaaggaagtattggagagactgcgttcctaa

SEQ ID NO: 118 - protein, 436 aa, Archaeoglobus fulgidus
MQVLRLDRRHYKSGKIRRAMSSRIPGFYKLSVEERLKKVAEFAGLSDEEVKAVLSQGLPLDVADRMIENVIGTFELPLGIATNFLIDGKDYLIPMAIEEPSVVAAASNAARMARESGGFTTDYTGSLMIGQIQVTKLLNPNAAKFEVLRQKDEIIERANECDPMLVNLGGGCKDIEARVIDTIMGKMLIVHLIVDVKDAMGANAVNTMCEKVAPFIERITGGKVYLRIISNLAAYRLARAKAVFDKDVIGGEEVVEGIMLAYAFAAADPFRCATHNKGIMNGISALMIATGNDFRAIEAGAHSYAAIGGYKPLTTYEVDRKGNLVGTIEIPMAVGVIGGATKVNPLAKISLKILGVNTAEELARVAAALGLAQNFAALRALATEGIQRGHMELHARNLAIMAGATGDEVDRVVEIMVRDGKIRLDYAKEVLERLRS

SEQ ID NO: 119 - DNA, 1287 bp, synthetic construct
  misc_feature 1..1287: note = Description of Artificial Sequence: Synthetic polynucleotide
  source 1..1287: mol_type = other DNA
atgtccttagattcaagactgccagctttcagaaatctgtctccagctgcaagactagatcacattggccaacttttgggactaagtcatgacgacgtttcccttttagcaaacgccggtgctttaccaatggatatcgctaatggtatgattgaaaatgtaatcgggacctttgaactgccatatgcagtggccagtaactttcagatcaatggccgtgacgtcttagtaccattagttgtggaggaacctagtatcgttgctgcagcctcttacatggcaaagttagctagagccaatggtgggttcactacatcttcatctgctccactaatgcatgcacaagtacaaattgtcggcattcaggatccactaaacgcaagattgtctttactgcgtagaaaggatgagatcatagaattagccaataggaaggaccaacttctgaattcattgggcggtggttgcagagacatagaggtgcatacatttgccgatactccaagaggaccaatgcttgtagcacaccttattgtcgatgtgcgtgatgccatgggagctaatactgttaacactatggctgaagcagtagcacctctgatggaagccataacaggtggccaggtaagattgagaatcctttccaatttggctgatcttagattggccagagcccaagtgagaatcactcctcagcaattggaaactgccgaattctcaggtgaggcagtaattgagggtatcttggacgcatatgcttttgccgctgtggacccttacagagccgctacccacaacaaaggcataatgaacggtatcgatcctttgatcgtcgctacaggaaatgattggagagctgttgaggcaggagctcatgcatacgcttgtagatccggacattacggttcattaacaacatgggaaaaagataacaatggacacttggtcgggacattggaaatgcctatgccagttggtttagttgggggtgctacaaaaacccatcctcttgctcaattgtctttgaggatacttggtgtcaaaactgctcaagcactagccgaaattgccgttgctgttggtttggcacaaaacttgggtgcaatgcgtgctttagctacagaaggcatccaaagaggacatatggctctacacgctagaaacattgcagttgttgcaggagccagaggtgatgaggttgattgggtggctagacaacttgtcgaatatcatgatgtcagagcagacagggctgtggcattactgaaacagaagagaggtcaataa

SEQ ID NO: 120 - protein, 428 aa, Pseudomonas mevalonii
MSLDSRLPAFRNLSPAARLDHIGQLLGLSHDDVSLLANAGALPMDIANGMIENVIGTFELPYAVASNFQINGRDVLVPLVVEEPSIVAAASYMAKLARANGGFTTSSSAPLMHAQVQIVGIQDPLNARLSLLRRKDEIIELANRKDQLLNSLGGGCRDIEVHTFADTPRGPMLVAHLIVDVRDAMGANTVNTMAEAVAPLMEAITGGQVRLRILSNLADLRLARAQVRITPQQLETAEFSGEAVIEGILDAYAFAAVDPYRAATHNKGIMNGIDPLIVATGNDWRAVEAGAHAYACRSGHYGSLTTWEKDNNGHLVGTLEMPMPVGLVGGATKTHPLAQLSLRILGVKTAQALAEIAVAVGLAQNLGAMRALATEGIQRGHMALHARNIAVVAGARGDEVDWVARQLVEYHDVRADRAVALLKQKRGQ

SEQ ID NO: 121 - protein, 361 aa, Stevia rebaudiana
MALVNPTALFYGTSIRTRPTNLLNPTQKLRPVSSSSLPSFSSVSAILTEKHQSNPSENNNLQTHLETPFNFDSYMLEKVNMVNEALDASVPLKDPIKIHESMRYSLLAGGKRIRPMMCIAACEIVGGNILNAMPAACAVEMIHTMSLVHDDLPCMDNDDFRRGKPISHKVYGEEMAVLTGDALLSLSFEHIATATKGVSKDRIVRAIGELARSVGSEGLVAGQVVDILSEGADVGLDHLEYIHIHKTAMLLESSVVIGAIMGGGSDQQIEKLRKFARSIGLLFQVVDDILDVTKSTEELGKTAGKDLLTDKTTYPKLLGIEKSREFAEKLNKEAQEQLSGFDRRKAAPLIALANYNAYRQN

SEQ ID NO: 122 - protein, 342 aa, Gibberella fujikuroi
MAEQQISNLLSMFDASHASQKLEITVQMMDTYHYRETPPDSSSSEGGSLSRYDERRVSLPLSHNAASPDIVSQLCFSTAMSSELNHRWKSQRLKVADSPYNYILTLPSKGIRGAFIDSLNVWLEVPEDETSVIKEVIGMLHNSSLIIDDFQDNSPLRRGKPSTHTVFGPAQAINTATYVIVKAIEKIQDIVGHDALADVTGTITTIFQGQAMDLWWTANAIVPSIQEYLLMVNDKTGALFRLSLELLALNSEASISDSALESLSSAVSLLGQYFQIRDDYMNLIDNKYTDQKGFCEDLDEGKYSLTLIHALQTDSSDLLTNILSMRRVQGKLTAQKRCWFWK

SEQ ID NO: 123 - protein, 300 aa, Mus musculus
MEKTKEKAERILLEPYRYLLQLPGKQVRSKLSQAFNHWLKVPEDKLQIIIEVTEMLHNASLLIDDIEDSSKLRRGFPVAHSIYGVPSVINSANYVYFLGLEKVLTLDHPDAVKLFTRQLLELHQGQGLDIYWRDTYTCPTEEEYKAMVLQKTGGLFGLAVGLMQLFSDYKEDLKPLLDTLGLFFQIRDDYANLHSKEYSENKSFCEDLTEGKFSFPTIHAIWSRPESTQVQNILRQRTENIDIKKYCVQYLEDVGSFAYTRHTLRELEAKAYKQIEACGGNPSLVALVKHLSKMFTEENK

SEQ ID NO: 124 - protein, 339 aa, Thalassiosira pseudonana
MARFYFLNALLMVISLQSTTAFTPAKLAYPTTTTALNVASAETSFSLDEYLASKIGPIESALEASVKSRIPQTDKICESMAYSLMAGGKRIRPVLCIAACEMFGGSQDVAMPTAVALEMIHTMSLIHDDLPSMDNDDLRRGKPTNHVVFGEDVAILAGDSLLSTSFEHVARETKGVSAEKIVDVIARLGKSVGAEGLAGGQVMDLECEAKPGTTLDDLKWIHIHKTATLLQVAVASGAVLGGATPEEVAACELFAMNIGLAFQVADDILDVTASSEDLGKTAGKDEATDKTTYPKLLGLEESKAYARQLIDEAKESLAPFGDRAAPLLAIADFIIDRKN

SEQ ID NO: 125 - protein, 355 aa, Streptomyces clavuligerus
MHLAPRRVPRGRRSPPDRVPERQGALGRRRGAGSTGCARAAAGVHRRRGGGEADPSAAVHRGWQAGGGTGLPDEVVSTAAALEMFHAFALIHDDIMDDSATRRGSPTVHRALADRLGAALDPDQAGQLGVSTAILVGDLALTWSDELLYAPLTPHRLAAVLPLVTAMRAETVHGQYLDITSARRPGTDTSLALRIARYKTAAYTMERPLHIGAALAGARPELLAGLSAYALPAGEAFQLADDLLGVFGDPRRTGKPDLDDLRGGKHTVLVALAREHATPEQRHTLDTLLGTPGLDRQGASRLRCVLVATGARAEAERLITERRDQALTALNALTLPPPLAEALARLTLGSTAHPA

SEQ ID NO: 126 - protein, 330 aa, Sulfulobus acidicaldarius
MSYFDNYFNEIVNSVNDIIKSYISGDVPKLYEASYHLFTSGGKRLRPLILTISSDLFGGQRERAYYAGAAIEVLHTFTLVHDDIMDQDNIRRGLPTVHVKYGLPLAILAGDLLHAKAFQLLTQALRGLPSETIIKAFDIFTRSIIIISEGQAVDMEFEDRIDIKEQEYLDMISRKTAALFSASSSIGALIAGANDNDVRLMSDFGTNLGIAFQIVDDILGLTADEKELGKPVFSDIREGKKTILVIKTLELCKEDEKKIVLKALGNKSASKEELMSSADIIKKYSLDYAYNLAEKYYKNAIDSLNQVSSKSDIPGKALKYLAEFTIRRRK

SEQ ID NO: 127 - protein, 297 aa, Synechococcus sp.
MVAQTFNLDTYLSQRQQQVEEALSAALVPAYPERIYEAMRYSLLAGGKRLRPILCLAACELAGGSVEQAMPTACALEMIHTMSLIHDDLPAMDNDDFRRGKPTNHKVFGEDIAILAGDALLAYAFEHIASQTRGVPPQLVLQVIARIGHAVAATGLVGGQVVDLESEGKAISLETLEYIHSHKTGALLEASVVSGGILAGADEELLARLSHYARDIGLAFQIVDDILDVTATSEQLGKTAGKDQAAAKATYPSLLGLEASRQKAEELIQSAKEALRPYGSQAEPLLALADFITRRQH

SEQ ID NO: 128 - protein, 371 aa, Arabidopsis thaliana
MASVTLGSWIVVHHHNHHHPSSILTKSRSRSCPITLTKPISFRSKRTVSSSSSIVSSSVVTKEDNLRQSEPSSFDFMSYIITKAELVNKALDSAVPLREPLKIHEAMRYSLLAGGKRVRPVLCIAACELVGGEESTAMPAACAVEMIHTMSLIHDDLPCMDNDDLRRGKPTNHKVFGEDVAVLAGDALLSFAFEHLASATSSDVVSPVRVVRAVGELAKAIGTEGLVAGQVVDISSEGLDLNDVGLEHLEFIHLHKTAALLEASAVLGAIVGGGSDDEIERLRKFARCIGLLFQVVDDILDVTKSSKELGKTAGKDLIADKLTYPKIMGLEKSREFAEKLNREARDQLLGFDSDKVAPLLALANYIAYRQN

SEQ ID NO: 129 - protein, 787 aa, Stevia rebaudiana
MKTGFISPATVFHHRISPATTFRHHLSPATTNSTGIVALRDINFRCKAVSKEYSDLLQKDEASFTKWDDDKVKDHLDTNKNLYPNDEIKEFVESVKAMFGSMNDGEINVSAYDTAWVALVQDVDGSGSPQFPSSLEWIANNQLSDGSWGDHLLFSAHDRIINTLACVIALTSWNVHPSKCEKGLNFLRENICKLEDENAEHMPIGFEVTFPSLIDIAKKLNIEVPEDTPALKEIYARRDIKLTKIPMEVLHKVPTTLLHSLEGMPDLEWEKLLKLQCKDGSFLFSPSSTAFALMQTKDEKCLQYLTNIVTKFNGGVPNVYPVDLFEHIWVVDRLQRLGIARYFKSEIKDCVEYINKYWTKNGICWARNTHVQDIDDTAMGFRVLRAHGYDVTPDVFRQFEKDGKFVCFAGQSTQAVTGMFNVYRASQMLFPGERILEDAKKFSYNYLKEKQSTNELLDKWIIAKDLPGEVGYALDIPWYASLPRLETRYYLEQYGGEDDVWIGKTLYRMGYVSNNTYLEMAKLDYNNYVAVLQLEWYTIQQWYVDIGIEKFESDNIKSVLVSYYLAAASIFEPERSKERIAWAKTTILVDKITSIFDSSQSSKEDITAFIDKFRNKSSSKKHSINGEPWHEVMVALKKTLHGFALDALMTHSQDIHPQLHQAWEMWLTKLQDGVDVTAELMVQMINMTAGRWVSKELLTHPQYQRLSTVTNSVCHDITKLHNFKENSTTVDSKVQELVQLVFSDTPDDLDQDMKQTFLTVMKTFYYKAWCDPNTINDHISKVFEIVI

SEQ ID NO: 130 - protein, 527 aa, Streptomyces clavuligerus
MPDAHDAPPPQIRQRTLVDEATQLLTESAEDAWGEVSVSEYETARLVAHATWLGGHATRVAFLLERQHEDGSWGPPGGYRLVPTLSAVHALLTCLASPAQDHGVPHDRLLRAVDAGLTALRRLGTSDSPPDTIAVELVIPSLLEGIQHLLDPAHPHSRPAFSQHRGSLVCPGGLDGRTLGALRSHAAAGTPVPGKVWHASETLGLSTEAASHLQPAQGIIGGSAAATATWLTRVAPSQQSDSARRYLEELQHRYSGPVPSITPITYFERAWLLNNFAAAGVPCEAPAALLDSLEAALTPQGAPAGAGLPPDADDTAAVLLALATHGRGRRPEVLMDYRTDGYFQCFIGERTPSISTNAHVLETLGHHVAQHPQDRARYGSAMDTASAWLLAAQKQDGSWLDKWHASPYYATVCCTQALAAHASPATAPARQRAVRWVLATQRSDGGWGLWHSTVEETAYALQILAPPSGGGNIPVQQALTRGRARLCGALPLTPLWHDKDLYTPVRVVRAARAAALYTTRDLLLPPL

SEQ ID NO: 131 - protein, 516 aa, Bradyrhizobium japonicum
MNALSEHILSELRRLLSEMSDGGSVGPSVYDTAQALRFHGNVTGRQDAYAWLIAQQQADGGWGSADFPLFRHAPTWAALLALQRADPLPGAADAVQTATRFLQRQPDPYAHAVPEDAPIGAELILPQFCGEAAWLLGGVAFPRHPALLPLRQACLVKLGAVAMLPSGHPLLHSWEAWGTSPTTACPDDDGSIGISPAATAAWRAQAVTRGSTPQVGRADAYLQMASRATRSGIEGVFPNVWPINVFEPCWSLYTLHLAGLFAHPALAEAVRVIVAQLEARLGVHGLGPALHFAADADDTAVALCVLHLAGRDPAVDALRHFEIGELFVTFPGERNASVSTNIHALHALRLLGKPAAGASAYVEANRNPHGLWDNEKWHVSWLYPTAHAVAALAQGKPQWRDERALAALLQAQRDDGGWGAGRGSTFEETAYALFALHVMDGSEEATGRRRIAQVVARALEWMLARHAAHGLPQTPLWIGKELYCPTRVVRVAELAGLWLALRWGRRVLAEGAGAAP

SEQ ID NO: 132 - protein, 784 aa, Stevia rebaudiana
MNLSLCIASPLLTKSNRPAALSAIHTASTSHGGQTNPTNLIIDTTKERIQKQFKNVEISVSSYDTAWVAMVPSPNSPKSPCFPECLNWLINNQLNDGSWGLVNHTHNHNHPLLKDSLSSTLACIVALKRWNVGEDQINKGLSFIESNLASATEKSQPSPIGFDIIFPGLLEYAKNLDINLLSKQTDFSLMLHKRELEQKRCHSNEMDGYLAYISEGLGNLYDWNMVKKYQMKNGSVFNSPSATAAAFINHQNPGCLNYLNSLLDKFGNAVPTVYPHDLFIRLSMVDTIERLGISHHFRVEIKNVLDETYRCWVERDEQIFMDVVTCALAFRLLRINGYEVSPDPLAEITNELALKDEYAALETYHASHILYQEDLSSGKQILKSADFLKEIISTDSNRLSKLIHKEVENALKFPINTGLERINTRRNIQLYNVDNTRILKTTYHSSNISNTDYLRLAVEDFYTCQSIYREELKGLERWVVENKLDQLKFARQKTAYCYFSVAATLSSPELSDARISWAKNGILTTVVDDFFDIGGTIDELTNLIQCVEKWNVDVDKDCCSEHVRILFLALKDAICWIGDEAFKWQARDVTSHVIQTWLELMNSMLREAIWTRDAYVPTLNEYMENAYVSFALGPIVKPAIYFVGPKLSEEIVESSEYHNLFKLMSTQGRLLNDIHSFKREFKEGKLNAVALHLSNGESGKVEEEVVEEMMMMIKNKRKELMKLIFEENGSIVPRACKDAFWNMCHVLNFFYANDDGFTGNTILDTVKDIIYNPLVLVNENEEQR

SEQ ID NO: 133 - protein, 784 aa, Stevia rebaudiana
MNLSLCIASPLLTKSSRPTALSAIHTASTSHGGQTNPTNLIIDTTKERIQKLFKNVEISVSSYDTAWVAMVPSPNSPKSPCFPECLNWLINNQLNDGSWGLVNHTHNHNHPLLKDSLSSTLACIVALKRWNVGEDQINKGLSFIESNLASATDKSQPSPIGFDIIFPGLLEYAKNLDINLLSKQTDFSLMLHKRELEQKRCHSNEIDGYLAYISEGLGNLYDWNMVKKYQMKNGSVFNSPSATAAAFINHQNPGCLNYLNSLLDKFGNAVPTVYPLDLYIRLSMVDTIERLGISHHFRVEIKNVLDETYRCWVERDEQIFMDVVTCALAFRLLRIHGYKVSPDQLAEITNELAFKDEYAALETYHASQILYQEDLSSGKQILKSADFLKGILSTDSNRLSKLIHKEVENALKFPINTGLERINTRRNIQLYNVDNTRILKTTYHSSNISNTYYLRLAVEDFYTCQSIYREELKGLERWVVQNKLDQLKFARQKTAYCYFSVAATLSSPELSDARISWAKNGILTTVVDDFFDIGGTIDELTNLIQCVEKWNVDVDKDCCSEHVRILFLALKDAICWIGDEAFKWQARDVTSHVIQTWLELMNSMLREAIWTRDAYVPTLNEYMENAYVSFALGPIVKPAIYFVGPKLSEEIVESSEYHNLFKLMSTQGRLLNDIHSFKREFKEGKLNAVALHLSNGESGKVEEEVVEEMMMMIKNKRKELMKLIFEENGSIVPRACKDAFWNMCHVLNFFYANDDGFTGNTILDTVKDIIYNPLVLVNENEEQR

SEQ ID NO: 134 - protein, 590 aa, Zea mays
MAMPVKLTPASLSLKAVCCRFSSGGHALRFGSSLPCWRRTPTQRSTSSSTTRPAAEVSSGKSKQHDQEASEATIRQQLQLVDVLENMGISRHFAAEIKCILDRTYRSWLQRHEEIMLDTMTCAMAFRILRLNGYNVSSDELYHVVEASGLHNSLGGYLNDTRTLLELHKASTVSISEDESILDSIGSRSRTLLREQLESGGALRKPSLFKEVEHALDGPFYTTLDRLHHRWNIENFNIIEQHMLETPYLSNQHTSRDILALSIRDFSSSQFTYQQELQHLESWVKECRLDQLQFARQKLAYFYLSAAGTMFSPELSDARTLWAKNGVLTTIVDDFFDVAGSKEELENLVMLVEMWDEHHKVEFYSEQVEIIFSSIYDSVNQLGEKASLVQDRSITKHLVEIWLDLLKSMMTEVEWRLSKYVPTEKEYMINASLIFGLGPIVLPALYFVGPKISESIVKDPEYDELFKLMSTCGRLLNDVQTFEREYNEGKLNSVSLLVLHGGPMSISDAKRKLQKPIDTCRRDLLSLVLREESVVPRPCKELFWKMCKVCYFFYSTTDGFSSQVERAKEVDAVINEPLKLQGSHTLVSDV

SEQ ID NO: 135 - protein, 743 aa, Populus trichocarpa
MQNFHGTKERIKKMFDKIELSVSSYDTAWVAMVPSPDCPETPCFPECTKWILENQLGDGSWSLPHGNPLLVKDALSSTLACILALKRWGIGEEQINKGLRFIELNSASVTDNEQHKPIGFDIIFPGMIEYAIDLDLNLPLKPTDINSMLHRRALELTSGGGKNLEGRRAYLAYVSEGIGKLQDWEMAMKYQRKNGSLFNSPSTTAAAFIHIQDAECLHYIRSLLQKFGNAVPTIYPLDIYARLSMVDALERLGIDRHFRKERKFVLDETYRFWLQGEEEIFSDNATCALAFRILRLNGYDVSLEDHFSNSLGGYLKDSGAALELYRALQLSYPDESLLEKQNSRTSYFLKQGLSNVSLCGDRLRKNIIGEVHDALNFSDHANLQRLAIRRRIKHYATDDTRILKTSYRCSTIGNQDFLKLAVEDFNICQSIQREEFKHIERWVVERRLDKLKFARQKEAYCYFSAAATLFAPELSDARMSWAKNGVLTTVVDDFFDVGGSEEELVNLIELIERWDVNGSADFCSEEVEIIYSAIHSTISEIGDKSFGWQGRDVKSQVIKIWLDLLKSMLTEAQWSSNKSVPTLDEYMTTAHVSFALGPIVLPALYFVGPKLSEEVAGHPELLNLYKVTSTCGRLLNDWRSFKRESEEGKLNAISLYMIHSGGASTEEETIEHFKGLIDSQRRQLLQLVLQEKDSIIPRPCKDLFWNMIKLLHTFYMKDDGFTSNEMRNVVKAIINEPISLDEL

SEQ ID NO: 136 - protein, 983 aa, Phomopsis amygdali
MEFDEPLVDEARSLVQRTLQDYDDRYGFGTMSCAAYDTAWVSLVTKTVDGRKQWLFPECFEFLLETQSDAGGWEIGNSAPIDGILNTAASLLALKRHVQTEQIIQPQHDHKDLAGRAERAAASLRAQLAALDVSTTEHVGFEIIVPAMLDPLEAEDPSLVFDFPARKPLMKIHDAKMSRFRPEYLYGKQPMTALHSLEAFIGKIDFDKVRHHRTHGSMMGSPSSTAAYLMHASQWDGDSEAYLRHVIKHAAGQGTGAVPSAFPSTHFESSWILTTLFRAGFSASHLACDELNKLVEILEGSFEKEGGAIGYAPGFQADVDDTAKTISTLAVLGRDATPRQMIKVFEANTHFRTYPGERDPSLTANCNALSALLHQPDAAMYGSQIQKITKFVCDYWWKSDGKIKDKWNTCYLYPSVLLVEVLVDLVSLLEQGKLPDVLDQELQYRVAITLFQACLRPLLDQDAEGSWNKSIEATAYGILILTEARRVCFFDRLSEPLNEAIRRGIAFADSMSGTEAQLNYIWIEKVSYAPALLTKSYLLAARWAAKSPLGASVGSSLWTPPREGLDKHVRLFHQAELFRSLPEWELRASMIEAALFTPLLRAHRLDVFPRQDVGEDKYLDVVPFFWTAANNRDRTYASTLFLYDMCFIAMLNFQLDEFMEATAGILFRDHMDDLRQLIHDLLAEKTSPKSSGRSSQGTKDADSGIEEDVSMSDSASDSQDRSPEYDLVFSALSTFTKHVLQHPSIQSASVWDRKLLAREMKAYLLAHIQQAEDSTPLSELKDVPQKTDVTRVSTSTTTFFNWVRTTSADHISCPYSFHFVACHLGAALSPKGSNGDCYPSAGEKFLAAAVCRHLATMCRMYNDLGSAERDSDEGNLNSLDFPEFADSAGNGGIEIQKAALLRLAEFERDSYLEAFRRLQDESNRVHGPAGGDEARLSRRRMAILEFFAQQVDLYGQVYVIRDISARIPKNEVEKKRKLDDAFN

SEQ ID NO: 137 - protein, 881 aa, Physcomitrella patens
MASSTLIQNRSCGVTSSMSSFQIFRGQPLRFPGTRTPAAVQCLKKRRCLRPTESVLESSPGSGSYRIVTGPSGINPSSNGHLQEGSLTHRLPIPMEKSIDNFQSTLYVSDIWSETLQRTECLLQVTENVQMNEWIEEIRMYFRNMTLGEISMSPYDTAWVARVPALDGSHGPQFHRSLQWIIDNQLPDGDWGEPSLFLGYDRVCNTLACVIALKTWGVGAQNVERGIQFLQSNIYKMEEDDANHMPIGFEIVFPAMMEDAKALGLDLPYDATILQQISAEREKKMKKIPMAMVYKYPTTLLHSLEGLHREVDWNKLLQLQSENGSFLYSPASTACALMYTKDVKCFDYLNQLLIKFDHACPNVYPVDLFERLWMVDRLQRLGISRYFEREIRDCLQYVYRYWKDCGIGWASNSSVQDVDDTAMAFRLLRTHGFDVKEDCFRQFFKDGEFFCFAGQSSQAVTGMFNLSRASQTLFPGESLLKKARTFSRNFLRTKHENNECFDKWIITKDLAGEVEYNLTFPWYASLPRLEHRTYLDQYGIDDIWIGKSLYKMPAVTNEVFLKLAKADFNMCQALHKKELEQVIKWNASCQFRDLEFARQKSVECYFAGAATMFEPEMVQARLVWARCCVLTTVLDDYFDHGTPVEELRVFVQAVRTWNPELINGLPEQAKILFMGLYKTVNTIAEEAFMAQKRDVHHHLKHYWDKLITSALKEAEWAESGYVPTFDEYMEVAEISVALEPIVCSTLFFAGHRLDEDVLDSYDYHLVMHLVNRVGRILNDIQGMKREASQGKISSVQIYMEEHPSVPSEAMAIAHLQELVDNSMQQLTYEVLRFTAVPKSCKRIHLNMAKIMHAFYKDTDGFSSLTAMTGFVKKVLFEPVPE

SEQ ID NO: 138 - protein, 513 aa, Stevia rebaudiana
MDAVTGLLTVPATAITIGGTAVALAVALIFWYLKSYTSARRSQSNHLPRVPEVPGVPLLGNLLQLKEKKPYMTFTRWAATYGPIYSIKTGATSMVVVSSNEIAKEALVTRFQSISTRNLSKALKVLTADKTMVAMSDYDDYHKTVKRHILTAVLGPNAQKKHRIHRDIMMDNISTQLHEFVKNNPEQEEVDLRKIFQSELFGLAMRQALGKDVESLYVEDLKITMNRDEIFQVLVVDPMMGAIDVDWRDFFPYLKWVPNKKFENTIQQMYIRREAVMKSLIKEHKKRIASGEKLNSYIDYLLSEAQTLTDQQLLMSLWEPIIESSDTTMVTTEWAMYELAKNPKLQDRLYRDIKSVCGSEKITEEHLSQLPYITAIFHETLRRHSPVPIIPLRHVHEDTVLGGYHVPAGTELAVNIYGCNMDKNVWENPEEWNPERFMKENETIDFQKTMAFGGGKRVCAGSLQALLTASIGIGRMVQEFEWKLKDMTQEEVNTIGLTTQMLRPLRAIIKPRI

SEQ ID NO: 139 - protein, 509 aa, Arabidopsis thaliana
MAFFSMISILLGFVISSFIFIFFFKKLLSFSRKNMSEVSTLPSVPVVPGFPVIGNLLQLKEKKPHKTFTRWSEIYGPIYSIKMGSSSLIVLNSTETAKEAMVTRFSSISTRKLSNALTVLTCDKSMVATSDYDDFHKLVKRCLLNGLLGANAQKRKRHYRDALIENVSSKLHAHARDHPQEPVNFRAIFEHELFGVALKQAFGKDVESIYVKELGVTLSKDEIFKVLVHDMMEGAIDVDWRDFFPYLKWIPNKSFEARIQQKHKRRLAVMNALIQDRLKQNGSESDDDCYLNFLMSEAKTLTKEQIAILVWETIIETADTTLVTTEWAIYELAKHPSVQDRLCKEIQNVCGGEKFKEEQLSQVPYLNGVFHETLRKYSPAPLVPIRYAHEDTQIGGYHVPAGSEIAINIYGCNMDKKRWERPEDWWPERFLDDGKYETSDLHKTMAFGAGKRVCAGALQASLMAGIAIGRLVQEFEWKLRDGEEENVDTYGLTSQKLYPLMAIINPRRS

SEQ ID NO: 140 - protein, 525 aa, Gibberella fujikoroi
MSKSNSMNSTSHETLFQQLVLGLDRMPLMDVHWLIYVAFGAWLCSYVIHVLSSSSTVKVPVVGYRSVFEPTWLLRLRFVWEGGSIIGQGYNKFKDSIFQVRKLGTDIVIIPPNYIDEVRKLSQDKTRSVEPFINDFAGQYTRGMVFLQSDLQNRVIQQRLTPKLVSLTKVMKEELDYALTKEMPDMKNDEWVEVDISSIMVRLISRISARVFLGPEHCRNQEWLTTTAEYSESLFITGFILRVVPHILRPFIAPLLPSYRTLLRNVSSGRRVIGDIIRSQQGDGNEDILSWMRDAATGEEKQIDNIAQRMLILSLASIHTTAMTMTHAMYDLCACPEYIEPLRDEVKSVVGASGWDKTALNRFHKLDSFLKESQRFNPVFLLTFNRIYHQSMTLSDGTNIPSGTRIAVPSHAMLQDSAHVPGPTPPTEFDGFRYSKIRSDSNYAQKYLFSMTDSSNMAFGYGKYACPGRFYASNEMKLTLAILLLQFEFKLPDGKGRPRNITIDSDMIPDPRARLCVRKRSLRDE

SEQ ID NO: 141 - protein, 499 aa, Trametes versicolor
MEDPTVLYACLAIAVATFVVRWYRDPLRSIPTVGGSDLPILSYIGALRWTRRGREILQEGYDGYRGSTFKIAMLDRWIVIANGPKLADEVRRRPDEELNFMDGLGAFVQTKYTLGEAIHNDPYHVDIIREKLTRGLPAVLPDVIEELTLAVRQYIPTEGDEWVSVNCSKAARDIVARASNRVFVGLPACRNQGYLDLAIDFTLSVVKDRAIINMFPELLKPIVGRVVGNATRNVRRAVPFVAPLVEERRRLMEEYGEDWSEKPNDMLQWIMDEAASRDSSVKAIAERLLMVNFAAIHTSSNTITHALYHLAEMPETLQPLREEIEPLVKEEGWTKAAMGKMWWLDSFLRESQRYNGINIVSLTRMADKDITLSDGTFLPKGTLVAVPAYSTHRDDAVYADALVFDPFRFSRMRAREGEGTKHQFVNTSVEYVPFGHGKHACPGRFFAANELKAMLAYIVLNYDVKLPGDGKRPLNMYWGPTVLPAPAGQVLFRKRQVSL

SEQ ID NO: 142 - protein, 525 aa, Stevia rebaudiana
MGLFPLEDSYALVFEGLAITLALYYLLSFIYKTSKKTCTPPKASGEIIPITGIILNLLSGSSGLPIILALASLADRCGPIFTIRLGIRRVLVVSNWEIAKEIFTTHDLIVSNRPKYLAAKILGFNYVSFSFAPYGPYWVGIRKIIATKLMSSSRLQKLQFVRVFELENSMKSIRESWKEKKDEEGKVLVEMKKWFWELNMNIVLRTVAGKQYTGTVDDADAKRISELFREWFHYTGRFVVGDAFPFLGWLDLGGYKKTMELVASRLDSMVSKWLDEHRKKQANDDKKEDMDFMDIMISMTEANSPLEGYGTDTIIKTTCMTLIVSGVDTTSIVLTWALSLLLNNRDTLKKAQEELDMCVGKGRQVNESDLVNLIYLEAVLKEALRLYPAAFLGGPRAFLEDCTVAGYRIPKGTCLLINMWKLHRDPNIWSDPCEFKPERFLTPNQKDVDVIGMDFELIPFGAGRRYCPGTRLALQMLHIVLATLLQNFEMSTPNDAPVDMTASVGMTNAKASPLEVLLSPRVKWS

SEQ ID NO: 143 - protein, 476 aa, Stevia rebaudiana
MIQVLTPILLFLIFFVFWKVYKHQKTKINLPPGSFGWPFLGETLALLRAGWDSEPERFVRERIKKHGSPLVFKTSLFGDRFAVLCGPAGNKFLFCNENKLVASWWPVPVRKLFGKSLLTIRGDEAKWMRKMLLSYLGPDAFATHYAVTMDVVTRRHIDVHWRGKEEVNVFQTVKLYAFELACRLFMNLDDPNHIAKLGSLFNIFLKGIIELPIDVPGTRFYSSKKAAAAIRIELKKLIKARKLELKEGKASSSQDLLSHLLTSPDENGMFLTEEEIVDNILLLLFAGHDTSALSITLLMKTLGEHSDVYDKVLKEQLEISKTKEAWESLKWEDIQKMKYSWSVICEVMRLNPPVIGTYREALVDIDYAGYTIPKGWKLHWSAVSTQRDEANFEDVTRFDPSRFEGAGPTPFTFVPFGGGPRMCLGKEFARLEVLAFLHNIVTNFKWDLLIPDEKIEYDPMATPAKGLPIRLHPHQV

SEQ ID NO: 144 - protein, 525 aa, Arabidopsis thaliana
MESLVVHTVNAIWCIVIVGIFSVGYHVYGRAVVEQWRMRRSLKLQGVKGPPPSIFNGNVSEMQRIQSEAKHCSGDNIISHDYSSSLFPHFDHWRKQYGRIYTYSTGLKQHLYINHPEMVKELSQTNTLNLGRITHITKRLNPILGNGIITSNGPHWAHQRRIIAYEFTHDKIKGMVGLMVESAMPMLNKWEEMVKRGGEMGCDIRVDEDLKDVSADVIAKACFGSSFSKGKAIFSMIRDLLTAITKRSVLFRFNGFTDMVFGSKKHGDVDIDALEMELESSIWETVKEREIECKDTHKKDLMQLILEGAMRSCDGNLWDKSAYRRFVVDNCKSIYFAGHDSTAVSVSWCLMLLALNPSWQVKIRDEILSSCKNGIPDAESIPNLKTVTMVIQETMRLYPPAPIVGREASKDIRLGDLVVPKGVCIWTLIPALHRDPEIWGPDANDFKPERFSEGISKACKYPQSYIPFGLGPRTCVGKNFGMMEVKVLVSLIVSKFSFTLSPTYQHSPSHKLLVEPQHGVVIRVV

SEQ ID NO: 145 - protein, 529 aa, Vitis vinifera
MYFLLQYLNITTVGVFATLFLSYCLLLWRSRAGNKKIAPEAAAAWPIIGHLHLLAGGSHQLPHITLGNMADKYGPVFTIRIGLHRAVVVSSWEMAKECSTANDQVSSSRPELLASKLLGYNYAMFGFSPYGSYWREMRKIISLELLSNSRLELLKDVRASEVVTSIKELYKLWAEKKNESGLVSVEMKQWFGDLTLNVILRMVAGKRYFSASDASENKQAQRCRRVFREFFHLSGLFVVADAIPFLGWLDWGRHEKTLKKTAIEMDSIAQEWLEEHRRRKDSGDDNSTQDFMDVMQSVLDGKNLGGYDADTINKATCLTLISGGSDTTVVSLTWALSLVLNNRDTLKKAQEELDIQVGKERLVNEQDISKLVYLQAIVKETLRLYPPGPLGGLRQFTEDCTLGGYHVSKGTRLIMNLSKIQKDPRIWSDPTEFQPERFLTTHKDVDPRGKHFEFIPFGAGRRACPGITFGLQVLHLTLASFLHAFEFSTPSNEQVNMRESLGLTNMKSTPLEVLISPRLSLNCFNLMKI

SEQ ID NO: 146 - protein, 479 aa, Medicago trunculata
MEPNFYLSLLLLFVTFISLSLFFIFYKQKSPLNLPPGKMGYPIIGESLEFLSTGWKGHPEKFIFDRMRKYSSELFKTSIVGESTVVCCGAASNKFLFSNENKLVTAWWPDSVNKIFPTTSLDSNLKEESIKMRKLLPQFFKPEALQRYVGVMDVIAQRHFVTHWDNKNEITVYPLAKRYTFLLACRLFMSVEDENHVAKFSDPFQLIAAGIISLPIDLPGTPFNKAIKASNFIRKELIKIIKQRRVDLAEGTASPTQDILSHMLLTSDENGKSMNELNIADKILGLLIGGHDTASVACTFLVKYLGELPHIYDKVYQEQMEIAKSKPAGELLNWDDLKKMKYSWNVACEVMRLSPPLQGGFREAITDFMFNGFSIPKGWKLYWSANSTHKNAECFPMPEKFDPTRFEGNGPAPYTFVPFGGGPRMCPGKEYARLEILVFMHNLVKRFKWEKVIPDEKIIVDPFPIPAKDLPIRLYPHKA

SEQ ID NO: 147 - protein, 710 aa, Stevia rebaudiana
MQSDSVKVSPFDLVSAAMNGKAMEKLNASESEDPTTLPALKMLVENRELLTLFTTSFAVLIGCLVFLMWRRSSSKKLVQDPVPQVIVVKKKEKESEVDDGKKKVSIFYGTQTGTAEGFAKALVEEAKVRYEKTSFKVIDLDDYAADDDEYEEKLKKESLAFFFLATYGDGEPTDNAANFYKWFTEGDDKGEWLKKLQYGVFGLGNRQYEHFNKIAIVVDDKLTEMGAKRLVPVGLGDDDQCIEDDFTAWKELVWPELDQLLRDEDDTSVTTPYTAAVLEYRVVYHDKPADSYAEDQTHTNGHVVHDAQHPSRSNVAFKKELHTSQSDRSCTHLEFDISHTGLSYETGDHVGVYSENLSEVVDEALKLLGLSPDTYFSVHADKEDGTPIGGASLPPPFPPCTLRDALTRYADVLSSPKKVALLALAAHASDPSEADRLKFLASPAGKDEYAQWIVANQRSLLEVMQSFPSAKPPLGVFFAAVAPRLQPRYYSISSSPKMSPNRIHVTCALVYETTPAGRIHRGLCSTWMKNAVPLTESPDCSQASIFVRTSNFRLPVDPKVPVIMIGPGTGLAPFRGFLQERLALKESGTELGSSIFFFGCRNRKVDFIYEDELNNFVETGALSELIVAFSREGTAKEYVQHKMSQKASDIWKLLSEGAYLYVCGDAKGMAKDVHRTLHTIVQEQGSLDSSKAELYVKNLQMSGRYLRDVW

SEQ ID NO: 148 - protein, 692 aa, Arabidopsis thaliana [sequence omitted]

SEQ ID NO: 149 - protein, 713 aa, Giberella fujikuroi
MAELDTLDIVVLGVIFLGTVAYFTKGKLWGVTKDPYANGFAAGGASKPGRTRNIVEAMEESGKNCVVFYGSQTGTAEDYASRLAKEGKSRFGLNTMIADLEDYDFDNLDTVPSDNIVMFVLATYGEGEPTDNAVDFYEFITGEDASFNEGNDPPLGNLNYVAFGLGNNTYEHYNSMVRNVNKALEKLGAHRIGEAGEGDDGAGTMEEDFLAWKDPMWEALAKKMGLEEREAVYEPIFAINERDDLTPEANEVYLGEPNKLHLEGTAKGPFNSHNPYIAPIAESYELFSAKDRNCLHMEIDISGSNLKYETGDHIAIWPTNPGEEVNKFLDILDLSGKQHSVVTVKALEPTAKVPFPNPTTYDAILRYHLEICAPVSRQFVSTLAAFAPNDDIKAEMNRLGSDKDYFHEKTGPHYYNIARFLASVSKGEKWTKIPFSAFIEGLTKLQPRYYSISSSSLVQPKKISITAVVESQQIPGRDDPFRGVATNYLFALKQKQNGDPNPAPFGQSYELTGPRNKYDGIHVPVHVRHSNFKLPSDPGKPIIMIGPGTGVAPFRGFVQERAKQARDGVEVGKTLLFFGCRKSTEDFMYQKEWQEYKEALGDKFEMITAFSREGSKKVYVQHRLKERSKEVSDLLSQKAYFYVCGDAAHMAREVNTVLAQIIAEGRGVSEAKGEEIVKNMRSANQYQVCSDFVTLHCKETTYANSELQEDVWS

SEQ ID NO: 150 - protein, 453 aa, Arabidopsis thaliana
MGGLKFHVLMYPWFATGHMTPFLFLANKLAEKGHTVTFLLPKKSLKQLEHFNLFPHNIVFRSVTVPHVDGLPVGTETASEIPVTSTDLLMSAMDLTRDQVEAVVRAVEPDLIFFDFAHWIPEVARDFGLKTVKYVVVSASTIASMLVPGGELGVPPPGYPSSKVLLRKQDAYTMKKLEPTNTIDVGPNLLERVTTSLMNSDVIAIRTAREIEGNFCDYIEKHCRKKVLLTGPVFPEPDKTRELEERWVKWLSGYEPDSVVFCALGSQVILEKDQFQELCLGMELTGSPFLVAVKPPRGSSTIQEALPEGFEERVKGRGLVWGGWVQQPLILSHPSVGCFVSHCGFGSMWESLLSDCQIVLVPQLGDQVLNTRLLSDELKVSVEVAREETGWFSKESLCDAVNSVMKRDSELGNLVRKNHTKWRETVASPGLMTGYVDAFVESLQDLVSGTTHD

SEQ ID NO: 151 - DNA, 1362 bp, Arabidopsis thaliana
  source 1..1362: mol_type = genomic DNA
atgggtggtttgaagtttcatgtacttatgtatccatggttcgcaacaggccatatgaccccgttcctttttcttgccaacaaattggctgagaaaggtcatacggtcactttcttgcttcccaagaaatctctgaaacagttggaacatttcaatctgtttccacacaacattgtctttcgctctgtcaccgtccctcatgtggatggtctccccgttggcacagagacagcctctgagatccctgtgacatcaactgatctcttgatgtctgctatggatctcacacgtgatcaagttgaagctgtggtccgagccgttgaaccggacctgatcttctttgactttgctcattggattccagaagtagctagggacttcggccttaagactgtaaagtacgtcgtggtgtctgcatcgactatagctagtatgcttgtcccaggtggtgagttaggtgttcctccaccgggatatccatcatcaaaggtgctgcttcgtaaacaagatgcttacactatgaagaaactggagcctacaaatacaatcgatgtcggaccaaacctcttggaacgagtcactacaagtcttatgaactctgatgtcattgcgataaggacagccagagaaatcgaaggaaacttttgcgactatatagaaaaacattgcaggaaaaaggttctcttgacaggtccggtgttccctgagccagacaagactagagagctagaggaacgatgggttaagtggctaagtgggtatgaaccagactcagtggtgttttgtgcactgggctcacaagtcattttagagaaagatcaattccaagaactctgcttaggaatggagctaacaggttcaccgtttcttgtagcggttaagccccctagaggctcatcaacgattcaagaagcacttcctgaaggattcgaagagcgggttaaaggaagaggccttgtttggggaggatgggttcaacaaccattgatattgtctcatccatcagtcgggtgctttgtgagccattgtgggtttggatcaatgtgggagtctttgctgagtgattgtcagatagtcttagtaccacagttgggtgatcaagtcctgaacacaagattgctgagtgacgaactcaaggtttcggttgaagtggcaagagaggaaacaggatggttctcgaaagagagcttgtgcgatgctgtcaatagtgtgatgaaaagggacagcgagctcgggaacctggtgaggaagaatcacaccaagtggagggagacagtagctagtcctggactaatgactggttatgtcgatgctttcgtagagtcattgcaggatcttgtctctgggaccacccatgactga

(19) United States
(12) Patent Application Publication
(10) Pub. No.: US 2024/0110215 A1
(43) Pub. Date: Apr. 4, 2024

(54) Recombinant Production of Steviol Glycosides

(71) Applicant: EVOLVA SA, Reinach (CH)

(72) Inventors: Ganesh M. Kishore, Saint Louis, MO (US); Michael Motion, San Francisco, CA (US); Paula M. Hicks, Bend, OR (US); Jorgen Hansen, Frederiksberg (DK); Jens Houghton Larsen, Birkerod (DK); Esben Halkjaer Hansen, Frederiksberg (DK); Michael Dalgaard Mikkelsen, Vaerlose (DK); Sabina Tavares, San Francisco, CA (US); Charlotte Blom, San Francisco, CA (US)

(21) Appl. No.: 18/470,785

(22) Filed: Sep. 20, 2023

Related U.S. Application Data
(63) Continuation of application No. 17/373,612, filed on Jul. 12, 2021, now abandoned.
Application No. 17/373,612 is a continuation of application No. 16/460,320, filed on Jul. 2, 2019, now abandoned.
Application No. 16/460,320 is a continuation of application No. 15/382,354, filed on Dec. 16, 2016, now Pat. No. 10,392,644.
Application No. 15/382,354 is a division of application No. 13/701,406, filed on Mar. 22, 2013, now Pat. No. 9,562,251.
(60) Provisional application No. 61/350,553, filed on Jun. 2, 2010.
(60) Provisional application No. 61/434,582, filed on Jan. 20, 2011.
(60) Provisional application No. 61/471,622, filed on Apr. 4, 2011.

(51) Int. Cl. C12P 19/56; A23L 2/60; A23L 27/30; C07H 15/256; C12N 9/02; C12N 9/04; C12N 9/10; C12N 9/88; C12N 9/90; C12N 15/52; C12N 15/82; C12P 7/42; C12P 15/00

(52) U.S. Cl. CPC C12P 19/56; A23L 2/60; A23L 27/36; C07H 15/256; C12N 9/0006; C12N 9/0042; C12N 9/0073; C12N 9/1051; C12N 9/1077; C12N 9/1085; C12N 9/88; C12N 9/90; C12N 15/52; C12N 15/8243; C12N 15/8245; C12P 7/42; C12P 15/00; C12Y 101/01088; C12Y 106/02004; C12Y 114/13078; C12Y 114/13079; C12Y 204/01126; C12Y 205/01029; C12Y 402/03019; C12Y 505/01012; Y02P 20/52

(57) ABSTRACT

Recombinant microorganisms, plants, and plant cells are disclosed that have been engineered to express novel recombinant genes encoding steviol biosynthetic enzymes and UDP-glycosyltransferases (UGTs). Such microorganisms, plants, or plant cells can produce steviol or steviol glycosides, e.g., rubusoside or Rebaudioside A, which can be used as natural sweeteners in food products and dietary supplements.

### SEQUENCE LISTING

The instant application contains a Sequence Listing which has been submitted in ASCII format via EFS-Web and is hereby incorporated by reference in its entirety. Said ASCII copy, created on Jun. 2, 2011, is named 25933WO1.txt and is 483,406 bytes in size.

### TECHNICAL FIELD

This disclosure relates to the recombinant production of steviol and steviol glycosides. In particular, this disclosure relates to the production of steviol and steviol glycosides such as rubusoside and/or rebaudioside A by recombinant hosts such as recombinant microorganisms, plants, or plant cells. This disclosure also provides compositions containing steviol glycosides.

### BACKGROUND

Sweeteners are well known as ingredients used most commonly in the food, beverage, or confectionary industries. The sweetener can either be incorporated into a final food product during production or for stand-alone use, when appropriately diluted, as a tabletop sweetener or an at-home replacement for sugars in baking. Sweeteners include natural sweeteners such as sucrose, high fructose corn syrup, molasses, maple syrup, and honey and artificial sweeteners such as aspartame, saccharine and sucralose. Stevia extract is a natural sweetener that can be isolated and extracted from a perennial shrub, Stevia rebaudiana. Stevia is commonly grown in South America and Asia for commercial production of stevia extract. Stevia extract, purified to various degrees, is used commercially as a high intensity sweetener in foods and in blends or alone as a tabletop sweetener.

Extracts of the Stevia plant contain rebaudiosides and other steviol glycosides that contribute to the sweet flavor, although the amount of each glycoside often varies among different production batches. Existing commercial products are predominantly rebaudioside A with lesser amounts of other glycosides such as rebaudioside C, D, and F. Stevia extracts may also contain contaminants such as plant-derived compounds that contribute to off-flavors. These off-flavors can be more or less problematic depending on the food system or application of choice. Potential contaminants include pigments, lipids, proteins, phenolics, saccharides, spathulenol and other sesquiterpenes, labdane diterpenes, monoterpenes, decanoic acid, 8,11,14-eicosatrienoic acid, 2-methyloctadecane, pentacosane, octacosane, tetracosane, octadecanol, stigmasterol, β-sitosterol, α- and β-amyrin, lupeol, β-amryin acetate, pentacyclic triterpene, centauredin, quercitin, epi-alpha-cadinol, carophyllenes and derivatives, beta-pinene, beta-sitosterol, and gibberellin.

### SUMMARY

Provided herein is a recombinant host, such as a microorganism, comprising one or more biosynthesis genes whose expression results in production of steviol. Such genes include a gene encoding a copalyl diphosphate synthase, a gene encoding a kaurene synthase, a gene encoding a kaurene oxidase; and a gene encoding a steviol synthetase. The recombinant host can include a gene encoding a bifunctional copalyl diphosphate synthase and kaurene synthase, in place of the genes encoding copalyl diphosphate synthase and kaurene synthase. At least one of the genes is a recombinant gene. In some embodiments the recombinant host further comprises a gene encoding a geranylgeranyl diphosphate synthase. The recombinant host can further comprise a gene encoding a truncated HMG-CoA reductase and/or a gene encoding a CPR. The expression of one or more of the genes can be inducible.

In one aspect, this document features a recombinant host that includes a recombinant gene encoding a UGT91D2 polypeptide (e.g., a UGT91D2e or UGT91D2m polypeptide). The UGT91D2 polypeptide can have at least 90% identity (e.g., at least 95% or 99% identity) to the amino acid sequence set forth in SEQ ID NO:5. The UGT91D2 polypeptide can include at least one amino acid substitution at residues 1-19, 27-38, 44-87, 96-120, 125-141, 159-184, 199-202, 215-380, or 387-473 of SEQ ID NO:5. For example, the UGT91D2 polypeptide can include an amino acid substitution at one or more residues selected from the group consisting of residues 30, 93, 99, 122, 140, 142, 148, 153, 156, 195, 196, 199, 206, 207, 211, 221, 286, 343, 427, and 438 of SEQ ID NO:5. In one embodiment, the UGT91D2 polypeptide includes an arginine at residue 206, a cysteine at residue 207, and an arginine at residue 343 relative to SEQ ID NO:5. In one embodiment, the UGT91D2 polypeptide includes a phenylalanine at residue 30, a glutamine at residue 93, a valine at residue 99, a phenylalanine at residue 122, a tyrosine at residue 140, a cysteine at residue 142, a threonine at residue 148, an alanine at residue 153, a serine at residue 156, a methionine at residue 195, a glutamic acid at residue 196, a glutamic acid at residue 199, a methionine at residue 211, a phenylalanine at residue 221, an alanine at residue 286, an asparagine at residue 427, or an alanine at residue 438 relative to SEQ ID NO:5. The polypeptide can have the amino acid sequence of SEQ ID NO:5 or SEQ ID NO:95.

A host described herein further can include a recombinant gene encoding a UGT85C polypeptide having at least 90% identity to the amino acid sequence set forth in SEQ ID NO:3. For example, the UGT85C polypeptide can include one or more amino acid substitutions at residues 9, 10, 13, 15, 21, 27, 60, 65, 71, 87, 91, 220, 243, 270, 289, 298, 334, 336, 350, 368, 389, 394, 397, 418, 420, 440, 441, 444, and 471 of SEQ ID NO:3.

A host described herein further can include a recombinant gene encoding a UGT76G polypeptide having at least 90% identity to the amino acid sequence set forth in SEQ ID NO:7. For example, the UGT76G polypeptide can have one or more amino acid substitutions at residues 29, 74, 87, 91, 116, 123, 125, 126, 130, 145, 192, 193, 194, 196, 198, 199, 200, 203, 204, 205, 206, 207, 208, 266, 273, 274, 284, 285, 291, 330, 331, and 346 of SEQ ID NO:7.

This document also features a recombinant host that includes a recombinant gene encoding a UGT85C polypeptide having at least 90% identity to the amino acid sequence set forth in SEQ ID NO:3, and having one or more amino acid substitutions at residues 9, 10, 13, 15, 21, 27, 60, 65, 71, 87, 91, 220, 243, 270, 289, 298, 334, 336, 350, 368, 389, 394, 397, 418, 420, 440, 441, 444, and 471 of SEQ ID NO:3. For example, the UGT85C polypeptide can include substitutions at residues 13, 15, 60, 270, 289, and 418 of SEQ ID NO:3. For example, the UGT85C polypeptide can include a) substitutions at residues 13, 60, and 270 of SEQ ID NO:3; b) substitutions at residues 60 and 87 of SEQ ID NO:3; c) substitutions at residues 65, 71, 220, 243, and 270 of SEQ ID NO:3; d) substitutions at residues 65, 71, 220, 243, 270, and 441 of SEQ ID NO:3; e) substitutions at residues 65, 71, 220, 389, and 394 of SEQ ID NO:3; f) substitutions at residues 65, 71, 270, and 289 of SEQ ID NO:3; g) substitutions at residues 15 and 65 of SEQ ID NO:3; h) substitutions at residues 65 and 270 of SEQ ID NO:3; i) substitutions at residues 65 and 440 of SEQ ID NO:3; j) substitutions at residues 65 and 441 of SEQ ID NO:3; k) substitutions at residues 65 and 418 of SEQ ID NO:3; l) substitutions at residues 220, 243, 270, and 334 of SEQ ID NO:3; or m) substitutions at residues 270 and 289 of SEQ ID NO:3.

In another aspect, this document features a recombinant host that includes a recombinant gene encoding a UGT76G polypeptide having at least 90% identity to the amino acid sequence set forth in SEQ ID NO:7, and having one or more amino acid substitutions at residues 29, 74, 87, 91, 116, 123, 125, 126, 130, 145, 192, 193, 194, 196, 198, 199, 200, 203, 204, 205, 206, 207, 208, 266, 273, 274, 284, 285, 291, 330, 331, and 346. For example, the UGT76G polypeptide can have a) substitutions at amino acid residues 74, 87, 91, 116, 123, 125, 126, 130, 145, 192, 193, 194, 196, 198, 199, 200, 203, 204, 205, 206, 207, 208, and 291; b) substitutions at residues 74, 87, 91, 116, 123, 125, 126, 130, 145, 192, 193, 194, 196, 198, 199, 200, 203, 204, 205, 206, 207, 208, 266, 273, 274, 284, 285, and 291; or c) substitutions at residues 74, 87, 91, 116, 123, 125, 126, 130, 145, 192, 193, 194, 196, 198, 199, 200, 203, 204, 205, 206, 207, 208, 266, 273, 274, 284, 285, 291, 330, 331, and 346.

Any of the hosts described herein further can include a gene encoding a UGT74G1 polypeptide (e.g., a recombinant gene encoding a UGT74G1 polypeptide).

Any of the hosts described herein further can include one or more of: (i) a gene encoding a geranylgeranyl diphosphate synthase; (ii) a gene encoding a bifunctional copalyl diphosphate synthase and kaurene synthase, or a gene encoding a copalyl diphosphate synthase and a gene encoding a kaurene synthase; (iii) a gene encoding a kaurene oxidase; (iv) a gene encoding a steviol synthetase; (v) a gene encoding a truncated HMG-CoA; (vi) a gene encoding a CPR; (vii) a gene encoding a rhamnose synthetase; (viii) a gene encoding a UDP-glucose dehydrogenase; and (ix) a gene encoding a UDP-glucuronic acid decarboxylase. At least one of the genes of (i), (ii), (iii), (iv), (v), (vi), (vii), (viii), or (ix) can be a recombinant gene. In some embodiments, each of the genes of (i), (ii), (iii), and (iv) is a recombinant gene.

This document also features an isolated nucleic acid encoding a polypeptide having at least 90% sequence identity (e.g., at least 95% or 99% sequence identity) to the amino acid sequence set forth in SEQ ID NO:5. The polypeptide can include at least one amino acid substitution at residues 1-19, 27-38, 44-87, 96-120, 125-141, 159-184, 199-202, 215-380, or 387-473 of SEQ ID NO:5. The polypeptide can include an amino acid substitution at one or more residues selected from the group consisting of residues 30, 93, 99, 122, 140, 142, 148, 153, 156, 195, 196, 199, 206, 207, 211, 221, 286, 343, 427, and 438 of SEQ ID NO:5. The polypeptide can include an arginine at residue 206, a cysteine at residue 207, and an arginine at residue 343 of SEQ ID NO:5. In some embodiments, the polypeptide includes a phenylalanine at residue 30, a glutamine at residue 93, a valine at residue 99, a phenylalanine at residue 122, a tyrosine at residue 140, a cysteine at residue 142, a threonine at residue 148, an alanine at residue 153, a serine at residue 156, a methionine at residue 195, a glutamic acid at residue 196, a glutamic acid at residue 199, a methionine at residue 211, a phenylalanine at residue 221, an alanine at residue 286, an asparagine at residue 427, or an alanine at residue 438 of SEQ ID NO:5.

In another aspect, this document features an isolated polypeptide having an amino acid sequence with at least 90% identity to the amino acid sequence of SEQ ID NO:5.

This document also features a recombinant host that includes (i) a gene encoding a geranylgeranyl diphosphate synthase; (ii) a gene encoding a bifunctional copalyl diphosphate synthase and kaurene synthase, or a gene encoding a copalyl diphosphate synthase and a gene encoding a kaurene synthase; (iii) a gene encoding a kaurene oxidase; and (iv) a gene encoding a steviol synthetase; wherein at least one of said genes. The host can produce steviol when cultured under conditions in which each of the genes is expressed, and can accumulate to at least 1 mg/L in the culture medium. The geranylgeranyl diphosphate synthase can have greater than 90% sequence identity to one of the amino acid sequences set forth in SEQ ID NOs: 121-128. The copalyl diphosphate synthase can have greater than 90% sequence identity to one of the amino acid sequences set forth in SEQ ID NOs:129-131. The kaurene synthase can have greater than 90% sequence identity to one of the amino acid sequences set forth in 132-135. The kaurene oxidase can have greater than 90% sequence identity to one of the amino acid sequences set forth in 138-141. The steviol synthetase can have greater than 90% sequence identity to one of the amino acid sequences set forth in SEQ ID NOs:142-146. The host further can include a gene encoding a truncated HMG-CoA and/or a gene encoding a CPR.

Any of the recombinant hosts further can include one or more of a gene encoding a UGT74G1 polypeptide, a UGT85C2 polypeptide, a UGT76G1 polypeptide, or a UGT91D2 polypeptide.

Any of the recombinant hosts can produce at least one steviol glycoside when cultured under conditions in which each of the genes is expressed. The steviol glycoside can be selected from the group consisting of steviol-13-O-glucoside, steviol-19-O-glucoside, rubusoside, rebaudioside A, rebaudioside B, rebaudioside C, rebaudioside D, rebaudioside E, rebaudioside F, and dulcoside A. The steviol glycoside can accumulate to at least 1 mg/liter (e.g., at least 10 mg/liter or 20 mg/liter) of culture medium when cultured under said conditions.

Any of the recombinant hosts further can include one or more of i) a gene encoding a deoxyxylulose 5-phosphate synthase (DXS); ii) a gene encoding a D-1-deoxyxylulose 5-phosphate reductoisomerase (DXR); iii) a gene encoding a 4-diphosphocytidyl-2-C-methyl-D-erythritol synthase (CMS); iv) a gene encoding a 4-diphosphocytidyl-2-C-methyl-D-erythritol kinase (CMK); v) a gene encoding a 4-diphosphocytidyl-2-C-methyl-D-erythritol 2,4-cyclodiphosphate synthase (MCS); vi) a gene encoding a 1-hydroxy-2-methyl-2(E)-butenyl 4-diphosphate synthase (HDS); or vii) a gene encoding a 1-hydroxy-2-methyl-2(E)-butenyl 4-diphosphate reductase (HDR).

Any of the recombinant hosts further can include one or more of ix) a gene encoding a acetoacetyl-CoA thiolase; x) a gene encoding a truncated HMG-CoA reductase; xi) a gene encoding a mevalonate kinase; xii) a gene encoding a phosphomevalonate kinase; or xiii) a gene encoding a mevalonate pyrophosphate decarboxylase.

In any of the hosts described herein, expression of one or more of the genes can be inducible.

Any of the hosts described herein can be a microorganism (e.g., a Saccharomycete such as Saccharomyces cerevisiae, or Escherichia coli), or a plant or plant cell (e.g., a Stevia such as a Stevia rebaudiana, Physcomitrella, or tobacco plant or plant cell).

In another aspect, this document features a method of producing steviol or a steviol glycoside. The method includes growing a host described herein in a culture medium, under conditions in which the genes are expressed; and recovering the steviol or steviol glycoside produced by the host. The growing step can include inducing expression of one or more of the genes. The steviol or steviol glycoside is selected from the group consisting of steviol-13-O-glucoside, steviol-19-O-glucoside, rubusoside, rebaudioside A, rebaudioside B, rebaudioside C, rebaudioside D, rebaudioside E, rebaudioside F, and dulcoside A.

Also provided herein is a method of producing steviol or a steviol glycoside. The method includes growing a microorganism in a culture medium, under conditions in which a geranylgeranyl diphosphate synthase, copalyl diphosphate synthase, kaurene synthase, kaurene oxidase, kaurenoic acid 13-hydroxylase gene and optionally a UGT74G1 and/or a UGT85C2 gene are expressed, and recovering the steviol or steviol glycoside produced by the microorganism. The microorganism can be a Saccharomyces spp. In some embodiments, the growing step comprises inducing expression of one or more of the geranylgeranyl diphosphate synthase, copalyl diphosphate synthase, kaurene synthase, kaurene oxidase, kaurenoic acid 13-hydroxylase, UGT74G1 and UGT85C2 genes. In some embodiments, the recovering step comprises purifying the steviol or steviol glycoside from the culture medium by HPLC. The steviol or steviol glycoside can be steviol, rubusoside, rebaudioside C, rebaudioside F, or dulcoside A.

Also provided herein is a recombinant Saccharomyces strain, comprising one or more biosynthesis genes whose expression results in production of ent-kaurene. The biosynthesis genes include a gene encoding a bifunctional copalyl diphosphate synthase and kaurene synthase, or a gene encoding a copalyl diphosphate synthase and a gene encoding a kaurene synthase. The strain produces ent-kaurene upon expression of the copalyl diphosphate synthase and the kaurene synthase.

In another aspect, this document features an isolated nucleic acid having greater than 90% sequence identity (e.g., greater than 95% or 99% sequence identity) to one of the nucleotide sequences set forth in SEQ ID NOs: 18-25, 34-36, 4-43, 48, 49, 52-55, 60-64, 70-72, 77, or 79.

This document also features a recombinant host that includes (i) a gene encoding a UGT74G1; (ii) a gene encoding a UGT85C2; (iii) a gene encoding a UGT76G1; and (iv) a gene encoding a UGT91D2, wherein at least one of said genes is a recombinant gene. In some embodiments, each of the genes is a recombinant gene. The host can produce at least one steviol glycoside when cultured under conditions in which each of the genes is expressed. The host further can include (a) a gene encoding a bifunctional copalyl diphosphate synthase and kaurene synthase, or a gene encoding a copalyl diphosphate synthase and a gene encoding a kaurene synthase; (b) a gene encoding a kaurene oxidase; (c) a gene encoding a steviol synthetase; and (d) a gene encoding a geranylgeranyl diphosphate synthase. The steviol glycoside can be rebaudioside A, rebaudioside D or rebaudioside E. This document also features a steviol glycoside composition produced by such a host. The composition can have greater than 4% rebaudioside D by weight of total steviol glycosides and a reduced level of stevia plant-derived contaminants relative to a stevia extract. The composition can have greater than 4% rebaudioside E by weight of total steviol glycosides and a reduced level of stevia plant-derived contaminants relative to a stevia extract.

Also featured herein is an isolated nucleic acid encoding a polypeptide having greater than 90% sequence identity to the amino acid sequences of UGT91D2e and UGT91D2m, excluding the amino acid sequence of UGT91D2m, as well as the isolated polypeptides having greater than 90% sequence identity to the amino acid sequence of UGT91D2e or UGT91D2m, excluding the amino acid sequence of UGT91D2m.

This document also features steviol glycoside composition produced by the host described herein. The composition having reduced levels of stevia plant-derived contaminants relative to a stevia extract.

In another aspect, this document features a recombinant host. The host includes (i) a recombinant gene encoding a UGT91D2; (ii) a recombinant gene encoding a UGT74G1; (iii) a recombinant gene encoding a UGT85C2; (iv) a recombinant gene encoding a UGT76G1; and (v) a gene encoding a rhamnose synthetase, wherein the host produces at least one steviol glycoside when cultured under conditions in which each of the genes is expressed. The host further can include (a) a gene encoding a bifunctional copalyl diphosphate synthase and kaurene synthase, or a gene encoding a copalyl diphosphate synthase and a gene encoding a kaurene synthase; (b) a gene encoding a kaurene oxidase; (c) a gene encoding a steviol synthetase; and (d) a gene encoding a geranylgeranyl diphosphate synthase. The steviol glycoside can be rebaudioside C or dulcoside A. This document also features a steviol glycoside composition produced by such a host. The composition has greater than 15% rebaudioside C by weight of total steviol glycosides and a reduced level of stevia plant-derived contaminants relative to a stevia extract. A steviol glycoside composition produced by such a host also is featured. The composition can have greater than 15% dulcoside A by weight of total steviol glycosides and a reduced level of stevia plant-derived contaminants relative to a stevia extract.

This document also features a recombinant host. The host includes (i) a recombinant gene encoding a UGT91D2; (ii) a recombinant gene encoding a UGT74G1; (iii) a recombinant gene encoding a UGT85C2; (iv) a recombinant gene encoding a UGT76G1; (v) a gene encoding a UDP-glucose dehydrogenase; and (vi) a gene encoding a UDP-glucuronic acid decarboxylase, wherein the host produces at least one steviol glycoside when cultured under conditions in which each of the genes is expressed. The host further can include (a) a gene encoding a bifunctional copalyl diphosphate synthase and kaurene synthase, or a gene encoding a copalyl diphosphate synthase and a gene encoding a kaurene synthase; (b) a gene encoding a kaurene oxidase; (c) a gene encoding a steviol synthetase; and (d) a gene encoding a geranylgeranyl diphosphate synthase. The steviol glycoside can be rebaudioside F. This document also features a steviol glycoside composition produced by such hosts. The composition can have greater than 4% rebaudioside F by weight of total steviol glycosides and a reduced level of stevia plant-derived contaminants relative to a stevia extract.

In another aspect, this document features a method of producing a steviol glycoside composition. The method includes growing a host described herein in a culture medium, under conditions in which each of the genes is expressed; and recovering the steviol glycoside composition produced by the host, wherein the recovered composition is enriched for rebaudioside A, rebaudioside C, rebaudioside D, rebaudioside E, rebaudioside F or dulcoside A relative to the steviol glycoside composition of a wild-type Stevia plant. The steviol glycoside composition produced by the host (e.g., microorganism) can have a reduced level of stevia plant-derived contaminants relative to a stevia extract.

This document also features a food product that includes a steviol glycoside composition enriched for rebaudioside A, rebaudioside C, rebaudioside D, rebaudioside E, rebaudioside F or dulcoside A relative to the steviol glycoside composition of a wild-type Stevia plant.

In another aspect, this document features a method of identifying whether a polymorphism is associated with variation in a trait. The method includes determining whether one or more genetic polymorphisms in a population of plants is associated with the locus for a polypeptide set forth in SEQ ID NO:5 and functional homologs thereof; and measuring the correlation between variation in the trait in plants of the population and the presence of the one or more genetic polymorphisms in plants of the population, thereby identifying whether or not the one or more genetic polymorphisms are associated with variation in the trait.

In yet another aspect, this document features a method of making a plant line. The method includes determining whether one or more genetic polymorphisms in a population of plants is associated with the locus for a polypeptide set forth in SEQ ID NO:5 and functional homologs thereof; identifying one or more plants in the population in which the presence of at least one of the genetic polymorphisms is associated with variation in a trait; crossing one or more of the identified plants with itself or a different plant to produce seed; crossing at least one progeny plant grown from the seed with itself or a different plant; and repeating the crossing steps for an additional 0-5 generations to make said plant line, wherein at least one of the genetic polymorphisms is present in the plant line.

This document also features a method for transferring a second sugar moiety to the C-2′ of a glucose in a steviol glycoside. The method includes contacting the steviol glycoside with a UGT91D2 polypeptide and a UDP-sugar under suitable reaction conditions for the transfer of the second sugar moiety to the steviol glycoside. The UGT91D2 polypeptide can have at least 90% sequence identity (e.g., at least 95% or 99%) to the amino acid sequence set forth in SEQ ID NO:5. The UGT91D2 polypeptide can include at least one amino acid substitution at residues 1-19, 27-38, 44-87, 96-120, 125-141, 159-184, 199-202, 215-380, or 387-473 of SEQ ID NO:5. The UGT91D2 polypeptide can include an amino acid substitution at one or more residues selected from the group consisting of residues 30, 93, 99, 122, 140, 142, 148, 153, 156, 195, 196, 199, 206, 207, 211, 221, 286, 343, 427, and 438 of SEQ ID NO:5. The steviol glycoside can be selected from the group consisting of steviol-13-O-glucoside, rubusoside, stevioside, and Rebaudioside A. The steviol glycoside can be rubusoside and the second sugar moiety is glucose, and stevioside is produced upon transfer of the second glucose moiety. The steviol glycoside can be stevioside and the second sugar moiety can be glucose, and Rebaudioside E is produced upon transfer of the second glucose moiety. The steviol glycoside can be stevioside, wherein stevioside is contacted with the UGT91D2 polypeptide and a UGT76G1 polypeptide under suitable reaction conditions to produce Rebaudioside D. The steviol glycoside can be steviol-13-O-glucoside and steviol-1,2 bioside is produced upon transfer of said second glucose moiety. The steviol glycoside can be steviol-13-O-glucoside and steviol-1,2-xylobioside is produced upon transfer of the second sugar moiety. The steviol glycoside can be steviol-13-O-glucoside and steviol-1,2-rhamnobioside can be produced upon transfer of the second sugar moiety. The steviol glycoside can be Rebaudioside A, and Rebaudioside D is produced upon transfer of a second glucose moiety.

In another aspect, this document features a method of determining the presence of a polynucleotide in a Stevia plant. The method includes contacting at least one probe or primer pair with nucleic acid from the Stevia plant, wherein the probe or primer pair is specific for a polynucleotide that encodes a UGT polypeptide, wherein the UGT polypeptide has at least 90% sequence identity to SEQ ID NO: 5, SEQ ID NO: 1, SEQ ID NO: 3 or SEQ ID NO:7 and determining whether or not the polynucleotide is present in said Stevia plant.

This document also features a kit for genotyping a Stevia biological sample. The kit includes a primer pair that specifically amplifies, or a probe that specifically hybridizes to, a polynucleotide that encodes a UGT polypeptide having at least 90% sequence identity to SEQ ID NO: 5, SEQ ID NO: 1, SEQ ID NO: 3 or SEQ ID NO:7.

Also provided herein is a recombinant microorganism, comprising one or more biosynthesis genes whose expression results in production of one or more steviol glycosides. The biosynthesis genes include a gene encoding a geranylgeranyl diphosphate synthase, a gene encoding a copalyl diphosphate synthase and a gene encoding a kaurene synthase, a gene encoding a kaurene oxidase, a gene encoding a steviol synthetase, and a gene encoding a UGT74G1 and/or a UGT85C2. At least one of the genes is a recombinant gene. The microorganism can comprise a gene encoding a bifunctional copalyl diphosphate synthase and kaurene synthase in place of the genes encoding copalyl diphosphate synthase and kaurene synthase.

The recombinant microorganism produces at least one steviol glycoside when cultured under conditions in which each of the genes is expressed. The steviol glycoside can be rubusoside, rebaudioside C, rebaudioside F, dulcoside B, or dulcoside A.

The recombinant microorganism can be a Saccharomycete, e.g., Saccharomyces cerevisiae, and can have one or more genetic modifications that reduce EXG1 and EXG2 glycoside hydrolase activity relative to a control microorganism that lacks such genetic modifications, and can have one or more genetic modifications that reduce ergosterol biosynthesis relative to a control microorganism that lacks such genetic modifications. The Saccharomycete produces rubusoside when cultured under conditions in which each of the genes is expressed. The rubusoside can accumulate to at least 10 mg/liter of culture medium. The Saccharomycete can be a Saccharomyces cerevisiae strain designated CEY171, CEY191, or CEY213.

The recombinant microorganism can further comprise a gene encoding an SM12UGT and a gene encoding a UGT76G1, and produce a steviol glycoside when cultured under conditions in which each of the genes is expressed. The steviol glycoside can be rebaudioside A.

Also provided herein is a recombinant microorganism, comprising one or more biosynthesis genes whose expression results in production of at least one steviol glycoside. The biosynthesis genes include a gene encoding an SM12UGT, a gene encoding a UGT74G1, a gene encoding a UGT76G1 and a gene encoding a UGT85C2. The recombinant microorganism produces rebaudioside A or rebaudioside B when cultured under conditions in which each of the genes is expressed. The rebaudioside A or rebaudioside B can accumulate to at least 1 mg/L in the culture medium.

Also featured herein is a recombinant microorganism, comprising a gene encoding a UGT91D2 polypeptide, e.g., a recombinant UGT91D2 gene.

Also featured herein is a recombinant microorganism, comprising a gene encoding a geranylgeranyl diphosphate synthase, a gene encoding a bifunctional copalyl diphosphate synthase and kaurene synthase (or a gene encoding a copalyl diphosphate synthase and a gene encoding a kaurene synthase), a gene encoding a kaurene oxidase, a gene encoding a steviol synthetase, a gene encoding a UGT74G1, a gene encoding a UGT85C2, a gene encoding a UGT76G1, and a gene encoding a UGT91D2. At least one of the genes is a recombinant gene. The recombinant microorganism can produce at least one steviol glycoside, e.g., rebaudioside A, rebaudioside B, and/or rebaudioside F, when cultured under conditions in which each of the genes is expressed. The recombinant microorganism can accumulate at least 20 mg of steviol glycoside per liter of culture medium when cultured under such conditions. The recombinant microorganism can be a Saccharomycete, e.g., Saccharomyces cerevisiae, and can have one or more genetic modifications that reduce EXG1 and EXG2 glycoside hydrolase activity relative to a control microorganism that lacks such genetic modifications, and can have one or more genetic modifications that reduce ergosterol biosynthesis relative to a control microorganism that lacks such genetic modifications.

Also featured herein is a recombinant microorganism, comprising a gene encoding a UGT74G1, a gene encoding a UGT85C2, a gene encoding a UGT76G1, and a gene encoding a UGT91D2. At least one of the genes is a recombinant gene. The recombinant microorganism can produce a steviol glycoside, e.g., rebaudioside A or rebaudioside B, when cultured under conditions in which each of the genes is expressed. The rebaudioside A or rebaudioside B can accumulate to at least 15 mg/L in the culture medium.

The recombinant microorganisms described above can further comprise a gene encoding a deoxyxylulose 5-phosphate synthase (DXS), and/or a gene encoding a D-1-deoxyxylulose 5-phosphate reductoisomerase (DXR), and/or a gene encoding a 4-diphosphocytidyl-2-C-methyl-D-erythritol synthase (CMS), and/or a gene encoding a 4-diphosphocytidyl-2-C-methyl-D-erythritol kinase (CMK), and/or a gene encoding a 4-diphosphocytidyl-2-C-methyl-D-erythritol 2,4-cyclodiphosphate synthase (MCS), and/or a gene encoding a 1-hydroxy-2-methyl-2(E)-butenyl 4-diphosphate synthase (HDS), and/or a gene encoding a 1-hydroxy-2-methyl-2(E)-butenyl 4-diphosphate reductase (HDR).

The recombinant microorganisms described above can further comprise a gene encoding a acetoacetyl-CoA thiolase, and/or a gene encoding a truncated HMG-CoA reductase, and/or a gene encoding a mevalonate kinase, and/or a gene encoding a phosphomevalonate kinase, and/or a gene encoding a mevalonate pyrophosphate decarboxylase.

Unless otherwise defined, all technical and scientific terms used herein have the same meaning as commonly understood by one of ordinary skill in the art to which the invention belongs. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and are not intended to be limiting. Other features and advantages of the invention will be apparent from the following detailed description. Applicants reserve the right to alternatively claim any disclosed invention using the transitional phrase “comprising,” “consisting essentially of,” or “consisting of,” according to standard practice in patent law.

### DESCRIPTION OF DRAWINGS

FIG. 1 is a scheme illustrating the biosynthesis of steviol from geranylgeranyl diphosphate.

FIGS. 2A-D show representative pathways for the biosynthesis of steviol glycosides from steviol.

FIG. 3 shows chemical structures for various steviol glycosides.

FIG. 4 is a schematic representation of rebA production in Saccharomyces cerevisiae.

FIG. 5 is a schematic representation of the concatenation of genes to form cYACs.

FIG. 6 shows rubusoside production by yeast strain CEY13 under various culture conditions.

FIG. 7 shows data obtained from ¹H and ¹³C NMR analysis of the compound produced by yeast strain CEY213, compared to literature values for rubusoside.

FIG. 8 is an alignment of UGT91D1 and UGT91D2 amino acid sequences (SEQ ID NOs:14, 16, 12, 5, and 10, respectively).

FIG. 9 shows Rebaudioside A, stevioside, and rubusoside production by yeast CEY213 containing plasmid pMUS47 after 24 and 99 hours of culture.

FIG. 10A is a graph illustrating the concentrations of RebA, rubusoside and 19-SMG in supernatants. FIG. 10B is a graph of the concentrations of RebA, rubusoside and 19-SMG measured in cell pellets, for experiments where yeast cells were fed with 100 μM steviol. In both graphs, the first set of bars represents the untagged control strains; the second set of bars represents the strain containing the UGT74G1, UGT76G1, and UGT91D2e fusion proteins in which the N-terminal 158 amino acids of the MDM2 protein are fused to each UGT, and a UGT85C2 fusion protein in which four repeats of the synthetic PMI peptide is fused in-frame to the N-terminus of 85C2. The y-axis is concentration in micromolar units.

Like reference symbols in the various drawings indicate like elements.

### DETAILED DESCRIPTION

Two glycosides, stevioside and rebaudioside A, are the primary compounds in commercially-produced stevia extracts. Stevioside is reported to have a more bitter and less sweet taste than rebaudioside A and, therefore, a higher proportion of rebaudioside A in an extract preparation is preferred. However, the composition of stevia extract can vary from lot to lot depending on the soil and climate in which the plants are grown. Depending upon the sourced plant, the climate conditions, and the extraction process, the amount of rebaudioside A in commercial preparations is reported to vary from 20 to 97% of the total steviol glycoside content, typically >50-80% and sometimes as high as >95-97% of the total steviol glycosides. Moreover, other steviol glycosides are present in varying amounts in stevia extracts, which further complicates the ability to produce a sweetener with a consistent taste profile by extraction and purification from Stevia plants. For example, Rebaudioside B is typically present at less than 1-2%, whereas Rebaudioside C can be present at levels as high as 7-15%. Rebaudioside D is typically present in levels of 2% or less, and Rebaudioside F is typically present in compositions at 3.5% or less of the total steviol glycosides. Even trace amounts of the minor steviol glycosides are reported to affect the flavor profile of a Stevia extract. Additionally, it is thought that some of the contaminants from the Stevia plant, even at very low concentrations, may also provide off-flavors to some of the commercially available plant extracts.

This document is based on the discovery that recombinant hosts such as plant cells, plants, or microorganisms can be developed that express polypeptides useful for the biosynthesis of steviol. Further, such hosts can express Uridine 5′-diphospho (UDP) glycosyl transferases suitable for producing steviol glycosides such as rubusoside and rebaudioside A. Recombinant microorganisms are particularly useful hosts. Expression of these biosynthetic polypeptides in various microbial chassis allows steviol and its glycosides to be produced in a consistent, reproducible manner from energy and carbon sources such as sugars, glycerol, CO₂, H₂, and sunlight. The proportion of each steviol glycoside produced by a recombinant host can be tailored by incorporating preselected biosynthetic enzymes into the hosts and expressing them at appropriate levels, to produce a sweetener composition with a consistent taste profile. Furthermore, the concentrations of steviol glycosides produced by recombinant hosts are expected to be higher than the levels of steviol glycosides produced in the Stevia plant, which improves the efficiency of the downstream purification. Such sweetener compositions contain little or no plant based contaminants, relative to the amount of contaminants present in Stevia extracts.

At least one of the genes is a recombinant gene, the particular recombinant gene(s) depending on the species or strain selected for use. Additional genes or biosynthetic modules can be included in order to increase steviol and glycoside yield, improve efficiency with which energy and carbon sources are converted to steviol and its glycosides, and/or to enhance productivity from the cell culture or plant. Such additional biosynthetic modules include genes involved in the synthesis of the terpenoid precursors, isopentenyl diphosphate and dimethylallyl diphosphate. Additional biosynthetic modules include terpene synthase and terpene cyclase genes, such as genes encoding geranylgeranyl diphosphate synthase and copalyl diphosphate synthase; these genes may be endogenous genes or recombinant genes.

### I. STEVIOL AND STEVIOL GLYCOSIDE BIOSYNTHESIS POLYPEPTIDES

### A. Steviol Biosynthesis Polypeptides

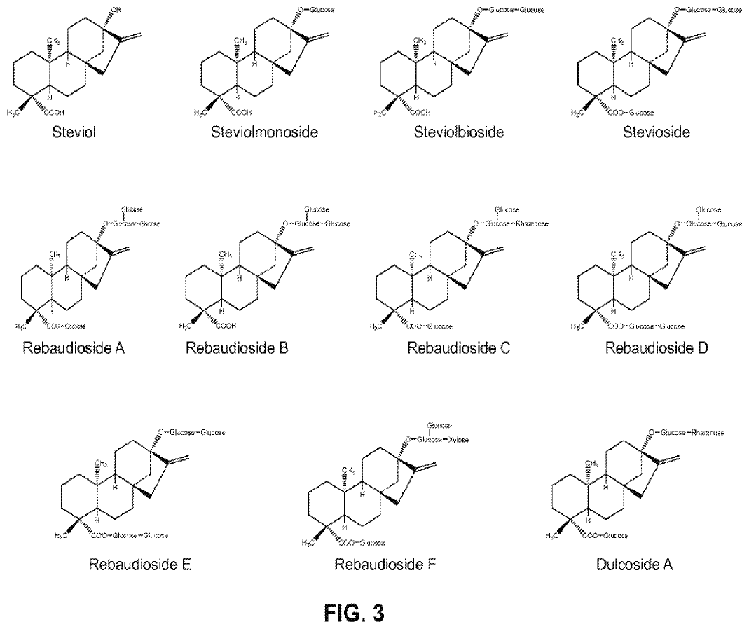

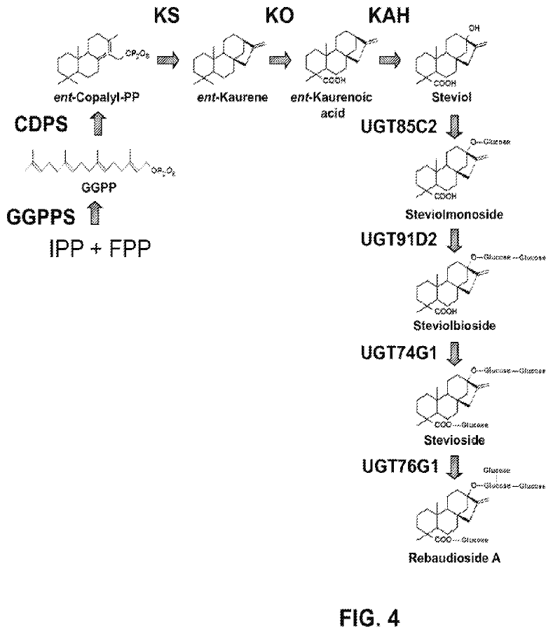

Chemical structures for several of the compounds found in Stevia extracts are shown in FIG. 3, including the diterpene steviol and various steviol glycosides. CAS numbers are shown in Table A below. See also, Steviol Glycosides Chemical and Technical Assessment 69th JECFA, prepared by Harriet Wallin, Food Agric. Org. (2007).

TABLE A

| COMPOUND | CAS# |
|---|---|
| Steviol | 471-80-7 |
| Rebaudioside A | 58543-16-1 |
| Steviolbioside | 41093-60-1 |
| Stevioside | 57817-89-7 |
| Rebaudioside B | 58543-17-2 |
| Rebaudioside C | 63550-99-2 |
| Rebaudioside D | 63279-13-0 |
| Rebaudioside E | 63279-14-1 |
| Rebaudioside F | 438045-89-7 |
| Rubusoside | 63849-39-4 |
| Dulcoside A | 64432-06-0 |

It has been discovered that expression of certain genes in a host such as a microorganism confers the ability to synthesize steviol upon that host. As discussed in more detail below, one or more of such genes may be present naturally in a host. Typically, however, one or more of such genes are recombinant genes that have been transformed into a host that does not naturally possess them.

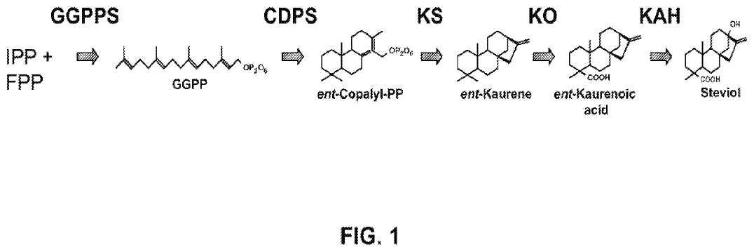

The biochemical pathway to produce steviol involves formation of geranylgeranyl diphosphate, cyclization to (−) copalyl diphosphate, followed by oxidation and hydroxylation to form steviol. See FIG. 1. Thus, conversion of geranylgeranyl diphosphate to steviol in a recombinant microorganism involves the expression of a gene encoding a kaurene synthase (KS), a gene encoding a kaurene oxidase (KO), and a gene encoding a steviol synthetase (KAH). Steviol synthetase also is known as kaurenoic acid 13-hydroxylase.

Suitable KS polypeptides are known. For example, suitable KS enzymes include those made by Stevia rebaudiana, Zea mays and Populus trichocarpa. See, SEQ ID NOs: 132-135. Nucleotide sequences encoding these polypeptides are described in more detail below. See, for example, Table 3 and SEQ ID NOs: 40-47.

Suitable KO polypeptides are known. For example, suitable KO enzymes include those made by Stevia rebaudiana, Arabidopsis thaliana, Gibberella fujikoroi and Trametes versicolor. See, SEQ ID NOs: 138-141. Nucleotide sequences encoding these polypeptides are described in more detail below. See, for example, Table 5 and SEQ ID NOs: 52-59.

Suitable KAH polypeptides are known. For example, suitable KAH enzymes include those made by Stevia rebaudiana, Arabidopsis thaliana, Vitis vinifera and Medicago trunculata. See, e.g., SEQ ID NOs: 142-146; U.S. Patent Publication No. 2008-0271205; U.S. Patent Publication No. 2008-0064063 and Genbank Accession No. gi 189098312. The steviol synthetase from Arabidopsis thaliana is classified as a CYP714A2. Nucleotide sequences encoding these polypeptides are described in more detail below. See, for example, Table 6 and SEQ ID NOs: 60-69.

In some embodiments, a recombinant microorganism contains a recombinant gene encoding a KO and/or a KAH polypeptide. Such microorganisms also typically contain a recombinant gene encoding a cytochrome P450 reductase (CPR) polypeptide, since certain combinations of KO and/or KAH polypeptides require expression of an exogenous CPR polypeptide. In particular, the activity of a KO and/or a KAH polypeptide of plant origin can be significantly increased by the inclusion of a recombinant gene encoding an exogenous CPR polypeptide. Suitable CPR polypeptides are known. For example, suitable CPR enzymes include those made by Stevia rebaudiana, Arabidopsis thaliana, and Giberella fujikuroi. See, e.g., SEQ ID NOs: 147-149. Nucleotide sequences encoding these polypeptides are described in more detail below. See, for example, Table 7 and SEQ ID NOs: 70-75.

Expression in a recombinant microorganism of these genes results in the conversion of geranylgeranyl diphosphate to steviol.

### B. Steviol Glycoside Biosynthesis Polypeptides

In some embodiments, a recombinant host described herein can convert steviol to a steviol glycoside. Such a host (e.g., microorganism) contains genes encoding one or more UDP Glycosyl Transferases, also known as UGTs. UGTs transfer a monosaccharide unit from an activated nucleotide sugar to an acceptor moiety, in this case, an —OH or —COOH moiety on steviol or steviol derivative. UGTs have been classified into families and subfamilies based on sequence homology. Li et al. J. Biol. Chem. 276:4338-4343 (2001).

### B. 1 Rubusoside Biosynthesis Polypeptides

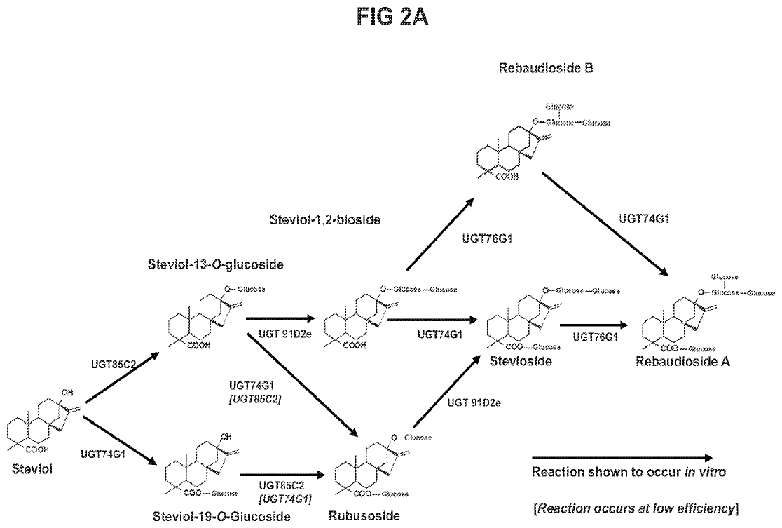

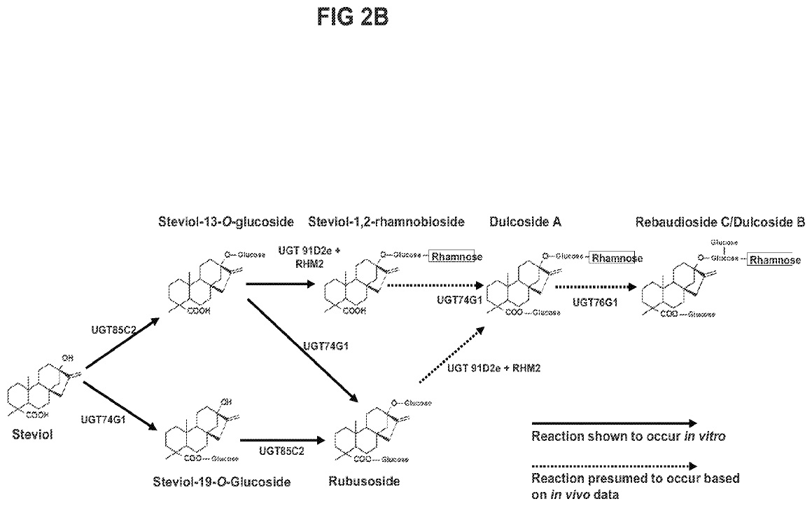

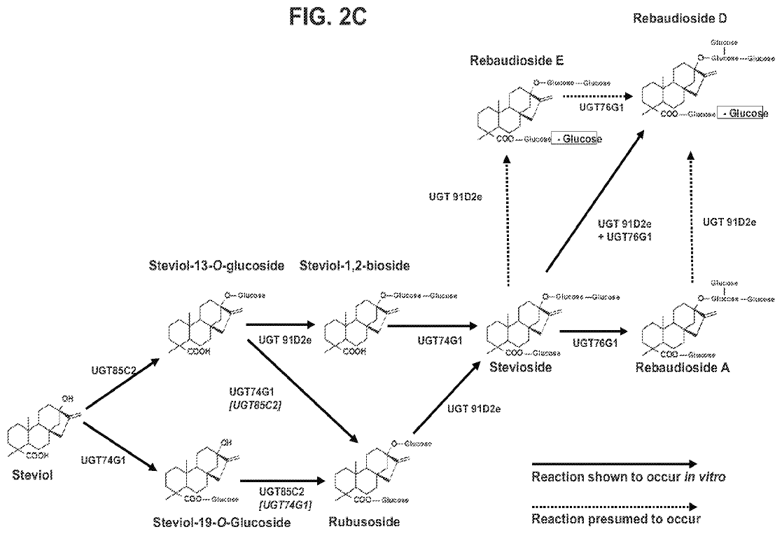

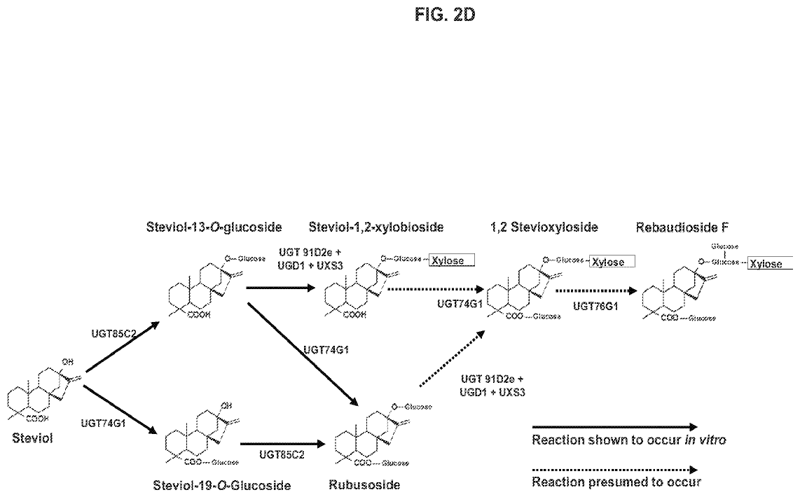

The biosynthesis of rubusoside involves glycosylation of the 13-OH and the 19-COOH of steviol. See FIG. 2A. It has been discovered that conversion of steviol to rubusoside in a recombinant host such as a microorganism can be accomplished by the expression of gene(s) encoding UGTs 85C2 and 74G1, which transfer a glucose unit to the 13-OH or the 19-COOH, respectively, of steviol.

Thus, a suitable UGT85C2 functions as a uridine 5′-diphospho glucosyl: steviol 13-OH transferase, and a uridine 5′-diphospho glucosyl: steviol-19-O-glucoside 13-OH transferase. Functional UGT85C2 polypeptides also may catalyze glucosyl transferase reactions that utilize steviol glycoside substrates other than steviol and steviol-19-O-glucoside.

A suitable UGT74G1 polypeptide functions as a uridine 5′-diphospho glucosyl: steviol 19-COOH transferase and a uridine 5′-diphospho glucosyl: steviol-13-O-glucoside 19-COOH transferase. Functional UGT74G1 polypeptides also may catalyze glycosyl transferase reactions that utilize steviol glycoside substrates other than steviol and steviol-13-O-glucoside, or that transfer sugar moieties from donors other than uridine diphosphate glucose.

A recombinant microorganism expressing a functional UGT74G1 and a functional UGT85C2 can make rubusoside and both steviol monosides (i.e., Steviol 13-O-monoglucoside and Steviol 19-O-monoglucoside) when fed steviol in the medium. One or more of such genes may be present naturally in the host. Typically, however, such genes are recombinant genes that have been transformed into a host (e.g., microorganism) that does not naturally possess them.

As used herein, the term recombinant host is intended to refer to a host, the genome of which has been augmented by at least one incorporated DNA sequence. Such DNA sequences include but are not limited to genes that are not naturally present, DNA sequences that are not normally transcribed into RNA or translated into a protein (“expressed”), and other genes or DNA sequences which one desires to introduce into the non-recombinant host. Tt will be appreciated that typically the genome of a recombinant host described herein is augmented through the stable introduction of one or more recombinant genes. Generally, the introduced DNA is not originally resident in the host that is the recipient of the DNA, but it is within the scope of the invention to isolate a DNA segment from a given host, and to subsequently introduce one or more additional copies of that DNA into the same host, e.g., to enhance production of the product of a gene or alter the expression pattern of a gene. In some instances, the introduced DNA will modify or even replace an endogenous gene or DNA sequence by, e.g., homologous recombination or site-directed mutagenesis. Suitable recombinant hosts include microorganisms, plant cells, and plants.

The term “recombinant gene” refers to a gene or DNA sequence that is introduced into a recipient host, regardless of whether the same or a similar gene or DNA sequence may already be present in such a host. “Introduced,” or “augmented” in this context, is known in the art to mean introduced or augmented by the hand of man. Thus, a recombinant gene may be a DNA sequence from another species, or may be a DNA sequence that originated from or is present in the same species, but has been incorporated into a host by genetic engineering methods to form a recombinant host. It will be appreciated that a recombinant gene that is introduced into a host can be identical to a DNA sequence that is normally present in the host being transformed, and is introduced to provide one or more additional copies of the DNA to thereby permit overexpression or modified expression of the gene product of that DNA.

Suitable UGT74G1 and UGT85C2 polypeptides include those made by Stevia rebaudiana. Genes encoding functional UGT74G1 and UGT85C2 polypeptides from Stevia are reported in Richman, et al. Plant J. 41: 56-67 (2005). Amino acid sequences of S. rebaudiana UGT74G1 and UGT85C2 polypeptides are set forth in SEQ ID NOs: 1 and 3, respectively. Nucleotide sequences encoding UGT74G1 and UGT85C2 that have been optimized for expression in yeast are set forth in SEQ ID NOs: 2 and 4, respectively. See also the UGT85C2 and UGT74G1 variants described in Examples 17 and 18, respectively.

In some embodiments, the recombinant host is a microorganism. The recombinant microorganism can be grown on media containing steviol in order to produce rubusoside. In other embodiments, however, the recombinant microorganism expresses one or more recombinant genes involved in steviol biosynthesis, e.g., a CDPS gene, a KS gene, a KO gene and/or a KAH gene. Thus, a microorganism containing a CDPS gene, a KS gene, a KO gene and a KAH gene in addition to a UGT74G1 and a UGT85C2 gene is capable of producing both steviol monosides and rubusoside without the necessity for including steviol in the culture media.

In some embodiments, the recombinant microorganism further expresses a recombinant gene encoding a geranylgeranyl diphosphate synthase (GGPPS). Suitable GGPPS polypeptides are known. For example, suitable GGPPS enzymes include those made by Stevia rebaudiana, Gibberella fujikuroi, Mus musculus, Thalassiosira pseudonana, Streptomyces clavuligerus, Sulfulobus acidocaldarius, Synechococcus sp. and Arabidopsis thaliana. See, SEQ ID NOs: 121-128. Nucleotide sequences encoding these polypeptides are described in more detail below. See Table 1 and SEQ ID NOs:18-33. In some embodiments, the recombinant microorganism further expresses recombinant genes involved in diterpene biosynthesis or production of terpenoid precursors, e.g., genes in the methylerythritol 4-phosphate (MEP) pathway or genes in the mevalonate (MEV) pathway discussed below.

### B. 2 Rebaudioside A Biosynthesis Polypeptides

The biosynthesis of rebaudioside A involves glucosylation of the aglycone steviol. Specifically, rebaudioside A can be formed by glucosylation of the 13-OH of steviol which forms the 13-O-steviolmonoside, glucosylation of the C-2′ of the 13-O-glucose of steviolmonoside which forms steviol-1,2-bioside, glucosylation of the C-19 carboxyl of steviol-1,2-bioside which forms stevioside, and glucosylation of the C-3′ of the C-13-O-glucose of stevioside. The order in which each glucosylation reaction occurs can vary. See FIG. 2A.

It has been discovered that conversion of steviol to rebaudioside A in a recombinant host can be accomplished by the expression of gene(s) encoding the following functional UGTs: 74G1, 85C2, 76G1 and 91D2. Thus, a recombinant microorganism expressing these four UGTs can make rebaudioside A when fed steviol in the medium. Typically, one or more of these genes are recombinant genes that have been transformed into a microorganism that does not naturally possess them. It has also been discovered that UGTs designated herein as SM12UGT can be substituted for UGT91D2.

Suitable UGT74G1 and UGT85C2 polypeptides include those discussed above. A suitable UGT76G1 adds a glucose moiety to the C-3′ of the C-13-O-glucose of the acceptor molecule, a steviol 1,2 glycoside. Thus, UGT76G1 functions, for example, as a uridine 5′-diphospho glucosyl: steviol 13-O-1,2 glucoside C-3′ glucosyl transferase and a uridine 5′-diphospho glucosyl: steviol-19-O-glucose, 13-O-1,2 bioside C-3′ glucosyl transferase. Functional UGT76G1 polypeptides may also catalyze glucosyl transferase reactions that utilize steviol glycoside substrates that contain sugars other than glucose, e.g., steviol rhamnosides and steviol xylosides. See, FIGS. 2A, 2B, 2C and 2D. Suitable UGT76G1 polypeptides include those made by S. rebaudiana and reported in Richman, et al. Plant J. 41: 56-67 (2005). The amino acid sequence of a S. rebaudiana UGT76G1 polypeptide is set forth in SEQ ID NO:7. The nucleotide sequence encoding the UGT76G1 polypeptide of SEQ ID NO:7 has been optimized for expression in yeast and is set forth in SEQ ID NO:8. See also the UGT76G1 variants set forth in Example 18.

A suitable UGT91D2 polypeptide functions as a uridine 5′-diphospho glucosyl: steviol-13-O-glucoside transferase (also referred to as a steviol-13-monoglucoside 1,2-glucosylase), transferring a glucose moiety to the C-2′ of the 13-O-glucose of the acceptor molecule, steviol-13-O-glucoside. Typically, a suitable UGT91D2 polypeptide also functions as a uridine 5′-diphospho glucosyl: rubusoside transferase transferring a glucose moiety to the C-2′ of the 13-O-glucose of the acceptor molecule, rubusoside.

Functional UGT91D2 polypeptides may also catalyze reactions that utilize steviol glycoside substrates other than steviol-13-O-glucoside and rubusoside, e.g., functional UGT91D2 polypeptides may utilize stevioside as a substrate, transferring a glucose moiety to the C-2′ of the 19-O-glucose residue to produce Rebaudioside E. Functional UGT91D2 polypeptides may also utilize Rebaudioside A as a substrate, transferring a glucose moiety to the C-2′ of the 19-O-glucose residue to produce Rebaudioside D. However, a functional UGT91D2 polypeptide typically does not transfer a glucose moiety to steviol compounds having a 1,3-bound glucose at the C-13 position, i.e., transfer of a glucose moiety to steviol 1,3-bioside and 1,3-stevioside does not occur.

Functional UGT91D2 polypeptides can transfer sugar moieties from donors other than uridine diphosphate glucose. For example, a functional UGT91D2 polypeptide can act as a uridine 5′-diphospho D-xylosyl: steviol-13-O-glucoside transferase, transferring a xylose moiety to the C-2′ of the 13-O-glucose of the acceptor molecule, steviol-13-O-glucoside. As another example, a functional UGT91D2 polypeptide can act as a uridine 5′-diphospho L-rhamnosyl: steviol-13-O-glucoside transferase, transferring a rhamnose moiety to the C-2′ of the 13-O-glucose of the acceptor molecule, steviol-13-O-glucoside

Suitable functional UGT91D2 polypeptides include those disclosed herein, e.g., the polypeptides designated UGT91D2e and UGT91D2m. The amino acid sequence of an exemplary UGT91D2e polypeptide from Stevia rebaudiana is set forth in SEQ ID NO: 5. SEQ ID NO:6 is a nucleotide sequence encoding the polypeptide of SEQ ID NO:5 that has been codon optimized for expression in yeast. The S. rebaudiana nucleotide sequence encoding the polypeptide of SEQ ID NO:5 is set forth in SEQ ID NO:9. The amino acid sequences of exemplary UGT91D2m polypeptides from S. rebaudiana are set forth in SEQ ID NOs: 10 and 12, and are encoded by the nucleic acid sequences set forth in SEQ ID NOs: 11 and 13, respectively. See also the UGT91D2 variants of Example 16, e.g., a variant containing a substitution at amino acid residues 206, 207, and 343.

As indicated above, UGTs designated herein as SM12UGT can be substituted for UGT91D2. Suitable functional SMI2UGT polypeptides include those made by Ipomoea purpurea (Japanese morning glory) and described in Morita et al. Plant J. 42, 353-363 (2005). The amino acid sequence encoding the I. purpurea IP3GGT polypeptide is set forth in SEQ ID NO:76. SEQ ID NO:77 is a nucleotide sequence encoding the polypeptide of SEQ ID NO:76 that has been codon optimized for expression in yeast. Another suitable SM12UGT polypeptide is a Bp94B1 polypeptide having an R25S mutation. See Osmani et al. Plant Phys. 148: 1295-1308 (2008) and Sawada et al. J. Biol. Chem. 280:899-906 (2005). The amino acid sequence encoding the Bellis perennis (red daisy) UGT94B1 polypeptide is set forth in SEQ ID NO:78. SEQ ID NO:79 is the nucleotide sequence encoding the polypeptide of SEQ ID NO:78 that has been codon optimized for expression in yeast.

In some embodiments, the recombinant microorganism is grown on media containing steviol-13-O-glucoside or steviol-19-O-glucoside in order to produce rebaudioside A. In such embodiments, the microorganism contains and expresses genes encoding a functional UGT91D2, a functional UGT74G1 and a functional UGT76G1, and is capable of producing rebaudioside A when it is fed steviol, one or both of the steviolmonosides, or rubusoside in the culture media.

In other embodiments, the recombinant microorganism is grown on media containing rubusoside in order to produce rebaudioside A. In such embodiments, the microorganism contains and expresses genes encoding a functional UGT91D2 and a functional UGT76G1, and is capable of producing rebaudioside A when it is fed rubusoside in the culture media.

In other embodiments the recombinant microorganism expresses one or more genes involved in steviol biosynthesis, e.g., a CDPS gene, a KS gene, a KO gene and/or a KAH gene. Thus, for example, a microorganism containing a CDPS gene, a KS gene, a KO gene and a KAH gene, in addition to a UGT74G1, a UGT85C2, a UGT91D2 gene and a UGT76G1 gene, is capable of producing rebaudioside A without the necessity for including steviol in the culture media.

In some embodiments, the recombinant microorganism further contains and expresses a recombinant GGPPS gene in order to provide increased levels of the diteipene precursor geranylgeranyl diphosphate, for increased flux through the rebaudioside A biosynthetic pathway. In some embodiments, the recombinant microorganism further contains and expresses recombinant genes involved in diterpene biosynthesis or production of terpenoid precursors, e.g., genes in the MEP or MEV pathway discussed below.

### B. 3 Dulcoside a and Rebaudioside C Biosynthesis Polypeptides

The biosynthesis of rebaudioside C and/or dulcoside A involves glucosylation and rhamnosylation of the aglycone steviol. Specifically, dulcoside A can be formed by glucosylation of the 13-OH of steviol which forms steviol-13-O-glucoside, rhamnosylation of the C-2′ of the 13-O-glucose of steviol-13-O-glucoside which forms the 1,2 rhamnobioside, and glucosylation of the C-19 carboxyl of the 1,2 rhamnobioside. Rebaudioside C can be formed by glucosylation of the C-3′ of the C-13-O-glucose of dulcoside A. The order in which each glycosylation reaction occurs can vary. See FIG. 2B.

It has been discovered that conversion of steviol to dulcoside A in a recombinant host can be accomplished by the expression of gene(s) encoding the following functional UGTs: 85C2, 91D2, and 74G1. Thus, a recombinant microorganism expressing these three UGTs and a rhamnose synthetase can make dulcoside A when fed steviol in the medium. Alternatively, a recombinant microorganism expressing two UGTs, 91D2 and 74G1, and rhamnose synthetase can make dulcoside A when fed the monoside, steviol-13-O-glucoside or steviol-19-O-glucoside, in the medium. Similarly, conversion of steviol to rebaudioside C in a recombinant microorganism can be accomplished by the expression of gene(s) encoding UGTs 85C2, 91D2, 74G1, and 76G1 and rhamnose synthetase when fed steviol, by the expression of genes encoding UGTs 91D2, 74G1 and 76G1, and rhamnose synthetase when fed steviol-13-O-glucoside, by the expression of genes encoding UGTs 85C2, 91D2 and 76G1, and rhamnose synthetase when fed steviol-19-O-glucoside, or by the expression of genes encoding UGTs 91D2 and 76G1 and rhamonse synthetase when fed rubusoside. Typically, one or more of these genes are recombinant genes that have been transformed into a microorganism that does not naturally possess them.

Suitable UGT91D2, UGT74G1, UGT76G1 and UGT85C2 polypeptides include the functional UGT polypeptides discussed herein. Rhamnose synthetase provides increased amounts of the UDP-rhamnose donor for rhamnosylation of the steviol compound acceptor. Suitable rhamnose synthetases include those made by Arabidopsis thaliana, such as the product of the A. thaliana RHM2 gene.

In some embodiments, a UGT79B3 polypeptide is substituted for a UGT91D2 polypeptide. Suitable UGT79B3 polypeptides include those made by Arabidopsis thaliana, which are capable of rhamnosylation of steviol 13-O-monoside in vitro. A. thaliana UGT79B3 can rhamnosylate glucosylated compounds to form 1,2-rhamnosides. The amino acid sequence of an Arabidopsis thaliana UGT79B3 is set forth in SEQ ID NO:150. The nucleotide sequence encoding the amino acid sequence of SEQ ID NO:150 is set forth in SEQ ID NO:151.

In some embodiments rebaudioside C can be produced using in vitro methods while supplying the appropriate UDP-sugar or a cell-free system for regeneration of UDP-sugars. See, for example, “An integrated cell-free metabolic platform for protein production and synthetic biology” by Jewett M C, Calhoun K A, Voloshin A, Wuu J J and Swartz J R in Molecular Systems Biology, 4, article 220 (2008). Reactions may be carried out together, or stepwise. For instance, rebaudioside C may be produced from rubusoside with the addition of stoichiometric amounts of UDP-rhamnose and UGT91d2e, followed by addition of UGT76G1 and an excess or stoichiometric supply of UDP-glucose. In some embodiments phosphatases are used to remove secondary products and improve the reaction yields.

In other embodiments, the recombinant host expresses one or more genes involved in steviol biosynthesis, e.g., a CDPS gene, a KS gene, a KO gene and/or a KAH gene. Thus, for example, a microorganism containing a CDPS gene, a KS gene, a KO gene and a KAH gene, in addition to a UGT85C2, a UGT74G1, a UGT91D2 gene and a UGT76G1 gene, is capable of producing rebaudioside C without the necessity for including steviol in the culture media. In addition, the recombinant host typically expresses an endogenous or a recombinant gene encoding a rhamnose synthetase. Such a gene is useful in order to provide increased amounts of the UDP-rhamnose donor for rhamnosylation of the steviol compound acceptor. Suitable rhamnose synthetases include those made by Arabidopsis thaliana, such as the product of the A. thaliana RHM2 gene.

One with skill in the art will recognize that by modulating relative expression levels of different UGT genes as well as modulating the availability of UDP-rhamnose, a recombinant host can be tailored to specifically produce steviol and steviol glycoside products in a desired proportion. Transcriptional regulation of steviol biosynthesis genes, and steviol glycoside biosynthesis genes can be achieved by a combination of transcriptional activation and repression using techniques known to those in the art. For in vitro reactions, one with skill in the art will recognize that addition of different levels of UGT enzymes in combination or under conditions which impact the relative activities of the different UGTS in combination will direct synthesis towards a desired proportion of each steviol glycoside.

In some embodiments, the recombinant host further contains and expresses a recombinant GGPPS gene in order to provide increased levels of the diterpene precursor geranylgeranyl diphosphate, for increased flux through the rebaudioside A biosynthetic pathway. In some embodiments, the recombinant host further contains a genetic construct to silence or reduce the expression of non-steviol pathways consuming geranylgeranyl diphosphate, ent-Kaurenoic acid or farnesyl pyrophosphate, thereby providing increased flux through the steviol and steviol glycosides biosynthetic pathways. For example, flux to sterol production pathways such as ergosterol may be reduced by downregulation of the ERGS gene. In cells that produce gibberellins, gibberellin synthesis may be downregulated to increase flux of ent-kaurenoic acid to steviol. In carotenoid-producing organisms, flux to steviol may be increased by downregulation of one or more carotenoid biosynthetic genes.

In some embodiments, the recombinant host further contains and expresses recombinant genes involved in diterpene biosynthesis or production of terpenoid precursors, e.g., genes in the MEP or MEV pathway discussed below.

In some embodiments, a recombinant host such as a microorganism produces steviol glycoside compositions that have greater than at least 15% rebaudioside C of the total steviol glycosides, e.g., at least 20% rebaudioside C, 30-40% rebaudioside C, 40-50% rebaudioside C, 50-60% rebaudioside C, 60-70% rebaudioside C, 70-80% rebaudioside C, 80-90% rebaudioside C. In some embodiments, a recombinant host such as a microorganism produces steviol glycoside compositions that have at least 90% rebaudioside C, e.g., 90-99% rebaudioside C. Other steviol glycosides present may include those depicted in FIGS. 2A and B such as steviol monosides, steviol glucobiosides, steviol rhamnobiosides, rebaudioside A, and Dulcoside A. In some embodiments, the rebaudioside C-enriched composition produced by the host can be further purified and the rebaudioside C or Dulcoside A so purified may then be mixed with other steviol glycosides, flavors, or sweeteners to obtain a desired flavor system or sweetening composition. For instance, a rebaudioside C-enriched composition produced by a recombinant microorganism can be combined with a rebaudioside A, F, or D-enriched composition produced by a different recombinant microorganism, with rebaudioside A, F, or D purified from a Stevia extract, or with rebaudioside A, F, or D produced in vitro.

### B. 4 Rebaudioside E and Rebaudioside D Biosynthesis Polypeptides

The biosynthesis of rebaudioside E and/or rebaudioside D involves glucosylation of the aglycone steviol. Specifically, rebaudioside E can be formed by glucosylation of the 13-OH of steviol which forms steviol-13-O-glucoside, glucosylation of the C-2′ of the 13-O-glucose of steviol-13-O-glucoside which forms the steviol-1,2-bioside, glucosylation of the C-19 carboxyl of the 1,2-bioside to form 1,2-stevioside, and glucosylation of the C-2′ of the 19-O-glucose of the 1,2-stevioside to form rebaudioside E. Rebaudioside D can be formed by glucosylation of the C-3′ of the C-13-O-glucose of rebaudioside E. The order in which each glycosylation reaction occurs can vary. For example, the glucosylation of the C-2′ of the 19-O-glucose may be the last step in the pathway, wherein Rebaudioside A is an intermediate in the pathway. See FIG. 2C.

It has been discovered that conversion of steviol to rebaudioside D in a recombinant host can be accomplished by the expression of gene(s) encoding the following functional UGTs: 85C2, 91D2, 74G1 and 76G1. Thus, a recombinant microorganism expressing these four UGTs can make rebaudioside D when fed steviol in the medium. Alternatively, a recombinant microorganism expressing two functional UGTs, 91D2 and 76G1, can make rebaudioside D when fed rubusoside or 1,2-stevioside in the medium. As another alternative, a recombinant microorganism expressing three functional UGTs, 74G1, 91D2 and 76G1, can make rebaudioside D when fed the monoside, steviol-13-O-glucoside, in the medium. Similarly, conversion of steviol-19-O-glucoside to rebaudioside D in a recombinant microorganism can be accomplished by the expression of genes encoding UGTs 85C2, 91D2 and 76G1 when fed steviol-19-O-glucoside. Typically, one or more of these genes are recombinant genes that have been transformed into a host that does not naturally possess them.

Suitable UGT91D2, UGT74G1, UGT76G1 and UGT85C2 polypeptides include the functional UGT polypeptides discussed herein. In some embodiments, a UGT79B3 polypeptide is substituted for a UGT91, as discussed above.

In some embodiments, rebaudioside D or rebaudioside E can be produced using in vitro methods while supplying the appropriate UDP-sugar or a cell-free system for regeneration of UDP-sugars. See, for example, Jewett M C, et al. Molecular Systems Biology, Vol. 4, article 220 (2008). Conversions requiring multiple reactions may be carried out together, or stepwise. Rebaudioside D may be produced from Rebaudioside A that is commercially available enriched extract or produced via biosynthesis, with the addition of stoichiometric or excess amounts of UDP-glucose and UGT91D2e. In some embodiments phosphatases are used to remove secondary products and improve the reaction yields.

One with skill in the art will recognize that by modulating relative expression levels of different UGT genes, a recombinant host can be tailored to specifically produce steviol and steviol glycoside products in a desired proportion. Transcriptional regulation of steviol biosynthesis genes and steviol glycoside biosynthesis genes can be achieved by a combination of transcriptional activation and repression using techniques known to those in the art. For in vitro reactions, one with skill in the art will recognize that addition of different levels of UGT enzymes in combination or under conditions which impact the relative activities of the different UGTS in combination will direct synthesis towards a desired proportion of each steviol glycoside. One with skill in the art will recognize that a higher proportion of rebaudioside D or E or more efficient conversion to rebaudioside D or E can be obtained with a diglycosylation enzyme that has a higher activity for the 19-O-glucoside reaction as compared to the 13-O-glucoside reaction (substrates rebaudioside A and stevioside).

In other embodiments, the recombinant host expresses one or more genes involved in steviol biosynthesis, e.g., a CDPS gene, a KS gene, a KO gene and/or a KAH gene. Thus, for example, a microorganism containing a CDPS gene, a KS gene, a KO gene and a KAH gene, in addition to a UGT85C2, a UGT74G1, a UGT91D2 gene and a UGT76G1 gene, is capable of producing rebaudiosides E and D without the necessity for including steviol in the culture media.

In some embodiments, the recombinant host further contains and expresses a recombinant GGPPS gene in order to provide increased levels of the diterpene precursor geranylgeranyl diphosphate, for increased flux through the steviol biosynthetic pathway. In some embodiments, the recombinant host further contains a genetic construct to silence the expression of non-steviol pathways consuming geranylgeranyl diphosphate, ent-Kaurenoic acid or farnesyl pyrophosphate, thereby providing increased flux through the steviol and steviol glycosides biosynthetic pathways. For example, flux to sterol production pathways such as ergosterol may be reduced by downregulation of the ERGS gene. In cells that produce gibberellins, gibberellin synthesis may be downregulated to increase flux of ent-kaurenoic acid to steviol. In carotenoid-producing organisms, flux to steviol may be increased by downregulation of one or more carotenoid biosynthetic genes. In some embodiments, the recombinant host further contains and expresses recombinant genes involved in diterpene biosynthesis or production of terpenoid precursors, e.g., genes in the MEP or MEV pathways discussed below.

In some embodiments, a recombinant host such as a microorganism produces rebaudioside D-enriched steviol glycoside compositions that have greater than at least 3% rebaudioside D by weight total steviol glycosides, e.g., at least 4% rebaudioside D at least 5% rebaudioside D, 10-20% rebaudioside D, 20-30% rebaudioside D, 30-40% rebaudioside D, 40-50% rebaudioside D, 50-60% rebaudioside D, 60-70% rebaudioside D, 70-80% rebaudioside D. In some embodiments, a recombinant host such as a microorganism produces steviol glycoside compositions that have at least 90% rebaudioside D, e.g., 90-99% rebaudioside D. Other steviol glycosides present may include those depicted in FIG. 2 C such as steviol monosides, steviol glucobiosides, rebaudioside A, rebaudioside E, and stevioside. In some embodiments, the rebaudioside D-enriched composition produced by the host (e.g., microorganism) can be further purified and the rebaudioside D or rebaudioside E so purified can then be mixed with other steviol glycosides, flavors, or sweeteners to obtain a desired flavor system or sweetening composition. For instance, a rebaudioside D-enriched composition produced by a recombinant host can be combined with a rebaudioside A, C, or F-enriched composition produced by a different recombinant host, with rebaudioside A, F, or C purified from a Stevia extract, or with rebaudioside A, F, or C produced in vitro.

### B. 5 Rebaudioside F Biosynthesis Polypeptides

The biosynthesis of rebaudioside F involves glucosylation and xylosylation of the aglycone steviol. Specifically, rebaudioside F can be formed by glucosylation of the 13-OH of steviol which forms steviol-13-O-glucoside, xylosylation of the C-2′ of the 13-O-glucose of steviol-13-O-glucoside which forms steviol-1,2-xylobioside, glucosylation of the C-19 carboxyl of the 1,2-xylobioside to form 1,2-stevioxyloside, and glucosylation of the C-3′ of the C-13-O-glucose of 1,2-stevioxyloside to form rebaudioside F. The order in which each glycosylation reaction occurs can vary. See FIG. 2D.

It has been discovered that conversion of steviol to rebaudioside F in a recombinant host can be accomplished by the expression of genes encoding the following functional UGTs: 85C2, 91D2, 74G1 and 76G1, along with endogenous or recombinantly expressed UDP-glucose dehydrogenase and UDP-glucuronic acid decarboxylase. Thus, a recombinant microorganism expressing these four UGTs along with endogenous or recombinant UDP-glucose dehydrogenase and UDP-glucuronic acid decarboxylase can make rebaudioside F when fed steviol in the medium. Alternatively, a recombinant microorganism expressing two functional UGTs, 91D2 and 76G1, can make rebaudioside F when fed rubusoside in the medium. As another alternative, a recombinant microorganism expressing a functional UGT 76G1 can make rebaudioside F when fed 1,2 steviorhamnoside. As another alternative, a recombinant microorganism expressing three functional UGTs, 74G1, 91D2 and 76G1, can make rebaudioside F when fed the monoside, steviol-13-O-glucoside, in the medium. Similarly, conversion of steviol-19-O-glucoside to rebaudioside F in a recombinant microorganism can be accomplished by the expression of genes encoding UGTs 85C2, 91D2 and 76G1 when fed steviol-19-O-glucoside. Typically, one or more of these genes are recombinant genes that have been transformed into a host that does not naturally possess them.

Suitable UGT91D2, UGT74G1, UGT76G1 and UGT85C2 polypeptides include the functional UGT polypeptides discussed herein. In some embodiments, a UGT79B3 polypeptide is substituted for a UGT91, as discussed above. UDP-glucose dehydrogenase and UDP-glucuronic acid decarboxylase provide increased amounts of the UDP-xylose donor for xylosylation of the steviol compound acceptor. Suitable UDP-glucose dehydrogenases and UDP-glucuronic acid decarboxylases include those made by Arabidopsis thaliana or Cryptococcus neoformans. For example, suitable UDP-glucose dehydrogenase and UDP-glucuronic acid decarboxylases polypeptides can be encoded by the A. thaliana UGD1 gene and UXS3 gene, respectively. See, Oka and Jigami, FEBS J. 273:2645-2657 (2006).

In some embodiments rebaudioside F can be produced using in vitro methods while supplying the appropriate UDP-sugar or a cell-free system for regeneration of UDP-sugars. See, for example, Jewett M C, et al. Molecular Systems Biology, Vol. 4, article 220 (2008). Reactions may be carried out together, or stepwise. For instance, rebaudioside F may be produced from rubusoside with the addition of stoichiometric amounts of UDP-xylose and UGT91D2e, followed by addition of UGT76G1 and an excess or stoichiometric supply of UDP-glucose. In some embodiments phosphatases are used to remove secondary products and improve the reaction yields.

In other embodiments, the recombinant host expresses one or more genes involved in steviol biosynthesis, e.g., a CDPS gene, a KS gene, a KO gene and/or a KAH gene. Thus, for example, a microorganism containing a CDPS gene, a KS gene, a KO gene and a KAH gene, in addition to a UGT85C2, a UGT74G1, a UGT91D2 gene and a UGT76G1 gene, is capable of producing rebaudioside F without the necessity for including steviol in the culture media. In addition, the recombinant host typically expresses an endogenous or a recombinant gene encoding a UDP-glucose dehydrogenase and a UDP-glucuronic acid decarboxylase. Such genes are useful in order to provide increased amounts of the UDP-xylose donor for xylosylation of the steviol compound acceptor. Suitable UDP-glucose dehydrogenases and UDP-glucuronic acid decarboxylases include those made by Arabidopsis thaliana or Cryptococcus neoformans. For example, suitable UDP-glucose dehydrogenase and UDP-glucuronic acid decarboxylases polypeptides can be encoded by the A. thaliana UGD1 gene and UXS3 gene, respectively. See, Oka and Jigami, FEBS J. 273:2645-2657 (2006).

One with skill in the art will recognize that by modulating relative expression levels of different UGT genes as well as modulating the availability of UDP-xylose, a recombinant microorganism can be tailored to specifically produce steviol and steviol glycoside products in a desired proportion. Transcriptional regulation of steviol biosynthesis genes can be achieved by a combination of transcriptional activation and repression using techniques known to those in the art. For in vitro reactions, one with skill in the art will recognize that addition of different levels of UGT enzymes in combination or under conditions which impact the relative activities of the different UGTS in combination will direct synthesis towards a desired proportion of each steviol glycosides.

In some embodiments, the recombinant host further contains and expresses a recombinant GGPPS gene in order to provide increased levels of the diterpene precursor geranylgeranyl diphosphate, for increased flux through the steviol biosynthetic pathway. In some embodiments, the recombinant host further contains a genetic construct to silence the expression of non-steviol pathways consuming geranylgeranyl diphosphate, ent-Kaurenoic acid or farnesyl pyrophosphate, thereby providing increased flux through the steviol and steviol glycosides biosynthetic pathways. For example, flux to sterol production pathways such as ergosterol may be reduced by downregulation of the ERGS gene. In cells that produce gibberellins, gibberellin synthesis may be downregulated to increase flux of ent-kaurenoic acid to steviol. In carotenoid-producing organisms, flux to steviol may be increased by downregulation of one or more carotenoid biosynthetic genes. In some embodiments, the recombinant host further contains and expresses recombinant genes involved in diterpene biosynthesis, e.g., genes in the MEP pathway discussed below.

In some embodiments, a recombinant host such as a microorganism produces rebaudioside F-enriched steviol glycoside compositions that have greater than at least 4% rebaudioside F by weight total steviol glycosides, e.g., at least 5% rebaudioside F, at least 6% of rebaudioside F, 10-20% rebaudioside F, 20-30% rebaudioside F, 30-40% rebaudioside F, 40-50% rebaudioside F, 50-60% rebaudioside F, 60-70% rebaudioside F, 70-80% rebaudioside F. In some embodiments, a recombinant host such as a microorganism produces steviol glycoside compositions that have at least 90% rebaudioside F, e.g., 90-99% rebaudioside F. Other steviol glycosides present may include those depicted in FIGS. 2 A and D such as steviol monosides, steviol glucobiosides, steviol xylobiosides, rebaudioside A, stevioxyloside, rubusoside and stevioside. In some embodiments, the rebaudioside F-enriched composition produced by the host can be mixed with other steviol glycosides, flavors, or sweeteners to obtain a desired flavor system or sweetening composition. For instance, a rebaudioside F-enriched composition produced by a recombinant microorganism can be combined with a rebaudioside A, C, or D-enriched composition produced by a different recombinant microorganism, with rebaudioside A, C, or D purified from a Stevia extract, or with rebaudioside A, C, or D produced in vitro.

### C. Other Polypeptides

Genes for additional polypeptides whose expression facilitates more efficient or larger scale production of steviol or a steviol glycoside can also be introduced into a recombinant host. For example, a recombinant microorganism, plant, or plant cell can also contain one or more genes encoding a geranylgeranyl diphosphate synthase (GGPPS, also referred to as GGDPS). As another example, the recombinant host can contain one or more genes encoding a rhamnose synthetase, or one or more genes encoding a UDP-glucose dehydrogenase and/or a UDP-glucuronic acid decarboxylase. As another example, a recombinant host can also contain one or more genes encoding a cytochrome P450 reductase (CPR). Expression of a recombinant CPR facilitates the cycling of NADP+ to regenerate NADPH, which is utilized as a cofactor for terpenoid biosynthesis. Other methods can be used to regenerate NADHP levels as well. In circumstances where NADPH becomes limiting; strains can be further modified to include exogenous transhydrogenase genes. See, e.g., Sauer et al., J. Biol. Chem. 279: 6613-6619 (2004). Other methods are known to those with skill in the art to reduce or otherwise modify the ratio of NADH/NADPH such that the desired cofactor level is increased.

As another example, the recombinant host can contain one or more genes encoding one or more enzymes in the MEP pathway or the mevalonate pathway. Such genes are useful because they can increase the flux of carbon into the diterpene biosynthesis pathway, producing geranylgeranyl diphosphate from isopentenyl diphosphate and dimethylallyl diphosphate generated by the pathway. The geranylgeranyl diphosphate so produced can be directed towards steviol and steviol glycoside biosynthesis due to expression of steviol biosynthesis polypeptides and steviol glycoside biosynthesis polypeptides.

### C. 1 MEP Biosynthesis Polypeptides

In some embodiments, a recombinant host contains one or more genes encoding enzymes involved in the methylerythritol 4-phosphate (MEP) pathway for isoprenoid biosynthesis. Enzymes in the MEP pathway include deoxyxylulose 5-phosphate synthase (DXS), D-1-deoxyxylulose 5-phosphate reductoisomerase (DXR), 4-diphosphocytidyl-2-C-methyl-D-erythritol synthase (CMS), 4-diphosphocytidyl-2-C-methyl-D-erythritol kinase (CMK), 4-diphosphocytidyl-2-C-methyl-D-erythritol 2,4-cyclodiphosphate synthase (MCS), 1-hydroxy-2-methyl-2(E)-butenyl 4-diphosphate synthase (HDS) and 1-hydroxy-2-methyl-2(E)-butenyl 4-diphosphate reductase (HDR). One or more DXS genes, DXR genes, CMS genes, CMK genes, MCS genes, HDS genes and/or HDR genes can be incorporated into a recombinant microorganism. See, Rodriguez-Concepción and Boronat, Plant Phys. 130: 1079-1089 (2002).

Suitable genes encoding DXS, DXR, CMS, CMK, MCS, HDS and/or HDR polypeptides include those made by E. coli, Arabidopsis thaliana and Synechococcus leopoliensis. Nucleotide sequences encoding DXR polypeptides are described, for example, in U.S. Pat. No. 7,335,815.

### C. 2 Mevalonate Biosynthesis Polypeptides

In some embodiments, a recombinant host contains one or more genes encoding enzymes involved in the mevalonate pathway for isoprenoid biosynthesis. Genes suitable for transformation into a host encode enzymes in the mevalonate pathway such as a truncated 3-hydroxy-3-methyl-glutaiyl (HMG)-CoA reductase (tHMG), and/or a gene encoding a mevalonate kinase (MK), and/or a gene encoding a phosphomevalonate kinase (PMK), and/or a gene encoding a mevalonate pyrophosphate decarboxylase (MPPD). Thus, one or more HMG-CoA reductase genes, MK genes, PMK genes, and/or MPPD genes can be incorporated into a recombinant host such as a microorganism.

Suitable genes encoding mevalonate pathway polypeptides are known. For example, suitable polypeptides include those made by E. coli, Paracoccus denitrificans, Saccharomyces cerevisiae, Arabidopsis thaliana, Kitasatospora griseola, Homo sapiens, Drosophila melanogaster, Gallus gallus, Streptomyces sp. KO-3988, Nicotiana attenuata, Kitasatospora griseola, Hevea brasiliensis, Enterococcus faecium and Haematococcus pluvialis. See, e.g., U.S. Pat. Nos. 7,183,089, 5,460,949, and 5,306,862.

### D. Functional Homologs

Functional homologs of the polypeptides described above are also suitable for use in producing steviol or steviol glycosides in a recombinant host. A functional homolog is a polypeptide that has sequence similarity to a reference polypeptide, and that carries out one or more of the biochemical or physiological function(s) of the reference polypeptide. A functional homolog and the reference polypeptide may be natural occurring polypeptides, and the sequence similarity may be due to convergent or divergent evolutionary events. As such, functional homologs are sometimes designated in the literature as homologs, or orthologs, or paralogs. Variants of a naturally occurring functional homolog, such as polypeptides encoded by mutants of a wild type coding sequence, may themselves be functional homologs. Functional homologs can also be created via site-directed mutagenesis of the coding sequence for a polypeptide, or by combining domains from the coding sequences for different naturally-occurring polypeptides (“domain swapping”). Techniques for modifying genes encoding functional UGT polypeptides described herein are known and include, inter alia, directed evolution techniques, site-directed mutagenesis techniques and random mutagenesis techniques, and can be useful to increase specific activity of a polypeptide, alter substrate specificity, alter expression levels, alter subcellular location, or modify polypeptide:polypeptide interactions in a desired manner. Such modified polypeptides are considered functional homologs. The term “functional homolog” is sometimes applied to the nucleic acid that encodes a functionally homologous polypeptide.

Functional homologs can be identified by analysis of nucleotide and polypeptide sequence alignments. For example, performing a query on a database of nucleotide or polypeptide sequences can identify homologs of steviol or steviol glycoside biosynthesis polypeptides. Sequence analysis can involve BLAST, Reciprocal BLAST, or PSI-BLAST analysis of nonredundant databases using a GGPPS, a CDPS, a KS, a KO or a KAH amino acid sequence as the reference sequence. Amino acid sequence is, in some instances, deduced from the nucleotide sequence. Those polypeptides in the database that have greater than 40% sequence identity are candidates for further evaluation for suitability as a steviol or steviol glycoside biosynthesis polypeptide. Amino acid sequence similarity allows for conservative amino acid substitutions, such as substitution of one hydrophobic residue for another or substitution of one polar residue for another. If desired, manual inspection of such candidates can be carried out in order to narrow the number of candidates to be further evaluated. Manual inspection can be performed by selecting those candidates that appear to have domains present in steviol biosynthesis polypeptides, e.g., conserved functional domains.

Conserved regions can be identified by locating a region within the primary amino acid sequence of a steviol or a steviol glycoside biosynthesis polypeptide that is a repeated sequence, forms some secondary structure (e.g., helices and beta sheets), establishes positively or negatively charged domains, or represents a protein motif or domain. See, e.g., the Pfam web site describing consensus sequences for a variety of protein motifs and domains on the World Wide Web at sanger.ac.uk/Software/Pfam/and pfam.janelia.org/. The information included at the Pfam database is described in Sonnhammer et al., Nucl. Acids Res., 26:320-322 (1998); Sonnhammer et al., Proteins, 28:405-420 (1997); and Bateman et al., Nucl. Acids Res., 27:260-262 (1999). Conserved regions also can be determined by aligning sequences of the same or related polypeptides from closely related species. Closely related species preferably are from the same family. In some embodiments, alignment of sequences from two different species is adequate.

Typically, polypeptides that exhibit at least about 40% amino acid sequence identity are useful to identify conserved regions. Conserved regions of related polypeptides exhibit at least 45% amino acid sequence identity (e.g., at least 50%, at least 60%, at least 70%, at least 80%, or at least 90% amino acid sequence identity). In some embodiments, a conserved region exhibits at least 92%, 94%, 96%, 98%, or 99% amino acid sequence identity.

For example, polypeptides suitable for producing steviol glycosides in a recombinant host include functional homologs of UGT91D2e, UGT91D2m, UGT85C, and UGT76G. Such homologs have greater than 90% (e.g., at least 95% or 99%) sequence identity to the amino acid sequence of UGT91D2e (SEQ ID NO:5), UGT91D2m (SEQ ID NO:10), UGT85C (SEQ ID NO:3), or UGT76G (SEQ ID NO:7). Variants of UGT91D2, UGT85C, and UGT76G polypeptides typically have 10 or fewer amino acid substitutions within the primary amino acid sequence, e.g., 7 or fewer amino acid substitutions, 5 or conservative amino acid substitutions, or between 1 and 5 substitutions. However, in some embodiments, variants of UGT91D2, UGT85C, and UGT76G polypeptides can have 10 or more amino acid substitutions (e.g., 10, 15, 20, 25, 30, 35, 10-20, 10-35, 20-30, or 25-35 amino acid substitutions). The substitutions may be conservative, or in some embodiments, non-conservative. Non-limiting examples of non-conservative changes in UGT91D2e polypeptides include glycine to arginine and tryptophan to arginine. Non-limiting examples of non-conservative substitutions in UGT76G polypeptides include valine to glutamic acid, glycine to glutamic acid, glutamine to alanine, and serine to proline. Non-limiting examples of changes to UGT85C polypeptides include histidine to aspartic acid, proline to serine, lysine to threonine, and threonine to arginine.

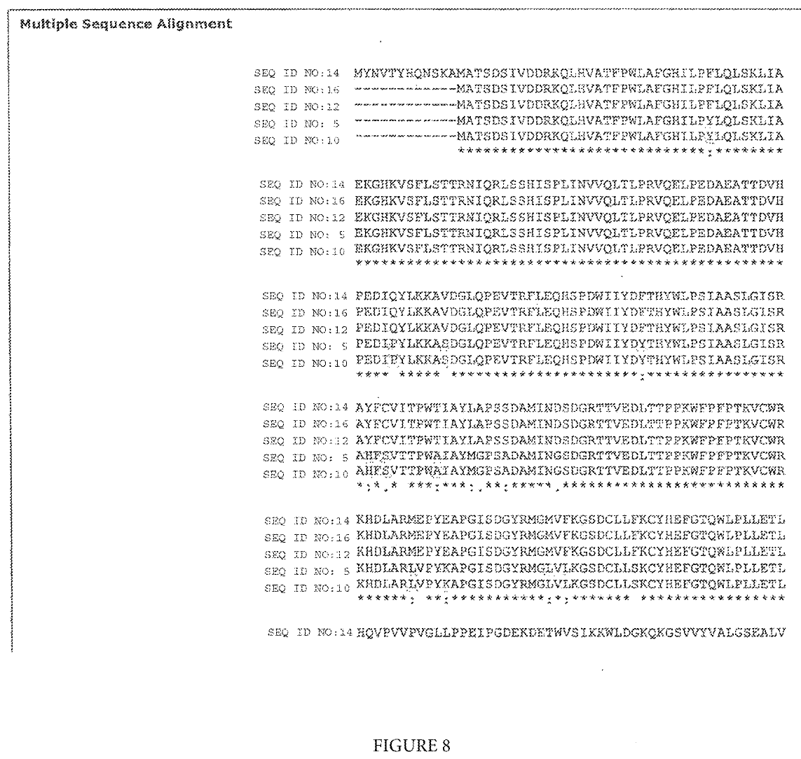

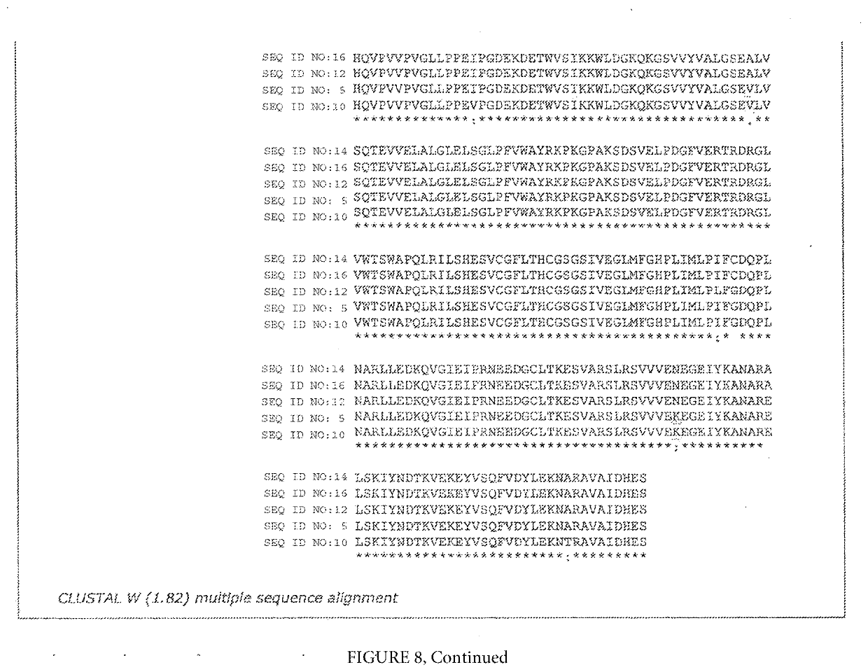

In some embodiments, a useful UGT91D2 homolog can have amino acid substitutions (e.g., conservative amino acid substitutions) in regions of the polypeptide that are outside of predicted loops, e.g., residues 20-26, 39-43, 88-95, 121-124, 142-158, 185-198, and 203-214 are predicted loops in the N-terminal domain and residues 381-386 are predicted loops in the C-terminal domain of SEQ ID NO:5. For example, a useful UGT91D2 homolog can include at least one amino acid substitution at residues 1-19, 27-38, 44-87, 96-120, 125-141, 159-184, 199-202, 215-380, or 387-473 of SEQ ID NO:5. In some embodiments, a UGT91D2 homolog can have an amino acid substitution at one or more residues selected from the group consisting of residues 30, 93, 99, 122, 140, 142, 148, 153, 156, 195, 196, 199, 206, 207, 211, 221, 286, 343, 427, and 438 of SEQ ID NO:5. For example, a UGT91D2 functional homolog can have an amino acid substitution at one or more of residues 206, 207, and 343, such as an arginine at residue 206, a cysteine at residue 207, and an arginine at residue 343 of SEQ ID NO:5. See, SEQ ID NO:95. Other functional homologs of UGT91D2 can have one or more of the following: a tyrosine or phenylalanine at residue 30, a proline or glutamine at residue 93, a serine or valine at residue 99, a tyrosine or a phenylalanine at residue 122, a histidine or tyrosine at residue 140, a serine or cysteine at residue 142, an alanine or threonine at residue 148, a methionine at residue 152, an alanine at residue 153, an alanine or serine at residue 156, a glycine at residue 162, a leucine or methionine at residue 195, a glutamic acid at residue 196, a lysine or glutamic acid at residue 199, a leucine or methionine at residue 211, a leucine at residue 213, a serine or phenylalanine at residue 221, a valine or isoleucine at residue 253, a valine or alanine at residue 286, a lysine or asparagine at residue 427, an alanine at residue 438, and either an alanine or threonine at residue 462 of SEQ ID NO:5. See, Examples 11 and 16, and Tables 12 and 14. A useful variant UGT91D2 polypeptide also can be constructed based on the alignment set forth in FIG. 8.

In some embodiments, a useful UGT85C homolog can have one or more amino acid substitutions at residues 9, 10, 13, 15, 21, 27, 60, 65, 71, 87, 91, 220, 243, 270, 289, 298, 334, 336, 350, 368, 389, 394, 397, 418, 420, 440, 441, 444, and 471 of SEQ ID NO:3. Non-limiting examples of useful UGT85C homologs include polypeptides having substitutions (with respect to SEQ ID NO:3) at residue 65; at residue 65 in combination with residue 15, 270, 418, 440, or 441; residues 13, 15, 60, 270, 289, and 418; substitutions at residues 13, 60, and 270; substitutions at residues 60 and 87; substitutions at residues 65, 71, 220, 243, and 270; substitutions at residues 65, 71, 220, 243, 270, and 441; substitutions at residues 65, 71, 220, 389, and 394; substitutions at residues 65, 71, 270, and 289; substitutions at residues 220, 243, 270, and 334; or substitutions at residues 270 and 289. See, Example 17 and Table 15.

In some embodiments, a useful UGT76G homolog can have one or more amino acid substitutions at residues 29, 74, 87, 91, 116, 123, 125, 126, 130, 145, 192, 193, 194, 196, 198, 199, 200, 203, 204, 205, 206, 207, 208, 266, 273, 274, 284, 285, 291, 330, 331, and 346 of SEQ ID NO:7. Non-limiting examples of useful UGT76G homologs include polypeptides having substitutions (with respect to SEQ ID NO:7) at residues 74, 87, 91, 116, 123, 125, 126, 130, 145, 192, 193, 194, 196, 198, 199, 200, 203, 204, 205, 206, 207, 208, and 291; residues 74, 87, 91, 116, 123, 125, 126, 130, 145, 192, 193, 194, 196, 198, 199, 200, 203, 204, 205, 206, 207, 208, 266, 273, 274, 284, 285, and 291; or residues 74, 87, 91, 116, 123, 125, 126, 130, 145, 192, 193, 194, 196, 198, 199, 200, 203, 204, 205, 206, 207, 208, 266, 273, 274, 284, 285, 291, 330, 331, and 346. See, Example 18 and Table 16.

Methods to modify the substrate specificity of, for example UGT91D2e, are known to those skilled in the art, and include without limitation site-directed/rational mutagenesis approaches, random directed evolution approaches and combinations in which random mutagenesis/saturation techniques are performed near the active site of the enzyme. For example see Sarah A. Osmani, et al. Phytochemistry 70 (2009) 325-347.

A candidate sequence typically has a length that is from 80 percent to 200 percent of the length of the reference sequence, e.g., 82, 85, 87, 89, 90, 93, 95, 97, 99, 100, 105, 110, 115, 120, 130, 140, 150, 160, 170, 180, 190, or 200 percent of the length of the reference sequence. A percent identity for any candidate nucleic acid or polypeptide relative to a reference nucleic acid or polypeptide can be determined as follows. A reference sequence (e.g., a nucleic acid sequence or an amino acid sequence) is aligned to one or more candidate sequences using the computer program ClustalW (version 1.83, default parameters), which allows alignments of nucleic acid or polypeptide sequences to be carried out across their entire length (global alignment). Chenna et al., Nucleic Acids Res., 31(13):3497-500 (2003).

ClustalW calculates the best match between a reference and one or more candidate sequences, and aligns them so that identities, similarities and differences can be determined. Gaps of one or more residues can be inserted into a reference sequence, a candidate sequence, or both, to maximize sequence alignments. For fast pairwise alignment of nucleic acid sequences, the following default parameters are used: word size: 2; window size: 4; scoring method: percentage; number of top diagonals: 4; and gap penalty: 5. For multiple alignment of nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method: percentage; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; residue-specific gap penalties: on. The ClustalW output is a sequence alignment that reflects the relationship between sequences. ClustalW can be run, for example, at the Baylor College of Medicine Search Launcher site on the World Wide Web (searchlauncher.bcm.tmc.edu/multi-align/multi-align.html) and at the European Bioinformatics Institute site on the World Wide Web (ebi.ac.uk/clustalw).

To determine percent identity of a candidate nucleic acid or amino acid sequence to a reference sequence, the sequences are aligned using ClustalW, the number of identical matches in the alignment is divided by the length of the reference sequence, and the result is multiplied by 100. It is noted that the percent identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

It will be appreciated that a functional UGT91D2 polypeptide can include additional amino acids that are not involved in glucosylation or other enzymatic activities carried out by UGT91D2, and thus such a polypeptide can be longer than would otherwise be the case. For example, a UGT91D2 polypeptide can include a purification tag, a chloroplast transit peptide, a mitochondrial transit peptide, an amyloplast peptide, signal peptide, or a secretion tag added to the amino or carboxy terminus. In some embodiments, a UGT91D2 polypeptide includes an amino acid sequence that functions as a reporter, e.g., a green fluorescent protein or yellow fluorescent protein.

### II. STEVIOL AND STEVIOL GLYCOSIDE BIOSYNTHESIS NUCLEIC ACIDS

A recombinant gene encoding a polypeptide described herein comprises the coding sequence for that polypeptide, operably linked in sense orientation to one or more regulatory regions suitable for expressing the polypeptide. Because many microorganisms are capable of expressing multiple gene products from a polycistronic mRNA, multiple polypeptides can be expressed under the control of a single regulatory region for those microorganisms, if desired. A coding sequence and a regulatory region are considered to be operably linked when the regulatory region and coding sequence are positioned so that the regulatory region is effective for regulating transcription or translation of the sequence. Typically, the translation initiation site of the translational reading frame of the coding sequence is positioned between one and about fifty nucleotides downstream of the regulatory region for a monocistronic gene.

In many cases, the coding sequence for a polypeptide described herein is identified in a species other than the recombinant host, i.e., is a heterologous nucleic acid. Thus, if the recombinant host is a microorganism, the coding sequence can be from other prokaryotic or eukaryotic microorganisms, from plants or from animals. In some case, however, the coding sequence is a sequence that is native to the host and is being reintroduced into that organism. A native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found.

“Regulatory region” refers to a nucleic acid having nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, introns, and combinations thereof. A regulatory region typically comprises at least a core (basal) promoter. A regulatory region also may include at least one control element, such as an enhancer sequence, an upstream element or an upstream activation region (UAR). A regulatory region is operably linked to a coding sequence by positioning the regulatory region and the coding sequence so that the regulatory region is effective for regulating transcription or translation of the sequence. For example, to operably link a coding sequence and a promoter sequence, the translation initiation site of the translational reading frame of the coding sequence is typically positioned between one and about fifty nucleotides downstream of the promoter. A regulatory region can, however, be positioned as much as about 5,000 nucleotides upstream of the translation initiation site, or about 2,000 nucleotides upstream of the transcription start site.

The choice of regulatory regions to be included depends upon several factors, including, but not limited to, efficiency, selectability, inducibility, desired expression level, and preferential expression during certain culture stages. It is a routine matter for one of skill in the art to modulate the expression of a coding sequence by appropriately selecting and positioning regulatory regions relative to the coding sequence. It will be understood that more than one regulatory region may be present, e.g., introns, enhancers, upstream activation regions, transcription terminators, and inducible elements.

One or more genes can be combined in a recombinant nucleic acid construct in “modules” useful for a discrete aspect of steviol and/or steviol glycoside production. Combining a plurality of genes in a module, particularly a polycistronic module, facilitates the use of the module in a variety of species. For example, a steviol biosynthesis gene cluster, or a UGT gene cluster, can be combined in a polycistronic module such that, after insertion of a suitable regulatory region, the module can be introduced into a wide variety of species. As another example, a UGT gene cluster can be combined such that each UGT coding sequence is operably linked to a separate regulatory region, to form a UGT module. Such a module can be used in those species for which monocistronic expression is necessary or desirable. In addition to genes useful for steviol or steviol glycoside production, a recombinant construct typically also contains an origin of replication, and one or more selectable markers for maintenance of the construct in appropriate species.

It will be appreciated that because of the degeneracy of the genetic code, a number of nucleic acids can encode a particular polypeptide; i.e., for many amino acids, there is more than one nucleotide triplet that serves as the codon for the amino acid. Thus, codons in the coding sequence for a given polypeptide can be modified such that optimal expression in a particular host is obtained, using appropriate codon bias tables for that host (e.g., microorganism). SEQ ID NOs:18-25, 34-36, 40-43, 48-49, 52-55, 60-64, and 70-72 set forth nucleotide sequences encoding certain enzymes for steviol and steviol glycoside biosynthesis, modified for increased expression in yeast. As isolated nucleic acids, these modified sequences can exist as purified molecules and can be incorporated into a vector or a virus for use in constructing modules for recombinant nucleic acid constructs.

In some cases, it is desirable to inhibit one or more functions of an endogenous polypeptide in order to divert metabolic intermediates towards steviol or steviol glycoside biosynthesis. For example, it may be desirable to downregulate synthesis of sterols in a yeast strain in order to further increase steviol or steviol glycoside production, e.g., by downregulating squalene epoxidase. As another example, it may be desirable to inhibit degradative functions of certain endogenous gene products, e.g., glycohydrolases that remove glucose moieties from secondary metabolites. As another example, expression of membrane transporters involved in transport of steviol glycosides can be inhibited, such that secretion of glycosylated steviosides is inhibited. Such regulation can be beneficial in that secretion of steviol glycosides can be inhibited for a desired period of time during culture of the microorganism, thereby increasing the yield of glycoside product(s) at harvest. In such cases, a nucleic acid that inhibits expression of the polypeptide or gene product may be included in a recombinant construct that is transformed into the strain. Alternatively, mutagenesis can be used to generate mutants in genes for which it is desired to inhibit function.

### III. HOSTS

### A. Microorganisms

A number of prokaryotes and eukaryotes are suitable for use in constructing the recombinant microorganisms described herein, e.g., gram-negative bacteria, yeast and fungi. A species and strain selected for use as a steviol or steviol glycoside production strain is first analyzed to determine which production genes are endogenous to the strain and which genes are not present. Genes for which an endogenous counterpart is not present in the strain are assembled in one or more recombinant constructs, which are then transformed into the strain in order to supply the missing function(s).

Exemplary prokaryotic and eukaryotic species are described in more detail below. However, it will be appreciated that other species may be suitable. For example, suitable species may be in a genus selected from the group consisting of Agaricus, Aspergillus, Bacillus, Candida, Corynebacterium, Escherichia, Fusarium/Gibberella, Kluyveromyces, Laetiporus, Lentinus, Phaffia, Phanerochaete, Pichia, Physcomitrella, Rhodoturula, Saccharomyces, Schizosaccharomyces, Sphaceloma, Xanthophyllomyces and Yarrowia. Exemplary species from such genera include Lentinus tigrinus, Laetiporus sulphureus, Phanerochaete chrysosporium, Pichia pastoris, Physcomitrella patens, Rhodoturula glutinis 32, Rhodoturula mucilaginosa, Phaffia rhodozyma UBV-AX, Xanthophyllomyces dendrorhous, Fusarium fujikuroi Gibberella fujikuroi, Candida utilis and Yarrowia lipolytica. In some embodiments, a microorganism can be an Ascomycete such as Gibberella fujikuroi, Kluyveromyces lactis, Schizosaccharomyces pombe, Aspergillus niger, or Saccharomyces cerevisiae. In some embodiments, a microorganism can be a prokaryote such as Escherichia coli, Rhodobacter sphaeroides, or Rhodobacter capsulatus. It will be appreciated that certain microorganisms can be used to screen and test genes of interest in a high throughput manner, while other microorganisms with desired productivity or growth characteristics can be used for large-scale production of steviol glycosides.

Saccharomyces cerevisiae

Saccharomyces cerevisiae is a widely used chassis organism in synthetic biology, and can be used as the recombinant microorganism platform. There are libraries of mutants, plasmids, detailed computer models of metabolism and other information available for S. cerevisiae, allowing for rational design of various modules to enhance product yield. Methods are known for making recombinant microorganisms.

A steviol biosynthesis gene cluster can be expressed in yeast using any of a number of known promoters. Strains that overproduce terpenes are known and can be used to increase the amount of geranylgeranyl diphosphate available for steviol and steviol glycoside production.

Aspergillus spp.

Aspergillus species such as A. oryzae, A. niger and A. sojae are widely used microorganisms in food production, and can also be used as the recombinant microorganism platform. Nucleotide sequences are available for genomes of A. nidulans, A. fumigatus, A. oryzae, A. clavatus, A. flavus, A. niger, and A. terreus, allowing rational design and modification of endogenous pathways to enhance flux and increase product yield. Metabolic models have been developed for Aspergillus, as well as transcriptomic studies and proteomics studies. A. niger is cultured for the industrial production of a number of food ingredients such as citric acid and gluconic acid, and thus species such as A. niger are generally suitable for the production of food ingredients such as steviol and steviol glycosides. Example 23 describes cloning methodology for production of steviol glycosides in Aspergillus nidulans.

Escherichia coli

Escherichia coli, another widely used platform organism in synthetic biology, can also be used as the recombinant microorganism platform. Similar to Saccharomyces, there are libraries of mutants, plasmids, detailed computer models of metabolism and other information available for E. coli, allowing for rational design of various modules to enhance product yield. Methods similar to those described above for Saccharomyces can be used to make recombinant E. coli microorganisms.

Agaricus, Gibberella, and Phanerochaete spp.

Agaricus, Gibberella, and Phanerochaete spp. can be useful because they are known to produce large amounts of gibberellin in culture. Thus, the terpene precursors for producing large amounts of steviol and steviol glycosides are already produced by endogenous genes. Thus, modules containing recombinant genes for steviol or steviol glycoside biosynthesis polypeptides can be introduced into species from such genera without the necessity of introducing mevalonate or MEP pathway genes.

Rhodobacter spp.

Rhodobacter can be use as the recombinant microorganism platform. Similar to E. coli, there are libraries of mutants available as well as suitable plasmid vectors, allowing for rational design of various modules to enhance product yield. Isoprenoid pathways have been engineered in membraneous bacterial species of Rhodobacter for increased production of carotenoid and CoQ10. See, U.S. Patent Publication Nos. 20050003474 and 20040078846. Methods similar to those described above for E. coli can be used to make recombinant Rhodobacter microorganisms.

Physcomitrella spp.

Physcomitrella mosses, when grown in suspension culture, have characteristics similar to yeast or other fungal cultures. This genera is becoming an important type of cell for production of plant secondary metabolites, which can be difficult to produce in other types of cells. Example 22 describes production of active UGT enzymes in the steviol glycoside pathway in P. patens.

### B. Plant Cells or Plants

In some embodiments, the nucleic acids and polypeptides described herein are introduced into plants or plant cells to increase overall steviol glycoside production or enrich for the production of specific steviol glycosides in proportion to others. Thus, a host can be a plant or a plant cell that includes at least one recombinant gene described herein. A plant or plant cell can be transformed by having a recombinant gene integrated into its genome, i.e., can be stably transformed. Stably transformed cells typically retain the introduced nucleic acid with each cell division. A plant or plant cell can also be transiently transformed such that the recombinant gene is not integrated into its genome. Transiently transformed cells typically lose all or some portion of the introduced nucleic acid with each cell division such that the introduced nucleic acid cannot be detected in daughter cells after a sufficient number of cell divisions. Both transiently transformed and stably transformed transgenic plants and plant cells can be useful in the methods described herein.

Transgenic plant cells used in methods described herein can constitute part or all of a whole plant. Such plants can be grown in a manner suitable for the species under consideration, either in a growth chamber, a greenhouse, or in a field. Transgenic plants can be bred as desired for a particular purpose, e.g., to introduce a recombinant nucleic acid into other lines, to transfer a recombinant nucleic acid to other species, or for further selection of other desirable traits. Alternatively, transgenic plants can be propagated vegetatively for those species amenable to such techniques. As used herein, a transgenic plant also refers to progeny of an initial transgenic plant provided the progeny inherits the transgene. Seeds produced by a transgenic plant can be grown and then selfed (or outcrossed and selfed) to obtain seeds homozygous for the nucleic acid construct.

Transgenic plants can be grown in suspension culture, or tissue or organ culture. For the purposes of this invention, solid and/or liquid tissue culture techniques can be used. When using solid medium, transgenic plant cells can be placed directly onto the medium or can be placed onto a filter that is then placed in contact with the medium. When using liquid medium, transgenic plant cells can be placed onto a flotation device, e.g., a porous membrane that contacts the liquid medium.

When transiently transformed plant cells are used, a reporter sequence encoding a reporter polypeptide having a reporter activity can be included in the transformation procedure and an assay for reporter activity or expression can be performed at a suitable time after transformation. A suitable time for conducting the assay typically is about 1-21 days after transformation, e.g., about 1-14 days, about 1-7 days, or about 1-3 days. The use of transient assays is particularly convenient for rapid analysis in different species, or to confirm expression of a heterologous polypeptide whose expression has not previously been confirmed in particular recipient cells.

Techniques for introducing nucleic acids into monocotyledonous and dicotyledonous plants are known in the art, and include, without limitation, Agrobacterium-mediated transformation, viral vector-mediated transformation, electroporation and particle gun transformation, U.S. Pat. Nos. 5,538,880; 5,204,253; 6,329,571; and 6,013,863. If a cell or cultured tissue is used as the recipient tissue for transformation, plants can be regenerated from transformed cultures if desired, by techniques known to those skilled in the art.

A population of transgenic plants can be screened and/or selected for those members of the population that have a trait or phenotype conferred by expression of the transgene. For example, a population of progeny of a single transformation event can be screened for those plants having a desired level of expression of a steviol or steviol glycoside biosynthesis polypeptide or nucleic acid. Physical and biochemical methods can be used to identify expression levels. These include Southern analysis or PCR amplification for detection of a polynucleotide; Northern blots, S1 RNase protection, primer-extension, or RT-PCR amplification for detecting RNA transcripts; enzymatic assays for detecting enzyme or ribozyme activity of polypeptides and polynucleotides; and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or nucleic acids. Methods for performing all of the referenced techniques are known. As an alternative, a population of plants comprising independent transformation events can be screened for those plants having a desired trait, such as production of a steviol glycoside or modulated biosynthesis of a steviol glycoside. Selection and/or screening can be carried out over one or more generations, and/or in more than one geographic location. In some cases, transgenic plants can be grown and selected under conditions which induce a desired phenotype or are otherwise necessary to produce a desired phenotype in a transgenic plant. In addition, selection and/or screening can be applied during a particular developmental stage in which the phenotype is expected to be exhibited by the plant. Selection and/or screening can be carried out to choose those transgenic plants having a statistically significant difference in a steviol glycoside level relative to a control plant that lacks the transgene.

The nucleic acids, recombinant genes, and constructs described herein can be used to transform a number of monocotyledonous and dicotyledonous plants and plant cell systems. Non-limiting examples of suitable monocots include, for example, cereal crops such as rice, rye, sorghum, millet, wheat, maize, and barley. The plant may be a non-cereal monocot such as asparagus, banana, or onion. The plant also may be a dicot such as stevia (Stevia rebaudiana), soybean, cotton, sunflower, pea, geranium, spinach, or tobacco. In some cases, the plant may contain the precursor pathways for phenyl phosphate production such as the mevalonate pathway, typically found in the cytoplasm and mitochondria. The non-mevalonate pathway is more often found in plant plastids [Dubey, et al., 2003 J. Biosci. 28 637-646]. One with skill in the art may target expression of steviol glycoside biosynthesis polypeptides to the appropriate organelle through the use of leader sequences, such that steviol glycoside biosynthesis occurs in the desired location of the plant cell. One with skill in the art will use appropriate promoters to direct synthesis, e.g., to the leaf of a plant, if so desired. Expression may also occur in tissue cultures such as callus culture or hairy root culture, if so desired.

In one embodiment, one or more nucleic acid or polypeptides described herein are introduced into Stevia (e.g., Stevia rebaudiana) such that overall steviol glycoside biosynthesis is increased or that the overall steviol glycoside composition is selectively enriched for one or more specific steviol glycosides. For example, one or more recombinant genes can be introduced into Stevia such that one or more of the following are expressed: a UGT91D enzyme such as UGT91D2e (e.g., SEQ ID NO:5 or a functional homolog thereof), UGT91D2m (e.g., SEQ ID NO:10); a UGT85C enzyme such as a variant set forth in Table 15, or a UGT76G1 enzyme such as a variant set forth in Example 18. Nucleic acid constructs typically include a suitable promoter (e.g., 35S, e35S, or ssRUBISCO promoters) operably linked to a nucleic acid encoding the UGT polypeptide. Nucleic acids can be introduced into Stevia by Agrobacterium-mediated transformation; electroporation-mediated gene transfer to protoplasts; or by particle bombardment. See, e.g., Singh, et al., Compendium of Transgenic Crop Plants: Transgenic Sugar, Tuber and Fiber, Edited by Chittaranjan Kole and Timothy C. Hall, Blackwell Publishing Ltd. (2008), pp. 97-115. For particle bombardment of stevia leaf derived callus, the parameters can be as follows: 6 cm distance, 1100 psi He pressure, gold particles, and one bombardment.

Stevia plants can be regenerated by somatic embryogenesis as described by Singh et al., 2008, supra. In particular, leaf segments (approximately 1-2 cm long) can be removed from 5 to 6-week-old in vitro raised plants and incubated (adaxial side down) on MS medium supplemented with B5 vitamins, 30 g sucrose and 3 g Gelrite. 2,4-dichlorophenoxyacetic acid (2,4-D) can be used in combination with 6-benzyl adenine (BA), kinetin (KN), or zeatin. Proembryogenic masses appear after 8 weeks of subculture. Within 2-3 weeks of subcultures, somatic embryos will appear on the surface of cultures. Embryos can be matured in medium containing BA in combination with 2,4-D, a-naphthaleneacetic acid (NAA), or indolbutyric acid (IBA). Mature somatic embryos that germinate and form plantlets can be excised from calli. After plantlets reach 3-4 weeks, the plantlets can be transferred to pots with vermiculite and grown for 6-8 weeks in growth chambers for acclimatization and transferred to greenhouses.

In one embodiment, steviol glycosides are produced in rice. Rice and maize are readily transformable using techniques such as Agrobacterium-mediated transformation. Binary vector systems are commonly utilized for Agrobacterium exogenous gene introduction to monocots. See, for example, U.S. Pat. Nos. 6,215,051 and 6,329,571. In a binary vector system, one vector contains the T-DNA region, which includes a gene of interest (e.g., a UGT described herein) and the other vector is a disarmed Ti plasmid containing the vir region. Co-integrated vectors and mobilizable vectors also can be used. The types and pretreatment of tissues to be transformed, the strain of Agrobacterium used, the duration of the inoculation, the prevention of overgrowth and necrosis by the Agrobacterium, can be readily adjusted by one of skill in the art Immature embryo cells of rice can be prepared for transformation with Agrobacterium using binary vectors. The culture medium used is supplemented with phenolic compounds. Alternatively, the transformation can be done in planta using vacuum infiltration. See, for example, WO 2000037663, WO 2000063400, and WO 2001012828.

### IV. METHODS OF PRODUCING STEVIOL AND STEVIOL GLYCOSIDES

Recombinant hosts described herein can be used in methods to produce steviol or steviol glycosides. For example, if the recombinant host is a microorganism, the method can include growing the recombinant microorganism in a culture medium under conditions in which steviol and/or steviol glycoside biosynthesis genes are expressed. The recombinant microorganism may be grown in a fed batch or continuous process. Typically, the recombinant microorganism is grown in a fermentor at a defined temperature(s) for a desired period of time. Depending on the particular microorganism used in the method, other recombinant genes such as isopentenyl biosynthesis genes and terpene synthase and cyclase genes may also be present and expressed. Levels of substrates and intermediates, e.g., isopentenyl diphosphate, dimethylallyl diphosphate, geranylgeranyl diphosphate, kaurene and kaurenoic acid, can be determined by extracting samples from culture media for analysis according to published methods.

After the recombinant microorganism has been grown in culture for the desired period of time, steviol and/or one or more steviol glycosides can then be recovered from the culture using various techniques known in the art. If the recombinant host is a plant or plant cells, steviol or steviol glycosides can be extracted from the plant tissue using various techniques known in the art. For example, a crude lysate of the cultured microorganism or plant tissue can be centrifuged to obtain a supernatant. The resulting supernatant can then be applied to a chromatography column, e.g., a C-18 column, and washed with water to remove hydrophilic compounds, followed by elution of the compound(s) of interest with a solvent such as methanol. The compound(s) can then be further purified by preparative HPLC. See also WO 2009/140394.

The amount of steviol or steviol glycoside produced can be from about 1 mg/I to about 1,500 mg/1, e.g., about 1 to about 10 mg/1, about 3 to about 10 mg/1, about 5 to about 20 mg/1, about 10 to about 50 mg/1, about 10 to about 100 mg/1, about 25 to about 500 mg/1, about 100 to about 1,500 mg/1, or about 200 to about 1,000 mg/l. In general, longer culture times will lead to greater amounts of product. Thus, the recombinant microorganism can be cultured for from 1 day to 7 days, from 1 day to 5 days, from 3 days to 5 days, about 3 days, about 4 days, or about 5 days.

It will be appreciated that the various genes and modules discussed herein can be present in two or more recombinant microorganisms rather than a single microorganism. When a plurality of recombinant microorganisms is used, they can be grown in a mixed culture to produce steviol and/or steviol glycosides. For example, a first microorganism can comprise one or more biosynthesis genes for producing steviol while a second microorganism comprises steviol glycoside biosynthesis genes. Alternatively, the two or more microorganisms each can be grown in a separate culture medium and the product of the first culture medium, e.g., steviol, can be introduced into second culture medium to be converted into a subsequent intermediate, or into an end product such as rebaudioside A. The product produced by the second, or final microorganism is then recovered. It will also be appreciated that in some embodiments, a recombinant microorganism is grown using nutrient sources other than a culture medium and utilizing a system other than a fermentor.

Steviol glycosides do not necessarily have equivalent performance in different food systems. It is therefore desirable to have the ability to direct the synthesis to steviol glycoside compositions of choice. Recombinant hosts described herein can produce compositions that are selectively enriched for specific steviol glycosides and have a consistent taste profile. Thus, the recombinant microorganisms, plants, and plant cells described herein can facilitate the production of compositions that are tailored to meet the sweetening profile desired for a given food product and that have a proportion of each steviol glycoside that is consistent from batch to batch. Microorganisms described herein do not produce the undesired plant byproducts found in Stevia extracts. Thus, steviol glycoside compositions produced by the recombinant microorganisms described herein are distinguishable from compositions derived from Stevia plants.

### V. FOOD PRODUCTS

The steviol and steviol glycosides obtained by the methods disclosed herein can be used to make food products, dietary supplements and sweetener compositions. For example, substantially pure steviol or steviol glycoside such as rebaudioside A can be included in food products such as ice cream, carbonated beverages, fruit juices, yogurts, baked goods, chewing gums, hard and soft candies, and sauces. Substantially pure steviol or steviol glycoside can also be included in non-food products such as pharmaceutical products, medicinal products, dietary supplements and nutritional supplements. Substantially pure steviol or steviol glycosides may also be included in animal feed products for both the agriculture industry and the companion animal industry. Alternatively, a mixture of steviol and/or steviol glycosides can be made by culturing recombinant microorganisms separately or growing different plants/plant cells, each producing a specific steviol or steviol glycoside, recovering the steviol or steviol glycoside in substantially pure form from each microorganism or plant/plant cells and then combining the compounds to obtain a mixture containing each compound in the desired proportion. The recombinant microorganisms, plants, and plant cells described herein permit more precise and consistent mixtures to be obtained compared to current Stevia products. In another alternative, a substantially pure steviol or steviol glycoside can be incorporated into a food product along with other sweeteners, e.g. saccharin, dextrose, sucrose, fructose, erythritol, aspartame, sucralose, monatin, or acesulfame potassium. The weight ratio of steviol or steviol glycoside relative to other sweeteners can be varied as desired to achieve a satisfactory taste in the final food product. See, e.g., U.S. Patent Publication No. 2007/0128311. In some embodiments, the steviol or steviol glycoside may be provided with a flavor (e.g., citrus) as a flavor modulator. For example, Rebaudioside C can be used as a sweetness enhancer or sweetness modulator, in particular for carbohydrate based sweeteners, such that the amount of sugar can be reduced in the food product.

Compositions produced by a recombinant microorganism, plant, or plant cell described herein can be incorporated into food products. For example, a steviol glycoside composition produced by a recombinant microorganism, plant, or plant cell can be incorporated into a food product in an amount ranging from about 20 mg steviol glycoside/kg food product to about 1800 mg steviol glycoside/kg food product on a dry weight basis, depending on the type of steviol glycoside and food product. For example, a steviol glycoside composition produced by a recombinant microorganism, plant, or plant cell can be incorporated into a dessert, cold confectionary (e.g., ice cream), dairy product (e.g., yogurt), or beverage (e.g., a carbonated beverage) such that the food product has a maximum of 500 mg steviol glycoside/kg food on a dry weight basis. A steviol glycoside composition produced by a recombinant microorganism, plant, or plant cell can be incorporated into a baked good (e.g., a biscuit) such that the food product has a maximum of 300 mg steviol glycoside/kg food on a dry weight basis. A steviol glycoside composition produced by a recombinant microorganism, plant, or plant cell can be incorporated into a sauce (e.g., chocolate syrup) or vegetable product (e.g., pickles) such that the food product has a maximum of 1000 mg steviol glycoside/kg food on a dry weight basis. A steviol glycoside composition produced by a recombinant microorganism, plant, or plant cell can be incorporated into a bread such that the food product has a maximum of 160 mg steviol glycoside/kg food on a dry weight basis. A steviol glycoside composition produced by a recombinant microorganism, plant, or plant cell can be incorporated into a hard or soft candy such that the food product has a maximum of 1600 mg steviol glycoside/kg food on a dry weight basis. A steviol glycoside composition produced by a recombinant microorganism, plant, or plant cell can be incorporated into a processed fruit product (e.g., fruit juices, fruit filling, jams, and jellies) such that the food product has a maximum of 1000 mg steviol glycoside/kg food on a dry weight basis.

For example, such a steviol glycoside composition can have from 90-99% rebaudioside A and an undetectable amount of stevia plant-derived contaminants, and be incorporated into a food product at from 25-1600 mg/kg, e.g., 100-500 mg/kg, 25-100 mg/kg, 250-1000 mg/kg, 50-500 mg/kg or 500-1000 mg/kg on a dry weight basis.

Such a steviol glycoside composition can be a rebaudioside B-enriched composition having greater than 3% rebaudioside B and be incorporated into the food product such that the amount of rebaudioside B in the product is from 25-1600 mg/kg, e.g., 100-500 mg/kg, 25-100 mg/kg, 250-1000 mg/kg, 50-500 mg/kg or 500-1000 mg/kg on a dry weight basis. Typically, the rebaudioside B-enriched composition has an undetectable amount of stevia plant-derived contaminants.

Such a steviol glycoside composition can be a rebaudioside C-enriched composition having greater than 15% rebaudioside C and be incorporated into the food product such that the amount of rebaudioside C in the product is from 20-600 mg/kg, e.g., 100-600 mg/kg, 20-100 mg/kg, 20-95 mg/kg, 20-250 mg/kg, 50-75 mg/kg or 50-95 mg/kg on a dry weight basis. Typically, the rebaudioside C-enriched composition has an undetectable amount of stevia plant-derived contaminants.

Such a steviol glycoside composition can be a rebaudioside D-enriched composition having greater than 3% rebaudioside D and be incorporated into the food product such that the amount of rebaudioside D in the product is from 25-1600 mg/kg, e.g., 100-500 mg/kg, 25-100 mg/kg, 250-1000 mg/kg, 50-500 mg/kg or 500-1000 mg/kg on a dry weight basis. Typically, the rebaudioside D-enriched composition has an undetectable amount of stevia plant-derived contaminants.

Such a steviol glycoside composition can be a rebaudioside E-enriched composition having greater than 3% rebaudioside E and be incorporated into the food product such that the amount of rebaudioside E in the product is from 25-1600 mg/kg, e.g., 100-500 mg/kg, 25-100 mg/kg, 250-1000 mg/kg, 50-500 mg/kg or 500-1000 mg/kg on a dry weight basis. Typically, the rebaudioside E-enriched composition has an undetectable amount of stevia plant-derived contaminants.

Such a steviol glycoside composition can be a rebaudioside F-enriched composition having greater than 4% rebaudioside F and be incorporated into the food product such that the amount of rebaudioside F in the product is from 25-1000 mg/kg, e.g., 100-600 mg/kg, 25-100 mg/kg, 25-95 mg/kg, 50-75 mg/kg or 50-95 mg/kg on a dry weight basis. Typically, the rebaudioside F-enriched composition has an undetectable amount of stevia plant-derived contaminants.

Such a steviol glycoside composition can be a dulcoside A-enriched composition having greater than 4% dulcoside A and be incorporated into the food product such that the amount of dulcoside A in the product is from 25-1000 mg/kg, e.g., 100-600 mg/kg, 25-100 mg/kg, 25-95 mg/kg, 50-75 mg/kg or 50-95 mg/kg on a dry weight basis. Typically, the dulcoside A-enriched composition has an undetectable amount of stevia plant-derived contaminants.

In some embodiments, a substantially pure steviol or steviol glycoside is incorporated into a tabletop sweetener or “cup-for-cup” product. Such products typically are diluted to the appropriate sweetness level with one or more bulking agents, e.g., maltodextrins, known to those skilled in the art. Steviol glycoside compositions enriched for rebaudioside A, rebaudioside C, rebaudioside D, rebaudioside E, rebaudioside F, or dulcoside A can be package in a sachet, for example, at from 10,000 to 30,000 mg steviol glycoside/kg product on a dry weight basis, for tabletop use.

### VI. PLANT BREEDING

A. Polymorphisms

Polymorphisms among the nucleic acids described herein (e.g., UGT91D2 nucleic acids) can be used as markers in plant genetic mapping and plant breeding programs in Stevia. See, e.g., Yao et al., Genome, 1999, 42:657-661. Thus, the polymorphisms described herein can be used in a method of identifying whether that polymorphism is associated with variation in a trait. The method involves measuring the correlation between variation in the trait in plants of a Stevia line or population and the presence of one or more genetic polymorphisms in those plants, thereby identifying whether or not the genetic polymorphisms are associated with variation in the trait. Typically, the trait is the total amount of steviol glycosides present in leaves of the plant, although the trait also can be the amount of a particular steviol glycoside, e.g., rebaudioside A, rebaudioside B, rebaudioside C, rebaudioside D, rebaudioside E, rebaudioside F, or dulcoside A. In some embodiments, the trait is the amount of steviol, or the amount of an isoprenoid precursor. A statistically significant correlation between the trait and the presence of the polymorphic marker is determined using an appropriate parametric or non-parametric statistic, e.g., Chi-square test, Student's t-test, Mann-Whitney test, or F-test. A statistically significant correlation between, for example, the amount of rebaudioside A in a plant and presence of a polymorphic marker indicates that the marker may be useful in a marker-assisted breeding program for selection of altered rebaudioside A levels.

Polymorphisms may be detected by means known in the art, including without limitation, restriction fragment length polymorphism (RFLP), random amplified polymorphic DNA detection (RAPD), amplified fragment length polymorphism (AFLP), simple sequence repeat (SSR) or microsatellites. Discovery, detection, and genotyping of polymorphisms have been described in the literature. See, e.g., Henry, ed. (2001) Plant Genotyping. The DNA Fingerprinting of Plants Wallingford: CABI Publishing; and Phillips and Vasil, eds. (2001) DNA-based Markers in Plants Dordrecht: Kluwer Academic Publishers. For example, a primer or probe derived from the nucleic acid sequences set forth in SEQ ID NO:6, SEQ ID NO:9, or SEQ ID NO:96, or the complements thereof, can be used to identify one or more individual plants that possess the polymorphic allele that is correlated with a desired steviol glycoside composition. Those plants then can be used in a breeding program to combine the polymorphic allele with a plurality of other alleles at other loci that are correlated with the desired steviol glycoside composition. As will be evident to one of skill, the number and type of markers required can differ, depending on the trait(s) to be selected for and the degree of correlation for each marker. The methods, therefore, involve detecting a plurality of polymorphisms in the genome of the plant in certain embodiments. Tt will be appreciated that the method may further comprise storing the results of the step of detecting the plurality of polymorphisms on a computer readable medium.

Thus, in some embodiments, a method for identifying Stevia plant lines or populations comprises supplying a nucleic acid sample for a Stevia plant, providing amplification primers for amplifying a region of a Stevia plant corresponding to a UGT gene having 90% or greater sequence identity to a nucleic acid encoding the polypeptides set forth in SEQ ID NOs: 1, 3, 5, or 7, present in the sample, applying the amplification primers to the nucleic acid sample such that amplification of the region occurs, and identifying plants having a desired trait based on the presence of one or more polymorphisms in the amplified nucleic acid sample that correlate with the trait.

In some embodiments, a method of determining the presence of a polynucleotide in a Stevia plant involves contacting at least one probe or primer pair with nucleic acid from the plant. The probe or primer pair is specific for a polynucleotide that encodes a UGT polypeptide having at least 90% sequence identity to SEQ ID NOs: 1, 3, 5, or 7. The presence or absence of the polynucleotide is then determined.

In addition to methods for detecting polymorphisms and determining the genotype of a Stevia plant, kits suitable for carrying out the methods are also described, as well as a computer readable medium produced by such methods that contains data generated by the methods. A kit for genotyping a Stevia biological sample includes a primer pair that specifically amplifies, or a probe that specifically hybridizes to, a polynucleotide that encodes a UGT polypeptide having at least 90% sequence identity to SEQ ID NOs: 1, 3, 5, or 7. Such kits typically have the primer or probe contained within suitable packaging material.

In some embodiments of the methods and kits described herein, one or more sets of oligonucleotides, each capable of recognizing the presence or absence of a specific and defined genomic position, is used. For polyploid Stevia lines or populations, more oligonucleotides are desirable. The lower limit is one oligonucleotide pair and the upper limit is set by the desired resolution capacity of the method and the test kit. Hybridization of the oligonucleotides to DNA from the Stevia plant is preferably recorded in situ by any conventional labelling system, applying for instance terminal transferase and conventional recordable labels. As an alternative to in situ labelling the hybridized sample DNA may be released from the solid support and subsequently hybridized with labelled polynucleotide sequences corresponding to each of the original oligonucleotide sequences attached to the solid support. Hybridization is optionally reversible and the solid support can be returned to its original state for reuse. A labelled dideoxynucleotide can be incorporated at the end of the oligonucleotide provided that the oligonucleotide is hybridized to genomic DNA as template. The nucleotide sequence at the genomic position adjacent to the region matching the oligonucleotide is known and therefore the particular nucleotide which will be incorporated (A, C, G, T or U) is known. Co-dominant scoring is achieved using paired, i.e. two or parallel, i.e. three, flanking oligonucleotide sequences. The results obtained are recorded as full, empty, failure or null alleles and can be used to distinguish between heterozygous and/or homozygous genotypes. Optional post-hybridization treatments, including washing and digestion, are provided in order to remove sample DNA not fully hybridized to the solid support-attached oligonucleotide sequences, for example before and after labelling. The presence or absence of hybridization is recorded using a method allowing the recording of the hybridization state, typically on a computer readable medium.

B. Breeding Programs

Stevia is typically an outcrossing species, although self-pollination is occasionally observed. Thus, a Stevia plant breeding program typically involves the use of one or more of: recurrent selection mass selection, bulk selection, and intercrossing. These techniques can be used alone or in combination with one or more other techniques in a breeding program. See, Yadav et al., Can. J. Plant Sci. 91: 1-27 (2011). Each identified plant can be crossed to a different plant to produce seed, which is then germinated to form progeny plants. Seed from one or more progeny plants possessing the desired phenotype(s) and desired polymorphism(s) is composited and then randomly mated to form a subsequent progeny generation. The breeding program can repeat these steps for an additional 0 to 5 generations as appropriate in order to achieve the desired stability in the resulting plant population, which retains the polymorphic allele(s). In most breeding programs, analysis for the particular polymorphic allele will be carried out in each generation, although analysis can be carried out in alternate generations if desired. Selfing of progeny plants may be carried out for those stevia lines and populations in which selfing is feasible.

Recurrent selection is a method used in a plant breeding program to improve a population of plants. The method entails individual plants cross pollinating with each other to form progeny. The progeny are grown and the superior progeny selected by any number of selection methods, which include individual plant, half-sib progeny, full-sib progeny and selfed progeny. The selected progeny are self pollinated or cross pollinated with each other to form progeny for another population. This population is planted and again superior plants are selected to self pollinate or cross pollinate with each other. Recurrent selection is a cyclical process and therefore can be repeated as many times as desired. The objective of recurrent selection is to improve the traits of a population. The improved population can then be used as a source of breeding material to obtain new varieties for commercial or breeding use, including the production of a synthetic cultivar. A synthetic cultivar is the resultant progeny formed by the intercrossing of several selected varieties. The number of parental plant varieties, populations, wild accessions, ecotypes, etc., that are used to generate a synthetic can vary from as little as 10 to as much as 500. Typically, about 100 to 300 varieties, populations, etc., are used a parents for the synthetic variety. Seed from the parental seed production plot of a synthetic variety can be sold to the farmer. Alternatively, seed from the parental seed production plot can subsequently undergo one or two generations of multiplication, depending on the amount of seed produced in the parental plot and the demand for seed.

Mass selection is a useful technique when used in conjunction with molecular marker-assisted selection. In mass selection, seeds from individuals are selected based on phenotype or genotype. These selected seeds are then bulked and used to grow the next generation. Bulk selection requires growing a population of plants in a bulk plot, allowing the plants to self-pollinate, harvesting the seed in bulk and then using a sample of the seed harvested in bulk to plant the next generation. Also, instead of self pollination, directed pollination could be used as part of the breeding program.

Thus, in some embodiments, a method of making a Stevia plant line or population involves identifying one or more plants in the line or population in which the presence of a polymorphism at a locus having nucleotide sequence encoding a polypeptide that is at least 90% identical to SEQ ID NOs: 1, 3, 5, or 7 is associated with variation in a trait of interest. The identified plant(s) is then crossed with itself or a different stevia plant to produce seed, and at least one progeny plant grown from the seed is again crossed with itself or a different stevia plant for an additional 0-5 generations to make a line or population that possesses the polymorphism.

In some cases, selection for other useful traits is also carried out, e.g., selection for disease resistance. Selection for such other traits can be carried out before, during or after identification of individual plants that possess the desired polymorphic allele.

Marker-assisted breeding techniques may be used in addition to, or as an alternative to, other sorts of identification techniques.

The invention will be further described in the following examples, which do not limit the scope of the invention described in the claims.

### VI. EXAMPLES

### Example 1—Construction of Kaurene Biosynthesis Pathway Genes

A nucleotide sequence encoding a truncated baker's yeast HMG CoA reductase was cloned into a yeast high copy episomal plasmid vector such that the coding sequence was operably linked to and under the transcriptional control of a promoter which can be repressed by the amino acid methionine. See, U.S. Pat. Nos. 5,460,949 and 5,306,862.

Nucleotide sequences encoding the GGPPS enzymes shown in Table 1 were modified for expression in yeast (see SEQ ID NOs:18-25) and cloned into an E. coli vector such that the coding sequence was operably linked to and under the transcriptional control of a yeast promoter which can be repressed by the amino acid methionine. The name for each expression cassette-containing plasmid (“entry vector”) is also shown in Table 1. The nucleotide sequences from the source organisms from which the polypeptides were originally identified are set forth in SEQ ID NOs: 26-33. Other entry vectors were constructed using GGPPS enzymes expressed by an unmodified nucleotide sequence from Catharanthus roseus designated EV270, an unmodified nucleotide sequence from Aspergillus nidulans designated C301 and an unmodified nucleotide sequence from Xanthophyllomyces dendrorhous designated C413.

TABLE 1
GGPPS Clones

| Enzyme Source | | Accession | Plasmid | Construct | Length | SEQ ID | SEQ ID |
|---|---|---|---|---|---|---|---|
| Organism | gi Number | Number | Name | Name | (nts) | (DNA) | (protein) |
| Stevia rebaudiana | 90289577 | ABD92926 | pMUS14 | MM-1 | 1086 | 18 | 121 |
| Gibberella fujikuroi | 3549881 | CAA75568 | pMUS15 | MM-2 | 1029 | 19 | 122 |
| Mus musculus | 47124116 | AAH69913 | pMUS16 | MM-3 | 903 | 20 | 123 |
| Thalassiosira pseudonana | 223997332 | XP_002288339 | pMUS17 | MM-4 | 1020 | 21 | 124 |
| Streptomyces clavuligerus | 254389342 | ZP_05004570 | pMUS18 | MM-5 | 1068 | 22 | 125 |
| Sulfulobus acidocaldarius | 506371 | BAA43200 | pMUS19 | MM-6 | 993 | 23 | 126 |
| Synechococcus sp. | 86553638 | ABC98596 | pMUS20 | MM-7 | 894 | 24 | 127 |
| Arabidopsis thaliana | 15234534 | NP_195399 | pMUS21 | MM-8 | 1113 | 25 | 128 |

Nucleotide sequences encoding the CDPS enzymes shown in Table 2 were modified for expression in yeast (see SEQ ID NOs: 34-36) and cloned into yeast entry vectors. The nucleotide sequences from the source organisms from which the polypeptides were originally identified are set forth in SEQ ID NOs: 37-39. Other entry vectors were constructed using CDPS enzymes expressed by an unmodified nucleotide sequence from Arabidopsis thaliana designated EV64, an unmodified nucleotide sequence from Zea mays designated EV65 and an unmodified nucleotide sequence from Lycopersicon esculentum designated EV66.

TABLE 2
CDPS Clones

| Enzyme Source | | Accession | Plasmid | Construct | Length | SEQ ID: | SEQ ID |
|---|---|---|---|---|---|---|---|
| Organism | gi Number | Number | Name | Name | (nts) | (DNA) | (protein) |
| Stevia rebaudiana | 2642661 | AAB87091 | pMUS22 | MM-9 | 2364 | 34 | 129 |
| Streptomyces clavuligerus | 197705855 | EDY51667 | pMUS23 | MM-10 | 1584 | 35 | 130 |
| Bradyrhizobium japonicum | 529968 | AAC28895.1 | pMUS24 | MM-11 | 1551 | 36 | 131 |

Nucleotide sequences encoding the KS enzymes shown in Table 3 were modified for expression in yeast (see SEQ ID NOs: 40-43) and cloned into yeast entry vectors. The nucleotide sequences from the source organisms from which the polypeptides were originally identified are set forth in SEQ ID NOs:44-47. Other entry vectors were constructed using KS enzymes expressed by an unmodified nucleotide sequence from Arabidopsis thaliana designated EV70, an unmodified nucleotide sequence from Cucurbita maxima designated EV71 and an unmodified nucleotide sequence from Cucumis sativus designated EV72.

TABLE 3
KS Clones

| Enzyme Source | | Accession | Plasmid | Construct | Length | SEQ ID | SEQ ID |
|---|---|---|---|---|---|---|---|
| Organism | gi Number | Number | Name | Name | (nts) | (DNA) | (protein) |
| Stevia rebaudiana | 4959241 | AAD34295 | pMUS25 | MM-12 | 2355 | 40 | 132 |
| Stevia rebaudiana | 4959239 | AAD34294 | pMUS26 | MM-13 | 2355 | 41 | 133 |
| Zea mays | 162458963 | NP_001105097 | pMUS27 | MM-14 | 1773 | 42 | 134 |
| Populus trichocarpa | 224098838 | XP_002311286 | pMUS28 | MM-15 | 2232 | 43 | 135 |

Nucleotide sequences encoding the CDPS-KS fusion enzymes shown in Table 4 were modified for expression in yeast (see SEQ ID NOs: 48 and 49) and cloned into yeast entry vectors. The nucleotide sequences from the source organisms from which the polypeptides were originally identified are set forth in SEQ ID NOs: 50 and 51.

TABLE 4
CDPS-KS Clones

| Enzyme Source | | Accession | Plasmid | Construct | Length | SEQ ID | SEQ ID |
|---|---|---|---|---|---|---|---|
| Organism | gi Number | Number | Name | Name | (nts) | (DNA) | (protein) |
| Phomopsis amygdali | 186704306 | BAG30962 | pMUS29 | MM-16 | 2952 | 48 | 136 |
| Physcomitrella patens | 146325986 | BAF61135 | pMUS30 | MM-17 | 2646 | 49 | 137 |

Nucleotide sequences encoding the KO enzymes shown in Table 5 were modified for expression in yeast (see SEQ ID NOs: 52-55) and cloned into yeast entry vectors. The nucleotide sequences from the source organisms from which the polypeptides were originally identified are set forth in SEQ ID NOs: 56-59.

TABLE 5
KO Clones

| Enzyme Source | | Accession | Plasmid | Construct | Length | SEQ ID | SEQ ID |
|---|---|---|---|---|---|---|---|
| Organism | gi Number | Number | Name | Name | (nts) | (DNA) | (protein) |
| Stevia rebaudiana | 76446107 | ABA42921 | pMUS31 | MM-18 | 1542 | 52 | 138 |
| Arabidopsis thaliana | 3342249 | AAC39505 | pMUS32 | MM-19 | 1530 | 53 | 139 |
| Gibberella fujikoroi | 4127832 | CAA76703 | pMUS33 | MM-20 | 1578 | 54 | 140 |
| Trametes versicolor | 14278967 | BAB59027 | pMUS34 | MM-21 | 1500 | 55 | 141 |

Nucleotide sequences encoding the KAH enzymes shown in Table 6 were modified for expression in yeast (see SEQ ID NOs: 60-64) and cloned into yeast entry vectors. The nucleotide sequences from the source organisms from which the polypeptides were originally identified are set forth in SEQ ID NOs: 65-69.

TABLE 6
KAH Clones

| Enzyme Source | | Accession | Plasmid | Construct | Length | SEQ ID | SEQ ID |
|---|---|---|---|---|---|---|---|
| Organism | gi Number | Number | Name | Name | (nts) | (DNA) | (protein) |
| Stevia rebaudiana | | —* | pMUS35 | MM-22 | 1578 | 60 | 142 |
| Stevia rebaudiana | 189418962 | ACD93722 | pMUS36 | MM-23 | 1431 | 61 | 143 |
| Arabidopsis thaliana | 15238644 | NP_197872 | pMUS37 | MM-24 | 1578 | 62 | 144 |
| Vitis vinifera | 225458454 | XP_002282091 | pMUS38 | MM-25 | 1590 | 63 | 145 |
| Medicago trunculata | 84514135 | ABC59076 | pMUS39 | MM-26 | 1440 | 64 | 146 |

*= Sequence is shown in U.S. Patent Publication No. 2008-0064063.

Nucleotide sequences encoding the CPR enzymes shown in Table 7 were modified for expression in yeast (see SEQ ID NOs: 70-72) and cloned into yeast entry vectors. The nucleotide sequences from the source organisms from which the polypeptides were originally identified are set forth in SEQ ID NOs:73-75.

TABLE 7
CPR Clones

| Enzyme Source | | Accession | Plasmid | Construct | Length | SEQ ID | SEQ ID |
|---|---|---|---|---|---|---|---|
| Organism | gi Number | Number | Name | Name | (nts) | (DNA) | (protein) |
| Stevia rebaudiana | 93211213 | ABB88839 | pMUS40 | MM-27 | 2133 | 70 | 147 |
| Arabidopsis thaliana | 15233853 | NP_194183 | pMUS41 | MM-28 | 2079 | 71 | 148 |
| Giberella fujikuroi | 32562989 | CAE09055 | pMUS42 | MM-29 | 2142 | 72 | 149 |

### Example 2—Construction of Steviol Glycoside Pathway Genes

Integration vectors containing nucleotide sequences encoding the UGT85C2 and UGT74G1 enzymes listed in Table 8 were transformed into yeast. Transformants were obtained that contained UGT85C2, or UGT85C2 and UGT74G1, integrated into the genome.

TABLE 8
UGT Clones

| Source | | | Accession | | Plasmid | Length | |
|---|---|---|---|---|---|---|---|
| Organism | UGT No. | gi Number | Number | Type | Name | (nucleotides) | SEQ ID |
| Stevia rebaudiana | UGT85C2 | 37993660 | AY345978.1 | Integration | pMUS11 | 1446 | 4 |
| | | | | vector | | | |
| Stevia rebaudiana | UGT74G1 | 37993668 | AY345982 | Integration | pMUS12 | 1383 | 2 |
| | | | | vector | | | |
| Stevia rebaudiana | UGT76G1 | 37993652 | AY345974 | Integration | pMUS13 | 1377 | 8 |
| | | | | vector | | | |
| Ipomoea purpurea | IP3GGT | 62857205 | AB192315.1 | High copy | pMUS10 | 1380 | 77 |
| | | | | vector | | | |
| Bellis perennis | UGT94B1 | 56550538 | AB190262.1 | High copy | pEF1156 | 1317 | 79 |
| | R25S mutant | (wild type) | (wild type) | vector | | (wild type) | |
| Arabidopsis thaliana | UGT79B3 | 28951020 | BT005370.1 | High copy | pEF1153 | 1362 | 151 |
| | | | | vector | | | |

Nucleotide sequences encoding the IP3GGT and UGT94B1 R25S enzymes were modified for expression in yeast (see SEQ ID NOs: 77 and 79) and cloned into yeast entry vectors. Amino acid sequences for IP3GGT and UGT94B1 R25S are set forth in SEQ ID NOs: 76 and 78, respectively. The high copy episomal vector containing a modified IP3GGT nucleotide sequence was designated pEF1155. The high copy episomal vector containing a modified UGT94B1 R25S nucleotide sequence was designated pEF1156.

### Example 3—Construction of Yeast Strains

A yeast strain designated EFSC301 was modified by replacing the endogenous ERG9 promoter with the copper inducible CUP1 promoter. Strain EFSC301 is a derivative of EUROSCARF collection yeast strain BY4742. See, the world wide web at uni-frankfurt.de/fb15/mikro/euroscarf/data/by.html. In standard yeast growth medium, the ERG9 gene is transcribed at very low levels, since the concentration of copper in such medium is low. The decrease in ergosterol production in this strain results in increased amounts of isoprene units available for steviol biosynthesis. The yeast strain was also modified by genomically integrating the Stevia UGT85C2 and UGT74G1 genes, each under the transcriptional control of the strong constitutive GPD promoter. See Table 8. The strain has one copy of each of the Stevia UGT85C2 and UGT74G1 genes integrated in the MUS1241 strain genome.

### Example 4—Analysis of Steviol Glycoside Pathway Gene Expression in Yeast

To examine steviol glycoside biosynthesis in yeast, the expression cassettes of the 36 entry vectors of Tables 1-7 and Example 1 were randomly concatenated in ligation reactions to create artificial yeast chromosomes (“eYACs”). The process is shown schematically in FIG. 5.

Two different sets of ligations were carried out. Ligation set A included all genes listed in Tables 1-7, except that no bi-functional CDPS-KS genes (Table 4) were included. Ligation set B included all genes listed in Tables 1-7 except that no mono-functional CDPS and KS genes (Tables 2-3) were included.

From 30 to 200 mg of DNA was prepared from each of the cassette-containing entry vectors. The gene expression cassettes were released from each vector by digestion with the restriction enzyme AscI. The cassettes were then randomly concatenated into eYACs by ligation with T4 ligase in a 3 hour reaction. The success of the concatenation reaction was assessed by the viscosity of the reaction mixture, since concatenated DNA is highly viscous. DNA fragments (“arms”) containing a centromere, two telomeres and the LEU2 and TRP1 selection markers were added to the end of the concatenated expression cassettes, thereby creating functional eYACs.

The eYACs were transformed into spheroplasts of the competent yeast strain MUS1243 by zymolyase digestion of the yeast cell wall, followed by treatment with a CaCl₂/PEG buffer, making the spheroplasts permeable to large molecules such as eYACs.

After transformation, the yeast spheroplasts were embedded in a noble agar based solid growth medium, in which regeneration of the cell wall can take place. Colonies appeared from 4-8 days after inoculation. The regeneration medium lacked the amino acids leucine and tryptophan, thus selecting for the presence of double-armed eYACs in the yeast cells.

About 3,000 transformants were obtained for each set. Each transformant was re-streaked and tested for yeast strain markers and the genetic presence of both arms of the eYAC, i.e., the LEU2 and TRP1 markers. More than 97% of the transformants had the correct genotype. Each transformant was given a CEY designation number.

Initially, 24 CEYs from each set were grown for 24 hours in 2 ml of Synthetic Complete medium (SC), without methionine, so as to induce gene expression from the eYACs. After 24 hours, the supernatant from each culture was collected and subjected to LC-MS (Liquid Chromatography-coupled Mass Spectrometry (Triple Quadropole)) analysis for the presence of rubusoside. Since the Stevia UGT74G1 and UGT85C2 genes are co-expressed in each CEY transformant, the expected end product when steviol is produced is rubusoside (steviol-(13-β-D-glucopyranosyloxy)-β-D-glucopyranosyl ester).

None of the CEYs from set B produced detectable levels of rubusoside, whereas 7 of the CEYs from set A did. Strain CEY19 was the top producer. CEY19 produced a compound with a mass of 665.2, which could correspond to a sodium adduct of rubusoside. A compound with a mass of 643.2 also was seen, and probably corresponds to protonated rubusoside. MS-MS-based molecular fractionation of the 665.2 mass compound resulted in a break down mass of 503.2, which corresponds to steviol monoside as a sodium adduct. Since the mass, the fractionation pattern, the HPLC spectrum, and the retention standard of this compound corresponded exactly to that of a rubusoside standard produced in vitro by the glucosylation of steviol using Stevia enzymes 85C2 and 74G1, the compound produced by CEY was determined to be rubusoside.

Additional Screening for Rubusoside Production

An additional 95 clones from set A and 95 clones from set B were grown in 96 deep-well trays in 1 ml SC medium without methionine. Supernatants from each of these cultures were combined in pools of two clones, analyzed by LC-MS, and the MS signal/noise ratio determined. The MS s/n ratio is an approximate measure of the relative rubusoside content. When a pool of 2 CEYs was found to produce rubusoside, each clone in that pool was analyzed separately. The results showed that no set B CEYs produced rubusoside, while at least 28 CEYs from set A produced detectable levels of rubusoside.

Identification of Genes Present in Rubusoside Producing CEY Clones

To correlate the gene content of eYACs to rubusoside production, a PCR protocol was developed in which similar sized fragments (0.5 kb) of all the possible eYAC-borne genes could be amplified. Internal primers of 20-25 nt were placed so that a similar annealing temperature could be used to amplify all genes. Genomic DNA, which includes eYAC DNA, was prepared from 4 CEYs with no rubusoside production, 4 with low rubusoside production and 6 with high to very high rubusoside production. Using equimolar amounts of these 14 DNA preparations, analytical PCR was performed for all 37 genes for these 14 CEYs, as well as positive and negative controls. All genes were amplified except one, apparently due to primer failure.

The genes present in the six high rubusoside-producing CEY strains are shown in Table 9. The genes present in the eight low or no rubusoside-producing CEY strains are shown in Table 10.

TABLE 9
Genes Present in High Rubusoside-Producing CEY Strains

| | HIGH production | | | VERY high production | | |
|---|---|---|---|---|---|---|
| Gene | CEY50 | CEY176 | CEY19 | CEY173 | CEY191 | CEY213 |
| tHMG1 | + | + | + | + | − | + |
| MM-1 | − | + | + | + | + | − |
| MM-2 | − | + | + | + | + | − |
| MM-3 | + | + | + | + | + | + |
| MM-4 | + | + | + | − | + | + |
| MM-5 | + | + | + | + | + | + |
| MM-6 | + | + | + | + | + | + |
| MM-7 | − | + | − | + | + | − |
| MM-8 | + | + | + | + | − | + |
| EV270 | + | + | − | + | + | + |
| C301 | + | + | + | + | + | + |
| C413 | + | + | − | + | + | + |
| MM-9 | + | + | + | + | + | + |
| MM-10 | + | − | − | + | + | + |
| MM-11 | + | + | − | + | + | + |
| EV64 | + | + | + | + | + | + |
| EV65 | − | − | + | + | + | + |
| EV66 | + | + | + | + | + | + |
| MM-12 | + | − | − | + | + | + |
| MM-13 | + | + | + | + | + | + |
| MM-14 | + | + | + | + | + | + |
| MM-15 | − | − | − | − | + | − |
| EV70 | − | + | + | + | − | − |
| EV71 | Primers failed | | | | | |
| EV72 | + | + | + | + | + | + |
| MM-18 | + | + | + | + | + | − |
| MM-19 | + | − | + | − | + | + |
| MM-20 | + | + | + | + | + | + |
| MM-21 | − | − | + | + | − | + |
| MM-22 | + | + | + | + | + | + |
| MM-23 | + | − | + | + | − | + |
| MM-24 | + | + | + | + | + | + |
| MM-25 | + | + | + | + | + | + |
| MM-26 | + | + | + | + | + | + |
| MM-27 | + | + | + | + | + | + |
| MM-28 | − | − | − | − | − | − |
| MM-29 | + | + | + | + | + | + |

TABLE 10
Genes Present in CEY Strains Producing Low or No Rubusoside

| | NO rubusoside production | | | | LOW production | | | |
|---|---|---|---|---|---|---|---|---|
| Gene | CEY162 | CEY169 | CEY171 | CEY188 | CEY75 | CEY147 | CEY214 | CEY87 |
| tHMG1 | − | − | − | − | − | − | + | + |
| MM-1 | + | + | + | + | − | + | − | − |
| MM-2 | + | − | + | + | + | + | + | + |
| MM-3 | + | + | + | + | + | + | + | + |
| MM-4 | − | − | + | − | − | + | − | + |
| MM-5 | + | + | + | + | + | + | + | + |
| MM-6 | + | + | + | − | + | + | + | + |
| MM-7 | + | − | + | + | + | + | + | + |
| MM-8 | + | + | + | + | + | + | + | + |
| EV270 | + | + | + | + | + | + | + | + |
| C301 | + | + | + | + | + | + | + | + |
| C413 | + | + | + | + | + | + | + | + |
| MM-9 | + | + | + | + | − | + | + | + |
| MM-10 | + | + | + | + | − | + | + | + |
| MM-11 | + | + | + | + | + | + | + | − |
| EV64 | + | + | + | + | − | + | + | + |
| EV65 | + | − | − | − | + | − | + | − |
| EV66 | + | + | + | + | + | + | + | + |
| MM-12 | + | + | + | + | + | + | + | + |
| MM-13 | + | + | + | + | + | + | + | + |
| MM-14 | + | + | + | + | + | + | + | + |
| MM-15 | + | − | + | − | + | + | − | + |
| EV70 | + | + | + | + | + | + | + | + |
| EV71 | Primers failed | | | | | | | |
| EV72 | + | + | + | + | + | + | + | + |
| MM-18 | + | + | + | + | + | + | + | + |
| MM-19 | + | + | + | + | + | + | + | + |
| MM-20 | + | + | + | + | + | + | + | + |
| MM-21 | − | + | − | − | − | + | − | + |
| MM-22 | + | + | + | + | + | − | + | + |
| MM-23 | + | − | + | − | + | + | − | + |
| MM-24 | + | − | + | + | + | + | + | + |
| MM-25 | + | − | + | + | + | + | + | + |
| MM-26 | + | + | + | + | + | − | + | + |
| MM-27 | + | + | + | + | + | + | + | + |
| MM-28 | − | − | + | − | − | − | − | + |
| MM-29 | + | + | + | + | − | + | + | + |

### Example 5—Modification of Yeast Culture Conditions

Experiments were carried out with strain CEY213 in order to determine culture conditions conducive to maximum rubusoside production. The starting material was a glycerol freezer stock (−80° C.) of CEY213. Frozen cells originally came from an agar plate containing SC yeast medium without tryptophan, leucine and histidine (SC-TLH), and containing 2 mM methionine. Five ml of liquid SC-TLH medium containing 2 mM methionine was inoculated with a loop-full of freeze stock CEY213 yeast cells. eYAC expression in CEY213 is repressed under these conditions. The cells were grown overnight at 30° C. with slow shaking (170 rpm) and were designated as “pre-cultures.”

The CEY 213 pre-cultures were used to inoculate 25-50 ml of SC media without methionine, in which the parameters indicated below were varied. Rubusoside production under each of the growth conditions was measured by centrifuging 500 μl of each culture medium, transferring 250 μl of the supernatant to a new tube, adding 250 μl methanol, shaking thoroughly and centrifuging for 10 minutes at maximum speed. An aliquot of the supernatant was analyzed for rubusoside production by LC-MS.

Copper Levels

CEY213 precultures were grown in SC medium to which 50 μM bathocuproinedisulfonic acid was added. Bathocuproinedisulfonic acid chelates copper in the growth medium. The ERG9 gene in CEY213 has been modified so that expression is controlled by the CUP1 promoter. A decrease in copper levels in the medium will further decrease ERG9 activity and thereby increase the amount of isoprene units available for steviol biosynthesis.

Chelation of copper ions in the growth medium had a detrimental effect on growth of the yeast culture and rubusoside production was decreased proportionally. These results suggested that even without copper chelation, strain CEY213 is at its minimum rate of ergosterol biosynthesis, and no more isoprene units can be diverted from ergosterol biosynthesis towards steviol glycoside production.

Glucose

Doubling the available glucose from 2 to 4% had a marginal effect on rubusoside production, about a 5-10% increase in rubusoside production.

Limiting Available Nitrogen

CEY213 pre-cultures were grown under conditions of limited available nitrogen. Limiting nitrogen during growth of yeast in culture is known to increase production of ergosterol. When the concentration of NH₄SO₄ was decreased from 4 g/l to 2, 1 or 0.4 g/l, the growth rate of CEY213 decreased in proportion to the amount of nitrogen. Rubusoside production decreased proportionally with the decrease in growth.

Aeration of Cultures

CEY213 was grown in Ehrlenmeyer flasks with or without baffles. The results indicated that there was at best a marginal effect of increased aeration via the use of baffles. If anything, the lack of aeration via the lack of baffles increased production.

Optical Density at Initiation, Fermentation Time and Growth Temperature

Cultures were initiated at two different optical densities, OD₆₀₀=0.1 or OD₆₀₀=1.0 of pre-cultured CEY213. Fermentation was then carried out for 24, 48, 72 or 144 hours at a temperature of 20, 25 or 30° C.

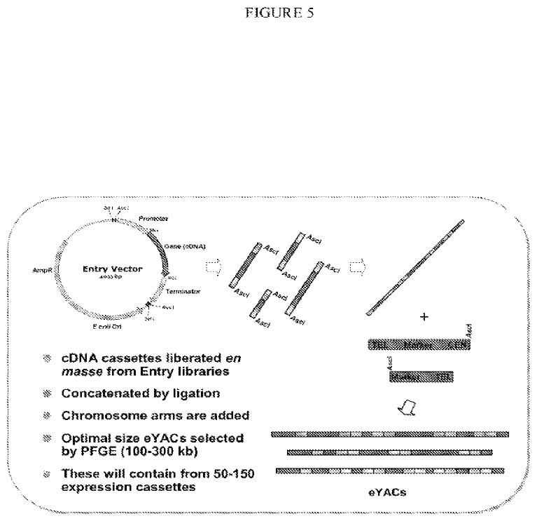

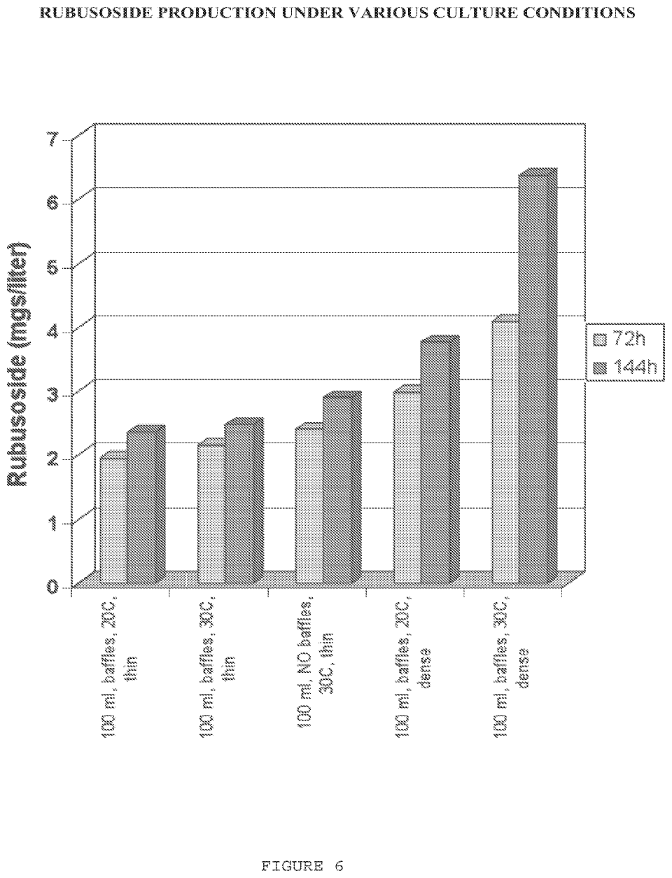

As shown in FIG. 6, the density of the batch culture at fermentation start, the culture temperature and the length of time in fermentation, in combination, had a significant effect on the amount of rubusoside produced by CEY213. Thus, 144 hours growth of a culture with a starting density of OD₆₀₀=1.0, at 30° C., resulted in the production of no less than 8.5 mgs/liter of rubusoside.

### Example 6—Large Scale Production of Rubusoside

A series of fermentation experiments with CEY213 were performed using 3 kinds of yeast medium (rich medium and two types of synthetic medium), varying inoculation density, and changing timing of eYAC gene cassette expression.

Batch Fermentation Conditions

Batch fermentation was carried out by centrifuging a CEY213 pre-culture, discarding the supernatant and re-suspending the cells in 6 liters of SC-TLH medium containing 100 μM methionine and 4% glucose. The OD₆₀₀ was adjusted to 1.0 in a 100 ml Ehrlenmeyer flask without baffles and the cells were allowed to grow for 144 hours at 30° C. with slow shaking.

Recovery of Rubusoside

After fermentation, the culture was centrifuged and the supernatant was mixed with an equal volume of methanol, shaken thoroughly, and centrifuged to remove precipitated material. The resulting supernatant was purified by flash C18-silica column chromatography with methanol as the eluent, followed by preparative HPLC to obtain one major compound, with one additional minor compound detected.

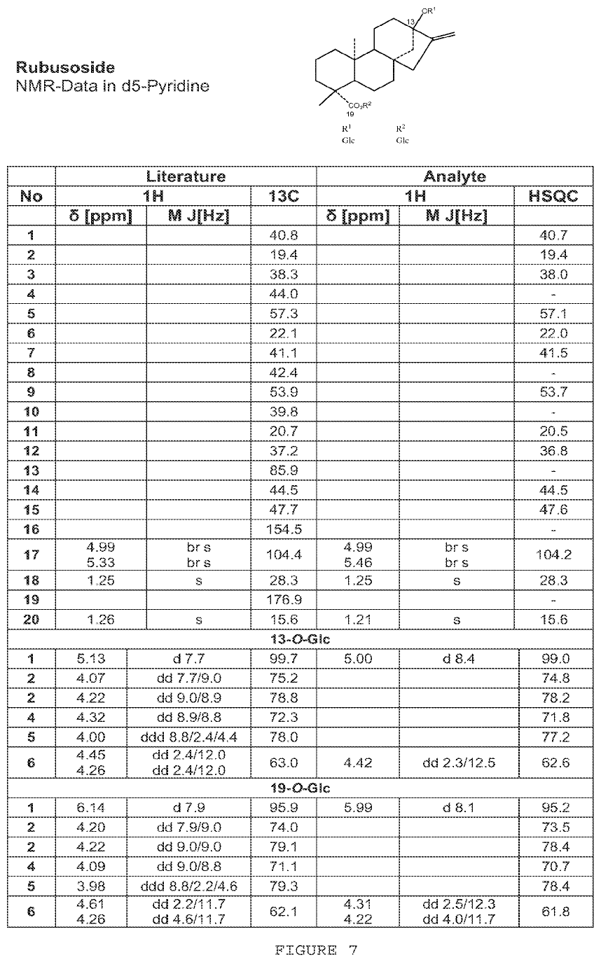

The purified compound was analyzed by ¹H and ¹³C NMR, and the data are shown in FIG. 7. The compound was confirmed to be rubusoside based on comparison to ¹H and ¹³C NMR literature values for rubusoside. Quantitative analysis indicated that CEY213 fermentation produced 12.8 mgs/liter of rubusoside.

### Example 7—IP3GGT Activity

1. Enzymatic Activity of Ipomoea purpurea 3GGT Glycosyltransferase In Vitro

The enzymatic activity of Ipomoea purpurea 3 GGT glycosyltransferase (IP3GGT) using steviol as a substrate was determined in vitro. Genes for Stevia rebaudiana UGT85C2 and IP3GGT glycosyltransferase were each expressed in E. coli and each enzyme was purified.

The enzymatic reaction was performed in two steps. First, 0.5 mM steviol (9.55 mgs total) was incubated with ca. 0.5 μg UGT85C2 enzyme for 16 hours at 30° C. in a reaction buffer (containing 1 mM UDP-glucose, 100 mM Tris-HCl (pH 8.0), 5 mM MgCl₂, 1 mM KCl, 0.1 U/ul calf intestine phosphatase). Then ca. 0.5 μg IP3GGT enzyme was added and the reaction mixture incubated for an additional 20 hours at 30° C.

Analysis of the reaction products indicated about 100% conversion of steviol to steviol-13-O-monoside, 25% of which was further glycosylated into stevio-13-O-1,2-bioside. The theoretical steviol-13-O-1,2-bioside yield was about 4.8 mg. The reaction mixture was then subjected to preparative HPLC, which yielded 2.5 mg steviol-13-O-1,2-bioside (52% purification yield). Using LC-MS, the mass of the purified compound had a different retention time than rubusoside and steviol-13-O-1,3-bioside. The purified compound was subjected to ¹H NMR, heteronuclear single quantum coherence (HSQC)-NMR and heteronuclear multiple bond correlation (HMBC)-NMR analysis, which confirmed that the compound was steviol-13-O-1,2-bioside.

2. In Vivo Expression of IP3GGT in Steviol- or Steviol Monoside-Fed Yeast

To determine whether the IP3GGT was active in yeast, the 2μ high copy (episomal) plasmid, pMUS10, containing an unmodified IP3GGT coding sequence operably linked to a strong GPD1 promoter was transformed into the yeast strain MUS1245. MUS1245 contains a genomically integrated UGT85C2 expression cassette. The resulting yeast strain was grown in SC medium without histidine to select for the continued presence of the IP3 GGT expression plasmid, at a starting density of OD₆₀₀=0.2. Steviol or steviol monoside was added to the medium at 3 mM. After growth for 72 hours at 30° C., culture supernatants were assayed for the presence of steviol and steviol glucosides by HPLC.

LC-MS analysis indicated that no 1,2-glucosylated steviol-13-O-glucoside was detected after feeding with steviol, although steviol-13-O-monoside could be detected. In contrast, low but detectable amounts of the steviol 1,2-bioside were produced by MUS1245 carrying pMUS10 after feeding with steviol-13-O-monoside. These results show that the native Ipomoea purpurea 3GGT coding sequence is expressed in yeast at levels sufficient to obtain detectable in vivo conversion of steviol monoside to steviol 1,2-bioside.

### Example 8—Modification of Yeast Strains

EXG1 and EXG2

S. cerevisiae may contain enzymes that degrade the 1,2 or 1,3 sugar bonds in steviol 1,2- and steviol 1,3-biosides. To test this possibility, yeast strain CEY213 was grown for 3 days at 30° C. on media containing 0.1 mM of each of the two biosides. LC-MS analysis of the culture showed the level of 1,2-bioside to be stable, whereas the 1,3-bond in the 1,3-bioside appeared to completely hydrolyse within the limits of detection of the assay.

Twenty-five S. cerevisiae mutants, each disrupted in one known or putative glycoside hydrolase gene, were examined for their ability to degrade steviol biosides. A culture of each yeast mutant was grown as described above on media containing steviol 1,3-bioside and analyzed by LC-MS. The yeast strain carrying a mutation in the EXG1 (exo-1,3-β-glucanase) gene was found to have lost most of the 1,3-bioside hydrolysing activity. The nucleotide sequence of the yeast EXG1 gene is reported in Vazquez de Aldana et al. Gene 97:173-182 (1991). The yeast strain carrying a mutation in the EXG2 gene (another exo-1,3-β-glucanase) showed a small decrease in hydrolysing activity. Correa, et al., Current Genetics 22:283-288 (1992).

A double mutant yeast strain (exg1 exg2) was made. When the double mutant strain was grown on media containing steviol 1,3-bioside, no hydrolysis of the bioside was detected.

### Example 9—Increased Titer of Steviol Biosynthesis

Individual clones of enzymes from each of the different enzyme classes tested in Example 4 (and Table 11) were examined using eYAC technology to identify particular clones that exhibited the greatest production of steviol from isopentenyl pyrophoshate and farnesyl pyrophosphate. The GGPPS, KO and KAH enzymes have been tested on eYACs, individually or in the case of GGPPS enzymes individually or in pools of two (e.g., Synechococcus sp.+S. acidocaldarius GGPPS or Aspergillus nidulans GGPPS alone), in a S. cerevisiae strain expressing all remaining enzymatic steps in the steviol pathway. The results indicated that the Synechococcus spp. GGPPS clone MM-7 (encoded by SEQ ID NO:24) was the most efficient. GGPPS clones from Aspergillus nidulans and Sulfulobus acidocaldarius also were quite active. The results also indicated that among the KO and KAH clones, the Stevia KO clone MM-18 (encoded by SEQ ID NO:52) and the A. thaliana KAH clone MM-24 (encoded by SEQ ID NO:62) resulted in the greatest steviol production.

TABLE 11

| | | | Accession | Coding | Coding Sequence |
|---|---|---|---|---|---|
| Source Organism | Enzyme | gi Number | Number | Sequence | Length (nucleotides) |
| Stevia rebaudiana | GGPPS-1 | 158104429 | ABD92926 | MM-1 | 1086 |
| Gibberella | GGPPS-2 | 3549881 | CAA75568 | MM-2 | 1029 |
| fujikoroi | | | | | |
| Mus musculus | GGPPS-3 | | BC069913.1 | MM-3 | 903 |
| Thalassiosira | GGPPS-4 | 223997332 | XP_002288339 | MM-4 | 1020 |
| pseudonana | | | | | |
| Sulfulobus | GGPPS-6 | 506371 | BAA43200 | MM-6 | 993 |
| acidocaldarius | | | | | |
| Synechococcus sp. | GGPPS-7 | 86553638 | ABC98596 | MM-7 | 894 |
| Cantharanthus | GGPPS-9 | 1063275 | X92893 | EV270 | 1074 |
| roseus | | | | | |
| Aspergillus | GGPPS-10 | 29468175 | AF479566 | C301 | 1191 |
| nidulans | | | | | |
| Xanthophyllomyces | GGPPS11 | 63145970 | DQ016502 | C413 | 1131 |
| dendrorhous | | | | | |
| Stevia rebaudiana | CDPS-1 | 2642661 | AAB87091 | MM-9 | 2364 |
| Streptomyces | CDPS-2 | 197705855 | EDY51667 | MM-10 | 1584 |
| clavuligerus | | | | | |
| Bradyrhizobium | CDPS-3 | 529968 | AAC28895.1 | MM-11 | 1551 |
| japonicum | | | | | |
| Arabidopsis | CDPS-4 | 18412041 | NM_116512 | EV-64 | 2409 |
| thaliana | | | | | |
| Zea mays | CDPS-5 | 50082774 | AY562490 | EV-65 | 2484 |
| Lycopersicon | CDPS-6 | 6009477 | AB015675 | EV-66 | 2403 |
| esculentum | | | | | |
| Stevia rebaudiana | KS-1 | 4959241 | AAD34295 | MM-12 | 2355 |
| Stevia rebaudiana | KS-2 | 4959239 | AAD34294 | MM-13 | 2355 |
| Zea mays | KS-3 | 162458963 | NP_001105097 | MM-14 | 1773 |
| Populus | KS-4 | 224098838 | XP_002311286 | MM-15 | 2232 |
| trichocarpa | | | | | |
| Arabidopsis | KS-5 | 3056724 | AF034774 | EV-70 | 2358 |
| thaliana | | | | | |
| Cucurbita maxima | KS-6 | 1431869 | U43904 | EV-71 | 2370 |
| Cucumis sativus | KS-7 | 21326756 | AB045310 | EV-72 | 2358 |
| Stevia rebaudiana | KO-1 | 76446107 | ABA42921 | MM-18 | 1542 |
| Arabidopsis | KO-2 | 3342249 | AAC39505 | MM-19 | 1530 |
| thaliana | | | | | |
| Gibberella | KO-3 | 74676162 | O94142 | MM-20 | 1578 |
| fujikoroi | | | | | |
| Trametes | KO-4 | 14278966 | AB057426 | MM-21 | 1500 |
| versicolor | | | | | |
| Stevia rebaudiana | KAH-1 | * | | MM-22 | 1578 |
| Stevia rebaudiana | KAH-2 | 189418962 | ACD93722 | MM-23 | 1431 |
| Arabidopsis | KAH-3 | 15238644 | NM_122399 | MM-24 | 1578 |
| thaliana | | | | | |
| Vitis vinifera | KAH4 | 225458453 | XM_002282055 | MM-25 | 1590 |
| Medicago | KAH5 | 84514134 | DQ335781 | MM-26 | 1440 |
| trunculata | | | | | |
| Stevia rebaudiana | CPR-1 | 189098311 | DQ269454.4 | MM-27 | 2133 |
| Arabidopis | CPR-2 | 145343899 | NM_118585 | MM-28 | 2079 |
| thaliana | | | | | |
| Gibberella | CPR-3 | 32562988 | AJ576025.1 | MM-29 | 2142 |
| fujikoroi | | | | | |

* U.S. Patent Publication No. 20080064063

S. cerevisiae strain CEY213, described in Example 4, was transformed with high copy plasmids carrying one of the CDPS or KS genes shown in Table 11, operably linked to the strong GPD1 promoter. Preliminary experiments indicated that overexpression of the Stevia rebaudiana CDPS (CDPS-1, encoded by SEQ ID NO:34) in CEY213 gave an increase in rubusoside production relative to CEY213 that lacked the high copy CDPS-1 overexpressing plasmid. The experiments also indicated that the Stevia rebaudiana KO (KO-1, encoded by SEQ ID NO:52) was the most active KO of the two tested.

To construct a yeast strain with consistently high levels of steviol glycoside production, expression cassettes containing the GGPPS-10 clone, the KO-1 clone (SEQ ID NO:52) and the KAH-3 clone (SEQ ID NO:62) were stably integrated into the genome of the S. cerevisiae strain CEN.PK 111-61A. Expression of these cassettes was driven by the constitutive GPD1 and TPI1 promoters. In addition, expression cassettes containing KS-1 (SEQ ID NO:40), CDPS-1 (SEQ ID NO:34) and UGT74G1 (SEQ ID NO:2) were stably integrated into the genome. The resulting yeast strain, EFSC1751, however, did not produce any steviol-19-O-monoside when grown at laboratory scale under the conditions described in Example 6.

To determine the basis for the lack of steviol glycoside production in EFSC1751, CDPS-3, CDPS-4, CDPS-5 and CPR-1 genes, alone or in combination, were expressed in strain EFSC1751. CPR-1 is from Stevia, rebaudiana and its sequence can be found at Genbank Accession DQ269454.4. The results showed that CPR-1, when expressed with either CDPS-3, CDPS-4 or CDPS-5, resulted in production of steviol-19-O-monoside in EFSC1751. None of these genes alone in the same strain resulted in any production. These results indicate that the genomically integrated copy of CDPS-1, Stevia enzyme, is non-functional in this yeast construct, whereas the Bradyrhizobium, Arabidopsis or Zea CDPS clones were functional in this construct. In addition, the plant-derived KAH and/or KO genes integrated into the chromosome for this construct appear to require an exogenous CPR for activity. The CPR from Giberella fujikuroi (MM-29) also appears to be able to work with plant-derived KAH and/or KO polypeptides.

The two leading GGPPS candidates, GGPPS-6 (encoded by SEQ ID NO:23) and GGPPS-7 (encoded by SEQ ID NO:24), were further expressed individually in a S. cerevisiae strain that has a functional steviol glycoside pathway (including UGT74G1) but no GGPPS genes. Transformants then were analyzed for the production of 19-SMG by LC-MS analysis of culture samples that had been boiled in 50% DMSO for 5 minutes and centrifuged at 16000 relative centrifugal force (RCF) for 5 minutes. It was found that many transformants containing the GGPPS-6-expressing plasmid did not produce 19-SMG.

Very few transformants were obtained containing GGPPS-7, indicating that GGPPS-7 (Synechococcus sp.) may be the more active of the two enzymes, and that the activity could be high enough to confer toxicity. For example, a dramatic increase in GGPP production could result in a drain on a downstream pathway such as ergosterol production. To test this hypothesis, a UPC2-1 gene was co-expressed with GGPPS-7, and ergosterol feeding of the cells was attempted to see if this would rescue growth of cells. However, cell growth was not rescued.

Cell toxicity also may be due to an accumulation of GGPP or a metabolite of GGPP. To test this hypothesis, CDPS-5 was further overexpressed in the GGPPS-7-expressing yeast strain to see if the toxicity could be alleviated by increased GGPP usage. CDPS5 over-expression did appear to rescue growth to some extent since transformants with a plasmid overexpressing this enzyme along with the GGPPS-7 gave rise to a few colonies. The number of transformants was still low. Over-expression of CDPS-5 in a similar strain but with GGPPS-10 instead of GGPPS-7 resulted in a doubling of steviol glycoside production, and these results together could suggest that CDPS is a limiting bottleneck in the introduced steviol glycoside biosynthesis pathway.

In summary, based upon production of 19-SMG or rubusoside in test tube cell cultures at 30° C. with yeast medium+2% glucose, for 24-72 hours, the following conclusions were made with the eYAC constructs: KS-1 (Stevia rebaudiana, encoded by SEQ ID NO:40), KO-1 (S. rebaudiana, encoded by SEQ ID NO:52) and KAH-1 (S. rebaudiana) or KAH-3 (Arabidopsis thaliana, encoded by SEQ ID NO:62) appear to be the best combinations for the steviol pathway. GGPPS-7 (Synechococcus sp.) appears to show the highest amount of activity for this step, but if downstream bottlenecks occur overexpression also could lead to toxicity and overall lower levels of steviol glycosides. All combinations of CDPS and CPR gene analogs were tested and it was found that all 3 CPRs in Table 11 were active, and that combinations of CPR-1 (S. rebaudiana, encoded by SEQ ID NO:70) or CPR-3 (Gibberella fujikuroi, encoded by SEQ ID NO:72) with either CDPS-5 (Zea mays) or CDPS-4 (A. thaliana) were particularly useful. CDPS-5 appears to be the optimal CDPS in the pathway. Combinations can be further tested in a reporter strain with reduced flux to sterol pathways.

To investigate the potential for even higher activity of the CDPS from Zea mays (CDPS-5), this gene was expressed from a 2 micron multicopy plasmid using the GPD promoter, with and without a plastid signal peptide, to determine if activity is higher in the cytoplasm when targeting sequences are removed. The nucleotide sequence and amino acid sequence of the CDPS-5 from Zea mays and containing the chloroplast signal peptide are set forth in SEQ ID NOs:80 and 81, respectively. The chloroplast signal peptide is encoded by nucleotides 1-150 of SEQ ID NO:80, and corresponds to amino acids 1 to 50 of SEQ ID NO:81. The plasmid was transformed into the stable rubusoside producer strain (EFSC1859) that has GGPPS-10, CDPS-5, KS-1, KO-1, KAH-3, CPR-1 and UGT74G1 (SEQ ID NO:2) integrated into the genome and expressed from the strong constitutive GPD and TPI promoters. Furthermore, in strain EFSC1859, expression of squalene synthase, which is encoded by ERGS, was downregulated by displacement of the endogenous promoter with the CUP1 inducible promoter. In addition to these genes, strain EFSC1859 also expresses UGT85C2 (SEQ ID NO:3) from a 2 micron multicopy vector using a GPD1 promoter. Rubusoside and 19-SMG production were measured by LC-MS to estimate the production level. The removal of the plastid leader sequence did not appear to increase steviol glycoside production as compared to the wild-type sequence. However, this work demonstrates that the leader sequences can be removed without causing a loss of steviol pathway function.

Similarly, plasmids were constructed for CPR-3, KAH-3 and KO-1 without membrane anchoring sequences (i.e., nucleotides 4-63 of SEQ ID NO:72; nucleotides 4-87 of SEQ ID NO:62; and nucleotides 1-117 of SEQ ID NO:52) and were transformed into strain EFSC1859 with the UGT85C2 integrated on the chromosome rather than on a plasmid. It is expected that these enzymes will be functional without the anchoring sequence.

### Example 10—Identification of Steviol-1,3-O-Monoglucoside 1,2-Glucosyltransferase Sequences

Stevia EST Analysis

A tBLASTN search of a Stevia (Stevia rebaudiana) leaf EST (Expressed Sequence Tags) database (Brandle et al., Plant Mol. Biol. 50:613-622, 2002) was carried out using complete Ipomoea (Ipomoea purpurea) UGT79 type UGT (IP3GGT), Bellis (Bellis perennis) UGT94B1, Stevia UGT79A2, Stevia UGT76G1 and Stevia UGT91D1 amino acid sequences as queries, thus representing UGTs from all Family 1 glycosyltransferase sub-families known to primarily contain diglycosyltransferases. Partial sequences for 9 previously undescribed UGT genes were identified. One of the partial sequences was from the UGT 79 sub-family (“79-EV1”), one from the UGT 76 sub-family (“76-EV1”) and two from the UGT 91 sub-family (“91-EV-1” and “91-EV2”), as well as members of the UGT 71, 72, 78, 84 and 88 sub-families. Seven of the partial sequences were isolated using Stevia cDNA or cDNA libraries as the PCR template for isolation. In addition, two Stevia members of the UGT 76 sub-family were isolated, GenBank accession ACT33422.1 which is a member of the 76G1 sub-family (Mohankumar), and GenBank accession ACM47734.1 which is a member of the 76G2 (Yang) sub-family.

Pyrosequencing

Additional UGT clones were identified and isolated by performing pyrosequencing with Stevia cDNA as follows. Stevia mRNA was prepared from Stevia leaves, using the Ambion® Micro Poly Purist™ mRNA preparation kit. As a quality control, reverse transcribed mRNA was tested for the presence of the Stevia Rebaudioside A pathway UGT genes 85C2, 74G1 and 76G1, by employing analytical PCR with oligonucleotide primers identical to 21 nucleotides at the 5′- and 3′-termini of each sequence. The amplified full length mRNA was then used for pyrosequencing and contig assembly (MOgene, St. Louis, MO USA). About 3.4 million reads of an average length of 393 nucleotides were performed, and the resulting raw sequences used to obtain 25907 sequence contigs. A database was constructed, containing publicly available amino acid sequences of a total of ca. 1,500 UGTs. About 150 of the sequenced UGTs were fully annotated UGTs from a wide variety of sub-families. The remaining sequenced UGTs were partially annotated homologs of these. A BLASTX search was performed (CLC Genomics, Muehltal, Germany), using the 25907 Stevia EST contigs as query, to the fabricated UGT database (Genetic code=1, Low complexity=Yes, Expect value=10.0, Word size=3, No of processors=2, Matrix=BLOSUM62, Gap cost (open)=11, Gap cost (extension)=1). The results suggested that sequences for more than 90 previously unknown UGTs from Stevia were present in the pyrosequencing database.

No additional members of the UGT 79 sub-family or the UGT 94 sub-family were identified in the pyrosequencing database. However, the analysis showed new members of the UGT 76 and 91 sub-families. For a few of the genes, full length sequence data was immediately available from the pyrosequencing EST data. A previously constructed Stevia plasmid cDNA library was used to obtain full-length sequences for those members for which partial sequence data was obtained. An oligonucleotide primer identical to each specific, partial UGT sequence was combined with an oligonucleotide primer identical to the library plasmid vector sequence. These primers were employed in PCR to obtain the full length product, which was subsequently sequenced. Based on the full length sequence, a second PCR was performed using a proof-reading PCR polymerase enzyme for amplification of the full length UGT gene from a Stevia cDNA library as the template for the reaction. Using this strategy, five members of the UGT 76 sub-family, six members of the UGT 91 sub-family, as well as ten members of other UGT sub-families were isolated.

Each of the 7 UGTs identified from the Stevia EST database, the 2 publicly available Stevia UGT 76 sequences, and the 21 UGTs identified from pyrosequencing was cloned into the E. coli expression vectors pET30A+ or pETDuet (making use of the HIS-tag for purification purposes) and expressed in the autolysis-prone E. coli strains XjA and XjB. For a large number of these UGTs, expression of the UGT protein resulted in the formation of inclusion bodies. In order to overcome formation of those inclusion bodies, some of these UGTs were expressed in the low temperature expression strain “Arctic Express” (Agilent Technologies). For those which failed to express in this system, coupled in vitro transcription-translation of PCR products (TNT® T7 Quick for PCR DNA kit, Promega) was attempted, allowing successful expression of the remaining UGTs. Efficiency of the reaction was ensured by labeling with ³⁵S-methionine, separation on SDS-PAGE and phosphorimaging detection of a protein band of the expected size for the UGT protein in question.

UGT polypeptides from each clone, expressed as described above, were tested for 1,2-glycosylation activity, using steviol-13-O-monoglucoside as substrate. In vitro transcribed/translated protein, corresponding to approximately one fifth of the total protein formed in a 25 μL reaction, was used in an in vitro reaction, using 0.5 mM steviol-13-O-monoglucoside (SMG) as substrate, in a reaction buffer (containing 1 mM UDP-glucose, 100 mM Tris-HCl (pH 8.0), 5 mM MgCl₂, 1 mM KCl, 0.1 U/μl calf intestine phosphatase). The reaction mixture was incubated at 30° C. for 20 hours. The reaction mixture was then analyzed by LC-MS analysis for the presence of Steviol-1,2-bioside. LC-MS analyses were performed using an Agilent 1100 Series HPLC system (Agilent Technologies) fitted with a Phenomenex® Synergy Hydro-RP column (250×3 mm, 3 μm particles, 80 A pore size) and hyphenated to a TSQ Quantum (ThermoFisher Scientific) triple quadropole mass spectrometer with electrospray ionization. Elution was carried out using a mobile phase (30° C.) containing MeCN (0.01% Formic acid) and H₂O (0.01% Formic acid) by applying a gradient composed of 0.6→0.4 ml/min, 5% MeCN for 4 min; 0.4 ml/min, 5→40% MeCN for 2 min; 0.4 ml/min, 40→55% MeCN for 11 min; 0.4→1.0 ml/min, 55→100% MeCN for 3 min. Steviol biosides were detected using SIM (Single Ion Monitoring) on Mw 665.2 [M+Na⁺]. None of the 30 UGT enzymes tested exhibited detectable steviol-13-O-monoglucoside glycosylation activity.

The nucleotide sequences of the six UGT91 members identified by pyrosequencing were compared to the sequence of Stevia UGT91D1 in Genbank Accession No. AY345980. It appeared that the GenBank sequence encoded 12 additional amino acids at the N-terminus, relative to the six sequences identified by pyrosequencing. To re-test UGT91D1 family members for activity, UGT91D1 sequences were re-isolated by PCR amplification of Stevia leaf cDNA. The resulting PCR products were cloned into a plasmid vector and enzymatic activity for each product was measured as described above by: GST-tagged expression in E. coli, coupled in vitro transcription-translation, and/or in vivo expression in yeast. Steviol 1,2-glucosylation activity was detected from one clone by all three methods. This clone was designated UGT91D2e. The amino acid sequence of UGT91D2e is set forth in SEQ ID NO:5. In contrast, no 1,2-glucosylation activity was detected from a clone having the same sequence as described by Accession No. AY345980 (Protein Accession number AAR06918), but lacking the 12 amino acids of the amino terminus.

### Example 11—Analysis of UGT91D2e Sequences

### Sequence Variants of UGT91D2e

As evidenced in FIG. 19B, a small number of amino acid modifications exist between the active (91D2e) variants and the closest inactive homologs (91D1). The 91D1 genes cloned by Ma et al., Shi Yan Sheng Wu Xue Bao. 2003 36(2):123-9 (Protein Accession number AAM53963, GI:21435782) and Brandle et al., supra (Protein Accession number AAR06918, GI:37993665) did not exhibit the 1,2-glycosylating activity required for RebA biosynthesis. To ascertain which amino acids are required for activity, 21 single site-directed mutants were created such that the amino acid in UGT91D2e (SEQ ID NO:5) was changed to the corresponding amino acid in an inactive homolog. See Table 12. In addition, a site-directed mutation was made such that position 364 (SP) also was changed. The mutants were made using the QuikChange® II Site-Directed Mutagenesis kit according to manufacturer's protocols (Agilent Technologies, Santa Clara, CA), and the pGEX-4TI vectors were transformed into a XJb Autolysis E. coli strain (ZymoResearch, Orange, CA). A mutant was not made to change residue 162 from a glycine to an aspartic acid.

In order to assess the activity of the mutant enzymes, a substrate-feeding experiment was performed in vitro using protein produced in E. coli. Initially, E. coli cells were grown overnight at 30° C., followed by induction with 3 mM arabinose and 0.1 mM IPTG, and further incubation at 20° C. For the in vitro assay, cells were induced overnight at 20° C., lysed by a freeze/thaw cycle, and the crude cell extract used for an enzymatic reaction in which the substrates were 0.5 mM steviol-13-O-glucoside and 0.5 mM rubusoside.

The results are shown in Table 12 for the steviol monoglucoside (SMG) and Rubusoside (Rub) substrates. A “+” indicates that diglycosylation activity was detected, a “−” indicates activity was not detected, and “NA” indicates the assay was not performed. The noted mutations are based on the numbering of the 91D2e sequence (SEQ ID NO:5).

As some of the genes have a tendency to express in inclusion bodies in E. coli, the coding sequences that did not show activity in the E. coli experiments also were produced by coupled in vitro transcription-translation of PCR products (TNT® T7 Quick for PCR DNA kit, Promega) as above in Example 10. Briefly, 2 μL of DNA from the PCR amplification of the five single mutants and the wild type enzyme were incubated for 90 minutes at 30° C. with the kit master mix and 1 μL L-[³⁵S]-Methionine, in a total of 25 μL reaction. For each sample, a volume of 2 μL final reaction was run on a SDS-PAGE gel. All six proteins showed similar levels of soluble recombinant protein as judged by visual observation of the SDS-PAGE gel. The results for the in vitro-translated proteins are shown on the right side of Table 12. The percentages in this table indicate the approximate amount of conversion of substrate to product based on relative peak areas of substrate and product.

TABLE 12

| | E. coli | E. coli | in vitro | in vitro |
|---|---|---|---|---|
| | protein | protein | protein | protein |
| Mutation | SMG | Rub | SMG | Rub |
| Y30→F | + | + | NA | NA |
| P93→Q | + | + | NA | NA |
| S99→V | + | + | NA | NA |
| Y122→F | + | + | NA | NA |
| H→140Y | + | + | NA | NA |
| S142→C | + | + | NA | NA |
| T144→I | − | − | 5.9% | 0.05% |
| A148→T | + | + | NA | NA |
| M152→L | − | − | 25.1% | 0.85% |
| G153→A | + | + | NA | NA |
| A156→S | + | + | NA | NA |
| L195→M | + | + | NA | NA |
| V196→E | + | + | NA | NA |
| K199→E | + | + | NA | NA |
| L211→M | + | + | NA | NA |
| L213→F | − | − | 29.4% | 1.59% |
| S221→F | + | + | NA | NA |
| V286→A | + | + | NA | NA |
| S364→P | − | − | 4.1% | 0.4% |
| G384→C | − | − | 14.1% | 1.28% |
| K427→N | + | + | NA | NA |
| E438→A | + | + | NA | NA |

The approximate amount of diglycosylation activity as compared to UGT91D2e (SEQ ID NO:5) was found to be: 6.1% for T144S, 26.2% for M152L, 30.7% for L213F, 4.3% for S364P, and 14.7% for G384C using 13-SMG as substrate. For rubusoside, the approximate amount of diglycosylation activity as compared to UGT91D2e was 1.4%, 23.4%, 43.7%, 10.9% and 35.2% for T144S, M152L, L213F, S364P, and G384C, respectively.

These results indicate that 5 of the 22 amino acid mutations were noticeably deleterious for activity when done in isolation. It is also possible that combinations of the other 17 mutations also could result in inactivity or loss of activity.

By aligning the 91D2e sequences and the variants described above with proteins termed At72B1, Mt85H2, VvGT1 and Mt71G1 (Osmani et al (2009) Phytochemistry 70, 325-347), and analyzing predicted tertiary structures (alpha helices, beta-sheets, and coil regions), regions can be identified where mutations are likely to result in loss of diglycosylation activity. The first three mutations that are deleterious are found in the N-terminal domain, in regions that are thought to be loops. The N-terminal domain (amino acid residues 1-240), in particular the predicted loop regions of the N-terminal domain (amino acids 20-26, 39-43, 88-95, 121-124, 142-158, 185-198, and 203-214), are thought to be primarily responsible for binding of the glucose acceptor molecule substrate. The fourth mutation that appears to be deleterious for activity is found in the C-terminal domain, in a region that is believed to be the C5 loop (corresponding to amino acids 381-386). This loop is also thought to be important for glucose acceptor substrate specificity. Nineteen of the twenty-two mutations that separate the inactive versus the active rubusoside diglycosylase enzymes are located within five amino acids of the predicted acceptor substrate binding regions of 91D2e. Therefore it is likely that the published 91D1 enzymes catalyze a glycosyl transferase reaction between UDP-glucose and an alternative acceptor substrate.

### Example 12—Production of Rebaudioside A in Yeast

### Production of Rebaudioside A in Steviol-Fed Yeast

The yeast strain EFSC1580, which contains a genomically integrated UGT74G1 expression cassette, was transformed with three different 2μ high copy (episomal) plasmids for co-expression of Stevia UGTs 91D2e (SEQ ID NO:5), 85C2 (SEQ ID NO:3), and 76G1 (SEQ ID NO:7). The three plasmids, designated pMUS44, pMUS7 and pMUS9, contain coding sequences for UGT91D2e, UGT85C2 and UGT76G1, respectively, operably linked to the strong GPD1 promoter. The resulting yeast strain was grown in SC medium without uracil, histidine, and leucine to select for the continued presence of the pMUS44, pMUS7 and pMUS9 expression plasmids. Steviol was added to the medium to a final concentration of 250 μM, and the strain was cultured at 30° C. At 18 hours and 72 hours of culture, aliquots of the supernatants and cell pellets were analyzed for the presence of Rebaudioside A by LC-MS. LC-MS analyses were performed using an Agilent 1100 Series HPLC system (Agilent Technologies, Wilmington, DE, USA) fitted with a Phenomenex® Synergy Hydro-RP column (250×3 mm, 3 μm particles, 80 Å pore size) and hyphenated to a TSQ Quantum (ThermoFisher Scientific) triple quadropole mass spectrometer with electrospray ionization. Elution was carried out using a mobile phase (30° C.) containing MeCN (0.01% Formic acid) and H₂O (0.01% Formic acid) by applying a gradient composed of 0.6→0.4 ml/min, 5% MeCN for 4 min; 0.4 ml/min, 5→40% MeCN for 2 min; 0.4 ml/min, 40→55% MeCN for 11 min; 0.4→1.0 ml/min, 55→100% MeCN for 3 min. Steviol biosides were detected using SIM (Single Ion Monitoring).

LC-MS results showed that detectable amounts of Rebaudioside A were found in the supernatant at 18 and 72 hours of culture when strain EFSC1580 containing pMUS44, pMUS7 and pMUS9 was grown in the presence of steviol. The product co-eluted with a Rebaudioside A standard and the expected mass was confirmed as the [M+Na]⁺=989. By comparing the absorbance of the product to the absorbance of a 10 μM Rebaudioside A standard, the accumulation in the supernatant of the cell culture was estimated to be more than 6 mg/L at 18 hours, and more than 15 mg/L at 72 hours.

### Production of Rebaudioside A and Rebaudioside D in Glucose-Fed Yeast

Yeast strain CEY213, described in Example 4, contains steviol biosynthetic pathway genes expressed from eYACs as well as genomically integrated UGT74G1 and UGT85C2 expression cassettes. Strain CEY213 produces rubusoside, as described in Example 6.

Strain CEY213 was transformed with a 2μ high copy (episomal) dual expression plasmid, pMUS47, for simultaneous expression of UGT91D2e (SEQ ID NO:5) and UGT76G1 (SEQ ID NO:7). The pMUS47 plasmid contains two expression cassettes, one having the coding sequence of UGT91D2e and the other having the coding sequence of UGT76G1. Both coding sequences are operably linked to the strong constitutive GPD1 promoter. The resulting yeast strain was pre-cultured overnight at 30° C. in SC medium without histidine, leucine and tryptophan in order to maintain selection for the presence of eYACs, without uracil in order to maintain selection for the presence pMUS47, and finally with methionine (2 mM) in order to suppress promoters present on the eYACs. The next day, the cells were washed and transferred to an identical medium, but without methionine, for induction of the eYAC promoters. Samples were collected after 24 hours and 99 hours of incubation, and supernatants and cell pellets analyzed for the presence of Rebaudioside A and Rebaudioside D, using LC-MS as described above.

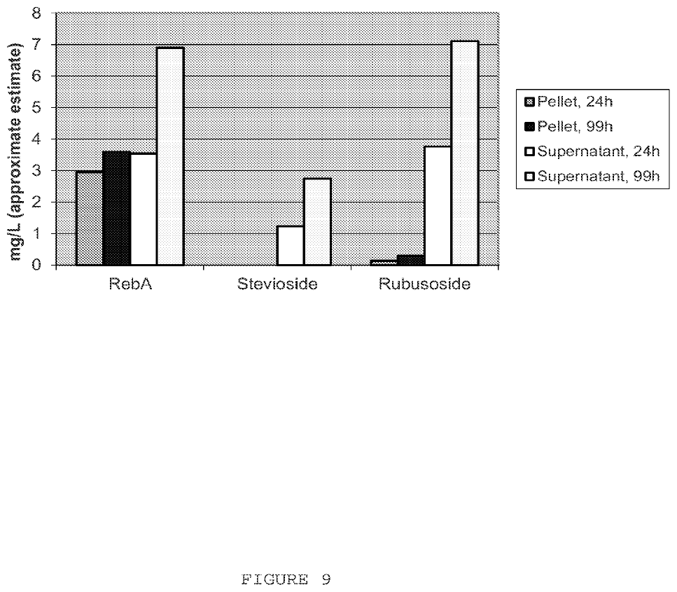

The results showed that detectable amounts of Rebaudioside A were found in the supernatants at both 24 and 99 hours. The product co-eluted with a Rebaudioside A standard and the expected mass was confirmed as the [M-FN]⁺=989. By comparing the absorbance of the product to a 101.1M Rebaudioside A standard, the accumulation of Rebaudioside A in the supernatant was estimated to be more than 3 mg/L at 24 hours and more than 6 mg/L at 99 hours. See FIG. 9. The results also indicated that small amounts of stevioside and rubusoside were present in the yeast cell pellet and that detectable amounts of stevioside and rubusoside were present in the culture supernatant. See FIG. 9.

The results also showed that small but detectable amounts of Rebaudioside D were produced, suggesting that UGT91D2e is capable of conjugating an additional glucose to the 19-O glucose of either stevioside producing Rebaudioside E or directly to the 19-O glucose of Rebaudioside A. These results also suggest that UGT76G1 may be capable of accepting Rebaudioside E as a substrate to produce Rebaudioside D. See FIG. 2C.

### Example 13—Production of Rebaudioside A with Codon Optimized Sequences for UGT Sequences

Optimal coding sequences for UGT 91d2e, 74G1, 76G1, and 85C2 were designed and synthesized for yeast expression using two methodologies, supplied by GeneArt (Regensburg, Germany) (SEQ ID NOs: 6, 2, 8, and 4, respectively) or DNA 2.0 (Menlo Park, CA) (SEQ ID NOs: 84, 83, 85, and 82, respectively). The amino acid sequences of UGT 91d2c, 74G1, 76G1, and 85C2 (SEQ ID NOs: 5, 1, 7, and 3, respectively) were not changed.

High copy number plasmids containing expression cassettes with all four optimized UGTs were constructed and expressed, and their activity compared to expression products of similar constructs containing wild-type sequences. The plasmids were transformed into the universal Watchmaker strain, EFSC301 (described in Example 3). UGTs were inserted in high copy (2μ) vectors and expressed from a strong constitutive promoter (GPD1) (vectors P423-GPD, P424-GPD, P425-GPD, and P426-GPD). After overnight growth and re-inoculation in fresh media at an OD₆₀₀ of 0.25, the culture medium (SC-leu-trp-ura-his) was supplemented with 25 μM steviol (final concentration), and production of Rubusoside (Rub), 19-SMG (19SMG) and RebA (RebA) was measured in the media after 24 h. The experiment was repeated, in part due to the fact that 19-SMG was undetectable in one of the first samples.

The results from the two separate studies, shown in Table 13 below, indicate that all eight of the codon-optimized UGTs were active. However, enzyme expression for at least one of the codon-optimized UGTs in each strategy was reduced by the new codon optimization algorithm used to make the constructs. It appears that in the GeneArt modified constructs (SEQ ID NOs: 6, 2, 8, and 4), a bottleneck was potentially created between rubusoside and RebA. It is expected that individual enzyme activity assays and expression analyses of these coding sequences expressed in the yeast strains will allow for the optimal combination of UGT genes in the pathway.

TABLE 13

| | RebA | 19SMG | Rub |
|---|---|---|---|
| | (μM) | (μM) | (μM) |
| Wild-type | 3.2 | 17.2 | 4.9 |
| | 1.7 | 14.0 | 3.2 |
| DNA2.0 | 4.4 | 12.4 | 4.6 |
| | 1.7 | 10.8 | 3.1 |
| GeneArt | 1.2 | nd | 4.6 |
| | 0.8 | 11.1 | 4.5 |

nd = below detection limit

### Example 14—Production of Rebaudioside A Using UGTs with Sequence Tags

Fusions of small peptides or protein binding domains with the UGT proteins 85C2, 91D2e, 74G1, and 76G1 can promote interactions between the UGTs (channeling) or aid in targeting/anchoring the UGTs to specific components of the yeast cells.

To assess if scaffolding of the UGTs in the RebA pathway could result in active pathway enzymes, the DNA 2.0 codon-optimized UGTs 85C2 and 74G1 were fused in-frame to a string of 4 high-affinity, short (also known as PMI) peptides that resemble the p53 protein motif. The p53 protein motif interacts with the MDM2 protein in humans (see Li et al., J Mol Biol. 2010, 398(2):200-13). DNA 2.0 codon-optimized UGTs 85C2, 91D2e, 74G1 and 76G1 (SEQ ID NOs: 82, 84, 83, and 85, respectively) were fused in-frame to the first 158 amino acids of the human protein MDM2 (gene accession number ABT17086). A small GS-rich linker region also was fused just prior to the N-terminal methionine of the UGTs. Unfused, the affinity of PMI/MDM2 binding is in the low nM range representing a high-affinity binding. Yeast cells transformed with the above constructs are expected to produce a UGT scaffold around the 4 X PMI (P53-like) peptide repeat fused N-terminally to the 85C2 protein (designated 85C2_P53) scaffold.

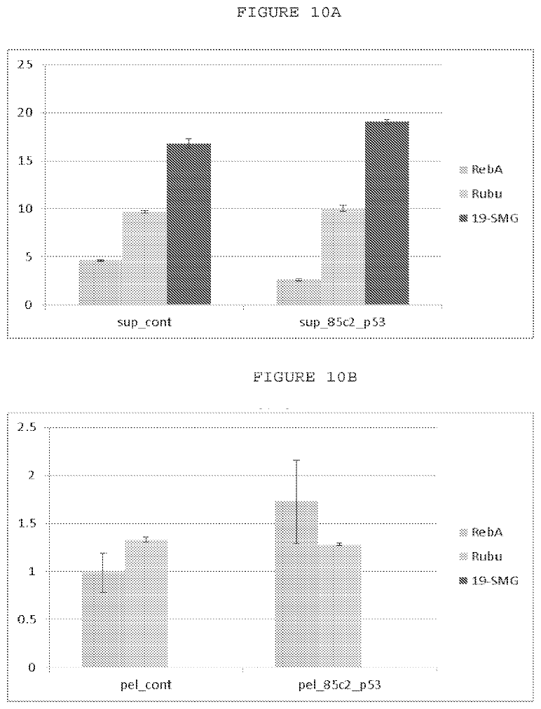

The laboratory yeast strain BY4741, deleted for TRP1, was transformed with expression plasmids p423-426 GPD (Mumberg et al, Gene, 156 (1995), 119-122) expressing Stevia rebaudiana UGTs 74G1,76G1 and 91D2e with N-terminal, in-frame fusions of the first 158 amino acids of human MDM2 protein, and expressing Stevia rebaudiana UGT85C2 with an N-terminal in-frame fusion of 4 repeats of the synthetic PMI peptide (4 X TSFAEYWNLLSP, SEQ ID NO:86). See SEQ ID NOs: 88, 90, 92, and 94 for the amino acid sequences of the 85C2, 74G1, 91D2e, and 76G1 fusion proteins, respectively; see SEQ ID NOs: 89, 92, 93, and 95 for the nucleotide sequences encoding the fusion proteins. This yeast strain and a control strain (expressing the four UGT's without any fusions) were grown overnight in synthetic yeast medium selecting for the presence of plasmids and then transferred the next day to a 96 deep-well tray containing synthetic yeast medium to a cell density giving an OD₆₀₀ of 1. A final concentration of 100 μM steviol was added. After 72 hours, samples were taken and analysed by LC-MS, as described in Example 12. As indicated in FIGS. 10A and 10B, the UGTs are active in yeast when expressed with the various fusion tags.

### Example 15—UGT91D2e Activity

Additional sub-family 91 UGTs were cloned using cDNA/library preparations made from 3 Stevia sources of different genetic backgrounds. Oligonucleotide primers identical to UGT91D1/91D2e were used for PCR amplification of the cDNA preparations, and the resulting PCR products of correct size were cloned into appropriate plasmid vectors. Numerous clones from each experiment were sequenced, and the sequencing results showed that UGT91D nucleic acids with slight variations in sequence could be amplified. The twenty UGT91D variants with the greatest differences in sequence relative to UGT91D2c were expressed by in vitro transcription-translation followed by enzymatic testing for steviol-13-O-monoglucoside-1,2-glucosylating activity. One of the variants showed weak 1,2-bioside glucosylation activity, while the reminder showed no detectable glucosylation activity. Tt therefore appears that UGT91D2 polypeptides are the primary steviol-13-O-monoglucoside-1,2-glucosylating enzymes in Stevia.

### Enzymatic Activity of UGT91D2e

UGT91D2e (SEQ ID NO:5), made by coupled in vitro transcription-translation, was tested for the ability to xylosylate and rhamnosylate steviol-13-O-monoglucoside in an in vitro enzyme assay, using UDP-xylose or UDP-rhamnose as the sugar donors rather than UDP-glucose.

The xylosylation assay was performed as follows: 3 mM UDP-glucuronic acid was mixed with ca. 1 μg Arabidopsis thaliana-encoded UDP-glucuronic acid decarboxylase UXS3 (produced in E. coli and then purified), 100 mM Tris-HCl (pH 8.0), 1 mM DTT, 6 μg BSA, 1 mM MgCl₂, and 1% calf intestine phosphatase. The reaction mixture was incubated for 30 minutes at 30° C., in order for UDP-glucuronic acid to be turned into UDP-xylose. Then 1.5 mM steviol-13-O-monoglucoside substrate and ca. 0.5 μg UGT91D2e enzyme made as described in Example 9 was added to the mixture, which was allowed to incubate at 30° C. for an additional 20 hours.

The rhamnosylation assay was performed in the following way: 3 mM UDP-glucose was mixed with 0.6 μg of each of the N-terminal and C-terminal parts of Arabidopsis thaliana-encoded RHM2 rhamnose synthetase (produced in E. coli and then purified), 100 mM Tris-HCl (pH 8.0), 1 mM DTT, 1.5 mM NADPH, 1.5 mM NAD+, 6 mg BSA, 1 mM MgCl₂, and 1% calf intestine phosphatase. The reaction mixture was incubated for 30 minutes at 30° C., in order for UDP-glucose to be turned into UDP-rhamnose. Then 1.5 mM steviol-13-O-monoglucoside substrate and ca. 0.5 μg UGT91D2e enzyme was added to the mixture, which was allowed to incubate at 30° C. for an additional 20 hours.

The results indicated that UGT91D2e was capable of carrying out xylosylation of the steviol-13-O-monoglucoside substrate at about one half to one third the rate observed with UDP-glucose, forming 1,2-xylosylated steviol-13-O-monoside, which is a precursor to Rebaudioside F. UGT91D2c was capable of carrying out rhamnosylation of the steviol-13-O-monoglucoside substrate at about the same rate as the rate observed with UDP-glucose, forming 1,2-rhamnosylated steviol-13-O-monoside, which is a precursor for Rebaudioside C (Dulcoside B). These results indicate that synthesis of appropriate precursor molecules and expression of appropriate UGTs in vivo should result in the production of Rebaudioside F and C in vivo. See FIGS. 2B and 2D.

UGT91D2e also was tested for its ability to 1,2-glucosylate substrates other than steviol-13-O-monoglucoside in vitro, i.e., rubusoside, steviol-1,3-bioside and 1,3-stevioside. The results indicated that UGT 91D2e was not active when a 1,3-bound glucose was present (e.g., steviol 1,3-bioside and 1,3-stevioside), while UGT 91D2e was active regardless of primary glucosylation at the 19-O position. These results suggest that steviol 1,3-bioside and 1,3-stevioside are likely not present in the in vivo Stevia pathway for rebA formation. See FIG. 2A and FIG. 3.

### Example 16—UGT91D Homologs

Different ecotypes of S. rebaudiana are genetically diverse. Investigation of 96 clones of 91Ds from different Stevia RNA accessions revealed many amino acid changes between six investigated ecotypes (e.g., at nucleotide 74 (resulting in an amino acid change of G to D), 89 (Y to F), 131 (V to A), 137 (F to S), 278 (P to Q), 295 (S to V or P), 331 (E to Q), 365 (Y to F), 395 (A to V), 418 (H to Y), 425 (S to G), 431 (T to 1), 442 (A to T), 454 (M to L), 458 (G to A), 466 (A to S), 485 (G to D), 583 (L to M), 587 (V to E), 595 (K to E), 614 (D to G), 616 (G to R), 631 (L to M), 637 (L to F), 662 (S to F), 664 (K to E), 671 (Y to C), 857 (V to A), 867 (S to R), 919 (F to L), 989 (V to A), 1000 (R to C), 1090 (S to P), 1150 (G to C), 1232 (L to S), 1281 (K to N), 1313 (E to A), 1354 (Q to R), and 1369 (V to I)), as numbered with respect to the nucleotide sequence of 91D2e set forth in SEQ ID NO:9. Some additional variation from these polymorphisms was noted, which is likely due to sequencing or PCR errors, particularly if the polymorphisms were found only once. Twenty coding regions were chosen for further analysis. See Table 14 for descriptions of clones that were isolated. The numbering of the amino acids in Table 14 is based on the amino acid sequence of UGT91D2e set forth in SEQ ID NO:5.

TABLE 14

| Clone | Mutations as compared to UGT91D2e (SEQ ID NO: 5) |
|---|---|
| 1 | +1 frameshift between residues 119-145 in the nucleotide sequence, |
| | G165V, I367V, L388P |
| 2 | 27 bp deletion starting at nucleotide 728, K214R |
| 3 | D205G, V286A, Y443C |
| 4 | L28P, Y30F, P93Q, S99V, E111Q, I118V, Y122F, H140Y, S142C, T144I, |
| | A148T, M152L, G153A, A156S, G162D, L195M, V196E, K199E, L211M, |
| | L213F, S221F, L411S, V425A |
| 5 | G206R, Y207C, W343R |
| 6 | Q13R, F46S, S99P, D395G |
| 7 | Y30F, S364P, G384C, K427N, E438A |
| 8 | Y94C, A132V, Y224C, G384C, K427N, E438A, Q455R |
| 9 | K222E, T341M, G384C |
| 10 | Y94C, A132V, Y224C, K313N, R334C, G384C |
| 11 | Y30F, K222E, V286A, G384C, K427N, E438A |
| 12 | Y30F, P93Q, S99V, Y122F, H140Y, S142C, T144I, T145N, A148T, |
| | M152L, G153A, A156S, G162D, L195M, V196E, K199E, L211M, L213F, |
| | S221F, V286A S289R, R334C, G384C, K427N, E438A |
| 13 | V44A, I136V, G374D, V457I, N463S |
| 14 | I60S, K97R, Q103R, F181S, L411S |
| 15 | V244A, F307L |
| 16 | H140Y, S142C, T144I, A148T, M152L, G153A, A156S |
| 17 | L195M, V196E, K199E, L211M, L213F, S221F, V286A, R334C, G384C, |
| | K427N, E438A |
| 18 | V169A, R334C, G384C, K427N, E438A |
| 19 | G25D, Y30F, P93Q, S99V, Y122F, H140Y, S142C, T144I, A148T, |
| | M152L, G153A, A156S, G162D, L195M, V196E, K199E, L211M, L213F, |
| | S221F, V286A, G384C |
| 20 | I64T, V323A, V330A, G384C, K427N, E438A |

All of the clones in Table 14 were tested for activity using 13-SMG as a substrate. Clone 5 had weak 1,2-glycosylating activity whereas the remaining nineteen did not appear to have activity under the conditions tested. The sequence of clone 5 is set forth in SEQ ID NO:95 and has the following mutations with respect to wild-type UGT92D2e (SEQ ID NO:5): G206R, Y207C, and W343R.

### Example 17—UGT85C Homologs

The genetic diversity of UGT85Cs from six different S. rebaudiana ecotypes was examined to identify homologs that have the same or enhanced activity in pathways for steviol glycoside production. PCR primers were designed that were specific for UGT85C genes, and PCR reactions were carried out on cDNA (some were done on cDNA libraries, some were done on cDNA preparations). The resulting PCR products were cloned and 96 clones were sequenced. Amino acid polymorphisms were mapped and 16 UGT 85C clones were chosen with varying common polymorphism representation. See Table 15. Additional modifications were also noted for some clones, but could be due to PCR errors or were not common polymorphisms. Polymorphisms are described with respect to the nucleotide and amino acid numbering of the wild-type S. rebaudiana UGT85C nucleotide sequence set forth in Accession No. AY345978.1 (see Table 8).

TABLE 15

| | Allele | | | | | | | | | | | | | | | | | |
|---|---|---|---|---|---|---|---|---|---|---|---|---|---|---|---|---|---|---|
| | 38 | 44 | 179 | 194 | 212 | 260 | 659 | 728 | 809 | 866 | 1001 | 1007 | 1116 | 1181 | 1190 | 1253 | 1319 | 1322 |
| | Amino Acid # | | | | | | | | | | | | | | | | | |
| Clone | 13 | 15 | 60 | 65 | 71 | 87 | 220 | 243 | 270 | 289 | 334 | 336 | 389 | 394 | 397 | 418 | 440 | 441 |
| 1 | V-F | F-L | H-D | | | | | | T-M | Q-H | | | | | | E-V | | |
| 2 | V-F | | H-D | | | | | | T-M | | | | | | | | | |
| 3 | | | H-D | | | | | | | | | | | | | E-V | | |
| 4 | | | H-D | | | I-F | | | | | | | | | | | | |
| 6 | | | H-D | | | | | | | | | | | | P-S | | | |
| 7 | | | H-D | | | | | | | | | | | | | | | |
| 8 | | | H-D | | | | | | T-M | | | | | | | | | |
| 13 | | | | A-S | | | | | T-M | | | | A-V | I-V | | | | |
| 16 | | | | A-S | E-Q | | K-T | R-W | T-R | | | | | | | | | |
| 17 | | | | A-S | E-Q | | K-T | R-W | T-R | | | | | | | | | H-N |
| 19 | | | | A-S | E-Q | | K-T | | | | | | A-V | I-V | | | | |
| 20 | | | | A-S | E-Q | | | | T-M | Q-H | | | | | | | | |
| 21 | | F-L | | A-S | | | | | | | | | | | | | | |
| 22 | | | | A-S | | I-F | | | | | | | | | | | G-D | |
| 23 | | | | A-S | | | | | T-A | | | | | | | | | |
| 24 | | | | A-S | | | | | T-M | Q-H | | | | | | | | |
| 26 | | | | A-S | | | | | T-M | | | | | | | | | |
| 27 | | | | A-S | | | | | | | | I-T | | | | | | |
| 28 | | | | A-S | | | | | | | | | | | | | | |
| 29 | | | | A-S | | | | | | | | | | | | | G-D | |
| 30 | | | | A-S | | | | | | | | | | | | | | H-N |
| 31 | | | | A-S | | | | | | | | | | | | E-V | | |
| 32 | | | | A-S | | | | | | | | | | | P-S | | | |
| 33 | | | | | | | K-T | R-W | T-R | | L-S | | | | | | | |
| 36 | | | | | | | | | T-M | | | | | | | | | |
| 37 | | | | | | | | | T-M | Q-H | | | | | | | | |
| 38 | | | | | | | | | T-M | | | I-T | | | | | | |
| 39 | | | H-D | | | | | | T-M | | | | | | | | | |
| 41 | | | | | | | | | | | L-S | | | | | | | |

The clones were expressed through coupled in vitro transcription-translation of PCR products (TNT® T7 Quick for PCR DNA kit, Promega) and assayed for glycosylation activity on the substrates steviol and steviol-19-O-glucoside (0.5 mM), as described in previous examples. The UGT85Cs produced from clones 1, 4, 16, 17, 19, 20, 21, 26, 29, 30, 31, 37, and 39 were soluble and were able to convert 19-SMG to rubusoside in a 90 min assay. The UGT85C produced from clone 27 was considered insoluble. Although UGT85Cs produced from clones 2 and 33 were considered insoluble, trace amounts of rubusoside were produced despite the protein band not being visible. These experiments were independently performed three times. The experiments showed that the following amino acid mutations did not result in a loss of activity: V13F, F15L, H60D, A65S, E71Q, I87F, K220T, R243W, T270M, T270R, Q289H, L334S, A389V, I394V, P397S, E418V, G440D, and H441N. Additional mutations that were seen in active clones include K9E in clone 37, K1 OR in clone 26, Q21H in clone 2, M27V in clone 30, L91P in clone 4, Y298C in clone 31, K350T in clone 37, H368R in clone 1, G420R in clone 19, L431P in clone 4, R444G in clone 16, and M471T in clone 30.

The only common polymorphisms that were not tested were T270A and I336T, which are both fairly conservative substitutions. Clone 17 had the most changes incorporated as compared to UGT85C, 6/480 amino acids. The 17-20 amino acids that appear to be changeable represent approximately a 4% difference at the amino acid level.

Generally, there is low genetic diversity among the 85Cs and it is likely that all of the 85C homologs with the common polymorphisms set forth in Table 15 will be active.

### Example 18—UGT76G Homologs

The genetic diversity of UGT76Gs from six different S. rebaudiana ecotypes was examined to identify homologs that have the same or enhanced activity in pathways for steviol glycoside production. PCR primers were designed that were specific for UGT76G, and PCR reactions were carried out on preparations of cDNA (cDNA libraries or cDNA preparations). The resulting PCR fragments were cloned and 96 clones were sequenced. Common amino acid polymorphisms were mapped and sixteen UGT76G clones chosen, with varying polymorphism representation, including (amino acid numbering): R10S, I16L, F22V, M291, K52S, V74K/E, P80S, L85A, V87S/G, L91P, I92F, I93F, H96Y, G97R, L108V, E113D, G116E, A123T, Q125A, I126L, Y128H, T130A, L142I, V145M, S147N, N151T, F1521, H153L, H155Y, V156D, Q160L, E163D, L167F, P169L, K188N, K191Q, C192S/F, S193G/A, F194Y, M196N, K198Q, K199(I, V, Q), Y200(L, A, G), Y2031, F204L, E205G, N206K, 1207M, T2081, V217I/F, E226Q, S228P, L230V, V233I, I234T, E236D, I237F, S253P, P266Q, S273P, R274S, G284T/A, T285S, 287-3 bp deletion, R298H, P326A, L330V, G331A, P341L, L3461, S376L, D377A, G379A, L380F, S438P, and K441N. Generally, there was very high diversity among the 76Gs.

The clones were expressed through in vitro translation and assayed for glycosylation activity using 0.5 mM steviol-13-O-glucoside and 0.5 mN stevioside as substrates, as described in previous examples. Reactions were carried out for 90 min at 30° C. The native 76G1 activity was found in thee new 76Gs designated 76G_C4, 76G_G7 and 76G_H12, by formation of 1,3-bioside when steviol-13-O-glucoside was used as substrate. Activity in this case was determined comparatively to the positive control, the functional 76G1. Clones 76G_G7 and 76G_H12 produced slightly higher levels of Reb A than the control but 76G_C4 had slightly less Reb A than the control. The number of changes in these clones represents a difference of about 7% at the amino acid level, from the control enzyme. SEQ ID NOs: 98, 100, and 102 set forth the amino acid sequence of 76G_C4, 76G_G7, and 76G_H12, respectively. SEQ ID NOs: 97, 99, and 101 set forth the nucleotide sequences encoding 76G_C4, 76G_G7, and 76G_H12, respectively. SEQ ID NOs: 98, 100, and 102 set forth the amino acid sequence of 76G_C4, 76G_G7, and 76G_H12, respectively. SEQ ID NOs: 97, 99, and 101 set forth the nucleotide sequences encoding 76G_C4, 76G_G7, and 76G_H12, respectively.

Table 16 summarizes the amino acid changes of the 76G clones that had activity, as compared to the wildtype enzyme. There are a large number of overlapping polymorphisms in the active clones, thus it is expected that these polymorphisms do not cause a loss of activity for the enzyme. It appears that certain mutations are frequent in inactive clones, such as the P→S mutation at position 80 or the F→V mutation at position 22.

TABLE 16

| Clone | Mutations |
|---|---|
| 76G_G7 | M29I, V74E, V87G, L91P, G116E, A123T, Q125A, I126L, T130A, |
| | V145M, C192S, S193A, F194Y, M196N, K198Q, K199I, Y200L, Y203I, |
| | F204L, E205G, N206K, I207M, T208I, P266Q, S273P, R274S, G284T, |
| | T285S, 287-3 bp deletion, L330V, G331A, L346I |
| 76G_H12 | M29I, V74E, V87G, L91P, G116E, A123T, Q125A, I126L, T130A, |
| | V145M, C192S, S193A, F194Y, M196N, K198Q, K199I, Y200L, Y203I, |
| | F204L, E205G, N206K, I207M, T208I, P266Q, S273P, R274S, G284T, |
| | T285S, 287-3 bp deletion |
| 76G_C4 | M29I, V74E, V87G, L91P, G116E, A123T, Q125A, I126L, T130A, |
| | V145M, C192S, S193A, F194Y, M196N, K198Q, K199I, Y200L, Y203I, |
| | F204L, E205G, N206K, I207M, T208I |

### Example 19—Expression of Truncated Yeast HMG-CoA Reductase and Other HMG-CoA Reductases

In S. cerevisiae, the mevalonate pathway is heavily regulated, for example, at the level of the enzyme 3-Hydroxy-3-methylglutaryl-coenzyme A (HMG-CoA) reductase. Expressing a truncated HMG-CoA reductase (tHMG1, encoding an enzyme stabilized from degradation) is one method in which flux towards PPP production can be increased in yeast. For example, expression of tHMG1 in yeast has led to dramatic overproduction of β-carotene. See, Verwaal et al., 2007, Appl. Environ. Microbiol. 73:4342. Interestingly, such yeast did not show a darker orange coloration on solid growth medium as was expected, but rather a stronger yellow color, likely due to even higher over-production of the intermediate phytoene.

To determine if expression of HMG-CoA reductase could be used to improve flux to the steviol and steviol glycoside pathways, a yeast reporter strain for testing isoprenoid flux was prepared by substituting the inherent promoter of the ERGS gene with a CUP1 promoter. See, U.S. Patent Application No. 61/346,853, filed May 20, 2010.

The genes used to produce the yeast strain are shown in Table 17. The genes from the source organisms were codon optimized according to DNA 2.0 Inc™. For the purpose of monitoring the cellular prenyl phosphate availability, a construct was produced which had a high copy number plasmid containing gene expression cassettes (methionine-repressible promoters) with the genes for the three enzymes needed to turn prenyl phosphates into β-carotene (GGPP synthase from Xanthophyllomyces dendrorhous, phytoene synthase and beta carotene synthase from X. dendrorhous, and zeta carotene synthase and delta carotene synthase from Neurospora crassa). See, Verwaal et al., 2007 supra; and U.S. Patent Application No. 61/346,853.

TABLE 17
Sources of HMG CoA Reductases and other Mevalonate Genes

| | | | | | SEQ ID | |
|---|---|---|---|---|---|---|
| | | | Size | Gene | (codon | SEQ ID |
| Accession# | Organism | Enzyme | (nt) | name | optimized) | (protein) |
| XM_001467423 | Leishmania infantum | Acetyl-CoA C- | 1323 | MEV-4 | 103 | 104 |
| | | acetyltransferase | | | | |
| YML075C | Saccharomyces cerevisiae | Truncated HMG | 1584 | tHMG1 | 105 | 106 |
| | | (tHMG1) | | | | |
| EU263989 | Ganoderma lucidum | 3-HMG-CoA | 3681 | MEV-11 | 107 | 108 |
| | | reductase | | | | |
| BC153262 | Bos taurus | 3-HMG-CoA | 2667 | MEV-12 | 109 | 110 |
| | | reductase | | | | |
| AAD47596 | Artemisia annua | 3-HMG-CoA | 1704 | MEV-13 | 111 | 112 |
| | | reductase | | | | |
| AAB62280 | Trypanosoma cruzi | 3-HMG-CoA | 1308 | MEV-14 | 113 | 114 |
| | | reductase | | | | |
| CAG41604 | Staph aureus | 3-HMG-CoA | 1281 | MEV-15 | 115 | 116 |
| | | reductase | | | | |
| DNA2.0 | Archaeoglobus fulgidus | 3-HMG-CoA | 1311 | HMG | 117 | 118 |
| sequence | | reductase | | reductase | | |
| DNA2.0 | Pseudomonas mevalonii | 3-HMG-CoA | 1287 | HMG | 119 | 120 |
| sequence | | reductase | | reductase | | |

The yeast tHMG1 was expressed in the CEN.PK-based yeast strain that produces β-carotene, resulting in a color change from orange to light yellow. Interestingly, expression of the full length HMGs from Artemisia annua, Trypanosoma cruzi and Staphylococcus aureus, as well as the NADH-dependent HMG's from Pseudomonas mevalonii and Archeoglobus fulgidus produced a similar result, indicating these genes also improve the flux through the mevalonate pathway in yeast (similar overexpression of Bos taurus HMG had no such effect). Finally, the same color change was seen after over-expression of Leishmania infantum acetyl-CoA C-acetyltransferase (first enzyme of mevalonate pathway, described in Tabc 17) or native S. cerevisiae (CAB1, YDR531W) or B. subtilis, (ace. No. YP004204141) pantothenate kinases (known to result in increased acetyl-CoA production).

To test if the color change in these experiments were indeed due to higher GGPP availability, the yeast tHMG1, P. mevalonii or S. aureus HMGs, or B. subtilis pantothenate kinase were expressed in a stable 19-SMG producer strain. None of these constructs appeared to produce an increase in 19-SMG or rubusoside production (UGT85C2 co-expressed) under the conditions tested. Mevalonate feeding to the yeast reporter strain also did not result in increased rubusoside production. The rubusoside reporter strain, however, has not been genetically modified to reduce the ERG9-encoded flux towards ergosterol biosynthesis. It is expected that control of flux to ergosterol production would result in increased steviol glycoside production using the HMG reductase genes and other mevalonate pathway genes found to be beneficial to beta-carotene production.

### Example 20—Production of RebC In Vivo

The synthesis of a precursor molecule to Rebaudioside C, steviol-13-O-glucopyranosyl-1,2-rhamnoside, was shown in vitro in Example 15. In that example steviol-13-O-monoglucoside was used as a substrate, along with UDP-glucose and the Arabidopsis thaliana RHM2 enzyme (locus tag AT1G53500) and UGT91D2e. To further demonstrate the pathway shown in FIG. 2B, production of Rebaudioside C from steviol was accomplished in vivo.

A yeast strain capable of producing Rebaudioside C was constructed, and production of rebaudioside C and rebaudioside A was assayed by LC-MS. A modified Saccharomyces cerevisiae strain BY4742 was constructed and designated EYS583-7A. The use of BY4742 has been described by Naesby et al., Microb Cell Fact. 8:45 (2009) All four UGTs (91D2d, 76G1, 74G1, and 85C2) were constitutively expressed in plasmids with GPD promoters. This type of strain has been described by Naesby et. al, Microb Cell Fact. 8:45 (2009). UGT85C2 was inserted in plasmid P423 GPD (ATCC #87355), UGT74G1 was cloned into P424 GPD (ATCC #87357) and both UGT91D2e and UGT76G1 were cloned into P425-GPD (ATCC #87359) with 91D2e in the original multiple cloning site (MCS), and 76G1 inserted with an additional GPD promoter and a CYC terminator. The resulting strain was transformed with plasmid P426 GPD (ATCC #87361) containing the RHM2 gene expressed from the GPD promoter. The strain was grown on SC medium lacking histidine, leucine, tryptophan and uracil for 24 hours. The culture was then re-inoculated to an OD₆₀₀ of 0.4 in fresh media containing 25 μM steviol, and the yeast was allowed to grow for 72 more hours before detecting if Rebaudioside C was present in the supernatant and the cell pellets. Rebaudioside C was quantified using an authentic Rebaudioside C standard (Chromadex, Irvine CA). A total of 1.27 μM±0.36 μM of RebC was detected in the supernatant. Similarly, 3.17 μM 1.09 μM RebA was detected in the cell pellet One of skill in the art will recognize that different ratios of RebC to RebA can be obtained by modulation of the activity of the RHM2 enzyme and/or by usage of UGT91D2e or UGT76G1-like enzymes with higher activity for the UDP-rhamnose reactions. The alternative UGTs can be mutagenized versions of the wildtype enzymes or unique enzymes that are obtained through discovery initiatives.

One of skill in the art will recognize that a yeast strain capable of production of Rebaudioside A from glucose, such as strain CEY213 transformed with a plasmid containing UGT91D2e and UGT76G1 in Example 12 would produce Rebaudioside C with the addition of the RHM2 gene either via a vector or integrated into the chromosome.

### Example 21—Production of Steviol Glycosides Using UGTs Expressed in Escherichia coli

Activity of UGT enzymes in gram negative bacteria

The wildtype genes for UGTs 91D2e, 74G1, 76G1, and 85C2 were cloned individually into E. coli XjB-autolysis BL21(DE3) cells using the pET30 vector system from Novagen (EMD4 Biosciences, Madison, WI), except for UGT91D2e, which was cloned into a pGEX 4T-1 (GE Healthcare, Uppsala, Sweden) vector. Similar cloning was described in Examples 7 and 10. All vectors use an IPTG-inducible promoter. Plasmid DNA was transformed into chemically competent cells as described by the vendor.

Transformants displaying the desired antibiotic resistance were grown overnight at 30° C. in 2 nth cultures using NZCYM-media and antibiotic. For in vivo feedings, 5 cultures were grown: UGT 91d2e, 74G1, 76G1, and 85C2 individually, and a mix of all 4 clones. The following day, the cultures were induced to a final concentration of 0.3 mM IPTG and 3 mM arabinose, and grown 2 days at 20° C. in the presence of 50 μM steviol (UGT74G1, UGT85C2 and the quadruple mix) or 50 μM rubusoside (UGT91D2e and UGT76G1). The temperature was raised to 30° C. and the cells were grown for one more day. The cells were then harvested by centrifugation at 4000 rpm for 5 min., and the supernatants were removed for LC-MS analysis. The cells were resuspended in 50% DMSO, lysed at 80° C. for 5 min and the lysates were analyzed by LC-MS.

For in vitro assays, transformants displaying the desired antibiotic resistance were grown overnight at 30° C. in 2 mL cultures using NZCYM-media and antibiotic. The following day, the cultures were induced to a final concentration of 0.3 mM IPTG and 3 mM arabinose, and grown for 24 h at 20° C. The cells were then harvested by centrifugation at 4000 rpm for 5 min and resuspended in 200 μL GT-buffer (RBC Bioscience) and 3 tablets/100 ml of Complete mini, protease inhibitor (Roche), transferred to Eppendorf tubes, vortexed and frozen at −80° C. for 1.5 hour. Cells were thawed on ice, and left at room temperature for 3 minutes. When approximately half-way thawed, 15 μl of 0.14 mg/ml H₂O DNase solution+30 μl 0.05M MgCl₂ was added to each tube and the samples were incubated for approximately 5 minutes at room temperature. The cells were centrifuged at maximum speed for 5 minutes. One-hundred μL of supernatant (lysate) was transferred to fresh microfuge tubes, and 100 μL of glycerol was added.

Enzyme assays were performed by adding 15.15 μL H₂O, 7.5 μL 4× Buffer (400 mM Tris, 20 mM MgCl2, 4 mM KCl), 0.3 μL FastAP™ (1 u/μL) from Fermentas, 0.45 μL of a 100 mM stock of UDP-glucose, 0.6 μL of substrate (steviol or rubusoside) and 6 μL of the crude enzyme preparations described above. UGT74G1, UGT85C2, as well as all four UGTs mixed were incubated with steviol. UGT 76G1 and 85C2 were incubated with rubusoside. The enzyme assays were incubated overnight at 37° C. Following centrifugation at 4000 rpm for 5 minutes, 30 μL samples were transferred to a fresh 96 well plate and 30 μL of DMSO was added. The samples were then subjected to LC-MS analysis. Similar in vitro experiments were also done using steviol 1,2-bioside (for UGT76G1 and UGT74G1) or Rebaudioside B (for UGT74G1) as substrates.

No activity was detected in the in vivo feedings. Table 18 illustrates the results for the in vitro assays.

TABLE 18

| Tube | UGT Clone(s) | Substrate fed | Product detected |
|---|---|---|---|
| 1 | 74G1 | Steviol | 19-SMG, low levels of rubusoside |
| 2 | 85G1 | Steviol | 13-SMG, low levels of rubusoside |
| 3 | 76G1 | Rubusoside | 1,3-stevioside, an unknown tetra- |
| | | | glycoside |
| 4 | 91D2c | Rubusoside | stevioside |
| 5 | Mix of 4 crude UGT | Steviol | Rubusoside, 1,3-stevioside, trace RebA |
| | preparations | | (no mono sides) |
| 6 | 76G1 | Steviol 1,2-bioside | Rebaudioside B |
| 7 | 74G1 | Steviol 1,2-bioside | Stevioside |
| 8 | 74G1 | Rebaudioside B | Rebaudioside A |

These results indicate that the UGT enzymes are all active in E. coli cells. However, the substrates may not be readily imported into the cytoplasm. It is expected that if the steviol were produced in E. coli from precursor pathways, the production of the various steviol glycoside products would be feasible from glucose. It is unexpected that the 74G1 and 85G1 UGTs, which have slightly overlapping substrate specificities, can produce rubusoside from steviol singly. The mix of the four crude enzyme preparations gave very low levels of the monosides, which indicates that the conversion to di- and tri-glycosides was efficient. With respect to UGT91D2e, the preparation that was used had lost some of its original activity after long-term storage. It is expected that a fresh preparation of the enzyme would have yielded higher levels of Rebaudioside A.

### Example 22—Production of Steviol Glycosides in Physcomitrella patens

### Feeding Experiments in Moss Cells

The genes for UGT 91d2e, 74G1, 76G1, and 85C2 were cloned into Physcomitrella patens using the pTHUbi:Gateway vector system described in U.S. Patent Publication No. 20100297722. This vector uses a strong maize Ubiquitin promoter. PCR primers were designed to amplify the coding regions in previous examples (native sequences) with the addition of “CACC” upstream of the start codon. Plasmid DNA was digested with SwaI and used for transformation into protoplasts (generally around 0.5×10⁶ protoplasts). Transformants displaying the desired resistance were grown 1 day in 10 mL cultures and then fed either steviol, rubusoside, or buffer+DMSO as indicated by Table 19. One-half mL of buffer containing substrate was added per 10 mL of culture, and final concentrations of 0.1% DMSO, 50 μM steviol or rubusoside, and 0.125 mM phosphate buffer were added to the cultures. A positive control was done where the YFP (yellow fluorescent protein) was expressed in the presence of steviol or just buffer and DMSO. Cultures were grown 2 more days prior to separation of cells and freezing in liquid nitrogen until further analysis. In some cases multiple UGT-containing plasmids were transformed into the same protoplast cells, to illustrate conversion of multiple steps within the moss cells.

TABLE 19

| Tube | UGT Gene(s) | Substrate fed |
|---|---|---|
| 1 | YFP (control) | none |
| 2 | YFP | Steviol (50 μM) |
| 3 | 74G1 | none |
| 4 | 76G1 | none |
| 5 | 85C2 | none |
| 6 | 91D2E | none |
| 7 | 74G1 | Steviol (50 μM) |
| 8 | 76G1 | Steviol (50 μM) |
| 9 | 85C2 | Steviol (50 μM) |
| 10 | 91D2E | Steviol (50 μM) |
| 11 | 74G1/85C2 | none |
| 12 | 74G1/85C2 | Steviol (50 μM) |
| 13 | 74G1/85C2/91D2E | none |
| 14 | 74G1/85C2/91D2E | Steviol (50 μM) |
| 15 | 76G1 | Rubusoside (50 μM) |
| 16 | 91D2E | Rubusoside (50 μM) |
| 17 | 76G1/91D2E | none |
| 18 | 76G1/91D2E | Rubusoside (50 μM) |

Expression was positive in the controls (tubes 1 and 2) as measured by fluorescent signal observation. The supernatants from the experiments were analyzed by LC-MS; 200 μL of each supernatant sample was mixed with an equal volume of 50 percent DMSO. The samples were spun (15,700 relative centrifugal force, 10 minutes) and 100 microliters of the resulting supernatant was analyzed by LC-MS.

Protoplast pellets were thawed on ice and 10 mM Tris-HCl pH 8 containing 3 tablets/100 ml of Complete Mini Protease Inhibitor (Roche) was added to reach a final volume of 150 μL. The solutions were divided in two: 75 μL was transferred to a new tube and protoplasts were pelleted (15,700 relative centrifugal force, 1 minute). Pellets were washed with 75 tit Milli-Q water before resuspension in 150 μL DMSO (50 percent). Samples were then heated (80 degrees Celsius, 10 minutes), vortexed and centrifuged (15,700 relative centrifugal force, 10 minutes). Fifty μL of the resulting supernatant was analyzed by LC-MS.

No steviol glycoside production was detectable in supernatants or pellets. It is unknown if the steviol and rubusoside can be transported into moss cells.

### In Vitro Feeding of Pellet Extracts

In vitro feeding experiments were conducted with samples 1, 3, 4, 5, 6, 11, 13 and 17). Glass beads (425-600 microns) were added to the remaining 75 μL of the original resuspensions and protoplasts were mechanically lysed by vortexing 3 times, 2 minutes each time, at 4 degrees Celsius and storage on ice in between vortexing. The samples were spun (15,700 relative centrifugal force, 10 minutes, 4 degrees Celsius) and 6 μL of resulting supernatants was used in in vitro enzyme reactions. For the enzyme reactions FastAP™ phosphatase (Fermentas) was used (0.3 U/reaction) and the UDP-glucose:substrate ratio was 5. The samples were fed either steviol or rubusoside according to Table 20.

TABLE 20

| Cell extract from tube | UGT Gene(s) | Substrate fed |
|---|---|---|
| 1 | YFP | None |
| 1 | YFP | 0.5 mM steviol |
| 1 | YFP | 0.5 mM rubusoside |
| 3 | 74G1 | 0.5 mM steviol |
| 4 | 76G1 | 0.5 mM rubusoside |
| 5 | 85C2 | 0.5 mM steviol |
| 6 | 91D2E | 0.5 mM rubusoside |
| 11 | 74G1/85C2 | 0.5 mM steviol |
| 13 | 74G1/85C2/91D2E | 0.5 mM steviol |
| 17 | 76G1/91D2E | 0.5 mM rubusoside |

Reactions were incubated at 30° C. overnight. After incubation, an equal amount of DMSO (100 percent) was added to the samples and mixed, then the sample was spun (15,700 relative centrifugal force, 10 minutes) and 30 μL of the resulting supernatant was analyzed by LC-MS.

LC-MS analysis showed conversion of rubusoside to 1,3-stevioside by UGT76G1. None of the other steviol glycosides were detectable. It is unknown if soluble expression of the UGTs occurred in Physcomitrella. It is expected if one UGT is active in the moss cells, the others would also be active if expression occurred. In addition, the cloning was done in a transient manner. Stable integration of the genes is expected to produce additional clones that are active for UGT activity when tested.

Methods are known to those with skill in the art for increasing soluble expression of recombinant proteins. Alternative promoters, ribosome binding sites, codon usage, co-expression with chaperones, and change in temperature are non-limiting examples of methods for increasing soluble expression of recombinant proteins.

### Example 23—Production of Steviol Glycosides in Aspergillus nidulans

### Activity of UGT Enzymes in Fungal Cells

The native genes for UGT 91D2e, 74G1, 76G1, and 85C2 were cloned into Aspergillus nidulans using a PCR-fabricated expression cassette and the USER vector system. Cloning methods are described in Hansen et al., Appl. Environ. Microbiol. 77: 3044-3051 (2011). Briefly, a nucleotide sequence encoding each UGT was inserted between the constitutive PgpdA promoter and the TtrpC terminator, in a vector containing additionally two targeting sequences for genomic integration and argB as selection marker. Plasmid DNA was transformed into A. nidulans protoplasts according to Nielsen et al., Fungal Genet. Biol. 43:54-64 (2006) and Johnstone et al., EMBO J. 4:1307-1311 (1985). Transformants displaying the desired resistance were grown for 48 hours in 150 ml, cultures using minimal media (1% Glucose; 10 mM NaNO₃; mineral mix).

Cell lysates prepared by disruption of the mycelia with glass beads were used to determine the activities of the individual UGTs in in vitro. The cell lysates of strains expressing 74G1 and 85C2 were incubated with 0.5 mM steviol and the strains expressing 76G1 and 91D2c were incubated with 0.5 mM steviol-13-O-glucoside for 24 hours, and the supernatants further analyzed using LC/MS. No steviol glycosides were detected.

It is unknown whether soluble expression of the UGT enzymes was achieved as these products are not typically visible on SDS-PAGE. Since Aspergillus and Saccharomyces are both fungi, it is expected that additional experimentation would result in active clones. Methods are known to those with skill in the art for increasing soluble expression of recombinant proteins. Alternative promoters, inducer levels, ribosome binding sites, codon usage, co-expression with chaperones, and change in temperature are non-limiting examples of methods for increasing soluble expression of recombinant proteins.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for producing Rebaudioside D (RebD), Rebaudioside E (RebE), or a mixture thereof, comprising contacting a precursor steviol glycoside having a 13-O-glucose, a 19-O-glucose, or both the 13-O-glucose and the 19-O-glucose with a polypeptide capable of beta 1,2 glycosylation of the C-2′ of the 13-O-glucose, 19-O-glucose, or both the 13-O-glucose and the 19-O-glucose of the precursor steviol glycoside and a UDP-glucose in a reaction mixture under suitable conditions for the transfer of one or more glucose moiety to the C2′ of the 13-O-glucose, 19-O-glucose or both 13-O-glucose and 19-O-glucose in the precursor steviol glycoside; thereby producing RebD, RebE, or a mixture thereof;
wherein the polypeptide comprises an amino acid motif AA1-AA2-AA3-AA4-AA5-AA6-AA7, corresponding to residues 20-26 in SEQ ID NO:5; and
wherein:
AA₁ is Proline
AA2 is one aromatic amino acid;
AA3 is one large hydrophobic amino acid;
AA4 is one small amino acid;
AA5 is one amino acid;
AA6 is one small amino acid; and
AA7 is Histidine.

2. The method of claim 1, wherein AA2 is:
(a) one amino acid having a van der Waals volume≥130 Å³ and a side chain hydrophobicity≥60 Δt_{R};
(b) one amino acid having a van der Waals volume≥130 Å³ and a side chain hydrophobicity≥80 Δt_{R};
(c) Tryptophan, Phenylalanine, or Tyrosine;
(d) Tryptophan or Phenylalanine; or
(e) Tryptophan.

3. The method of claim 1, wherein AA3 is:
(a) one amino acid having a van der Waals volume≥100 Å³ and a side chain hydrophobicity≥60 Δt_{R}
(b) one amino acid having a van der Waals volume of 100 to 160 Å³ and a side chain hydrophobicity≥60 Δt_{R};
(c) one amino acid having a van der Waals volume of 100 to 140 Å³ and a side chain hydrophobicity≥60 Δt_{R};
(d) Leucine, Valine, Isoleucine, Methionine, Phenylalanine, Tyrosine, or Tryptophan;
(e) Leucine, Valine, Isoleucine, Methionine, Phenylalanine, or Tyrosine;
(f) Leucine, Valine, Isoleucine, Methionine, or Phenylalanine; or
(g) Leucine or Phenylalanine.

4. The method of claim 1, wherein AA4 is:
(a) one amino acid having a van der Waals volume≤70 Å³ or
(b) Alanine or Glycine.

5. The method of claim 1, wherein AA6 is:
(a) one amino acid having a van der Waals volume≤80 Å³;
(b) Alanine, Glycine, or Serine; or
(c) Glycine or Serine.

6. The method of claim 1, comprising further contacting the reaction mixture with:
(a) a polypeptide capable of glycosylating a precursor steviol glycoside having a C-13 hydroxyl group present in the reaction mixture at its C-13 hydroxyl group; and/or
(b) a polypeptide capable of glycosylating a precursor steviol glycoside having a C-19 carboxyl group present in the reaction mixture at its C-19 carboxyl group; and/or
(c) a polypeptide capable of beta 1,3 glycosylation of the C3′ of the 13-O-glucose, of the 19-O-glucose or both the 13-O-glucose and the 19-O-glucose of the precursor steviol glycoside having a 13-O-glucose, a 19-O-glucose, or both the 13-O-glucose and the 19-O-glucose present in the reaction mixture.

7. The method of claim 1, which is an in vitro method comprising supplying the UDP-glucose or a cell-free system for regeneration of the UDP-glucose.

8. The method of claim 6, which is an in vitro method comprising supplying one or more UDP-sugars or supplying a cell-free system for regeneration of the one or more UDP-sugars, and wherein the conversion of the precursor steviol glycoside into RebD, RebE, or a mixture thereof requires multiple reactions that can be carried out together or stepwise.

9. The method of claim 1, wherein RebD is produced from Rebaudioside A (RebA) upon transfer of the glucose moiety from the UDP-glucose to the 19-O-glucose of RebA.

10. The method of claim 9, wherein RebA is provided as a plant extract.

11. The method of claim 1, wherein RebE is produced from stevioside upon transfer of the glucose moiety from the UDP-glucose to the 19-O-glucose of stevioside.

12. The method of claim 7, wherein phosphatases are added to the reaction mixture.

13. The method of claim 6, wherein RebD is produced from stevioside, and stevioside is contacted with the polypeptide capable of transferring the glucose moiety to the C-2′ of the 19-O-glucose in the precursor steviol glycoside and with the polypeptide capable of beta 1,3 glycosylation of the C3′ of the 13-O-glucose in the precursor steviol glycoside.

14. The method of claim 1, wherein the polypeptide capable of transferring the glucose moiety to the C-2′ of the 13-O-glucose, 19-O-glucose or both 13-O-glucose and 19-O-glucose in the precursor steviol glycoside is expressed by a recombinant microorganism comprising a gene coding for the polypeptide.

15. The method of claim 6, wherein one or more of the polypeptides are expressed by a recombinant microorganism comprising one or more genes coding for the one or more polypeptides.

16. The method of claim 14, wherein the microorganism belongs to the species Saccharomyces cerevisiae, Escherichia coli, Yarrowia lipolytica, or Pichia pastoris.

17. The method of claim 1, wherein the polypeptide has a higher activity for beta 1,2 glycosylation of the C-2′ of the 19-O-glucose of the precursor steviol glycoside as compared to the beta 1,2 glycosylation of the C-2′ of the 13-O-glucose of the precursor steviol glycoside.

18. A method of producing Rebaudioside D (RebD), Rebaudioside E (RebE), or a composition thereof, comprising growing a recombinant microorganism comprising a recombinant gene encoding a polypeptide capable of beta 1,2 glycosylation of the C-2′ of the 13-O-glucose, 19-O-glucose, or both the 13-O-glucose and the 19-O-glucose of the precursor steviol glycoside having a 13-O-glucose, a 19-O-glucose, or both the 13-O-glucose and the 19-O-glucose, in a culture medium comprising a precursor steviol glycoside under conditions in which the gene encoding the polypeptide is expressed and RebD, RebE, or a composition thereof is produced
wherein the polypeptide comprises an amino acid motif AA1-AA2-AA3-AA4-AA5-AA6-AA7, corresponding to residues 20-26 in SEQ ID NO:5; and
wherein:
AA₁ is proline
AA₂ is one aromatic amino acid;
AA₃ is one large hydrophobic amino acid;
AA₄ is one small amino acid;
AA₅ is one amino acid;
AA₆ is one small amino acid; and
AA₇ is histidine.

19. The method of claim 18, wherein RebA is the precursor steviol glycoside and RebD or a composition thereof is produced.

20. The method of claim 18, wherein the microorganism further comprises a gene encoding a polypeptide capable of beta 1,3 glycosylation of the C3′ of the 13-O-glucose, of the 19-O-glucose or both the 13-O-glucose and the 19-O-glucose of the precursor steviol glycoside having a 13-O-glucose, a 19-O-glucose, or both the 13-O-glucose and the 19-O-glucose, wherein the microorganism is grown under conditions wherein the genes coding for the polypeptides are expressed, wherein rubusoside or 1,2-stevioside is the precursor steviol glycoside and RebD is produced.

21. The method of claim 20, wherein the microorganism further comprises a gene encoding a polypeptide capable of glycosylating a precursor steviol glycoside having a C-19 carboxyl group, wherein the microorganism is grown under conditions wherein the genes coding for the polypeptides are expressed, wherein steviol-13-O-glucoside (13-SMG) is the precursor steviol glycoside and RebD is produced.

22. The method of claim 20, wherein the microorganism further comprises a gene encoding a polypeptide capable of glycosylating a precursor steviol glycoside at its C-13-hydroxyl group, wherein the microorganism is grown under conditions wherein the genes coding for the polypeptides are expressed, wherein steviol-19-O-glucoside (19-SMG) is the precursor steviol glycoside and RebD is produced.

23. The method of claim 18, wherein the polypeptide has a higher activity for beta 1,2 glycosylation of the C-2′ of the 19-O-glucose of the precursor steviol glycoside as compared to the beta 1,2 glycosylation of the C-2′ of the 13-O-glucose of the precursor steviol glycoside.

24. The method of claim 18, wherein the microorganism belongs to the species Saccharomyces cerevisiae, Escherichia coli, Yarrowia lipolytica, or Pichia pastoris.
